(19) United States
(12) Patent Application Publication
(10) Pub. No.: US 2021/0063410 A1
(43) Pub. Date: Mar. 4, 2021

(54) AUTOMATED SAMPLE WORKFLOW GATING AND DATA ANALYSIS

(71) Applicant: DISCERNDX, INC., Palo Alto, CA (US)

(72) Inventors: Bruce WILCOX, Palo Alto, CA (US); Lisa CRONER, Palo Alto, CA (US); John BLUME, Palo Alto, CA (US); Ryan BENZ, Palo Alto, CA (US); Jeffrey JONES, Palo Alto, CA (US); Scott SCHRECKENGAUST, Palo Alto, CA (US); William SMITH, Palo Alto, CA (US); Athit KAO, Palo Alto, CA (US); Jia YOU, Palo Alto, CA (US)

(21) Appl. No.: 16/644,099

(22) PCT Filed: Sep. 5, 2018
(86) PCT No.: PCT/US2018/049574

§ 371 Date: Mar. 3, 2020

Related U.S. Application Data
(60) Provisional application No. 62/554,437, filed on Sep. 5, 2017.
(60) Provisional application No. 62/554,441, filed on Sep. 5, 2017.
(60) Provisional application No. 62/554,444, filed on Sep. 5, 2017.
(60) Provisional application No. 62/554,445, filed on Sep. 5, 2017.
(60) Provisional application No. 62/554,446, filed on Sep. 5, 2017.
(60) Provisional application No. 62/559,335, filed on Sep. 15, 2017.
(60) Provisional application No. 62/559,309, filed on Sep. 15, 2017.
(60) Provisional application No. 62/560,066, filed on Sep. 18, 2017.
(60) Provisional application No. 62/560,068, filed on Sep. 18, 2017.
(60) Provisional application No. 62/560,071, filed on Sep. 18, 2017.
(60) Provisional application No. 62/568,192, filed on Oct. 4, 2017.
(60) Provisional application No. 62/568,194, filed on Oct. 4, 2017.
(60) Provisional application No. 62/568,241, filed on Oct. 4, 2017.
(60) Provisional application No. 62/568,197, filed on Oct. 4, 2017.

(51) Int. Cl. G01N 33/68; G06T 7/00; G06F 9/30

(52) U.S. Cl. CPC G01N 33/6848; G01N 33/6842; G06T 2207/20024; G06T 7/0012; G06F 9/3005; G01N 33/6818

(57) ABSTRACT

A number of methods and computer systems related to mass spectrometric data analysis are disclosed. Adoption of the disclosure herein facilitates automated, high throughput, rapid analysis of complex datasets such as datasets generated through mass spectrometric analysis, so as to reduce or eliminate the need for oversight in the analysis process while rapidly yielding accurate results. In some cases, identification of a health condition indicator is carried out based on information relating a predetermined association between an input parameter and a health condition indicator.

### CROSS-REFERENCE

This application claims the benefit of U.S. Prov. App. Ser. No. 62/554,437, filed Sep. 5, 2017, which is hereby explicitly incorporated herein by reference in its entirety; this application claims the benefit of U.S. Prov. App. Ser. No. 62/554,441 filed Sep. 5, 2017, which is hereby explicitly incorporated herein by reference in its entirety; this application claims the benefit of U.S. Prov. App. Ser. No. 62/554,444, filed Sep. 5, 2017, which is hereby explicitly incorporated herein by reference in its entirety; this application claims the benefit of U.S. Prov. App. Ser. No. 62/554,445, filed Sep. 5, 2017, which is hereby explicitly incorporated herein by reference in its entirety; this application claims the benefit of U.S. Prov. App. Ser. No. 62/554,446, filed Sep. 5, 2017, which is hereby explicitly incorporated herein by reference in its entirety; this application claims the benefit of U.S. Prov. App. Ser. No. 62/559,309, filed Sep. 15, 2017, which is hereby explicitly incorporated herein by reference in its entirety; this application claims the benefit of U.S. Prov. App. Ser. No. 62/559,335, filed Sep. 15, 2017, which is hereby explicitly incorporated herein by reference in its entirety; this application claims the benefit of U.S. Prov. App. Ser. No. 62/560,066, filed Sep. 18, 2017, which is hereby explicitly incorporated herein by reference in its entirety; this application claims the benefit of U.S. Prov. App. Ser. No. 62/560,068, filed Sep. 18, 2017, which is hereby explicitly incorporated herein by reference in its entirety; this application claims the benefit of U.S. Prov. App. Ser. No. 62/560,071, filed Sep. 18, 2017, which is hereby explicitly incorporated herein by reference in its entirety; this application claims the benefit of U.S. Prov. App. Ser. No. 62/568,192, filed Oct. 4, 2017, which is hereby explicitly incorporated herein by reference in its entirety; this application claims the benefit of U.S. Prov. App. Ser. No. 62/568,194, filed Oct. 4, 2017, which is hereby explicitly incorporated herein by reference in its entirety; this application claims the benefit of U.S. Prov. App. Ser. No. 62/568,241, filed Oct. 4, 2017, which is hereby explicitly incorporated herein by reference in its entirety; this application claims the benefit of U.S. Prov. App. Ser. No. 62/568,197, filed Oct. 4, 2017, which is hereby explicitly incorporated herein by reference in its entirety.

### BACKGROUND

Mass spectrometric analysis shows promise as a diagnostic tool; however, challenges remain relating to the development of high throughput data analysis workflows.

### SUMMARY OF THE INVENTION

Provided herein are methods and systems that rely upon or benefit from intermingling of laboratory processes and computational processes in single workflows for sample analysis, such as sample analysis related to automated mass spectroscopy. Practice of some methods and systems disclosed herein facilitates or allows non-technical operators to produce accurate, precise, automated, repeatable mass spectrometric results. In some cases, the workflow encompasses a sequence of computational data processing steps such as data acquisition, workflow determination, data extraction, feature extraction, proteomic processing, and quality analysis. Marker candidates are generated manually or through automated art searches, and are assessed through analysis of sample data concurrently or previously generated. Various aspects of the disclosure herein benefit in part from reliance upon automated gating of sequential steps in a mass spectrometric workflow such that a sample is assessed repeatedly throughout the workflow progression. Samples or machine operations that fail a gated quality assessment result in the sample run being variously terminated, tagged as deficient, or paused so as to allow sample clearance, instrument recalibration or correction, or otherwise to address the low quality control outcome. Thus, gated sample output datasets are assembled and compared as having a common level of statistical confidence.

Provided herein are noninvasive methods of assessing a biomarker indicative of a health status in an individual, for example using a blood sample of an individual. Some such methods comprise the steps of obtaining a circulating blood sample from the individual; obtaining a biomarker panel level for a biomarker panel using an automated or partially automated system, and using said panel information to make a health assessment. Also provided herein are methods and systems related to automated mass spectroscopy. Practice of some methods and systems disclosed herein facilitates or allows non-technical operators to produce accurate, precise, automated, repeatable mass spectrometric results. These benefits are conveyed in part through reliance upon automated gating of sequential steps in a mass spectrometric workflow such that a sample is assessed repeatedly throughout the workflow progression. Samples or machine operations that fail a gated quality assessment result in the sample run being variously repeated, terminated, tagged as deficient, or paused so as to allow sample clearance, instrument recalibration or correction, or otherwise to address the low quality control outcome.

Provided herein are methods and systems related to identification of one or more of a biomarker or portion thereof, biological pathway and health condition status, and use in patient health classification. Some methods and systems herein facilitate identifying interrelationships among disorders, pathways, proteins, genes, available information from art references and from previously or concurrently run experiments, and available markers such as polypeptide markers, metabolite markers, lipid markers or other biomolecules assayable in a sample through approaches disclosed herein. Mass spectrometry data analyzed according to these methods and systems can be obtained using the mass spectrometric workflows described herein. In some cases, the biomarker or biological pathway and/or health condition status is evaluated using data analysis carried out according to the computational workflows described herein, which optionally work in combination or alongside wet lab workflows.

Provided herein are systems for automated mass spectrometric analysis, comprising a plurality of protein or other biomolecule processing modules positioned in series; and a plurality of mass spectrometric sample analysis modules; wherein each of said protein processing modules is separated by a mass spectrometric sample analysis module; and wherein each mass spectrometric sample analysis module operates without ongoing supervision.

Provided herein are systems for automated mass spectrometric analysis comprising: a plurality of workflow planning modules positioned in series; a plurality of protein or other biomolecule processing modules positioned in series; and a plurality of mass spectrometric sample analysis modules; wherein each of said protein or other biomolecule processing modules is separated by a mass spectrometric sample analysis module; and at least one of said modules is separated by a gating module; wherein the output data of at least one module has passed a gating module evaluation prior to becoming input data for a subsequent module.

Provided herein are computer-implemented methods for automated mass spectrometric workflow planning, comprising: a) receiving operating instructions, wherein the operating instructions comprise a study question; b) generating a plurality of candidate biomarker proteins or other biomarker molecules by searching at least one database; and c) designing a mass spectrometric study workflow using the candidate biomarker proteins or other biomarkers; wherein the method does not require supervision.

Provided herein are methods for automated mass spectrometric analysis comprising a) defining a transition pool; b) optimizing a mass spectrometric method, wherein optimizing comprises a maximizing signal to noise, reducing method time, minimizing solvent usage, minimizing coefficient of variation, or any combination thereof; c) selecting final transitions; and d) analyzing a mass spectrometric experiment using the final transitions and the optimized mass spectrometric method; wherein at least one of the steps are further separated by a gating step, wherein the gating step evaluates the outcome of a step before performing the next step.

Provided herein are computer-implemented methods for automated mass spectrometric analysis, comprising: a) receiving operating instructions, wherein the operating instructions comprise variables informative of at least 50 biomarker protein or other biomolecule peak quality assignments; b) automatically translating the variables into a machine-learning algorithm; and c) automatically assigning peak quality assignments of subsequent samples using the machine-learning algorithm.

Provided herein are methods for automated mass spectrometric analysis, comprising: a) acquiring at least one mass spectrometric data set from at least two different sample runs; b) generating a visual representation of the data comprising identified features from the at least two sample runs; c) defining an area of the visual representation comprising at least a portion of the identified features; and d) discontinuing analysis because a threshold of at least one QC metric is not met based on a comparison between features of the sample runs; wherein the method is performed on a computer system without user supervision. In some cases, the threshold of at least one QC metric is not met when no more than 10 non-corresponding features between the sample runs is identified. The identified features can comprise charge state, chromatographic time, overall peak shape, analyte signal strength, presence of known contaminants, or any combination thereof.

Provided herein are systems for feature processing, comprising: a) a plurality of visualization modules positioned in series; and b) a plurality of feature processing modules positioned in series; wherein at least one of the feature processing modules is separated by a gating module; wherein the output data of at least some feature processing modules has passed a gating module evaluation prior to becoming input data for a subsequent feature processing module; wherein the output data of at least some visualization modules has passed a gating evaluation prior to becoming input data for a subsequent visualization module, and wherein at least some gating evaluation occurs without user supervision.

Provided herein are systems for proteome visualization, comprising: a) a proteomics data set obtained from any of the preceding embodiments; and b) a human interface device capable of visualizing the proteomics data set.

Provided herein are systems for marker candidate identification, comprising: a) an input module configured to receive a condition term; b) a search module configured to identify text reciting the condition term and to identify marker candidate text in proximity to the condition term; and c) an experimental design module configured to identify a reagent suitable for detection of the marker candidate.

Provided herein are systems for automated mass spectrometric analysis, comprising a plurality of workflow planning modules positioned in series; a plurality of protein processing modules positioned in series; and a plurality of mass spectrometric sample analysis modules; wherein each of said protein processing modules is separated by a mass spectrometric sample analysis module; and wherein each mass spectrometric sample analysis module operates without ongoing supervision.

Provided herein are methods of mass spectrometric sample analysis, comprising subjecting a mass spectrometric sample to a series of manipulations pursuant to workflow planning; wherein at least some of said manipulations pursuant workflow planning are gated by automated evaluation of an outcome of a prior step.

Provided herein are methods of mass spectrometric sample analysis, comprising subjecting a mass spectrometric sample to a series of manipulations pursuant to mass spectrometric analysis; wherein at least some of said manipulations pursuant to mass spectrometric analysis are gated by automated evaluation of an outcome of a prior step.

Provided herein are systems for automated mass spectrometric analysis, comprising a plurality of protein processing modules positioned in series; and a plurality of mass spectrometric sample analysis modules; wherein at least some of said protein processing modules are separated by a mass spectrometric sample analysis module; and wherein at least some mass spectrometric sample analysis modules operate without ongoing supervision.

Provided herein are methods of mass spectrometric sample analysis, comprising subjecting a mass spectrometric sample to a series of manipulations pursuant to mass spectrometric analysis; wherein at least some of said manipulations pursuant to mass spectrometric analysis are gated by automated evaluation of an outcome of a prior step.

Provided herein are systems comprising a) a marker candidate generation module configured to receive a condition input, to search a literature database to identify references reciting the condition, to identify marker candidates recited in the references, and to assemble the marker candidates into a marker candidate panel; and 2) a data analysis module, configured to assess a correlation between the condition and the marker candidate panel in at least one gated mass spectrometric dataset.

Provided herein are systems for automated mass spectrometric analysis, comprising a plurality of protein processing modules positioned in series; and a plurality of mass spectrometric sample analysis modules; wherein each of said protein processing modules is separated by a mass spectrometric sample analysis module; and wherein each mass spectrometric sample analysis module operates without ongoing supervision.

Provided herein are methods of mass spectrometric sample analysis, comprising subjecting a mass spectrometric sample to a series of manipulations pursuant to mass spectrometric analysis, wherein at least some of said manipulations pursuant to mass spectrometric analysis are gated by automated evaluation of an outcome of a prior step.

Provided herein are systems for automated mass spectrometric analysis of a data set, comprising: a) a plurality of mass spectrometric data processing modules; and b) a workflow determination module generating a computational workflow comprising the plurality of data processing modules positioned in series to analyze the data set, wherein the computational workflow is configured based on at least one of a worklist and at least one quality assessment performed during mass spectrometric sample processing.

Provided herein are systems for automated mass spectrometric analysis of a data set, comprising: a) a plurality of mass spectrometric data processing modules; and b) a workflow determination module extracting a mass spectrometric method and parameters from a worklist associated with the data set and using the mass spectrometric method and parameters to generate a computational workflow comprising the plurality of data processing modules positioned in series to analyze the data set.

Provided herein are systems for automated mass spectrometric analysis of a data set, comprising: a) a plurality of mass spectrometric data processing modules; and b) a workflow determination module generating a computational workflow comprising the plurality of data processing modules positioned in series to analyze the data set, wherein at least one of the plurality of data processing modules in the workflow is selected based on quality assessment information obtained during mass spectrometric sample processing.

Provided herein are systems for automated mass spectrometric analysis of a data set obtained from a sample, comprising: a) a plurality of mass spectrometric data processing modules; and b) a workflow determination module generating a computational workflow comprising the plurality of data processing modules positioned in series to perform data analysis of the data set, wherein the data analysis is informed by at least one automated quality assessment carried out during sample processing.

Provided herein are systems for automated mass spectrometric analysis of a data set obtained from a sample, comprising: a) a plurality of mass spectrometric data processing modules; and b) a workflow determination module generating a computational workflow comprising the plurality of data processing modules positioned in series to perform data analysis of the data set, wherein the data analysis is informed by at least one quality control metric generated by at least one quality assessment carried out during sample processing.

Provided herein are systems for automated mass spectrometric analysis of a data set, comprising: a) a plurality of mass spectrometric data processing modules for carrying out a computational workflow analyzing the data set; and b) a quality control module performing a quality assessment for a data analysis output of at least one of the plurality of data processing modules, wherein the output failing the gated quality assessment results in at least one of the computational workflow being paused, the output being flagged as deficient, and the output being discarded.

Provided herein are systems for automated mass spectrometric analysis of a data set comprising a plurality of mass spectrometric data processing modules; a workflow determination module parsing a worklist associated with the data set to extract parameters for a workflow for downstream data analysis of the data set by the plurality of data processing modules; and a quality control module assessing at least one quality control metric for some of the plurality of data processing modules and tagging the output when the output fails the at least one quality control metric, wherein the tagging informs downstream data analysis.

Provided herein are systems for automated mass spectrometric analysis, comprising a plurality of mass spectrometric data processing modules for processing mass spectrometric data; wherein each mass spectrometric data processing module operates without ongoing supervision.

Provided herein are computer-implemented methods for carrying out the steps according to any of the preceding systems.

Provided herein are methods for automated mass spectrometric analysis of a data set, comprising: a) providing a plurality of mass spectrometric data processing modules; and b) providing a workflow determination module generating a computational workflow comprising the plurality of data processing modules positioned in series to analyze the data set, wherein the computational workflow is configured based on at least one of a worklist and at least one quality assessment performed during mass spectrometric sample processing.

Provided herein are methods for automated mass spectrometric analysis of a data set, comprising: a) providing a plurality of mass spectrometric data processing modules; and b) providing a workflow determination module extracting a mass spectrometric method and parameters from a worklist associated with the data set and using the mass spectrometric method and parameters to generate a computational workflow comprising the plurality of data processing modules positioned in series to analyze the data set.

Provided herein are methods for automated mass spectrometric analysis of a data set, comprising: a) providing a plurality of mass spectrometric data processing modules; and b) providing a workflow determination module generating a computational workflow comprising the plurality of data processing modules positioned in series to analyze the data set, wherein at least one of the plurality of data processing modules in the workflow is selected based on quality assessment information obtained during mass spectrometric sample processing.

Provided herein are methods for automated mass spectrometric analysis of a data set obtained from a sample, comprising: a) providing a plurality of mass spectrometric data processing modules; and b) providing a workflow determination module generating a computational workflow comprising the plurality of data processing modules positioned in series to perform data analysis of the data set, wherein the data analysis is informed by at least one automated quality assessment carried out during sample processing.

Provided herein are method for automated mass spectrometric analysis of a data set obtained from a sample, comprising: a) providing a plurality of mass spectrometric data processing modules; and b) providing a workflow determination module generating a computational workflow comprising the plurality of data processing modules positioned in series to perform data analysis of the data set, wherein the data analysis is informed by at least one quality control metric generated by at least one quality assessment carried out during sample processing.

Provided herein are methods for automated mass spectrometric analysis of a data set, comprising: a) providing a plurality of mass spectrometric data processing modules for carrying out a computational workflow analyzing the data set; and b) providing a quality control module performing a quality assessment for a data analysis output of at least one of the plurality of data processing modules, wherein the output failing the gated quality assessment results in at least one of the computational workflow being paused, the output being flagged as deficient, and the output being discarded.

Provided herein are methods for automated mass spectrometric analysis of a data set, comprising: providing a plurality of mass spectrometric data processing modules; providing a workflow determination module parsing a worklist associated with the data set to extract parameters for a workflow for downstream data analysis of the data set by the plurality of data processing modules; and providing a quality control module assessing at least one quality control metric for some of the plurality of data processing modules and tagging the output when the output fails the at least one quality control metric, wherein the tagging informs downstream data analysis.

Provided herein are methods for automated mass spectrometric analysis, comprising providing a plurality of mass spectrometric data processing modules for processing mass spectrometric data; wherein each mass spectrometric data processing module operates without ongoing supervision.

Provided herein are health condition indicator identification processes, comprising: receiving an input parameter; accessing a dataset in response to receiving the input, the dataset comprising information relating to at least one predetermined association between the input parameter and at least one health condition indicator; and generating an output comprising a health condition indicator having a predetermined association with the input parameter.

Provided herein are tangible storage medium comprising instructions configured to: receive an input parameter; access a dataset in response to receiving the input, the dataset comprising information relating to at least one predetermined association between the input parameter and at least one health condition indicator; and generate an output comprising a health condition indicator having a predetermined association with the input parameter.

Provided herein are health condition indicator identification processes, comprising: receiving an input parameter; transmitting the input parameter to a server; receiving an output generated in response to the input parameter, the output comprising a health condition indicator comprising a predetermined association with the input parameter; and displaying the output to a user.

Provided herein are display monitors configured to present biological data, said display monitor presenting at least two disorder nodes, at least one gene node, at least one protein node, at least one pathway node, and markings indicating relationships among at least some of said nodes.

Throughout the disclosure of the present specification, reference is made to proteins or to polypeptides. It is understood that polypeptides refers to molecules having a plurality of peptide bonds, and encompasses fragments up to and including full length proteins. It is also understood that the methods, markers, compositions, systems and devices disclosed and referred to herein are often compatible with analysis of not only polypeptides but also a number of biomolecules consistent with the detection approaches herein, such as lipids, metabolites and other sample molecules.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Some understanding of the features and advantages of the present invention will be obtained by reference to the following detailed description that sets forth illustrative embodiments, in which the principles of the invention are utilized, and the accompanying drawings of which:

FIG. 1 shows an embodiment of a planning workflow for profile proteomics studies.

FIG. 2 shows an embodiment of a planning workflow for DPS proteomics studies.

FIG. 3 shows an embodiment of a planning workflow for targeted proteomics and iMRM studies.

FIG. 4 shows an embodiment of a study analysis workflow for profile proteomics studies.

FIG. 5 shows an embodiment of a study analysis workflow for DPS proteomics studies.

FIG. 6 shows an embodiment of a study analysis workflow for targeted proteomics and iMRM studies.

FIG. 7 shows an embodiment of a low-resolution pipeline-generated starfield image.

FIG. 8 shows an embodiment of a high-resolution starfield image.

FIG. 9 shows an embodiment of a high-resolution 3-D starfield images being visually assessed using a 3-D viewing platform.

FIG. 10 shows an embodiment of a visualization to assess and filter standard curves from multiple injections based on measures of spike-in standards (SIS).

FIG. 11 shows an embodiment of an interactive high-resolution starfield image on a touchable computer system.

FIG. 12 shows embodiment of a starfield thumbnail images across samples grouped and filtered by sample annotation using an Om—The API Data Exploration Center computer program.

FIG. 13 shows an embodiment of a visual exploration of longitudinal data with a feature explorer computer program.

FIG. 14 shows an embodiment of a visual exploration of comparative data with a proteomic barcode browser computer program.

FIG. 15 shows an embodiment of a visual exploration of longitudinal data with a personal proteomics data computer browser program.

FIG. 16 shows an embodiment of a visual exploration of longitudinal data with a personal proteomics data sphere computer program.

FIG. 17 shows an embodiment of a mass spectrometric workflow for fractionated proteomics studies.

FIG. 18 shows an embodiment of a mass spectrometric workflow for depleted proteomics studies.

FIG. 19 shows an embodiment of a mass spectrometric workflow for dried blood spot proteomics studies with optional SIS spike-in.

FIG. 20 shows an embodiment of a mass spectrometric workflow for targeted, depleted proteomics studies.

FIG. 21 shows an embodiment of a mass spectrometric workflow.

FIG. 22 shows an embodiment of a mass spectrometric workflow for iMRM proteomics studies.

FIG. 23 shows an embodiment of a mass spectrometric workflow for dilute proteomics studies.

FIG. 24 illustrates an exemplary series of standard curves.

FIG. 25 illustrates an exemplary series of quality control metrics.

FIG. 26 illustrates an exemplary trace from a depletion and fractionation experiment.

FIG. 27A illustrates an exemplary computational workflow for data analysis in accordance with an embodiment.

FIG. 27B illustrates an exemplary computational workflow for data analysis in accordance with an embodiment.

FIG. 28 shows an embodiment of a software application for carrying out the computational workflow described herein.

FIG. 29 is a process flow diagram of an example of a health condition indicator identification process.

FIG. 30 is a process flow diagram of another example of a health condition indicator identification process.

FIG. 31 is a schematic diagram of an example of a network layout comprising a health condition indicator identification system.

FIG. 32 is a schematic diagram of an example of a user interface for implementing a health condition indicator identification process.

FIG. 33 is a schematic diagram of an example of a computer system that is programmed or otherwise configured to perform at least a portion of the health condition indicator identification process as described herein.

FIG. 34A is a depiction of a display indicating interrelatedness among disorders (pink), genes (green), pathways (blue), proteins (blue), peptide markers (purple) and peptide collections stored in common or available from a common source (grey).

FIG. 34B shows a close-up of the display from FIG. 34A.

FIG. 34C shows a close-up of the display from FIG. 34A.

FIG. 34D shows a simplified representative diagram corresponding to a display such as seen in FIG. 34A that can be generated according to the systems and methods disclosed herein.

### DETAILED DESCRIPTION

Disclosed herein are methods, systems, automated processes and workflows for experimental design of and execution of mass spectrometric analysis of samples such as biological samples comprising biomolecules such as proteins, metabolites, lipids or other molecules conducive to mass spectrometric or comparable detection and analysis. Through practice of the disclosure herein, one variously identifies candidate markers and performs mass spectrometric analysis on as sample or assesses previously generated data of sufficient quality, for example so as to assess the utility of these markers as a diagnostic panel for a disorder, condition, or status. Practice of some part of the disclosure herein achieves automated candidate panel generation, such that a user may enter a disorder, condition or status, and an automated search of that entry identifies associated terms in the relevant literature, such as proteins likely to be present in a certain tissue to assay such as plasma, serum, whole blood, spit, urine or other easily assessed sample sources as suitable candidate constituents. Practice of some part of the disclosure herein achieves partially or completely automated mass spectrometric analysis such that a mass spectrometric analysis run or collection of runs performed pursuant to, for example, diagnosis or biomarker development, is accomplished without reliance upon an operator having particular expertise in the performance of particular steps in a mass spectrometric analysis workflow. In some cases, the automated and partially automated systems and methods are useful in obtaining data for a panel of biomarkers, such as proteins, polypeptides derived from proteins, metabolites, lipids or other biomolecules informative of a condition or status and measurable using approaches consistent with the disclosure herein. Such methods, devices, compositions, kits, and systems are used to determine a likelihood that a subject has a health condition or status. The assays are generally noninvasive or minimally invasive and can be determined using a variety of samples, including blood and tissue.

Automation is accomplished so as to span multiple steps in marker panel development or mass spectrometry analysis. Variously, steps comprising marker candidate selection through a survey of relevant literature or otherwise, mass spectrometric sample analysis, and data analysis are partially or totally automated such that no operator supervision is required from identification of a disease to be studied through to assessment of mass spectrometric data, such that a disease is inputted by a user, and a validated output panel is provided without user supervision of automated intermediate steps. Alternately, automated steps are interrupted by steps having user interaction or user oversight, but such that automated steps constitute a substantial part of at least one of marker candidates identified through a survey of the art, mass spectrometric analysis comprising sample manipulation modules separated by gating evaluation modules, and sample data output and analysis.

The systems can be automated, for example by connecting at least some of the individual modules to one another such that the samples produced or manipulated by a module are automatically fed to a subsequent module in a particular work flow. This is done through any number of automated approaches, such as using sample handling robots or by connecting the fluidics between modules. As another example, the system can be automated by connecting at least one of the individual sample handling modules to a module comprising a detector that evaluates the quality of the output of the previous step in a particular work flow and flags or gates a sample based on the results of that analysis.

Thus, practice of some methods, systems, automated processes and workflows for mass spectrometric analysis consistent with the disclosure herein facilitates the broad application of mass spectrometric analysis of samples, such as biological samples comprising proteins or protein fragments metabolites, lipids or other biomolecules measurable using approaches consistent with the disclosure herein, to address biological problems. Automation in various embodiments of the disclosure herein facilitates rapid marker candidate identification, mass spectrometric analysis to generate quality-gated data for a given sample analysis run such that the outcome of that run is comparable in statistical confidence to samples run at different times or even to address different biological questions, and analysis of gated-sample analysis outcomes, so as to identify panel constituents related to a particular disease or condition that are reliably assayed through mass spectrometry or through antibody based or other assay approach.

The disclosure herein substantially facilitates the application of mass spectrometric analysis approaches to biological problems of diagnosis and disease marker panel development. The incorporation of an automated search for candidate panel constituents allows one to replace or supplement manual searches of the literature. Alternately, manual search results are used as a starting point for partially or fully automated, gated analysis of samples, for example to validate or assess the utility of candidate marker panels.

The systems and methods described herein can provide several advantages. First, the systems and methods can ensure that instrumentation is working correctly and alert operators to problems related to the processing or analysis of samples prior to those samples moving on in the workflow. For example, the incorporation of automated gating between physical manipulation steps allows one to identify defective steps in certain runs, such that samples or sample runs not meeting a threshold, surpassing a threshold, cumulatively indicating a defect in a workflow, or otherwise exhibiting an property casting doubt on a final mass spectrometric outcome, are identified. Identified samples or sample analysis runs are variously flagged as failing a manipulation assessment, discarded, subjected to a pause or cessation of an analysis workflow, or otherwise addressed such that sample integrity or workflow constituent operation may be assessed or addressed prior to continuing an analysis workflow. Thus, assessing samples at a variety of checkpoints throughout the workflow to determine the quality of the sample after specific processing steps can also ensure that samples are produced, processed, and measured consistently as to, for example, their polypeptides, metabolites, lipids or other biomolecules measurable using approaches consistent with the disclosure herein. Consistency can help to reduce problems with detection and quantification of analytes of interest, which can often be affected by interferences or suppression.

The incorporation of automated gating between physical manipulation steps allows unflagged, completed mass spectrometric analysis to be confidently assessed as being clear of technical defects in generation without ongoing user assessment of either the output or the intermediate steps involved in the process. Accordingly, mass spectrometric analyses outputs through the present disclosure are assessed by experts in a given field of research much like, for example, nucleic acid sequence information or other biological information for which automated data generation is routinely generated by or under the direction of researchers having expertise in a field of research rather than in the technical details of mass spectrometric sample processing and data analysis.

Furthermore, unflagged or otherwise statistically confident results are in many cases statistically comparable, such that results of separate sample analysis runs are readily combined in later data analyses. That is, a first set of sample run data that is unflagged or otherwise statistically acceptable upon being subjected to gating assessment at various stages of its generation is readily combined with a second set of sample run data that is comparably unflagged but arising from a separate original experiment. Unflagged samples can, therefore, be more easily compared to other samples analyzed during the same or different experiment or run. As an example, data from one patient sample may be more easily compared to data from a different patient analyzed the same day, on a different day, or on a different machine. Likewise, data from a patient sample collected or analyzed at one time point can be more easily compared to data from the same patient collected or analyzed at a different time point including, for example, when monitoring the progression or treatment of a disease or condition.

Methods, systems, automated processes and workflows such as those disclosed herein for analysis such as mass spectrometric analysis of samples, for example biological samples comprising proteins, metabolites, lipids or other biomolecules measurable using approaches consistent with the disclosure herein, are in some cases characterized by a particular disease or condition for which informative information such as diagnostic markers are sought. Diagnostic markers are often selected from candidate pools, such as candidate pools derived from published art related to a condition or disease. Candidate pools are identified manually, through surveys of art related to a disease or condition of interest. Alternately or in combination, candidate pools are identified through an automated process whereby, for example, a condition or disease-related term is searched in relevant art databases, and text reciting a particular search terms are surveyed automatically for recitation of proteins or other biomarkers that may be included in a candidate pool. Thus, candidate pools are generated either through manual inspection of relevant art, or through an automated survey of art reciting particular terms and from which related terms relevant to a candidate pool are extracted, or through a combination of automated and manual approaches.

Methods, systems, automated processes and workflows such as those disclosed herein for analysis such as mass spectrometric analysis of samples, for example biological samples comprising proteins, metabolites, lipids or other biomolecules measurable using approaches consistent with the disclosure herein, are characterized by a series of physical manipulations of a sample such as a biological sample. Samples are collected, subjected to a series of step such as quality assessment and physical manipulation, and are assessed so as to obtain mass spectrometric information. Data generated from samples subjected to mass spectrometric analysis are evaluated using a computational workflow that is optionally tailored to the type of mass spectrometric analysis such as Profile/DPS or Targeted/MRM mass spectrometry. At various steps in the process, samples or sample manipulation processes are subjected to quality assessment, such as automated quality assessment, and sample progression through mass spectrometric analysis is ‘gated’ such that unflagged progression through a workflow is conditioned on quality assessment outcome. Samples or sample manipulation steps failing an automated assessment variously results in flagging the sample, for example so as to indicate in its output that there is an issue in the analysis, or may result in pausing or cancelling the analysis workflow so as to address the workflow or sample issue, for example by cleaning or recalibrating an apparatus, by supplementing a sample, repeating a step in the work flow, or by discarding the sample from the workflow. Alternately flagged samples are subjected to a completed run, but their resultant data is subjected to a revised data analysis, such as one reflective of deficiencies in the workflow. Such a revised workflow may, for example, provide lower significance to the absence of a marker in light of a gating outcome indicative of decreased sensitivity in at least one gating assessment module of a sample analysis workflow. In some cases, data flagged by a gating step effects subsequent sample analysis. For example a sample failing a gating step is flagged, and subsequent samples are normalized, which allows for later comparison of data sets. Alternatively or in combination, flagged data is presented in a final analysis that allows the researcher to assess the validity or accuracy of the collected data in forming conclusions. In some aspects the presence of flagged data informs future experiments and future workflow planning.

In some cases, a computational process or pipeline for analyzing/processing the samples is restarted or rebooted upon failure of an automated assessment. As an example, the failure to populate a data file due to file mislabeling or data corruption can result in the computational workflow being paused or terminated without expending further resources attempting to perform downstream data processing or analysis. In the case when a portion of the data set is evaluated to be unreliable (e.g., has a low quality control metric such as high SNR), that portion is optionally flagged to identify the deficiency, which can inform downstream or future analysis (e.g., the portion of the data set is excluded from further analysis). Alternatively or in combination, the computational workflow is informed by upstream quality assessments performed during sample processing such as modifying or altering the data analysis (such as altering the sequence of computational workflow modules used to perform the analysis) based on the results of the quality assessments. In this way, the data output or data analysis can be gated to remove some or all of the output from downstream analysis and/or terminate the computational workflow such as when the quality assessment indicates a failure at one or more data processing steps. Thus, the computational workflow disclosed herein is capable of being integrated into an overall mass spectrometric workflow that variously incorporated one or more of marker candidate identification through a survey of the art (e.g., experimental design and setup), mass spectrometric analysis comprising sample manipulation modules separated by gating evaluation modules (e.g., wet lab steps), and sample data output and analysis (e.g., computational workflow for data analysis), which steps are partially or fully automated.

In various embodiment of the disclosure herein, one, two, more than two, three, four or more up to and including all buy three, all but two, all but one, or all steps of an analysis workflow are gated by an assessment step such as an automated assessment step. Some workflows consist exclusively of automated workflow assessment steps, such that no sample assessment by a user is required to generate a mass spectrometric output of a known, predetermined or previously set level of quality. Alternately, some workflows comprise automated workflow assessment steps and also comprise workflow assessment steps involving or requiring user oversight or assessment. In some such cases, user assessment is limited to initial, final, or initial and final steps, such that intermediate steps do not involve sample or apparatus assessment by a user. Alternately, user oversight may be present at various steps of the mass spectrometric analysis, separated by automated gating steps not requiring user oversight. Consistent with the specification, a workflow comprises in some instances some steps that are automated. For example, a workflow comprises 1, 2, 3, 4, 5, 6, 7, 8, 9, 10, 15, 20, 50, 75, or more than 75 steps that are automated. In some cases, a workflow comprises at least 1, 2, 3, 4, 5, 6, 7, 8, 9, 10, 15, 20, 50, or at least 75 steps that are automated. In other various aspects, at least 10%, 20%, 30%, 40%, 50%, 60%, 70%, 80%, 90%, or 100% of the steps in a workflow are automated. In other cases, about 10%, 20%, 30%, 40%, 50%, 60%, 70%, 80%, 90%, or 100% of the steps in a workflow are automated. In some instances, no more than 10%, 20%, 30%, 40%, 50%, 60%, 70%, 80%, 90%, or 100% of the steps in a workflow are automated. In some cases, some of the steps are automated or gated. In various aspects, “some” is more than one, such as at least two.

Similarly, some workflows consist exclusively of manipulation steps in series, each gated by a quality assessment step, either automated or otherwise. In some cases, all manipulation steps are gated by automated quality assessment steps. Alternately, some workflows consistent with the disclosure herein comprise both gated and ungated manipulation steps, at least some of said gated manipulation steps, or all of said gating steps, being gated by automated quality assessment steps, or in some cases all of said of said gated manipulation steps being gated by automated quality assessment steps.

Some workflows are generated through an automated candidate marker or panel pool identification process, such that a disorder, disease condition or status is entered and subjected to an automated marker assessment protocol, and candidate markers are automatically identified prior to sample analysis or prior sample gated data reanalysis.

Candidate pools are assessed using either an untargeted or a targeted analysis, or a combination of the two. Through an untargeted analysis, gated mass spectrometric sample analysis is performed, and peaks corresponding to markers of interest are assessed for condition or disease or other status-dependent variations that suggest utility of the marker alone or in a panel indicative of a disease, condition or status in an individual. Through a targeted analysis, a sample is supplemented by addition of reagents such as mass-shifted peptides, for example so as to facilitate identify native peptides corresponding to the mass shifted peptides in a mass spectrometric output. Heavy isotope, chemically modified, homologue or otherwise mass-shifted polypeptides or other biomarkers are suitable to facilitate identification of native polypeptide presence or quantitative level in a sample.

Practice of the disclosure allows generation of data of a known, consistent level of quality from a number of disparate sources. When output quality is consistently assessed throughout, such as through the automated gating approaches of the methods, systems, and workflows herein, variations in sample source, collection protocols, storage or extraction are readily identified, and sample runs for which defects in collection or processing are identified through gating such as automated, and flagged or otherwise treated herein so as not to be confused with data, independent of source, that satisfies all quality assessments. Sample assessment runs satisfying all data assessments are therefore readily analyzed by a researcher as having comparable quality levels, such that biologically relevant variations among sample runs of samples from various sources (such as healthy vs disease positive sources) may be identified without being confused for or obstructed by variations in data quality resulting from progression of unassessed samples through an analysis workflow unflagged or uncorrected.

Accordingly, so long as gating assessment such as automated gated assessment indicates that sample quality satisfied a threshold or is otherwise satisfactory, disparate sample sources may be relied upon for mass spectrometric data that are nonetheless mutually comparable. Accordingly, a number of sample collection sources and samples are consistent with the methods, systems, workflows and apparatuses of the present disclosure. Samples are for example collected directly from a tissue such as a tumor tissue, for comparison to samples from elsewhere in the same tumor, from the same tumor at different times, elsewhere in the tissue distinct from the tumor, other tissue of the same individual, circulating samples from the same individual, healthy and/or tumor tissue collected from a second individual collected concurrently or at a different time and subjected to the same or to different collection or storage treatments, or otherwise differing from one another.

Similarly, sample runs from different times or different sources, or originally targeting different conditions, disorders, or statuses are nonetheless combinable in follow-on ‘in silico’ or semi in silico analyses to identify relevant markers or marker panels. That is, an automated survey of available data identifies in some cases a data set that is informative of a condition, for example because individuals varying at that condition or disease or status is present in the sample. When the pre-existing data is insufficient to provide a desired level of sensitivity, specificity or other measure of statistical confidence, the data is supplemented by sample analysis performed to address the question at hand. The newly run sample, provided that it satisfies gating assessments performed during processing, is readily combined to previous gated datasets so as to add statistical confidence to a particular analysis related to a particular disease, condition or status, even when some or all of the data is generated for a different disease, condition or status.

A number of sample collection methods are consistent with the disclosure herein. Provided that sample processing survives gating at a sufficient level of quality, data from multiple experiments are readily combined, even when they arise from different sample types. In some exemplary cases, samples are collected from patient blood by depositing blood onto a solid matrix such as is done by spotting blood onto a paper or other solid backing, such that the blood spot dries and its biomarker contents are preserved. The sample can be transported, such as by direct mailing or shipping, or can be or stored without refrigeration. Alternately, samples are obtained by conventional blood draws, saliva collection, urine sample collection, by collection of exhaled breath, or from other source suitable for analysis. Through practice of the disclosure herein, such samples are readily analyzed in isolation or compared to samples collected directly from a tissue source to be studied, even when the collection and storage protocols differ.

Methods, systems, automated processes and workflows such as those disclosed herein for analysis such as mass spectrometric analysis of samples, for example biological samples comprising proteins, are often configured to integrate quality control samples for concurrent or successive analysis. In some cases, the analysis enables identification of candidate marker pools, assessment of candidate marker pools. Some quality control samples are constructed to be informative as to performance of at least one sample manipulation step, multiple steps or up to an including in some cases an entire workflow. Some quality control samples comprise molecules to facilitate identification of candidate markers in a sample, such as by including mass-shifted versions of polypeptides of interest to or representative of candidate pool markers. A quality control sample variously comprises a bulk sampling of known sample pools at known or expected concentrations, such that outcome of a manipulation occurring during at least one step in a workflow is analyzed. Manipulation outcome is then gated by sample output measurement, by quality control sample output measurement, by a combination of sample output measurement and quality control sample output measurement, or otherwise, such as by comparison to a standard or to a predetermined value.

Accordingly, gating through automated manipulation assessment is accomplished through a number of approaches consistent with the disclosure herein. Manipulation module outputs are variously compared to a set or predetermined threshold, or are compared to an internal quality control standard, or both. Gating is done in isolation or in light of additional factors, such as amount of a reagent from a prior step. Thus, in some cases, presence of a particular yield following a manipulation step is sufficient to satisfy a gating step. Alternately, independent of or in addition to an absolute value assessment, a sample run manipulation step is gated through assessing relative yield from one step to another, such that a decline in yield from one step to another will flag a sample or a manipulation step as deficient, even if (due to the initial sample level being particularly high) the yield of that step remains above an absolute level sufficient for gating. Gating in some cases comprises assessing the repeatability of measurements made on aliquots of a particular sample following or prior to a particular manipulation, for example as an assessment of sample homogeneity, so as to assess whether the sample is likely to yield repeatable results in downstream analysis. Gating in some cases comprises assessing equipment accuracy, repeatability, or preparedness prior to contacting to sample.

Sample gating, particularly early in a workflow but also throughout, optionally comprises assessment of yield-independent sample metrics such as metrics indicative of likely sample output or performance. Examples of such metrics include evidence for hyperlipidemia, large amounts of hemoglobin in a sample, or other sample constituents indicative of likely problematic analysis.

Accordingly, gating variously comprises a number of sample or manipulation module assessment approaches consistent with the disclosure herein. A common aspect of many gating steps is that they are positioned prior to, subsequent to or between manipulation modules, so as to assess individual modules rather than, or in addition to, the workflow as a whole, and that many gating steps are automated so as to be performable without oversight by a user.

Practice of the disclosure allows generation of data of a known, consistent level of quality from a number of disparate sample analysis platforms. Much like sample collection, above, sample analysis platforms may substantially impact outcome. In situations where sample manipulation modules of a given sample analysis platform are not gated by assessment module such as automated analysis modules, variations in data output arising from sample analysis platform variation are often not readily distinguished from biologically relevant differences between or among samples, such as differences that serve as basis for a diagnosis or development of a diagnosis panel.

Through the automated gated analysis of mass proteomic samples such as samples arising from disparate sources and subjected to disparate sample processing platforms, one readily identifies systemic or structural variations through automated gated assessment. Accordingly, systemic defects arising through sample collection variation, sample variation, processing platform variation, or otherwise, are in some cases addressed through modifications in a workflow, such as through selecting an alternative device, reagent set or module workflow to perform a workflow step resulting in an ungated outcome. Identification of a manipulation module as leading to gate-blocked output facilitates replacing or altering that manipulation module, or at least one upstream manipulation module, so as to increase the frequency of ungated or threshold-satisfying data being generated through that manipulation step or at least one manipulation step upstream therefrom.

Alternatively or in combination, manipulation steps that demonstrate a comparable performance across sample input types but that differ in reagent cost, time, durability, or any other relevant parameter are identified, such that one may select the manipulation step device, reagents or protocol having the preferred parameter, such as cost, processing time, or other parameter. That is, automated gating of manipulation step facilitates both assessment of sample output quality for comparison to other sample outputs generated for example under uncontrolled conditions, and assessment of sample manipulation modules, such that particular modules are identified as being underperforming for a particular sample or as being otherwise undesirable for a given protocol, such as too expensive, too slow, faster or more expensive than necessary in light of other steps or otherwise suboptimal for a workflow, method or system as disclosed herein.

In some cases, automated gating of at least some steps in an output facilitates identifying samples or sample sources for which analysis is unsuitable, for example because a given workflow is unlikely to produce unflagged, readily comparable data that is clear of systemic biases in data output. Such samples or sample sources identified as unsuitable is often flagged or otherwise marked to enable the computational workflow to discard a portion of the data set or the entire data set based on what data is marked as unsuitable.

Automated gating and/or quality assessment of at least some manipulation or data processing steps facilitates reliable, rapid execution of mass spectrometric analysis of a sample such as a biological protein sample. In part because there are not the delays associated with user evaluation of intermediate manipulation or data processing or analysis steps when evaluation of those steps is automated, automated gating reduces delays in mass spectrometric analysis and increases throughput. Moreover, termination of data analysis for a given data file or data set (or a portion thereof) allows the computational workflow to proceed to the next data file or data set, thus enabling efficient use of computational resources. Accordingly, practice of the methods, use of the systems, or employment of workflows as disclosed herein results in mass spectrometric analysis being completed in no more than 95%, 90%, 85%, 80%, 75%, 70%, 65%, 60%, 55%, 50%, 45%, 40%, 35%, 30%, 25%, 20%, 15%, 10%, or less than 10% of the time taken to execute a workflow for which automated gating and/or quality assessment is replaced by user evaluation. Similarly, practice of the methods, use of the systems, or employment of workflows as disclosed herein results in mass spectrometric analysis being completed in no more than 3 days, 2 days, 1 day, 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, 3 hours, 2 hours, 1 hour, 50 minutes, 40 minutes, 30 minutes, 20 minutes, or 10 minutes for mass spectrometric data sets having at least 1000 features, 2000 features, 3000 features, 4000 features, 5000 features, 10000 features, 20000 features, 30000 features, 40000 features, 50000 features, 100000 features, 200000 features, 300000 features, 400000 features, or at least 500000 features or more.

Automated gating and/or quality assessment of at least some manipulation or data processing steps facilitates comparison of results obtained through mass spectrometric workflows comprising differing manipulation steps or through analysis of different sample sources or treatments, or both differing manipulation steps and analysis of different sample sources or treatments. For example, data sets obtained from different experimental procedures may be gated, filtered, or normalized to obtain a subset of each data set that is suitable for analysis together. Accordingly, a researcher using the disclosure herein is able to perform mass spectrometric analysis on samples collected through differing protocols, or on mass spectrometric workflows using differing manipulation step instruments, and nonetheless compare some resulting data with confidence.

Furthermore, in addition to facilitating comparison, automated gating of at least some manipulation steps facilitates generation of results that are in some cases combinable so as to increase the statistical confidence of the conclusions made from either result set individually. That is, subjecting workflows to uniform gating such as automated gating at various steps throughout a sample manipulation workflow generates data which, if surviving the gating assessments, is confidently assigned to be of a uniform quality so as to be added to at least one later or earlier generated results set without normalization factors specific to any particular sample such as sample source specific or sample processing workflow specific normalization factors.

Also disclosed herein are databases comprising workflow-gated mass spectrometric results, such that said individual result sets of said databases are readily compared and combined to one another so as to yield searchable, analyzable database results. Such databases are used alone or in combination with automated or manual marker candidate generation and optionally with subsequent sample analysis to generate a separable or continuous, partially or totally automated workflow for condition, disease or status evaluation to form systems of mass spectrometric data analysis. A condition, disease, status or other term is entered into a search module, which identifies by automated word association term corresponding to potential marker candidates, such as proteins that appear in proximity to the search terms in academic texts such as PubMed or other academic, medical or patent art or other databases. Marker candidates are identified for further analysis. The condition, disease, status or other term is searched against inputs for comparable, gated sets in a database stored in a database module so as to identify sets having sample inputs that vary for the condition, disease, status or other term. Levels of marker candidates are assessed in the datasets, in some cases as if the sets are combined into a single run, and results are subjected to downstream analysis. When downstream analysis results in validation of marker candidates from the gated sets previously generated, one is able to obtain a marker set for a condition, disease, status or other term through automated assessment of previously generated, gated data without performing additional sample manipulation.

Alternately, when previously generated gated datasets do not yield a desired level of confidence or do not include a marker candidate, one may generate at least one additional dataset using samples obtained as of relevance to the condition, disease, status or other term. Samples are subjected to gated analysis, such as automated gated analysis, so as to generate gate-cleared data that is readily combined to previously generated data. Thus, additional sample analysis is in some cases generated only as needed to supplement previously existing gated data, rather than to provide sufficient statistical confidence as a stand-alone dataset. Alternately, de novo sample analysis is performed so as to generate marker candidate validation information for a condition, disease, status or other term. Gated information thus generated is readily added to a database so as to be available for further automated assessment.

### Study Planning

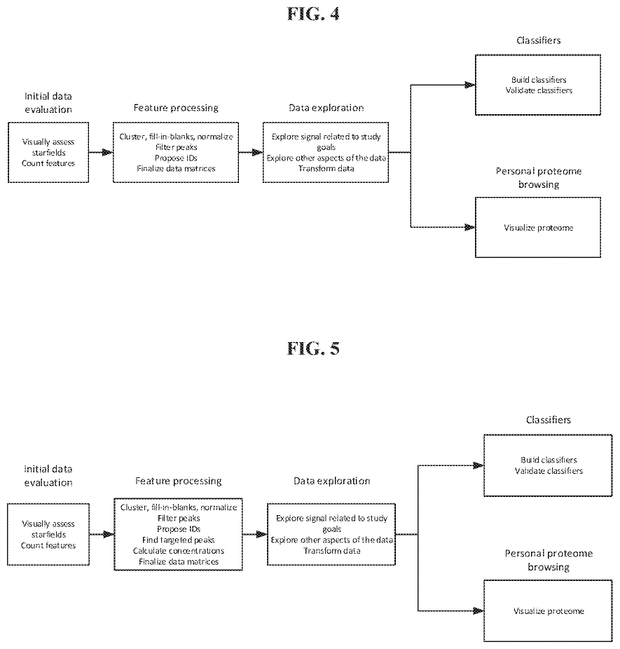

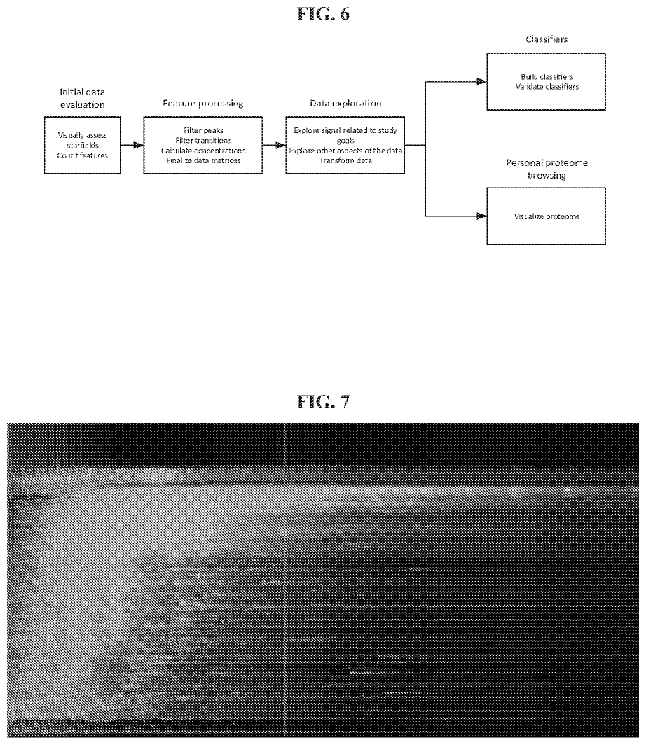

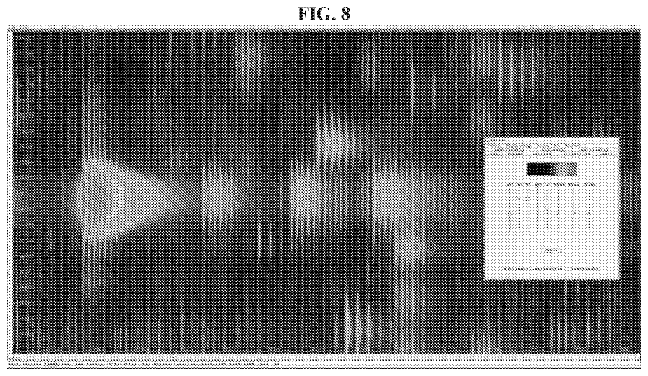

Disclosed herein are methods, systems, automated processes and workflows for the planning of experiments and studies. The experiments and studies often are mass spectrometric and proteomics studies. Proteomics studies include DPS, targeted, iMRM (immunoaffinity coupled with multiple reaction monitoring), a protein quantification assay such as SISCAPA or other antibody based or antibody-independent protein quantification assay or a number of other types and designs of proteomics studies. In some cases, this involves multiple steps or modules for planning and/or executing a study. A gating analysis is present in between at least some of the modules. For example a study plan comprises modules of defining a question, designing a study, and obtaining samples. A study design often comprises a series of considerations, parameters, or operations to be considered prior to obtaining samples. In some cases, this involves considering additional factors relevant to the statistical analysis of data. For example, this often involves (by non-limiting example) analyzing the presence or absence of compounding factors, the structure of experimental groups, and alternately or in combination involves performing one or more analyses, such as power analyses, or any other analysis of additional factors consistent with the specification. After designing the study, the next step often is obtaining samples for analysis. Considerations, parameters, or operations involving sample acquisition are important for reducing potential problems prior to executing a full study. Alternately or in combination this involves identifying sample sources, evaluating and planning data collection, evaluating early samples, or other processes or operations relevant to sample collection. After one or more planning steps are executed, in some cases samples are randomized. Workflow plans also in some cases include developing a mass spectrometric method. An exemplary study plan workflow is illustrated in FIG. 4. Different workflow plans comprises one or more steps consistent with the specification are also used to plan proteomics experiments. For example, a DPS proteomics study comprises steps of initiating the study, identifying protein marker candidates, designing the study, obtaining samples, and randomizing samples (FIG. 2). An iMRM study further comprises a step of developing an MS method prior to randomizing samples (FIG. 3). A workflow plan may omit or include additional steps depending on the specific application of the workflow. Optionally, workflow plans are generated automatically using a set of initial input parameters.

Planning workflows in some cases comprise a series of steps designed to facilitate the preparation and execution of a mass spectrometry proteomics experiment. For example, a first step comprises defining a question to be explored. In some instances, a question is defined by studying health and market interests related to various sources of information available in mass spectrometry (MS) studies. A second step often is to identify candidate markers, such as biomarker proteins related to the question to be explored. The workflows described herein allow analysis of mass spectrometric data for biomarker proteins. In some cases at least 1, 2, 5, 10, 20, 50, 100, 200, 500, 1000, 5000, 10000, 20000 or more than 20000 biomarkers are analyzed. In some cases no more than 1, 2, 5, 10, 20, 50, 100, 200, 500, 1000, 5000, 10000, or no more than 1,000 biomarkers are analyzed. In some cases about 1 to about 5 biomarkers, about 3 to about 10, about 5 to about 50, about 15 to about 100 biomarkers are identified.

Identifying markers in some cases involves review of any number of sources related to biomarkers, such as literature, public/published databases, proprietary databases, or any other sources consistent with specification that aid in the identification of candidate markers. In some cases, databases are obtained from previous proteomics studies and/or personal proteomes. This often includes use of a module, such as a Data Integration Workbench to explore biological pathways' signals in existing internal datasets. Optionally, the quality of the data in a data source such as a database is checked and flagged. In some cases databases are not used if the source data is judged to be insufficient or of low quality for the study. Data judged to be sufficient is integrated with a data portal for subsequent retrieval in some cases. Methods of literature review include but are not limited to text mining using the question to be explored by the study with specific search terms (or condition terms), such as a disease name, symptom, protein name or other identifier. Candidate biomarker identification determines method development in some cases, for proteomics studies, such as for SIS, targeted proteomics, a protein quantification assay such as SISCAPA, or other antibody based or antibody-independent protein quantification assay. Searches in some embodiments comprise keyword searches (or condition terms) for a disease. In some aspects, searching comprises identifying text recited in the condition term in proximity to biomarker candidate text (such as a protein, pathway, or related disease). In some cases, proximity comprises the same paragraph, sentence, pathway, figure, or document. Searching is in some cases conducted on abstracts, full text, websites, or any other sources comprising text fields. In some aspects, keywords are used to identify genes and pathways from literature references, which are then further evaluated to identify related proteins. After each search is performed, gating functions often evaluate the quality of the search. For example, a semi-automated ontology construction relating to a specific question such as a diseases or protein. In some cases this includes an automated search of a database, such as PubMed. Gating functions evaluate a number of different factors relating to the search quality, such as but not limited to specificity and sensitivity relating to search terms. After results are obtained, results are optionally filtered to provide data most relevant to the question being explored. In some instances, this involves filtering co-occurrences for protein-disease associations with high likely validity. In some instances, the quality of references is determined by the number of citations. In some instances, a reference must have at least 1, 2, 5, 10, 20, 50, or at least 100 citations to be examined as a search result. In some instances, a reference must have no more than 1, 2, 5, 10, 20, 50, or no more than 100 citations to be examined as a search result. In some cases about 1 to about 5 citations, about 3 to about 10, about 5 to about 50, about 15 to about 100 citations are required to pass the gating quality control function. In some instances, the quality of references is determined by the impact factor of the journal publishing the reference. The quality of the references often is determined by age of the publication, for example, references published more than 1, 2, 5, 10, 20, or more than 50 years ago are discarded. In some instances references published at least 1, 2, 5, 10, 20, or at least 50 years ago are discarded. In some cases, the quality of references is determined by specific variables of the study, such as sample size, methods used, statistical parameters/correlations of the peptide with a disease, or other variable affecting the quality of the data in the reference. In some aspects, literature searching is completely automated. In some cases, literature searching is partially automated. Other search analysis operations and quality control evaluations consistent with the specification are also utilized to plan a study workflow. Once candidate biomarkers are identified, in some instances reagents suitable for detection of the marker candidate are identified and optionally located in an inventory. In some cases reagents suitable for detection are mass-shifted peptides.

Designing a study workflow in some cases includes statistical and experimental workflow steps. For example, this often involves (by non-limiting example) analyzing the presence or absence of compounding factors, the structure of experimental groups, and alternately or in combination involves performing one or more statistical analyses, such as power analyses, or any other analysis of additional factors consistent with the specification that are helpful for experimental design. After an analysis is carried out, the design is optionally modified to address factors that may influence the outcome and/or validity of the study results. For example, the presence of confounding factors is addressed by adjusting experimental design structures, or adding appropriate controls. Study designs include but are not limited to simple two-group studies, nested designs, or other custom designs that are used in scientific experiments. In some cases, each design requires additional modification depending on the study. In some aspects, a standard two-group design requires balancing for confounding factors. In another example, nested design comprising a planned series of analyses across which the integrity of discovery and validation sets must be maintained is used.

In some embodiments statistical analysis tools are used to design a workflow plan. Statistical power analysis in some cases provides tools to determine 1) the probability that a statistical test will be able to detect a significant difference and 2) the minimum sample size required to detect a significant difference of a certain size. In some instances, the probability of the statistical test is at least 0.01, 0.05, 0.1, 0.2, 0.3 or at least 0.5. In some instances, the probability of the statistical test is no more than 0.01, 0.05, 0.1, 0.2, 0.3 or no more than 0.5. Study plans not meeting a pre-determined statistical probability are in some cases flagged or discarded. The power of a significance test may be affected by four main parameters that are all linked mathematically: effect size, sample size, alpha level (false positive rate), and beta level (false negative rate, related to power defined as 1−beta). If any three of these parameters are set, the fourth parameter can be found using closed-form solutions or through unique bootstrapping techniques. Non-limiting examples of common statistical analyses that employ power analysis include: difference in means testing (rank tests, t-test, ANOVA); regression analysis (linear, logistic); and ROC curves. In some aspects, bootstrapping methods are used to design study workflows. Other statistical analysis tools consistent with the specification are also utilized in study design workflows, and each step of statistical analysis is optionally checked for quality control. Steps failing a quality control gate are in some cases flagged, the step is not used in the workflow, or additional modules and submodules are used as a result of the gate outcome.

Designing a study workflow in some cases comprises steps for obtaining samples for analysis. Considerations, parameters, or operations involving sample acquisition are important for reducing potential problems prior to executing a full study. Alternately or in combination sample acquisition involves identifying sample sources, evaluating and planning data collection, evaluating early samples, or other processes or operations relevant to sample collection. For sample collection, different methods of sample collection and evaluation are used. For example, retrospective studies involve evaluating the methods that were used to collect data and prospective studies require planning methods of sample collection. The quality and source of sample collection plan is evaluated, and specific samples are optionally flagged or removed from the data pool if quality goals are not met. Samples often are flagged or removed if they are stored for at least 6 months, 1 year, 2 years, 5 years, or 10 years. Samples in some instances flagged or removed if they are stored for less than 6 months, 1 year, 2 years, 5 years, or 10 years. Samples in some cases are flagged or removed if they are stored at temperatures of at least −80 degrees C., −50 degrees C., −20 degrees C., 0 degrees C., or 25 degrees C. Samples often are flagged or removed if they are stored at temperatures of no more than −80 degrees C., −50 degrees C., −20 degrees C., 0 degrees C., or 25 degrees C. A sample collection plan in some cases includes collection methodology, inclusion/exclusion criteria, a case report form (CRF), stopping criteria, a sample naming plan, or other information related to sample collection used to plan a study. For example, a case report form is designed to ensure that all required annotations are obtained using a sensible and simple CRF that is easy for clinical personnel to understand and use. In another example, a sample naming plan is designed such that samples are given randomized anonymous IDs that contain no clinically relevant information. In some instances, a sample naming plan comprising identifying information is discarded. Evaluation of early samples is often conducted by using a subset of (early, if prospective study) samples to perform a pilot study. This allows quality control checks of assumptions used in experimental design (e.g. effect size, noise, etc.), checks of sample quality, checks of annotation quality, or other quality control-related factors to be evaluated. Evaluation of sample collection factors in some aspects is utilized in study planning, and sample collection methods that fail quality control gate standards are flagged or optionally removed from the workflow. For example, blood samples obtained from a source were improperly stored (e.g., improper temperature), and these samples are discarded from the workflow. In some cases, other sample properties such as the method of sample collection or sample age is used to determine whether a sample will be used in the workflow. In some aspects variables such as sample size or other design parameters are altered based on the gating result. For example, the number of samples obtained is insufficient to accurately assess the correlation of biomarkers for a disease, and additional samples or sample sources are automatically integrated into the workflow to compensate. In some instances, at least 1, 2, 5, 10, 20, 50, 100, 200, 500, 1,000, 2,000, 5,000 or more than 5,000 samples are added to the workflow. In some cases, no more than 1, 2, 5, 10, 20, 50, 100, 200, 500, 1,000, 2,000, or no more than 5,000 samples are added to the workflow.

In some instances, a study plan further comprises developing an analytical method, such as a mass spectrometric method. In some aspects, these methods are used for targeted and iMRM proteomics studies, in which MS methods are tailored to the specific transitions being targeted in the study. Steps related to this goal are in some cases performed while samples are being obtained. Developing an MS method in some cases further comprises defining a transition pool, optimizing an MS method, selecting final transitions, or other operation to aid in the development of an MS method. Defining a transition pool comprises a number of operations such as performing an in-silico tryptic digestion, selecting proteotypic peptides, predicting peptide ionization and fragmentation in MS, peptide filtering to ensure effective ionization and fragmentation in MS, generating a model of MS performance for some peptides (obtain crude peptides, empirically determine or measure performance, analyze, etc.), iteratively model, buy, test to refine an SIS or peptide panel, or other process helpful for defining a transition pool. Predicting peptide ionization often comprises applying an in-house model to predict MS ionization and fragmentation of peptides, wherein the model is based on patterns observed in early datasets.

In some cases, peptide filtering utilizes a predictive model based on previous empirical observations. Optimization of an MS method in some instances comprises obtaining stable isotope-labeled standard (SIS) peptides from a QC-controlled source, optimizing an LC (liquid chromatography) gradient, collision energy, or other mass spectrometric variable related to experimental data quality or outcome. In one example, steps include optimizing an LC gradient to obtain a desired signal/noise, for criterion #transitions per peptide and #peptides per protein, and with concurrency below criterion. For example, signal to noise ratios often are optimized to at least 2:1, 5:1, 10:1, 20:1, 50:1, 100:1, 200:1, 500:1 or more than 500:1. In some instances, signal to noise ratios often are optimized to no more than 2:1, 5:1, 10:1, 20:1, 50:1, 100:1, 200:1, or no more than 500:1. In another example, steps include varying LC time and the amount of organic solvent while keeping dwell time, cycle time, gradient time within limits, or any other variable affecting LC results. In some instances the LC time is optimized to no more than 2 minutes, 5 minutes, 10 minutes, 20 minutes, or no more than 50 minutes. In some instances the LC time is optimized to at least 2 minutes, 5 minutes, 10 minutes, 20 minutes, or at least 50 minutes. In some cases the MS collision energy (CE) for each transition is optimized to ensure signal with sufficient amplitude and low CV (coefficient of variation). In some cases an optimized CV is no more than 10%, 8%, 6%, 5%, 4%, 3%, 2%, or no more than 1%. In some instances the collision energy is at least 10 volts, 20 volts, 50 volts, 100 volts, 200 volts, 500 volts, 1,000 volts, 2,000 volts, 5,000 volts, or more than 5,000 volts. The collision energy often is no more than 10 volts, 20 volts, 50 volts, 100 volts, 200 volts, 500 volts, 1,000 volts, 2,000 volts, or no more than 5,000 volts. In other cases collision energy is varied in a number of steps among a number of methods/instrument, depending on the array size. The number of steps in some instances is at least about 7, or at least about 1, 2, 3, 4, 5, 6, 7, 8, 10, 20, 50, or more than about 50 steps. Final transitions are selected by a series of criteria, such as ranking and selecting. In one aspect, automated transition (heavy and light) rankings are based on transition specificity, linearity across standard curves, LloQs (lower limit of quantitation), precision, and dynamic range, or other variable specific in describing transitions. Once, transitions are evaluated, in some cases semi-automated and iterative selection of transitions from top rankings are performed, for example 2 peptides per protein, and 2 transitions per peptide. In some cases no more than about 1, 2, 3, 4, 5, 10, 20, 50, or 100 peptides per protein rankings are performed. In some cases no more than about 1, 2, 3, 4, 5, 10, 20, 50, or 100 transitions per peptide rankings are performed. Alternately or in combination, each iteration considers concurrency and transition rankings for transition selection.

Different samples comprise large amounts of undesired protein that interferes with sample measurement and analysis. In some aspects, a workflow planning module identifies proteins based on a given sample source (e.g. saliva, plasma, whole blood, etc.), and adjusts the study plan to selectively remove interfering signals (e.g. transitions, peaks, etc.) related to these undesired proteins. Sample sources in some aspects are evaluated by organism for the prediction of interfering signals. Alternately or in combination, in some instances a gating function identifies signals that are overrepresented in data of previous studies, and uses this information to inform the current workflow plan.

Study workflows often comprise a step of randomization of sample order. Randomization considers any parameters that may affect the appearance of a signal related to an outcome class including but not limited to the outcome class itself, confounding clinical factors, and laboratory factors (e.g. plate position, day, time, instrument, technician, environment, etc.). A run order is devised to randomize sample order while avoiding situations in which laboratory factors alone could result in apparent signal due to outcome class or to confounding clinical factors. In an exemplary randomization, two sample run order files are produced in order to ensure blinded measurements. One file lists samples with their IDs, clinical annotations, run order, and other relevant information to be used in later analyses—this file is not made available to any lab personnel or analysts until the study runs are complete. The second file lists samples by IDs and order information only—this file is used by lab personnel to prepare samples for the study. Other randomization protocols, procedures, and techniques consistent with the specification are also utilized for sample randomization. If randomization is not accomplished to a desired level of stringency, the study plan may be flagged, abandoned, or restarted. Alternately or in combination, samples may be randomized two or more times and analyzed to eliminate any bias in sample order.

### Study Analyses

After data from a study workflow is obtained, this data is organized and analyzed to evaluate the outcome of the study. The experiments and studies often are mass spectrometric and proteomics studies. Proteomics studies include DPS, targeted, iMRM, a protein quantification assay such as SISCAPA or other antibody based or antibody-independent protein quantification assay or a number of other types and designs of proteomics studies. Analyses of a study may comprise a number of analyses modules, including but not limited to initial data evaluation, feature processing, data exploration, classifiers identification, and visualization. Each module may comprise one or more sub-modules specific to an experiment type. For example, various exemplary study analyses workflows comprising modules and submodules are illustrated in FIGS. 4-6. Between modules a gating method in some cases will evaluate the quality of the data, and optionally discard, repeat or flag for later review steps or data which do not meet predetermined standards.

Data from studies may be visualized through different media, representations, and organizational constructs to evaluate the data for quality and determine the outcome of the study. In some cases, data from a study such as a proteomics study is assessed through a visual representation. For example, data are assessed using a starfield representation, an example of which is show in FIG. 7. Data from the starfield is assessed for quality control, and actions taken based on identifiable aberrations. The visual representation may include identified features from the samples such as, for example, identified analytes such as peptides/lipids/metabolites, and/or QC metrics or other information related to the analytes. For example, a features may include charge state, chromatographic time, overall peak shape, analyte signal strength, and presence of known contaminants. In one aspect, low-resolution pipeline-generated starfield images are visually assessed to identify runs with obvious large-scale aberrations. If any aberrant runs are found, root cause analysis is performed. Aberrant runs are then reprocessed through the pipeline, repeated, removed from further analysis, or flagged for later evaluation depending on the outcome of the root cause analysis. Data in some aspects is also visualized with a medium-resolution starfield images that are scrolled through quickly, with their order determined by a selected annotation field. Sequential images are viewed independently and well-aligned, so that visual persistence enables comparison of feature groups across images. This allows exploration of feature cluster patterns associated with annotations. In some cases, high-resolution starfield images are visually assessed to check that peaks have expected isotope structure, and appear with the expected density across the image FIG. 8. Different interaction tools are also available for viewing or interacting with starfield or other data representations. In one case, a high-resolution 3-D starfield image is viewed using a 3D viewing platform, as shown in FIG. 9. Starfields in some aspects are also used to count features for quality evaluation of the data. In some instances, data is discarded or flagged if the starfield comprises no more than 5,000, 7,000, 10,000, 15,000, 20,000, 25,000, 30,000, 40,000, 50,000 or no more than 100,000 features. In some instances, data is discarded or flagged if the starfield comprises at least 5,000, 7,000, 10,000, 15,000, 20,000, 25,000, 30,000, 40,000, 50,000 or at least 100,000 features. For example, the pipeline-based feature count for each starfield is checked to ensure it is within expected ranges. In some instances, the starfield data is flagged or discarded if there are no more than 5,000, 7,000, 10,000, 15,000, 20,000, 25,000, 30,000, 40,000, 50,000 or no more than 100,000 matching features between identical sample runs. In some instances, the starfield data is flagged or discarded if there are at least 5,000, 7,000, 10,000, 15,000, 20,000, 25,000, 30,000, 40,000, 50,000 or at least 100,000 matching features between identical sample runs. The outcome of this quality check optionally controls downstream changes to the analyses workflow, such as removing or adding submodules, flagging data, or removal of data from the analysis. Consistent with the specification are other representations of data visualized with alternative interactive platforms. Evaluation of data is accomplished through user interaction, or optionally in an automated fashion.

Another module for analyses of proteomics experiments uses process features for a proteomics experiment. Submodules may vary based on the type of proteomics experiment being analyzed, and steps may be omitted or added depending on the nature of the data and experiment.

Feature processing submodules for an experiment such as a profile or DPS proteomics experiment often are cluster, fill-in-the-blanks (FIB), normalize, handle multiple peak clusters, filter peaks, assign IDs, or other module used to process proteomics data. In some aspects, features that appear to arise from the same analytes in separate injections are associated, and clustering is done based on each feature's LC and m/z positions. Each cluster is then assigned a unique ID. A fill-in-the-blanks module in some instances comprises proposing a peak area value for any cluster that is missing from any starfield, and if a cluster is not detected as a peak in all starfields, the intensity measure at the cluster LC and m/z location in each starfield from which the cluster is missing is obtained. A normalization module often is used to normalize peak areas across starfields so that peaks from different starfields can be usefully compared. If the normalization module fails to normalize peak values across two starfields, the starfields are flagged for additional analysis. In the event that a cluster will be assigned more than one measure per starfield, a handle multiple peak clusters module is optionally used. Usually these clusters are omitted from further analysis, but are alternatively or in combination flagged. In special cases, additional processing is performed to resolve the multiple cluster peak areas into a single value to be used in further analysis. Data may also be filtered to exclude certain values based on quality. For example, a module selects clusters with FIB rates below a specified maximum, and these clusters will be included in further analyses. Other clusters will be flagged or discarded from the analysis. In some instances the analysis is altered to account for filtered data.

Feature processing submodules for an experiment such as a DPS in some cases comprise identifying targeted SIS peaks, identifying endogenous peaks, or other steps to process features in the experiment. In one example, SIS peaks are found at specified m/z and RT locations and have areas that increase with standard concentration. In some instances endogenous peaks are found at specified m/z offsets relative to corresponding SIS peaks.

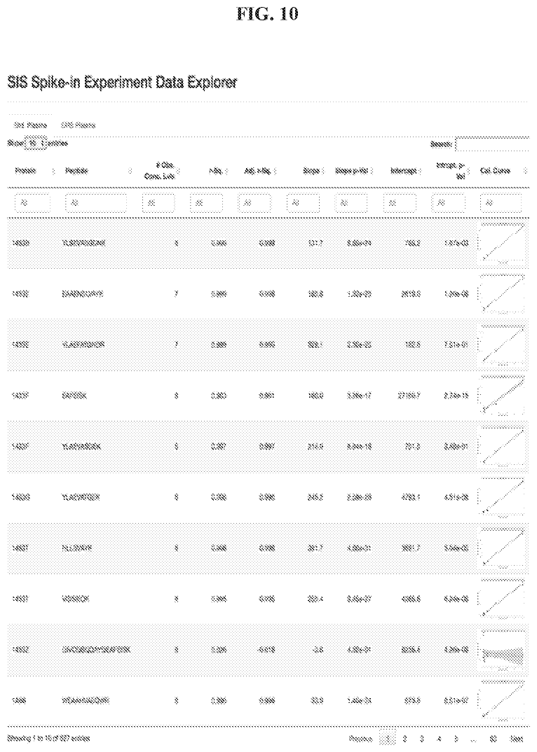

Feature processing submodules for an experiment such as a DPS, targeted, or iMRM proteomics in some cases comprise filtering peaks, filtering transitions, calculating concentrations, or other process used to evaluate features in a mass spectrometric experiment data set. Filter parameters may be determined by a visualization tool. For example, FIG. 10 shows an exemplary chart obtained from an SIS Spike-In experiment to visually assess and filter standard curves from multiple injections based on measures of spike-in standards (proteins or peptides) is shown. This visualization tool allows filtering along a variety of criteria (number of standards, R², adjusted R², slope, intercept, slope p value, intercept p value). In some cases, at least 10, 20, 50, 100, 200, 500, 1,000, 2,000, 5,000, 10,000, 20,000, or at least 50,000 transitions are filtered. In some cases, no more than 10, 20, 50, 100, 200, 500, 1,000, 2,000, 5,000, 10,000, 20,000, or no more than 50,000 transitions are filtered. Transition filtering may depend on a number of variables specific to transitions. For example, transitions are filtered by CV, linearity of standard curve, dynamic range, LLoQ, or other variable so that only transitions with high-quality quantitative measurements are used in further analyses. In some instances concentrations are calculated based a comparison of known and unknown sample amounts, such as a comparison of endogenous and labelled peak areas.

Feature processing submodules for an experiment such as a targeted or iMRM proteomics in some cases comprise peak shape filtering, signal quality evaluation, or other process used to evaluate features in a mass spectrometric experiment data set. Automated peak shape evaluation in some cases comprises an automated tool that evaluates peaks based on aspects of their shape. Another processing submodule is for signal quality evaluation. In one embodiment, a machine learning tool to selects the best quality peaks, where quality relates to signal strength combined with consistency along a variety of parameters. An initial expert review of several hundred peaks assigns them to three quality groups. Consultation with the expert reviewer reveals a set of parameters driving the group assignments; these parameters are then translated into computed predictor variables. Using these predictors, a random forest classifiers are developed and tested on a hold-out test set, assigning peak quality groups with 91% accuracy (98% sensitivity and 85% specificity in separating groups 1 and 2 from group 3). Additional assignments with different accuracy, sensitivity, and specificity can also be used. For example, assigning peak quality groups with at least 60%, 65%, 70%, 75%, 85%, 90%, 95%, or at least 98% accuracy. Sensitivity separating groups 1 and 2 from group 3 is in some cases at least 60%, 65%, 70%, 75%, 85%, 90%, 95%, or at least 98% sensitivity. In some aspects, specificity is at least 60%, 65%, 70%, 75%, 85%, 90%, 95%, or at least 98%. Signal quality evaluation in some embodiments is automated without user monitoring or input.

Feature processing submodules for an experiment such as iMRM proteomics in some cases comprise calculating concentrations, or other process used to evaluate features in a mass spectrometric experiment data set. In some instances, this involves a module for corrected lookup concentration. For examples, iMRM proteomics uses an additional calculation of endogenous concentration, based on analysis of forward and reverse curves. In some cases, an endogenous protein concentration that does not meet predetermined standards in some aspects results in flagging of the data, discarding of the data, or other change to the analyses workflow.

Additional submodules for feature processing often include finalizing data matrices, exploring data, transforming data, building classifiers, proteome browsing, or other feature processing. Finalizing data matrices may comprise compiling/reshaping data into standard classifier data matrices, such as by putting data into wide matrix format, with one line per sample and one column per predictor. In some instances, discovery and validation (test) sets are kept separated.

Exploring data may involve a series of submodules designed to explore signals in the data set related to study goals. These submodules include examining a univariate signal in the discovery set, PCA in the discovery set, or other goal/outcome data discovery modules. Analysis of a univariate signal often comprises examining each single predictor's signal in relation to the main outcome variable, in the discovery set. PCA comprises performing a principal components analysis to determine if linear combinations of cluster concentration measures are related to the main outcome variable. Other methods targeting the main outcome variable consistent with the specification are also utilized. In some instances, variables having weak correlation to the main outcome variable are flagged or discarded.

Additional data exploration of the data is also accomplished by additional modules that examine correlations in the data, clustering, and methods to visual the data. An example correlation includes exploring pair-wise correlations among all cluster concentration measures. These correlations in some cases point the way to cluster combinations that may be useful in building new predictor variables. Hierarchical clustering is in some cases used to explore groups of discovery set samples that have similar concentration profiles, and this is used to determine whether these groups can be explained by sample annotations (e.g. demographic factors, medications, comorbidities, or other sample annotations).

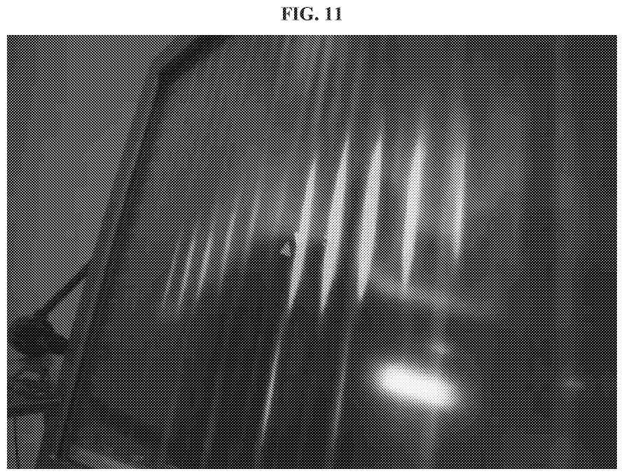

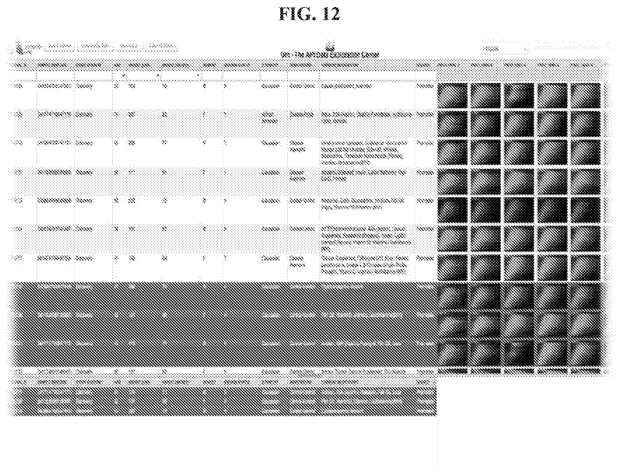

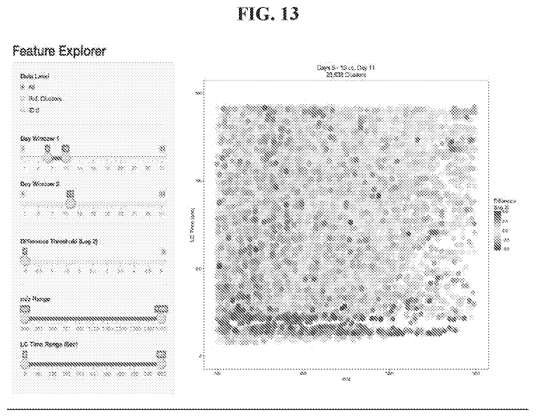

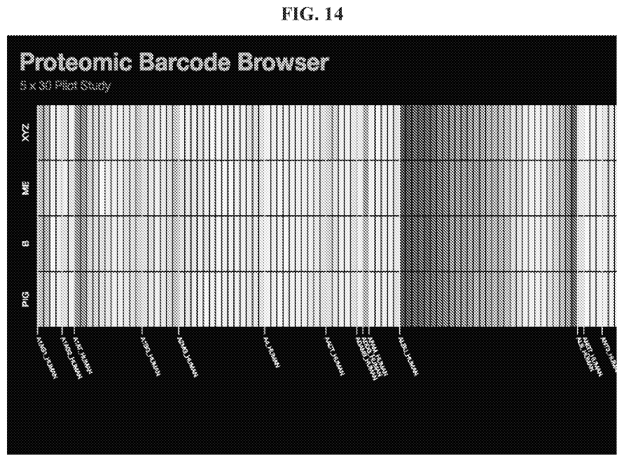

Data can also be explored visually, through many various interfaces that are used to visualize data, such as mass spectrometric or proteomics data. In one case, a touchable interface, such as a TouchTable device is used to visually explore data (FIG. 11). Interfaces allow confirmation that clusters appearing to carry outcome-related signal are from high quality peaks, and to visually compare such clusters' signal across samples from different outcome classes (FIG. 12). In another example, low-resolution starfield thumbnail images across samples are grouped and filtered by sample annotations. This allows images to be viewed simultaneously to enable comparison; this allows identification of large-scale patterns associated with annotations. Additional visualization methods allow exploration of features, such as features over time. FIG. 13 illustrates abstracted and filtered features from one individual over time, which allows exploration of temporal patterns by comparing average intensities from at least two user-selected time slices. In some cases at least 2, 3, 5, 7, 10, 20, 50, 100, 200, 500, or more than 500 time slices are compared.

Data transformations are another aspect of the data analyses, and involve automated manipulation of large data sets. One exemplary transformation involves transforming predictor concentration values as needed to enhance comparisons between predictors and to inform construction of novel predictors based on predictor combinations. Typical transformations are Log 2, and standardization (mean of 0, standard deviation of 1), but may include additional transformations such as ratios or feature combinations.

In yet another aspect of workflow analyses, modules may include build, validation, or other classifiers. A build classifier in some cases comprises a focused classifier approach: a one feature selection approach combined with one classifier algorithm. In some instances, builds are stored on an internal database server. In another aspect, build classifiers comprise creating a grid. A simple grid module comprises in some cases a system of automated tools to examine a grid of feature selection and classifier settings. In some aspects, at least 10, 20, 50, 100, 200, 500, 1000, 2000, 5000, or at least 10,000 builds are analyzed for a simple grid module. An expanded grid module comprises in some cases a system of semi-automated tools to examine a grid of feature selection and classifier settings, with more options for feature selection and classifiers than used in the simple grid module. In some aspects, at least 1000, 2000, 5000, 10,000, 20,000, 50,000, 100,000, or at least 200,000 builds are analyzed for an expanded grid module. Additionally, a module comprising a system of semi-automated tools to perform exhaustive searches of all possible predictor combinations is used for one selected classifier configuration. In some aspects, at least 1 million, 2 million, 5 million, 7 million, 10 million or at least 20 million builds are using in the exhaustive search module. Classifiers may also comprise a variety of structures, such as a SUn structure. For example, SUn (Status of Univariates) is a conditional classifier algorithm in which a conventional multivariate classifier determines outcome call in some cases, but can be superseded by a single-predictor-based call if the single predictor value exceeds a specified criterion. Alternative structures are also often developed guided by insights and observations about patterns apparent in the discovery set. Model refinement algorithms addressing indeterminate score regions, are also used to enhance discovery set signals. When a final discovery classifier has been optimized and locked, in some aspects it is tested it by applying it once to the full validation set. Consistent with the specification, other modules and methods for working with classifiers are used. If a classifier fails against a full validation set, alternative analyses are optionally carried out to provide an improved classifier.

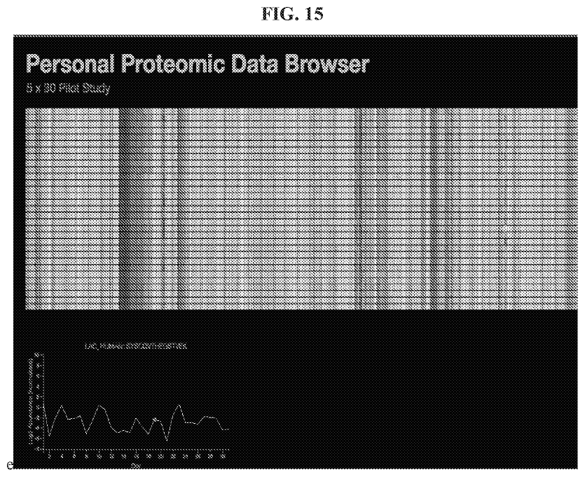

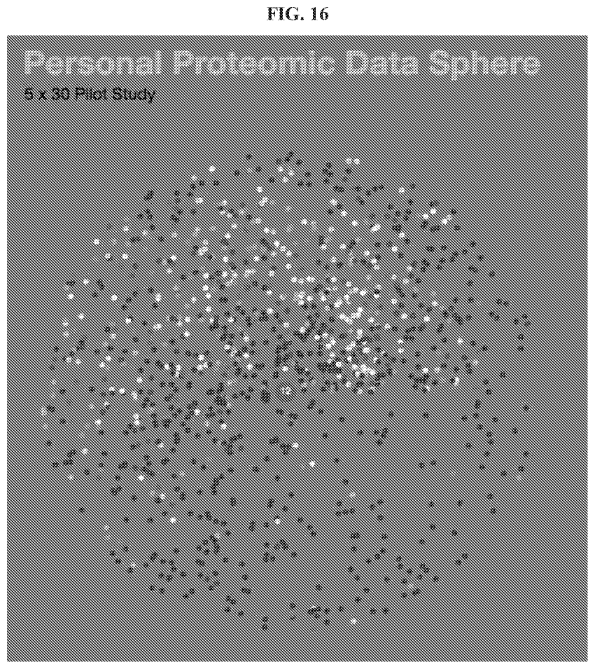

A number of different interface systems, modules and methods are used to interact with data acquired from experiments, such as mass proteomics experiments. These methods allow for exploration of a single proteome, or of multiple proteomes. Proteomes are obtained from a single individual, or from multiple individuals. An exemplary proteomic barcode browser is depicted in FIG. 14. In some instances, the browser identifies protein abundance (normalized) from multiple individuals in a graphical format which enables ready visual detection of individual differences. In some cases, proteomics data is observed longitudinally over time, as shown in FIG. 15. Often proteomics data is observed by reviewing identified peptide/protein abundance (normalized) for a single individual over a study period. A graphical format enables ready visual detection of time-related changes, and a line plot of a given peptide's abundance over the entire study period often is generated for a more detailed examination. In yet another exemplary visualization, data may be observed through a person and population proteome viewer (FIG. 16). This alternate visualization method allows analysis of one individual's MS features, using polar coordinates, with m/z as the angle and LC as the radius. In some instances, multi-day data is displayed by stepping through one day at a time. Other visualizations consistent with the specification are also utilized to visual MS and mass spectrometric data over time and across individuals or populations.

This allows exploration of an individual's or group of personal proteomes by comparing the individual's concentrations of functionally-grouped proteins (e.g. cardiac-related, inflammation-related), to the distributions of concentrations for the same functionally-grouped proteins across the larger population. This system allows a user to view concentrations of functionally-related proteins relative to large population distributions; in some aspects this view pinpoints the biological functions where the individual's proteome differs from the bulk of the population.

### Algorithm-Based Methods

Methods, compositions, kits, and systems described herein are compatible with an algorithm-based diagnostic assay for predicting a presence or absence of at least one health status or condition in a subject. Expression level of one or more protein biomarker, and optionally one or more subject characteristics, such as, for example, age, weight, gender, medical history, risk factors, or family history are used alone or arranged into functional subsets to calculate a quantitative score that is used to predict the likelihood of a presence or absence of the at least one health condition or status. Although lead embodiments herein focus upon biomarker panels that are predominantly protein or polypeptide panels, the measurements of any of the biomarker panels may comprise protein and non-protein components such as RNA, DNA, organic metabolites, or inorganic molecules or metabolites (e.g. iron, magnesium, selenium, calcium, or others).

The algorithm-based assay and associated information provided by the practice of any of the methods described herein can facilitate optimal treatment decision-making in subjects. For example, such a clinical tool can enable a physician or caretaker to identify patients who have a low likelihood of having an advanced disease and therefore would not need treatment or increased monitoring for advanced disease, or who have a high likelihood of having an advanced disease and therefore would need treatment or increased monitoring.

A quantitative score is determined by the application of a specific algorithm in some cases. The algorithm used to calculate the quantitative score in the methods disclosed herein may group the expression level values of a biomarker or groups of biomarkers. The formation of a particular group of biomarkers, in addition, can facilitate the mathematical weighting of the contribution of various expression levels of biomarker or biomarker subsets (for example classifier) to the quantitative score.

### Exemplary Subjects

Biological samples are collected from a number of eligible subjects, such as subjects who want to determine their likelihood of having at least one health status, condition, or disease. The subject is in some cases healthy and asymptomatic. The subject's age is not constrained. For example, the subject is between the ages of 0 to about 30 years, about 20 to about 50 years, or about 40 or older. In various cases, the subject is healthy, asymptomatic and between the ages of 0-30 years, 20-50 years, or 40 or older. In various examples, the subject is healthy and asymptomatic. In various examples, the subject has no family history of the health condition or disease.

In some cases, a subject presents at least one of a health condition or disease. In some cases, a subject is identified through screening assays or scans as being at high risk for or having the health condition or disease. In some cases, the subject is already undergoing treatment of the health condition or disease. For example, one or more methods described herein are applied to a subject undergoing treatment to determine the effectiveness of the therapy or treatment they are receiving.

### Automated Devices and Workflows for Biomarker Assessment

The present disclosure provides for devices and methods for measuring one or more biomarker panels in biological samples. The devices are generally able to perform some or all of the tasks associated with preparing and analyzing a sample for a panel of biomarkers. Exemplary functions of the devices include tracking and organizing experiments, preparing samples, preparing reagents for use in the devices and methods, configuring instrumentation for a particular protocol, tracking samples, aliquoting samples, assessing the quality of samples, processing steps, reagents, and instrumentation, quantify samples and reagents, provide samples and reagents to detectors, detecting biomarkers, recording data, uploading data to systems for analysis, assessing samples or results, assessing controls and results obtained therefrom, flagging samples or results, and modifying any of the operating parameters or functions described herein based on the detection of specific parameters or quality characteristics.

(a) Control Systems and Databases

The devices and processes described herein are often tracked, automated, and organized by a control system. Exemplary systems include laboratory management information systems (LIMS). The LIMS are often configured to automate transmission of data related to processes and samples. Exemplary functions of the LIMS provided herein include workflow and data tracking support. This can include transmission of experimental tracking data and worklists. LIMS can also be configured to manage the transmission of sample processing instructions and protocols. Some LIMS can transmit and record of results. Some LIMS calculate, track, and set up the ordering and randomization of samples. This can include tracking sample positions on plates or cards throughout an experiment. Some LIMS can process, record and normalize data from liquid chromatography devices. Some LIMS can process, record and normalize data from mass spectrometers. Some LIMS can flag samples, sample intermediates, or results.

Control systems often store or determine “worklists” or protocols. The worklists serve to provide instructions for any or each step in a process and can also record experiment-specific data for samples. In some cases, worklists contain scripts used by the devices. These worklists can be prepared from a template. Templates often include random sample ordering and appropriate volumes to be used. Randomization need not be complete randomization. The process of randomizing samples can take into account any parameters that may affect the appearance of signal related to outcome class. Examples include the outcome class itself, confounding clinical factors, and laboratory factors (e.g. plate position, day, reagents used, etc.). A run order is frequently devised to randomize sample order while avoiding situations in which laboratory factors alone could result in apparent signal due to outcome class or to confounding clinical factors. In order to ensure blinded measurements, two sample run order files are often produced. A first file lists samples with their IDs, clinical annotations, run order, and other relevant information to be used in later analyses. This first file is frequently not made available to lab personnel or analysts until the study runs are complete. The second file lists samples by IDs and order information only and is often used by lab personnel to prepare samples for the study. Results can be flagged, for example, if the samples are run in an order that is not sufficiently randomized or that does not comply with requirements or parameters of a specific protocol.

Control samples are often processed in the same order for every worklist. This order can include control samples being used at the beginning, middle, and end of specific steps within the experiment. As such, control samples can help normalize samples and worklists during data analysis. This can include sample label information and reagents information, including concentrations and lot numbers used with a particular set of samples. Worklists used with a particular process can be stored with, archived, or associated with the corresponding experiment for later reference. Data can be flagged if control samples are not run in a particular order or at the specified times.

The incorporation of automated gating between physical manipulation steps allows one to identify defective steps in certain runs, such that samples or sample runs not meeting a threshold, surpassing a threshold, cumulatively indicating a defect in a workflow, or otherwise exhibiting an property casting doubt on a final mass spectrometric outcome, are identified. Identified samples or sample analysis runs are variously flagged as failing a manipulation assessment, repeated, discarded, subjected to a pause or cessation of an analysis workflow, or otherwise addressed such that sample integrity or workflow constituent operation may be assessed or addressed prior to continuing an analysis workflow.

Some systems or modules can adjust parameters based on a variety of inputs. For example, some systems use optical density measurements to determine protein concentration estimates. Such estimated can be measured from known concentrations in control samples. The systems are configured to determine the parameters applied when computing sample concentrations, manipulations, and analysis.

Likewise, the systems or modules can determine and process protein mass. Such determinations can be made using known control proteins, which can be fractionated, diluted, and then measured to determine the parameters applied when computing fraction mass distribution.

Such systems or modules can comprise an application program interface (API), process controls, quality controls, custom software, and combinations thereof.

(b) Reagent Preparation

The devices, systems, and modules described herein can also be configured to prepare, dispense and assess or control the quality of reagents and solutions useful in the provided methods. Failure of any one of these steps can result in the associated samples being flagged during a gating event. Such reagents can include the detergents, chaotropes, denaturants, reducing or oxidizing agents, alkylating agents, enzymes, salts, solutions, buffers, or other reagents and items useful in the described methods. The devices can store and dispense these reagents as needed during the course of one or more experiments. Dispensing can be accomplished through a series of tubes and fluidic controls. Some variants of the devices include temperature-controlled storage devices. Such experiments can sometimes last hours, days or weeks.

(c) Plate Preparation

The devices, systems, and modules described herein can also be configured to prepare plates used to process and analyze the samples. The devices can optionally include or add control samples to the plates. The control samples can be, for example, samples derived from known sample pools or samples with known concentrations. Some experiments include the use of 1, 2, 3, 4, 5, 6, 7, 8, 9, 10 or more controls. Each of the controls can also be plated as a series of dilutions with known variations in concentration, such as a serial dilution. These controls can serve to verify that the devices and processes are working as expected and that the quality of particular steps is sufficient to yield results that are accurate and precise. Some of the quality control samples are added to assess the quality of specific processes or steps. Other control samples are added to assess overall quality. Some controls serve as negative controls. The control samples are generally processed in parallel with study samples so they undergo the same or similar laboratory actions as the sample. Some control samples are prepared from a stock solution with standardized properties, such as known concentrations of particular components. An example stock solution includes one containing heavy peptides of interest at known concentration, as described below.

The devices and processes generally determine the sample mixture and determine the aliquot count and volume. Processes and samples that do not meet specific criteria can be flagged, for example, by one of the modules described herein. For example, control samples that do not meet specific quality control criteria or are improperly prepared or dispensed can be flagged. This includes determining if the variability of a particular experiment is within acceptable levels.

Controls can be used to create calibration curves. Calibration curves can be used to map mass spectrometry data to peptides with known concentrations. In some such experiments, peptides with known concentrations or dilution factors are used to estimate sample peptides with unknown concentrations. Such controls can be stored as frozen stocks, thawed, and diluted to build curves of known concentrations. These controls can also be spiked with stable isotope standards. In some embodiments, the stable isotope standards comprise hundreds of peptides comprising stable isotopes, including 100, 200, 300, 400 or more peptides. The stable isotopes can be suspended in a plasma background. These peptides can include heavy versions of peptides that are known biomarkers for a particular disease or condition. The control samples are often processed in order from lowest to highest concentrations, which can help determine the daily instrument performance and individual sample concentrations. Calibration curves can include 1, 2, 3, 4, 5, 6, 7, 8, 9, or more points generated by standards containing different concentrations of known solutions, including solutions containing stable isotope standards. These curves can be automatically evaluated by software without the need for user assistance. Data or samples that are not run in the proper order or which are outside of an expected range can be flagged.

(d) Dried Plasma Spot Proteomics Sample Preparation

Also provided herein are systems, methods, and modules that utilize dried plasma spot proteomics. In some such studies, samples and controls prepared as described herein are transferred to a dried plasma spot card for subsequent analysis. The samples often comprise liquid plasma or whole blood. The systems and methods described herein can determine an appropriate amount of sample to transfer to or from the card. Some embodiments of dried plasma spot proteomics comprise spiking the samples and/or controls with stabile isotope standards (SIS). The SIS can comprise heavy peptides of interest and can be prepared as stock solutions with known concentrations. The SIS can also be stored as frozen or lyophilized samples. Lyophilized samples can be reconstituted in an appropriate buffer at an appropriate amount of volume. Such reconstitution can be determined by and controlled by a LIMS. The samples or data can be flagged if the modules detect that not enough sample was transferred to the card, too much sample was transferred to the card, a sample did or did not include the appropriate amount of SIS, or if the sample was stored or reconstituted improperly.

(e) Exemplary Biological Samples and Sample Preparation

Some of the devices, methods, and modules described herein are designed to process biological samples. Biological samples are frequently circulating blood samples or are samples obtained from the vein or artery of an individual. Samples are optionally processed by the devices or modules described herein, which are configured to isolate plasma, circulating free proteins, or a whole protein fraction from the blood sample.

As a representative sample collection protocol, blood samples for serum, EDTA plasma, citrate plasma and buffy-coats are collected with light tournique from an antecubital vein using endotoxin-, deoxyribonuclease (DNAse-) and ribonuclease (RNAse-) free collection and handling equipment, collection tubes and storage vials from Becton-Dickinson, Franklin Lakes, N.J., USA and Almeco A/S, Esbjerg, Denmark. The blood samples are frequently centrifuged at 3,000×G for 10 mins at 21° C. and serum and plasma are immediately separated from the red cell and buffy-coat layers. Contamination by white cells and platelets can be reduced by leaving 0.5 cm of untouched serum or plasma above the buffy-coat, which can be separately transferred for freezing. Samples with too many contaminating white blood cells and platelets can be flagged. Separated samples are optionally marked with unique barcodes for storage identification, which can be performed using the FreezerWorks®, Seattle, Wash., USA tracking system. Some samples are often treated to facilitate storage or to allow shipment at room temperature, although in preferred embodiments samples are shipped frozen, for example with or on dry ice, to preserve the samples for analysis at a processing center separate from a phlebotomist's office. Separated samples are often frozen at −80° C. under continuous electronic surveillance. Samples that are not continuously frozen at a desired temperature can be flagged. The entire procedure is often completed within 2 hours of initial sample draw.

Additional biological samples include one or more of, but are not limited to: urine, stool, tears, whole blood, serum, plasma, blood constituent, bone marrow, tissue, cells, organs, saliva, cheek swab, lymph fluid, cerebrospinal fluid, lesion exudates and other fluids produced by the body. The biological sample is in some cases a solid biological sample, for example, a tissue biopsy. The biopsy can be fixed, paraffin embedded, or fresh. In many embodiments herein, a preferred sample is a blood sample drawn from a vein or artery of an individual, or a processed product thereof.

The devices, methods, and modules described herein can be configured to optionally process the biological samples using any approach known in the art or otherwise described herein to facilitate measurement of one or more biomarkers as described herein. Sample preparation operations comprise, for example, extraction and/or isolation of intracellular material from a cell or tissue such as the extraction of nucleic acids, protein, or other macromolecules. The devices are generally configured to assess the quality of the extraction and/or isolation of the materials. For example, the device can be configured with a spectrophotometer, instrumentation to determine protein concentration, and/or instrumentation to detect contaminants. Samples that fail to meet desired characteristics or standards can be flagged.

The device and modules can also be configured to prepare the sample using centrifugation, affinity chromatography, magnetic separation, immunoassay, nucleic acid assay, receptor-based assay, cytometric assay, colorimetric assay, enzymatic assay, electrophoretic assay, electrochemical assay, spectroscopic assay, chromatographic assay, microscopic assay, topographic assay, calorimetric assay, radioisotope assay, protein synthesis assay, histological assay, culture assay, and combinations thereof. Each of these modules or steps can include a gating step. Samples assessed by any of these means that do not meet desired characteristics or standards can be flagged.

Sample preparation optionally includes dilution by an appropriate solvent and amount to ensure the appropriate range of concentration level is detected by a given assay. Samples that do not fall within the appropriate range can be flagged.

Accessing the nucleic acids and macromolecules from the intercellular space of the sample is performed by either physical methods, chemical methods, or a combination of both. In some applications of the methods, following the isolation of the crude extract, it will often be desirable to separate the nucleic acids, proteins, cell membrane particles, and the like. The separation of nucleic acids, proteins, cell membrane particles, and the like can be assessed by any means known in the art. Samples that are deemed to have suboptimal separation or isolation can be flagged. In some applications of the methods it will be desirable to keep the nucleic acids with its proteins, and cell membrane particles.

In some applications of the devices, methods and modules provided herein, the devices or modules extract nucleic acids and proteins from a biological sample prior to analysis using methods of the disclosure. Extraction is accomplished, for example, through use of detergent lysates, sonication, or vortexing using glass beads.

Molecules can be isolated using any technique suitable in the art including, but not limited to, techniques using gradient centrifugation (for example, cesium chloride gradients, sucrose gradients, glucose gradients, or other gradients), centrifugation protocols, boiling, purification kits, and the use of liquid extraction with agent extraction methods such as methods using Trizol or DNAzol. Samples or processes that yield suboptimal isolation can be flagged.

Samples are prepared according to standard biological sample preparation depending on the desired detection method. For example, for mass spectrometry detection, biological samples obtained from a patient may be centrifuged, filtered, processed by immunoaffinity, separated into fractions, partially digested, and combinations thereof. For example, peptides of interest can be reversibly bound to selective antibodies while other constituents of the samples are washed out. The peptides can be released from the antibodies, resulting in a sample enriched for peptides of interest. In some examples, antibodies can be bound to beads, including magnetic beads, or columns. The samples and controls can be mixed with the bound antibodies, the complexes can be washed, and the peptides eluted off the antibodies. In some embodiments, the devices disclosed herein are configured to perform these tasks with no or minimal human supervision or intervention. Various resulting fractions may be resuspended by the devices and systems described herein in appropriate carrier such as buffer or other type of loading solution for detection and analysis, including LCMS loading buffer.

Sometimes the samples are assessed before being analyzed for features that can compromise the ability to analyze the samples using the intended protocol. Non-limiting examples of such assessments include hyperlipidemia or the presence of large amounts of hemoglobin. Samples that are determined to be out of desired ranges can be flagged.

Samples can also be purified or isolated before they are analyzed. An exemplary system is the Multiple Affinity Removal System from Agilent. Particles and lipids can also be removed by filtration.

Samples can be assessed for protein content. Such determinations are useful in order to ensure that the correct amounts of reagents and buffers are used in subsequent steps. The amount of total protein in each sample can also be used to automate fractionation, digestion, and reconstitution steps for each sample. The devices and processes described herein can be configured to determine the total amount of protein contained in each sample. For example, the devices and systems can include an optical scanner or an instrument configured to determine optical density. The measurements taken can include measuring a plurality of replicates for each sample, which can include measuring a plurality of aliquots of the same sample. The measurements may also include diluting the sample, including serially diluting the sample, prior to assessing protein content.

These data can then be uploaded to the LIMS. The LIMS can assess the protein measurements and detect samples that are consistent with predetermined or calculated parameters. Samples that fail to meet these parameters can be flagged, adjusted, or discarded under some circumstances. In some cases, the system can automatically correct samples by concentration, dilution, or other method. The coefficient of variation can also be calculated for replicates derived from the same sample to determine if the measurements are accurate or consistent. The LIMS may also calculate a dilution curve based upon the known dilution factors between serially diluted samples. Samples that generate curves that do not fall within specified tolerances can be flagged.

The LIMS can also flag samples that do not contain a desired amount of total protein. Samples that do not contain enough total protein can be concentrated prior to subsequent processing steps, while samples that contain too much total protein can be diluted.

An exemplary adjustment includes the computation of an amount of each sample to subject to protein digestion. This can increase reproducibility of subsequent steps and overall results, including depletion. Such digestion can be accomplished in an immuno-depletion fractionation (IDFC) liquid chromatography system.

Samples are optionally depleted and fractionated. Such steps can increase the likelihood of identifying as many proteins as possible in some methods. Generally, depletion removes the most abundant proteins from the sample. In some cases, this includes removal of 1, 2, 3, 4, 5, 6, 7, 8, 9, 10, 11, 12, 13, 14, 15, 20, 25, 30, or more of the most abundant proteins from the sample. Examples of proteins that may be abundant in samples and that may be removed are one or more of albumin, IgG, antitrypsin, IgA, transferrin, haptoglobin, fibrinogen, alpha 2-macroglobulin, alpha1-acid glycoprotein, IgM, apolipoprotein A1, apolipoprotein A2, complement C3, transthyretin and combinations thereof. This can allow for the detection of proteins at lower concentrations by increasing their representation in the sample. Alternatively or in combination, pre-selected proteins may be depleted. For example, sometimes non-abundant proteins are pre-selected for depletion based on the goals of the analysis (e.g., depleting a biomolecule that tends to interfere with quantification of a target biomarker due to structural similarities). Fractionation can divide each sample by biophysical properties, which can reduce the complexity of a sample and increase the representation of particular proteins in each fraction. Some of these properties include size, charge, hydrophobicity, cellular location, and solubility. Fractionation can separate isoforms for individual proteins. Some methods use fractionation alone, some methods use depletion alone, some methods use both depletion and fractionation, and other methods use neither depletion nor fractionation.

Modules can assess depleted and/or fractionated samples. In one exemplary advantage, such assessments can optimize the fractionation and depletion of samples in order to ensure that such steps selectively reduce the number of interfering peptides that are analyzed by the LCMS. Samples that do not meet specific depletion and/or fractionation standards can be flagged. For example, a module can include one or more detectors on a liquid chromatograph used to fractionate a sample. One exemplary detector includes a thermometer, which can measure the temperature of the fluids entering the column, exiting the column, and/or of the column itself. Another exemplary detector can comprise a pH meter to ensure that fluid passing through the column is within the ranges necessary to retain or elute the analytes at the appropriate time and to ensure that any pH gradient is varying at an appropriate rate. Analyte solubility can often depend on the degree of ionization (dissociation) in the solvent. Neutral non-polar analytes may pass into the organic solvent, whereas ionic or fully dissociated polar analytes may not. The pH of solvents can be manipulated to encourage dissociation.

Likewise, a detector can also detect the ionic strength of the solution flowing through the column and adjust partitioning salts as necessary. Pressure gages can detect the pressure within the column. Flow meters can detect the flow rate to ensure that sample retention and elution is optimized. Samples being processed at under the wrong conditions can be flagged and adjustments can be made to ensure consistency across experiments.

Another exemplary detector can detect absorbance electromagnetic radiation. Examples include absorbance UV, visible, infrared, or combinations thereof, such as a UV/visible radiation absorbance detector. Other examples of detectors include charged aerosol detectors. Such detectors often produce data in the form of traces or peaks that correspond to matter eluting off the chromatography column. The raw traces can be processed into files, including comma separated values (CSV) files. The files can be uploaded to a database or LIMS. The uploaded data can also be archived automatically. The LIMS can be configured to analyze the data produced by the module and flag samples that do not meet certain standards. Examples include samples that do not contain expected peaks, samples that contain peaks that are too high or too small, etc.

Samples can be loaded onto the plates at various points in the process. The devices and processes described herein can feed the samples onto the plates described above. This process can include ordering the samples according to data preloaded into a database or a system controlling the workflow, devices, and methods. Such systems include laboratory information management systems, including those described above. The sample tubes often contain sample labels, which can include barcodes. Barcodes are frequently checked and double-checked throughout the process. Sample labels are often checked before the samples are loaded onto plates. Samples that are improperly loaded can be flagged. Improper loading can include loading into the wrong well location or loading an incorrect volume of sample.

The systems and modules can compute the protein masses from the fractionated samples. In some cases, the system uses the data collected from the liquid chromatography column to compute the protein masses from the fractionated samples. In some cases, the computer uses data from an estimate of total sample protein mass to distribute it among the various sample fractions. Fractionated samples determined to have protein masses outside of desired ranges can be flagged. The estimated protein masses can be computed as concentrations. Samples can be flagged if they contain protein concentrations of less than 1000, 900, 800, 700, 600, 500, 400, 300, 200, 100, 50, or 25 μg/μL. Likewise, samples can also flagged if they contain protein concentrations of greater than 1000, 900, 800, 700, 600, 500, 400, 300, 200, 100, 50, or 25 μg/μL. The estimated protein masses can be computed as a percent recovery. Samples that contain less than 99%, 98%, 97%, 96%, 95%, 90%, 80%, 70%, 60%, 50%, 40%, 30%, 20%, 10%, 5%, 1% recovery can be flagged.

The system or module can also compute the appropriate amounts of protease to use in each sample, sample fraction, or well based on a variety of criteria, including the total estimated protein computed earlier. The protease may include Glu-C, LysN, Lys-C, Asp-N or Chymotrypsin. The protease is often trypsin. The samples are often digested in a solvent or a buffer, the amount of which can be calculated by the system automatically based on, for example, the amount of protein in the sample or the amount of protease to be used. The amount of protease, solvent, or buffer can also be the same for each well. The devices can add the amount of solvent or buffer to the samples and fractions automatically. The buffer can be a reconstitution buffer. In some embodiments, the device includes a liquid handler, such as a Tecan liquid handler. Some of the devices and methods described herein use chemicals to break proteins into peptides. The systems and modules can assess the amount of protease added to each sample and flag samples that receive too much or too little protease.

The devices can then incubate the samples or fractions with the proteases to break the proteins contained therein into peptides. Digested samples can be assessed for a variety of characteristics, including the range of sizes of peptides produced by the digest. Exemplary characteristics include samples that are not fully digested, samples containing disproportionately small or large peptide fragments, samples containing the wrong average fragments size, or other problems associated with suboptimal digestion. Examples of conditions that might generate a flag include when less than 99%, 98%, 97%, 96%, 95%, 90%, 80%, 70%, 60%, 50%, 40%, 30%, 20%, 10%, 5%, 1% of the peptides in the sample are within a certain fragment size window. Exemplary windows include peptide lengths of 1-30 amino acids, 3-25 amino acids, 5-20 amino acids, 10-20 amino acids, 5-15 amino acids, 15-25 amino acids, 8-12 amino acids, and others. Such samples can be flagged. Some methods include re-digesting the original sample using a different protease or for a different amount of time to obtain a more suitable result.

The protease-treated samples can then be prepared for analysis by mass spec or for storage for use at a later time. Samples are often quenched using a multi-step transfer. Samples can be extracted using solid phase extraction. This often involves a solid phase extraction buffer. The buffer can wash the samples to maximize recovery.

Samples can also be lyophilized. Methods for lyophilizing samples are known in the art. Lyophilized samples can optionally be frozen for use at a later time.

(f) Mass Spectroscopy

Samples prepared as described above are generally analyzed via mass spectrometry, including liquid chromatography mass spectrometry. One or more biomarkers can be measured using mass spectroscopy (alternatively referred to as mass spectrometry). Mass spectrometry (MS) can refer to an analytical technique that measures the mass-to-charge ratio of charged particles. It can be primarily used for determining the elemental composition of a sample or molecule, and for elucidating the chemical structures of molecules, such as peptides and other chemical compounds. MS works by ionizing chemical compounds to generate charged molecules or molecule fragments and measuring their mass-to-charge ratios MS instruments typically consist of three modules (1) an ion source, which can convert gas phase sample molecules into ions (or, in the case of electrospray ionization, move ions that exist in solution into the gas phase) (2) a mass analyzer, which sorts the ions by their masses by applying electromagnetic fields and (3) detector, which measures the value of an indicator quantity and thus provides data for calculating the abundances of each ion present.

Suitable mass spectrometry methods to be used with the present disclosure include but are not limited to, one or more of electrospray ionization mass spectrometry (ESI-MS), ESI-MS/MS, ESI-MS/(MS)ₙ, matrix-assisted laser desorption ionization time-of-flight mass spectrometry (MALDI-TOF-MS), surface-enhanced laser desorption/ionization time-of-flight mass spectrometry (SELDI-TOF-MS), tandem liquid chromatography-mass spectrometry (LC-MS/MS) mass spectrometry, desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS), atmospheric pressure photoionization mass spectrometry (APPI-MS), APPI-MS/MS, and APPI-(MS)ₙ, quadrupole mass spectrometry, Fourier transform mass spectrometry (FTMS), and ion trap mass spectrometry, where n can be an integer greater than zero.

LC-MS can be commonly used to resolve the components of a complex mixture. LC-MS method generally involves protease digestion and denaturation (usually involving a protease, such as trypsin and a denaturant such as, urea to denature tertiary structure and iodoacetamide to cap cysteine residues) followed by LC-MS with peptide mass fingerprinting or LC-MS/MS (tandem MS) to derive sequence of individual peptides. LC-MS/MS can be used for proteomic analysis of complex samples where peptide masses may overlap even with a high-resolution mass spectrometer. Samples of complex biological fluids like human serum may be first separated on an SDS-PAGE gel or HPLC-SCX and then run in LC-MS/MS allowing for the identification of over 1000 proteins. In addition to peptide analysis, LC-MS can also be used for evaluating lipids such as generating lipid profiles. For example, HPLC-Chip/MS, UPLC/MS, UPLC/FT-MS, and LC-TOF/MS can be used to generate high resolution lipid profiles. In some cases, the lipids that can be analyzed using these approaches are in a particular mass range such as, for example, from about 100 to about 2000 Daltons, from about 200 to about 1900 Daltons, or from about 300 to about 1800 Daltons. GC-MS such as GC-TOF can also be used for lipid analysis. Accordingly, samples comprising lipids can be processed and/or analyzed pursuant to the systems and methods described herein to evaluate one or more lipid biomarkers. Likewise, other biomolecules such as metabolites can also be evaluated using various mass spectrometry instruments and systems. Examples of MS instruments suitable for processing samples for detection and analysis of metabolites include gas chromatography/MS (GC/MS), liquid chromatography/MS, or capillary electrophoresis/MS (CE/MS). Various sample fractionation methods can be utilized in the systems and methods described herein. Examples of fractionation methods include gas chromatography, liquid chromatography, capillary electrophoresis, or ion mobility. Ion mobility can include differential ion mobility spectrometry (DMS) and asymmetric ion mobility spectrometry.

While multiple mass spectrometric approaches are compatible with the methods of the disclosure as provided herein, in some applications it is desired to quantify proteins in biological samples from a selected subset of proteins of interest. One such MS technique that is compatible with the present disclosure is Multiple Reaction Monitoring Mass Spectrometry (MRM-MS), or alternatively referred to as Selected Reaction Monitoring Mass Spectrometry (SRM-MS).

The MRM-MS technique involves a triple quadrupole (QQQ) mass spectrometer to select a positively charged ion from the peptide of interest, fragment the positively charged ion and then measure the abundance of a selected positively charged fragment ion. This measurement is commonly referred to as a transition and/or transition ion.

Alternately or in combination, a sample prepared for MS analysis is supplemented with at least one labeled protein or polypeptide, such that the labeled protein or polypeptide migrates with or near a protein or fragment in a sample. In some cases a heavy-isotope labeled protein or fragment is introduced into a sample, such that the labeled protein or fragment migrates near but not identically to an unlabeled, native version of the protein in the sample. With an understanding of the position of the labeled protein and the impact of its labeling on MS migration, one can readily identify the corresponding native protein in the sample. In some cases a panel of labeled proteins or protein fragments are adopted, so that a panel of proteins is readily assayed from MS data but, concurrently, untargeted data of a broad range of proteins or fragments is also obtained.

In some applications the MRM-MS is coupled with High-Pressure Liquid Chromatography (HPLC) and more recently Ultra High-Pressure Liquid Chromatography (UHPLC). In other applications MRM-MS can be coupled with UHPLC with a QQQ mass spectrometer to make the desired LC-MS transition measurements for all of the peptides and proteins of interest.

In some applications the utilization of a quadrupole time-of-flight (qTOF) mass spectrometer, time-of-flight time-of-flight (TOF-TOF) mass spectrometer, Orbitrap mass spectrometer, quadrupole Orbitrap mass spectrometer or any Quadrupolar Ion Trap mass spectrometer can be used to select for a positively charged ion from one or more peptides of interest. The fragmented, positively charged ions can then be measured to determine the abundance of a positively charged ion for the quantitation of the peptide or protein of interest.

In some applications the utilization of a time-of-flight (TOF), quadrupole time-of-flight (qTOF) mass spectrometer, time-of-flight time-of-flight (TOF-TOF) mass spectrometer, Orbitrap mass spectrometer or quadrupole Orbitrap mass spectrometer is used to measure the mass and abundance of a positively charged peptide ion from the protein of interest without fragmentation for quantitation. In this application, the accuracy of the analyte mass measurement can be used as selection criteria of the assay. An isotopically labeled internal standard of a known composition and concentration can be used as part of the mass spectrometric quantitation methodology.

In some applications, time-of-flight (TOF), quadrupole time-of-flight (qTOF) mass spectrometer, time-of-flight time-of-flight (TOF-TOF) mass spectrometer, Orbitrap mass spectrometer or quadrupole Orbitrap mass spectrometer is used to measure the mass and abundance of a protein of interest for quantitation. In this application, the accuracy of the analyte mass measurement can be used as selection criteria of the assay. Optionally this application can use proteolytic digestion of the protein prior to analysis by mass spectrometry. An isotopically labeled internal standard of a known composition and concentration can be used as part of the mass spectrometric quantitation methodology.

In some applications, various ionization techniques can be coupled to the mass spectrometers provide herein to generate the desired information. Non-limiting exemplary ionization techniques that are used with the present disclosure include but are not limited to Matrix Assisted Laser Desorption Ionization (MALDI), Desorption Electrospray Ionization (DESI), Direct Assisted Real Time (DART), Surface Assisted Laser Desorption Ionization (SALDI), or Electrospray Ionization (ESI).

In some applications, HPLC and UHPLC can be coupled to a mass spectrometer a number of other peptide and protein separation techniques can be performed prior to mass spectrometric analysis. Some exemplary separation techniques which can be used for separation of the desired analyte (for example, lipid, metabolyte, or polypeptide such as a protein) from the matrix background include but are not limited to Reverse Phase Liquid Chromatography (RP-LC) of proteins or peptides, offline Liquid Chromatography (LC) prior to MALDI, 1 dimensional gel separation, 2-dimensional gel separation, Strong Cation Exchange (SCX) chromatography, Strong Anion Exchange (SAX) chromatography, Weak Cation Exchange (WCX), and Weak Anion Exchange (WAX). One or more of the above techniques can be used prior to mass spectrometric analysis.

The methods, devices, and modules described herein can be optimized for increased throughput. Some of the methods can be performed at a rate of 1, 2, 3, 4, 5, 6, 7, 8, 9, or 10 injections per hour. As such, the methods allow for near real-time analysis of quality controls and data, enabling users to make decisions rapidly.

Prior to loading the samples onto the mass spectrometer for analysis, the device is often assessed beforehand with a quality control run to determine if the machine is operating within appropriate parameters. The quality control run can include assessing a curve generated using standard control samples. The samples often comprise aliquots of a known sample that has been previously characterized. In some cases, using aliquots of the same sample across multiple experiments or runs can allow for data generated in each experiment or run to be compared to data generated in other experiments or runs. In some cases, the use of aliquots of the same sample for the quality control runs allows for data between runs to be normalized for comparison. In some cases, the quality control run allows for the assessment of the sensitivity of the instrument. Quality control runs can be repeated using the same sample to determine if the machine is accurately and reproducibly assessing samples.

Alternatively or in addition, assessing the quality control run can include determining if the run detected and correctly identified or classified a percentage of standard features, such as peptides, known to be in the sample, in the stable isotope control spike, or at known concentrations. For example, the run can be flagged if less than 99%, 98%, 97%, 96%, 95%, 90%, 80%, 70%, 60%, 50%, or 25% of known peptides or features are detected. The run can also be flagged if it does not detect a minimum acceptable number of features with specific charge states, such as 1, 2, 3, 4, 5, or more. Assessing the quality control can also include determining the concentration of peptides or proteins known to be present in the sample. The run can be flagged if the calculated concentration is within has a percent error of 1%, 2%, 3%, 4%, 5%, 10%, 15%, 20%, 25%, 30%, 40%, 50% or 75% compared to a known sample. In some cases, quality control is assessed by determining the detection of a minimum number of features that possess a specific charge state, a minimum number of features, selected analyte signal that meets at least one threshold, presence of known contaminants, mass spectrometer peak shape, chromatographic peak shape, or any combination thereof. For example, an analyte signal may be evaluated to determine if the signal exceeds a minimum threshold or is above a maximum threshold. In some cases, the peak shape is evaluated to determine whether the peak corresponds to a certain desired data quality level, e.g., based on previous analyses. The right can be flagged if the overall retention times are inconsistent with those determined from previous runs or other runs within the same experiment. Retention time can be combined with total ion current as part of the comparison. Major shifts in retention time may be caused by leaky chromatography systems. In some cases, some shifting is expected due to the variability of conditions between runs. Liquid chromatography pressure traces can also be compared to those from previous runs or runs within the same experiment. In some cases, retention time and pressure trace analysis are used to assess the status of the liquid chromatography column. In some cases, the system will alert the operator to replace the column. The quality control runs can also be used to determine if the instrument is detecting an acceptable number of features with desired charge states or m/z ranges.

Such assessments can be automated. These parameters can include predetermined tolerances. The system can notify the appropriate user or supervisor if the quality control run does not perform as expected. The sample run can be postponed if the instrument is outside of defined performance tolerances. Such quality control runs can flag subsequent sample runs.

The methods and workflows described herein can be implemented using a series of sample processing modules and sample analysis modules. Sample processing modules such as protein processing modules or lipid processing modules can comprise or control one or more physical devices or instruments and obtain output from the devices or instruments. This output may be evaluated by a corresponding sample analysis module for one or more quality control metrics. For example, a processing module configured to determine protein concentration may process a sample to determine protein concentration using a protein concentration analyzer. A corresponding analysis module may then apply a tag and/or rule to terminate, suspend, restart, or modify the workflow (e.g., changing or restarting one or more steps in the workflow) based on an analysis of the output. For example, a rule may specify terminating the workflow when the protein concentration is below a minimum threshold concentration.

Worklists for quality control and sample runs can be generated automatically, as described above. Worklists can include ordering samples and the appropriate injection volumes used for each sample to standardize the mass loaded onto the liquid chromatography column. As described above, the worklist can process the quality control samples in the same order for each worklist (e.g. first, middle, and last) to provide sample and/or worklist normalization during data analysis.

The instrumentation generally downloads the worklist and imports it into the software controlling the LCMS. The user can manually verify that the appropriate worklist and sample injection order has been loaded into the software, if desired.

The system can process run data and develop quality control metrics. The system can flag or tag samples or data that do not meet desired quality control metrics. The tags can inform downstream sample processing and/or data evaluation or analysis. Tags can comprise rules that dictate downstream steps in the workflow. In some cases, a sample analysis module that assesses may comprise one or more rules. For example, a sample analysis module may be configured to evaluate successful ionization of the sample for mass spectrometry analysis (e.g., electrospray ionization). If the ionization signal is below a first threshold, one of the rules may cause the workflow to be shutdown. For example, the rule may be a configurable rule established based on prior experiments/sample analysis or a preset rule that determined a signal below a certain threshold would produce a data set that is inadequate for the experimental goal (e.g., when the experiment is for detection of a low abundance protein/peptide). Alternatively, the rule may specify continued sample processing and/or analysis when the experimental goal is to detect a high abundance protein/peptide. The rules and/or rule parameters (e.g., signal threshold that determines whether the sample or data is flagged/tagged) may be altered depending on the specific experimental goal or target protein/peptide. A sample analysis module can apply a tag with no rules (e.g., the sample or data is marked with a tag for information purposes only). Alternatively, a sample analysis module may apply a tag with a plurality of rules determining downstream processing or analysis. The rules can include terminating the workflow, suspending the workflow (e.g., for instrument calibration), restarting the workflow (optionally altering the workflow and restarting, e.g., restarting workflow while increasing duration of protease digestion due to detection of inefficient digestion), or altering the workflow (e.g., injecting more sample due to lower than expected signal strength). In some cases, a sample analysis module evaluates signal strength in mass spectrometry analysis such as, for example, tandem mass spectrometry. Sometimes, a sample analysis module evaluates successful digestion of a sample. A sample analysis module can evaluate sample concentration and apply a tag comprising one or more rules based on the determined concentration. For example, a low sample concentration may trigger a rule that terminates or suspends the workflow or subsequent sample processing and/or analysis such as when the workflow is attempting to identify a low abundance biomarker. As another example, a sample analysis module detects the presence of a normally high abundance protein or peptide above a predefined threshold (e.g., an abundant cellular protein such as actin, tubulin, or heat shock protein, or polypeptide thereof or abundant serum proteins such as immunoglobulins and albumin in serum samples). In this example, the sample is tagged because the workflow is a depleted proteomics workflow that attempts to amplify or enhance the signal of a low abundance protein by depleting certain high abundance proteins. Accordingly, a rule is applied that causes the workflow to terminate or suspend in the case that the protein or polypeptide that exceeds the threshold was depleted. For example, a different rule may be applied for a sample depending on whether it is a serum sample, a cell sample, a saliva sample, or other biological sample described herein. In some instances, a rule can specify terminating, suspending, or restarting the workflow when the quality control metric indicates an insufficient quantity, insufficient concentration, insufficient signal strength, background, or contamination that disrupts detection of at least one target peptide.

In some cases, a tag, rule, or gating module is configured based on other sample data or data analysis. The rule may be trained or configured according to user-specified outcomes. For example, past samples may be analyzed using at least one algorithm such as a predictive model or classifier based on features corresponding to QC control metrics and a user-defined outcome. In some cases, the algorithm is a machine learning algorithm that can be trained with a training data set using supervised learning to generate a trained machine learning model or classifier. For example, a user may label previously processed/analyzed samples with an outcome such as, for example, useful/not useful/inconclusive, failure to detect one or more targeted biomarker(s), etc. The algorithm can then be trained using the feature set comprising QC metrics and the outcomes to then generate predictions on sample processing/analysis outcome based on QC metrics evaluated by sample analysis module(s). In some cases, this is an ongoing analysis while the workflow is in operation, and one or more gating steps along the workflow, a rule may be applied to determine whether to continue, terminate, suspend, restart, or alter the workflow. For example, a trained model or classifier may be used to predict a likelihood of sample processing/analysis outcome failure at one or more steps along the workflow. Early on, the QC metrics may be not generate sufficiently reliable predictions, leading the rules to continue the workflow (e.g., the rule requires a certain threshold confidence of a predicted failure in order to terminate a workflow). Later on in the workflow, sufficient QC metrics may have been assessed such that a model that incorporates these features may generate an outcome prediction with sufficient reliability. For example, in some cases, a rule for terminating, suspending, restarting, or altering a workflow (e.g., modifying downstream processing and/or analysis) is triggered by a predicted outcome (e.g., outcome failure) having a confidence interval of at least about 70%, 75%, 80%, 85%, 90%, 95%, or 99%.

The speed and efficiency of mass spectrometric workflows are greatly improved at least in part through the application of these data analysis modules that assess for successful sample processing throughout various steps of the mass spectrometric workflow and automatically respond to the assessment using special rules that modulate the workflow based on the measured quality control metrics. The rules enable a streamlined and automated methodology through at least a part or all of the mass spectrometric workflow. Thus, the systems and methods disclosed herein provide a technical solution that improves the functioning of mass spectrometric systems and instruments for carrying out sample processing and analysis workflows.

If the sample is not yet in liquid form, the system can put the sample back into liquid form. This can include reconstituting the sample, including lyophilized samples. This process can include reconstituting the sample in a buffer, such as a buffer suitable for injection into the LCMS. In some embodiments, 6PRB buffer is used. The system can compute the amount of sample buffer volume to use when reconstituting each sample. The amount of buffer can be calculated in some circumstances to yield standardized peptide loading across all samples into the LCMS. In other examples, the amount of buffer is the same across some or all of the wells independent of peptide loading. The amount of buffer can also be controlled to match instrument configurations. Such calculations can be processed as a worklist, which can be archived automatically. The worklist can control a liquid handling station processing the samples. The liquid handling station can dispense the appropriate amount of reconstitution buffer into each sample or well. This can include standard or control wells containing known peptides for quality control assessments. Samples and controls that do not receive the appropriate amount of sample buffer can be flagged.

In some embodiments, the samples are spiked with stable isotope samples, as described above. Some of the devices and methods described herein comprise spiking the samples during the sample reconstitution step. Samples that are spiked with the wrong stable isotope sample, the wrong amount of stable isotope sample, or samples that improperly receive or do not receive stable isotope samples can be flagged.

Plates and samples are often centrifuged prior to being loaded onto the LCMS. Such steps serve to standardize reconstituted samples to the bottom of the well or container. Centrifugation can also help to remove or minimize bubbles in each sample. Modules or systems can therefore include centrifuges. Samples that are determined to contain bubbles or that were centrifuged improperly, for example because they were centrifuged for the wrong amount of time or at the wrong speed, can be flagged.

Samples can then be fed into a module comprising LCMS for analysis. The LIMS can use a template to create a worklist for the mass spec. The worklist can contain appropriate settings for each well. Blanks can be inserted into the process as appropriate. Sample position can be randomized or partially randomized using certain criteria to prevent plate position effects. The LCMS workstation can import the worklist automatically for each well. The system can begin processing the samples by injecting the samples into the liquid chromatograph, which can inject the samples into the mass spectrometer. The module can assess the rate of injection into the liquid chromatograph, the rate of liquid passing through each phase, the rate of separation, and the rate of elution. Each of these measurements can cause a sample or step to be flagged.

Data from each run can be analyzed automatically or manually. The data are frequently analyzed for quality control purposes. If the quality of the data do not meet certain criteria, root cause analysis can be performed. The affected samples can also be run again, if necessary. Controls can be used to determine if an experiment's variability is within acceptable limits. Failure of any quality control analysis can cause a sample or an experiment to be flagged.

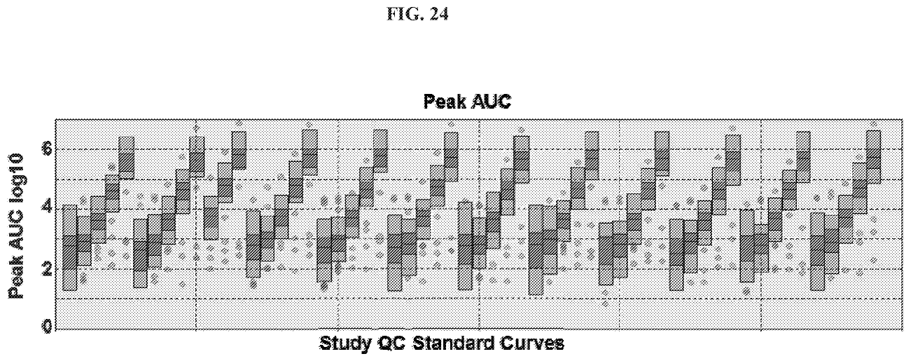

One example of data quality includes analysis of standard curves for spiked standards, if used. If the areas under the curve for spiked samples fall within the expected ranges, the sample passes one quality control check. This analysis can include a check to ensure that peak areas under the curve increase with spike-in concentration. In addition, whether RTs or other values fall within expected range can also be assessed as a quality control check. This is often done using a visual assessment of plots generated with API code. Alternatively or in addition, standard curve data evaluation can be automated using software that can, for example, generate an email or alert if the data fail to pass standard curve tests. Exemplary standard curve data is shown in FIG. 24.

Another example of data quality includes analysis of the processes and methods. The processes can pass quality controls if Coefficients of Variation are acceptable, and if peak areas are within expected ranges. In addition, RTs should fall within expected ranges in some of the disclosed methods. This can be accomplished with a visual assessment of plots generated with API code.

In some cases, only values falling within specific ranges are reported. For example, assayed protein concentrations or other biomarker levels below a given cutoff indicate a failed assay in some cases, while assayed protein concentrations or other biomarker levels above a threshold may indicate a suspect or inaccurate reading.

Useful analyte capture agents used in practice of methods and devices described herein include but are not limited to antibodies, such as crude serum containing antibodies, purified antibodies, monoclonal antibodies, polyclonal antibodies, synthetic antibodies, antibody fragments (for example, Fab fragments); antibody interacting agents, such as protein A, carbohydrate binding proteins, and other interactants; protein interactants (for example avidin and its derivatives); peptides; and small chemical entities, such as enzyme substrates, cofactors, metal ions/chelates, aptamers, and haptens. Antibodies may be modified or chemically treated to optimize binding to targets or solid surfaces (for example biochips and columns).

### Computational Pipeline for Profile and DPS Proteomics

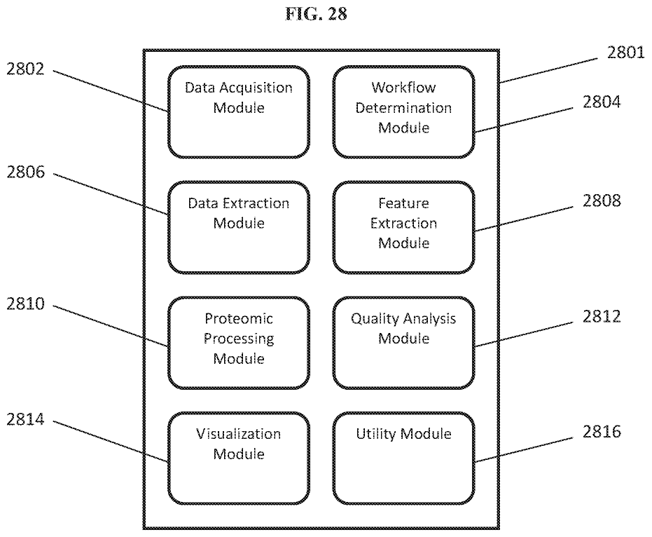

Disclosed herein are computational pipelines for analysis of data generated from methods such as profile and DPS proteomics. The computational pipeline comprises a plurality of data processing modules that transform, convert, or otherwise manipulate data. The data is often mass spectrometric data such as protein mass spectrometric data generated from a sample. The data processing modules carry out computational steps that process the data from the preceding module. Data processing modules perform various data manipulation functions such as data acquisition, workflow determination, data extraction, data preparation, feature extraction, proteomic processing, quality analysis, data visualization, and other functions for data exploration, visualization, and/or monitoring. The computational pipeline can utilize two or more of the data processing modules to generate usable data. In some instances, the computational pipeline uses at least 1, 2, 3, 4, 5, 6, 7, 8, 9, 10, 11, 12, 13, 14, 15, 16, 17, 18, 19, 20, 30, 40, 50, 60, 70, 80, 90, or 100 or more data processing modules, and/or no more than 1, 2, 3, 4, 5, 6, 7, 8, 9, 10, 11, 12, 13, 14, 15, 16, 17, 18, 19, 20, 30, 40, 50, 60, 70, 80, 90, or 100 or more data processing modules. As shown in FIG. 28, the computational pipeline or workflow can be performed by a series of data processing modules such as one or more of a data acquisition module 2802, a workflow determination module 2804, a data extraction module 2806, a feature extraction module 2808, a proteomic processing module 2810, a quality analysis module 2812, a visualization module 2814, a utility module 2816, or any other data processing module. The modules can be part of a software application or package 2801.

### Data Acquisition

Provided herein are systems, devices, and methods implementing computational pipelines for processing of data such as data generated by profile and DPS proteomics. Computational pipelines often comprise a data acquisition process carried out by a data acquisition module. The data acquisition module carries out one or more computational steps for acquiring data such as mass spectrometric data. The acquired data can be passed on to at least one subsequent data processing module for further manipulation and/or analysis. Sample data that is processed by the data acquisition module can be acquired and/or stored by the module as a data file such as a single LCMS data file. Multiple data sets corresponding to different samples are sometimes acquired together or sequentially. The data acquisition module optionally generates a single LCMS data file for each sample such as for each sample well for a registered study.

Data acquisition can be initiated as part of a computational workflow. The workflow or the data acquisition is optionally queued by registered instruments such as mass spectrometric or data analysis instruments. When data acquisition is initiated or instructed, software such as an application programming interface (API) is usually tasked with performing the requisite computational steps. The data acquisition process is often performed by at least one software module in a product package. In various cases, the API comprises the data acquisition module that carries out data acquisition. The data is typically acquired from a data source such as a mass spectrometry machine.

The data acquisition module optionally includes a data transfer process following data acquisition. The data transfer process often entails copying and/or storage of the acquired data into a storage or memory (e.g., a database). The storage is sometimes shared primary data storage. The transferred data can be stored in various formats compatible with data storage such as a LCMS data file for each sample. In some instances, the data acquisition undergoes verification to confirm that each LCMS data file was copied to storage such as shared primary data storage. The verification can be a quality assessment that includes a process control step to ensure the data acquisition and/or data transfer was carried out. The quality assessment can also include a quality control step for evaluating quality of the acquired data. Sample data failing the quality assessment variously results in flagging the sample data, for example so as to indicate in its output that there is an issue in the analysis, or may result in pausing or cancelling the computational workflow so as to address the workflow or sample data issue, for example by re-attempting data acquisition (or any of the steps comprising data acquisition), or by discarding the sample data from the computational workflow. The data transfer process is often performed by at least one software module in a product package.

### Determining Workflow

Provided herein are systems, devices, and methods implementing computational pipelines (also referred to as computational workflow) for processing of data such as data generated by profile and DPS proteomics. Computational pipelines often comprise a workflow determination process carried out by a workflow module. The workflow module carries out one or more steps for determining a computational workflow for processing and/or analysis of data such as mass spectrometric data. The workflow module can carry out any of the steps described herein as part of a product package (e.g., a package for an end-to-end mass spectrometric workflow that includes study planning/experimental design, mass spectrometric sample processing and concurrent quality assessments, and computational workflow for data analysis). The workflow module often performs a parsing step such as on a worklist, also referred to as a protocol. The worklist serves to provide instructions for any or each step in a process and can also record experiment-specific data for samples. In some cases, worklists contain scripts used by devices such as computational devices and mass spectrometric devices. Worklists can include various workflow parameters or information relevant to workflow parameters such as random sample ordering and appropriate volumes used. Control samples are often processed in the same order for every worklist. This order can include control samples being used at the beginning, middle, and end of specific steps within the experiment. As such, control samples can help normalize samples and worklists during data analysis. This can include sample label information and reagents information, including concentrations and lot numbers used with a particular set of samples. Worklists used with a particular process can be stored with, archived, or associated with the corresponding experiment for later reference. In some instances, the worklist includes various parameters from a preceding experimental design workflow and/or a sample processing workflow. The parameters can include any of biomarkers or biomarker candidates, the method used to generate the biomarkers or biomarker candidates (e.g., manually curated, automated, or a combination thereof), precursors and/or ion transitions selected for mass spectrometric analysis, desired or threshold statistical metrics (e.g., p-value, CV) for the study result/output, number of samples, number of replicates, depletion of abundant proteins, the identity of depleted proteins, protein enrichment (e.g., by purification such as immunoprecipitation), liquid chromatography parameters, mass spectrometric instrument parameters, and other parameters relating to the overall mass spectrometric workflow. Alternatively, the preceding parameters can be obtained separately from the worklist and used to generate a corresponding computational workflow suitable for carrying out data analysis based on the parameters.

The workflow module can read the worklist by parsing the worklist to extract workflow parameters and/or information relating to workflow parameters. Following parameter extraction, the workflow module usually sets the parameters for the workflow. The workflow module optionally determines appropriate parameters based on information extracted from the worklist. For example, a workflow parameter may be adjusted to account for worklist information indicating the sample is a dried blood spot or that the sample comprises reference biomarkers that require certain computational steps for accurate detection. Workflow parameters can include the mass spectrometric method, pump model number, sample type, sample name, data acquisition rate minimum and/or maximum, concentration, volume, plate position, plate barcode, and/or other parameters related to sample processing and/or analysis.

The workflow module often performs a controller step for determining the pipeline computations and steps to run based on the method (e.g., LCMS method) used to generate the data file and parameters gathered from parsing the worklist. In some cases, the data file and parameters are defined in the instrument method and study such as a LCMS method. The pipeline computations and steps constitute a computation flow that is optionally set in a computational group. Computational groups allow modularization of pipeline computational flow such that each computational flow can be reconfigured, for example, by combining various computation flow modules. The modularization allows the reconfiguration of computational flow to be performed more easily compared to non-modular computational flow configurations. For example, the computational groups can be reconfigured depending on study requirements and/or the nature of the sample being processed such as whether the sample is a blank or QC sample.

Workflow determination can be initiated as part of a computational workflow. The computational workflow or the workflow determination is optionally queued by registered instruments such as mass spectrometric or data analysis instruments. When workflow determination is initiated or instructed, software such as an application programming interface (API) is usually tasked with performing the requisite computational steps. In various cases, the API comprises the workflow module that carries out workflow determination. The worklist is typically acquired from a data source such as a mass spectrometry machine or computing device.

The workflow module optionally includes a quality assessment process following workflow determination. In some instances, the workflow determination comprises a quality assessment step to confirm that the computation flow has been properly configured. The quality assessment can include a process control step to ensure the workflow determination step is carried out. The quality assessment can also include a quality control step for evaluating quality of the workflow determination. For example, information from the worklist may indicate issues such as incompatibility between information from the worklist and available workflow parameters or options. Workflow parameters failing the quality assessment variously results in flagging the sample data, for example so as to indicate in its output that there is an issue in the analysis, or may result in pausing or cancelling the computational workflow so as to address the workflow issue, for example by re-attempting workflow determination (or any of the steps comprising workflow determination), or by discarding the sample data from the computational workflow.

The workflow determination module may configure a computational workflow to perform a quality assessment on at least one of the subsequent data processing or computational steps carried out during the execution of the computational workflow. In some cases, the quality assessment evaluates the data output for a particular data processing step such as by using a quality control metric (e.g., elution time, signal-to-noise ratio (SNR), signal strength/intensity, pairwise fragment ratios, and various other QC metrics). The quality assessment can include an evaluation of the data processing step itself and/or the performance of a data processing module such as identifying an expected output or metric indicative of successful data processing/manipulation. In some cases, a mislabeled or corrupted file can result in the data not being correctly saved or rendered inaccessible.

The computational workflow can be informed by upstream quality assessments carried out during sample processing such as during mass spectrometric evaluation of a sample. For example, a quality assessment for elution time can be performed for one or more samples during mass spectrometric analysis. The elution time for measured sample proteins or peptides may vary between samples such as sample replicates or experimental and control samples. Accordingly, a quality assessment that measures or otherwise accounts for elution time can enable the computational workflow to normalize or adjust one or more data sets.

### Data Extraction

Provided herein are systems, devices, and methods implementing computational pipelines for processing of data such as data generated by profile and DPS proteomics. Computational pipelines often include a data extraction module. Computational pipelines often comprise a data extraction process carried out by a data extraction module. The data extraction module carries out one or more computational steps for extracting data such as mass spectrometric data. The extracted data can be passed on to subsequent data processing modules for further manipulation and/or analysis. Sample data that is extracted by the data extraction module can be obtained from each LCMS data file for downstream processing. In some instances, the total ion chromatogram (TIC) is extracted, optionally using calculations determined by the chromatography group. Sample data is sometimes extracted from multiple data files corresponding to different samples that are acquired together or sequentially.

The data extraction module can perform one or more computational steps to carry out data extraction of instrument data (e.g., an MSActuals step). In some cases, the MSActuals step includes extracting LCMS instrument chromatograms into a file such as an “actuals” file. The data extraction module sometimes performs at least one computational step for extracting and converting spectral data into a different format (e.g., an MS1Converter step). For example, an internal spectral data stored using a first format may be converted into a second format such as APIMS1. In some cases, internal spectral data is converted into APIMS1 format for at least one of acquired time range, device name and type, fragment voltage, ionization mode, ion polarity, mass units, scan type, spectrum type, threshold, sampling period, total data point, total scan counts, and other information relevant to the spectral data. The data extraction module can carry out any of the computation steps described herein as part of a product package.

The data extraction module optionally performs data extraction for MS2 data (e.g., in the case of tandem mass spectrometry) and conversion into a different format (e.g., a tandem data extraction step). For example, the MS2 data stored in a first spectral data format may be converted by the data extraction module into a second data format such as Mascot generic format (MGF). The conversion is often performed using an application library.

Next, the data extraction module can determine the chromatography group collected from a prior step. In some cases, the data extraction module then performs at least one computational step extracting total ion chromatograms (TIC) using an algorithm and saves it in a database.

Data acquisition can be initiated as part of a computational workflow. The workflow or the data extraction is optionally queued by registered instruments such as mass spectrometric or data analysis instruments. When data extraction is initiated or instructed, software such as an application programming interface (API) is usually tasked with performing the requisite computational steps. In various cases, the API comprises the data extraction module that carries out data extraction. The data is typically acquired from a data source such as a mass spectrometry machine.

In some instances, the data extraction process undergoes a quality assessment step to assess successful data extraction and/or quality of the extracted data. The quality assessment can include a process control step to ensure the data extraction was carried out. The quality assessment can also include a quality control step for evaluating quality of the acquired data. Sample data failing the quality assessment variously results in flagging the sample data, for example so as to indicate in its output that there is an issue in the analysis, or may result in pausing or cancelling the computational workflow so as to address the workflow or sample data issue, for example by re-attempting data extraction (or any of the steps comprising data extraction), or by discarding the sample data from the computational workflow.

### Data Preparation

Provided herein are systems, devices, and methods implementing computational pipelines for processing of data such as data generated by profile and DPS proteomics. Computational pipelines often comprise a data preparation process carried out by a data preparation module. The data preparation module carries out one or more computational steps for preparing data such as mass spectrometric data for further analysis. Following data preparation, the sample data can be passed on to subsequent data processing modules for further manipulation and/or analysis. Sample data that is prepared by the data preparation module can be obtained from a preceding module such as the data extraction module. Data preparation is sometimes performed on sample data obtained from multiple data files corresponding to different samples that are acquired together or sequentially. The data preparation module can carry out any of the computation steps described herein as part of a product package.

The data preparation module can perform one or more computational steps to carry out data preparation. Sometimes, the data preparation module performs a step creating serialized MS1. This step often entails converting a spectral data file into a new format for analysis. For example, the data preparation module can convert spectral data in an APIMS1 file format into a java serialized format suitable for downstream processing. Sometimes, the data preparation module performs one or more computation steps for loading actuals into a database. For example, the data preparation module can put scans and read backs during those scans into the database.

Data preparation can be initiated as part of a computational workflow. The workflow or the data preparation is optionally queued by registered instruments such as mass spectrometric or data analysis instruments. When data preparation is initiated or instructed, software such as an application programming interface (API) is usually tasked with performing the requisite computational steps. In various cases, the API comprises the data preparation module that carries out data preparation. The data is typically obtained from a data source such as a mass spectrometry machine.

In some instances, the data preparation process undergoes a quality assessment step to assess successful data preparation and/or quality of the prepared data. The quality assessment can include a process control step to ensure the data preparation was carried out. The quality assessment can also include a quality control step for evaluating quality of the prepared data. Sample data failing the quality assessment variously results in flagging the sample data, for example so as to indicate in its output that there is an issue in the analysis, or may result in pausing or cancelling the computational workflow so as to address the workflow or sample data issue, for example by re-attempting data preparation (or any of the steps comprising data extraction), or by discarding the sample data from the computational workflow.

### Feature Extraction

Provided herein are systems, devices, and methods implementing computational pipelines for processing of data such as data generated by profile and DPS proteomics. Computational pipelines often comprise a feature extraction process carried out by a feature extraction module. The feature extraction module carries out one or more computational steps for extracting features from data. For example, initial molecular features can be extracted using an algorithm for peak detection. Sometimes, the extracted features are stored in parallel sections to a java serialized file for downstream processing. The initial molecular features can then be refined using LC and isotopic profiles. Next, the properties of the refined molecular features can be computed. Following feature extraction, the sample data comprising extracted features can be passed on to subsequent data processing modules for further manipulation and/or analysis. Sample data that undergoes feature extraction by the feature extraction module can be obtained from a preceding module such as the data preparation module. Feature extraction is sometimes performed on sample data obtained from multiple data files corresponding to different samples that are acquired together or sequentially. The feature extraction module can carry out any of the computation steps described herein as part of a product package.

The feature extraction module can perform one or more computational steps to carry out feature extraction. Often, each molecular feature extraction that has been obtained using any of the preceding steps is then combined for analysis. Sometimes, the feature extraction module performs a step combining MS1 peak detection files (e.g., detected MS1 peaks). In certain cases, the feature extraction module performs a step filtering and/or deisotoping MS1 peaks after the features have been combined. For example, a combination of filtering and clustering techniques are applicable to raw peaks for evaluation of the peaks, and the evaluated peaks may be subsequently written to a database. Sometimes, the feature extraction module performs a step computing the MS1 properties associated with a given set of molecular features, which are optionally stored in a database. In many instances, the feature extraction module performs at least one step obtaining and/or calculating the ms1p total read back. For example, the feature extraction module can interpolate the MS1 data points, set the quality data for each, and save to the database. Sometimes, the feature extraction module performs at least one step cleaning up the MS1 peak detection files. Alternatively or in combination, the feature extraction module performs at least one step for computation of MS1 peak cleanup. Finally, the feature extraction module often performs at least one step carrying out removal of temporary files such as from the memory of the computing machines used for the computational workflow.

Feature extraction can be initiated as part of a computational workflow. The workflow or the feature extraction is optionally queued by registered instruments such as mass spectrometric or data analysis instruments. When feature extraction is initiated or instructed, software such as an application programming interface (API) is usually tasked with performing the requisite computational steps. In various cases, the API comprises the feature extraction module that carries out feature extraction. The data is typically acquired from a data source such as a mass spectrometry machine.

In some instances, the feature extraction process undergoes a quality assessment step to assess successful feature extraction and/or quality of the extracted features. The quality assessment can include a process control step to ensure the feature extraction was carried out. The quality assessment can also include a quality control step for evaluating quality of the extracted features. Sample data failing the quality assessment variously results in flagging the sample data, for example so as to indicate in its output that there is an issue in the analysis, or may result in pausing or cancelling the computational workflow so as to address the workflow or sample data issue, for example by re-attempting feature extraction (or any of the steps comprising feature extraction), or by discarding the sample data from the computational workflow.

### Proteomic Processing

Provided herein are systems, devices, and methods implementing computational pipelines for processing of data such as data generated by profile and DPS proteomics. Computational pipelines often comprise a proteomic processing process carried out by a proteomic processing module. The proteomic processing module carries out one or more computational steps for proteomic processing of data such as mass spectrometric data. The proteomic processing module is able to propose peptide sequences and possible protein matches for spectral data such as MS2 data. Following proteomic processing, the sample data can be passed on to subsequent data processing modules for further manipulation and/or analysis. Sample data that undergoes proteomic processing by the proteomic processing module can be obtained from a preceding module such as the feature extraction module. Proteomic processing is sometimes performed on sample data obtained from multiple data files corresponding to different samples that are acquired together or sequentially. The proteomic processing module can carry out any of the computation steps described herein as part of a product package.

The proteomic processing module can perform one or more computational steps to carry out proteomic processing. Sometimes, the proteomic processing module performs at least one computational step creating at least one list for targeted data acquisition such as for neutral mass clustering and/or molecular feature extractions. The proteomic processing module can perform at least one computational step accessing the mass differences and charge, and optionally performing a correction on the data file such as an MGF file by incorporating mass differences, charge, or other information relating to the proteomic data. For example, the precursor masses and charges from the MGF file can be matched to the refined values developed during the molecular feature extraction carried out by the feature extraction module (e.g., the refined values are the refined molecular features generated by refining initial molecular features using LC and isotopic profiles). The MGF file values can be corrected when they differ from the refined values developed by the feature extraction module.

In certain instances, the proteomic processing module performs at least one computational step carrying out a proteomic data search. Typically, this step includes searching for proteins and/or peptides against a protein database. An example includes searching for proteins using an OMSSA engine against the UniProt Human/Mouse/Rat/Bovine (HMRB) FASTA database. Later validation steps can be prepared for by matching against the database itself and a reversed version, and results from the latter search are usable for developing false discovery rate (FDR) statistics. Searching for proteins against a protein database can include performing at least one of the following steps: setting the search mode to OMSSA, setting up the forward database (e.g., HMRB) for search in OMSSA, performing the forward OMSSA search, setting up the reversed database (HMRB reversed) for the search in OMSSA, and performing the reverse search in OMSSA.

Sometimes, the proteomic processing module performs at least one of the above computational steps for searching for proteins by using a different search engine. Examples of search engines suitable for searching for proteins against a database include the OMSSA engine and the X! tandem engine. Searching for proteins against a protein database using the X! tandem engine can include performing at least one of the following steps: setting the search mode to X! Tandem, setting up the forward database (e.g., HMRB) for search in X! Tandem, performing the forward X! Tandem search, setting up the reversed database (HMRB reversed) for the search in X! Tandem, and performing the reverse search in X! Tandem.

Next, the proteomic processing module can validate the proteomic data. In some instances, the proteomic processing module filters the results of the protein search such as results generated by OMSSA. Filtering the results of the protein search can include computing the expectation values for a range of FDRs for peptides identified within a sample. The proteomic processing module can model RTs for proposed peptides and filter out those which are at significant variance with the model. Proteomic data validation for OMSSA forward and reverse search results can include performing at least one of the following steps: setting the search mode to OMSSA, setting up the forward database (e.g., HMRB) for validation, calculating the FDR and associated expectation values, developing an RT model from the sample's data, and performing RT filtering to reject proposed peptides that differ from the model.

Alternatively or in combination, the proteomic processing module validates the results of the protein search such as the results generated by X! Tandem. Filtering the results of the protein search can include performing at least one of the following steps: setting the search mode to X! Tandem, setting up the forward database (e.g., HMRB) for validation, calculating the FDR and associated expectation values, developing an RT model from the sample's data, and performing RT filtering to reject proposed peptides that differ from the model.

It is understood that any of the proteomic processing steps of the present disclosure can be carried out using various search engines including but not limited to OMSSA and X! Tandem, which are used in certain embodiments disclosed herein.

The proteomic processing module can perform at least one computational step carrying out analysis of the proteomic data to analyze the validation results, which are optionally saved to a database. The analysis of the proteomic data can include at least one of the following steps: setting up the forward database (e.g., HMRB) for review, evaluating the OMSSA and X! Tandem search, validate the search results, and report filtering statistics.

The proteomic processing module can perform at least one computational step mapping the peptide results (e.g., results from X! Tandem and/or OMSSA searches) to proteins in a database such as UniProt HMRB FASTA (e.g., using BlastP). The hit scores and/or ranks from the mapping step are optionally saved by the proteomic processing module. Mapping the sample data can include performing at least one of the following steps: searching for protein matches to the OMSSA-based peptides using BlastP, assigning BlastP scores and ranks to the OMSSA-based peptides, summarizing and saving information about the protein matches found for OMSSA-based peptides, searching for protein matches to the X! Tandem-based peptides using BlastP, assigning BlastP scores and ranks to the X! Tandem-based peptides, and summarizing and saving information about the protein matches found for X! Tandem-based peptides.

Sometimes, the proteomic processing module can perform at least one computational step determining the targeted proteomic results for statistical review.

Proteomic processing can be initiated as part of a computational workflow. The workflow or the proteomic processing is optionally queued by registered instruments such as mass spectrometric or data analysis instruments. When proteomic processing is initiated or instructed, software such as an application programming interface (API) is usually tasked with performing the requisite computational steps. In various cases, the API comprises the proteomic processing module that carries out proteomic processing. The data is typically acquired from a data source such as a mass spectrometry machine.

In some instances, the proteomic processing steps undergo quality assessment steps to assess successful proteomic processing and/or quality of the processed data. The quality assessment can include process control steps to ensure one or more of the various computational steps have been successfully carried out. The quality assessment can also include quality control steps for evaluating quality of the data generated by the various steps of proteomic processing. Sample data failing the quality assessment variously results in flagging the sample data, for example so as to indicate in its output that there is an issue in the analysis, or may result in pausing or cancelling the computational workflow so as to address the workflow or sample data issue, for example by re-attempting proteomic processing (or any of the steps comprising proteomic processing), or by discarding the sample data from the computational workflow.

### Quality Analysis

Provided herein are systems, devices, and methods implementing computational pipelines for processing of data such as data generated by profile and DPS proteomics. Computational pipelines often comprise quality analysis carried out by a quality control module. The quality control module carries out one or more computational steps for analyzing the quality of data such as mass spectrometric data. Following quality analysis, the sample data can be passed on to subsequent data processing modules for further manipulation and/or analysis. Sample data that undergoes quality analysis by the quality control module can be obtained from a preceding module such as the proteomic processing module. Quality analysis is sometimes performed on sample data obtained from multiple data files corresponding to different samples that are acquired together or sequentially. The quality control module can carry out any of the computation steps described herein as part of a product package.

The quality control module can perform one or more computational steps to carry out analyzing data quality. The quality control module can perform at least one of the following steps: making total ion chromatogram (TIC) comparisons, generating a protein map, calculating molecular feature tolerance validations, peptide clustering, or other quality control assessments. Sometimes, the quality control module performs at least one computational step calculating each scan's quality. Scan quality (e.g., MS1, MS2, or both) can be evaluated by various factors such as at least one of number of peaks, peak relative ratios, abundance ratios, signal to noise ratio (SNR), and sequence tag length. Such factors are often derived from MGF and/or spectral features files. Next, the proteomic processing module optionally performs at least one computational step determining the standard quality metrics.

Quality analysis can be initiated as part of a computational workflow. The workflow or the quality analysis is optionally queued by registered instruments such as mass spectrometric or data analysis instruments. When quality analysis is initiated or instructed, software such as an application programming interface (API) is usually tasked with performing the requisite computational steps. In various cases, the API comprises the quality control module that carries out quality analysis. The data is typically acquired from a data source such as a mass spectrometry machine.

In some instances, the quality analysis steps constitute quality assessment steps for assessing quality of the processed data. The quality assessment can include process control steps to ensure one or more of the various quality analysis steps have been successfully carried out. The quality assessment can also include quality control steps for evaluating quality of the data as described herein. Sample data failing the quality assessment variously results in flagging the sample data, for example so as to indicate in its output that there is an issue in the analysis, or may result in pausing or cancelling the computational workflow so as to address the workflow or sample data issue, for example by re-attempting data analysis (or any of the steps comprising data analysis), or by discarding the sample data from the computational workflow.

### Visualization

Provided herein are systems, devices, and methods implementing computational pipelines for processing of data such as data generated by profile and DPS proteomics. Computational pipelines often comprise a visualization process carried out by a visualization module. The visualization module carries out one or more computational steps for visualizing data such as mass spectrometric data. For example, data visualization can include creating a star field thumbnail. The star field thumbnail can provide a visualization of signal intensity plotted for LC RT vs. m/z, in which low resolution isotopic features appear as points of light resembling stars. Alternatively or in combination, the star field thumbnail provides a visualization view of a 4-Dimensional m/z over LC time perspective showing the isotopic feature views of the peaks as a “star.” Following data visualization, the sample data can be passed on to subsequent data processing modules for further manipulation and/or analysis. Sample data that undergoes visualization by the proteomic processing module can be obtained from a preceding module such as the quality control module. Visualization is sometimes performed on sample data obtained from multiple data files corresponding to different samples that are acquired together or sequentially. The visualization module can carry out any of the computation steps described herein as part of a product package.

Data visualization can be initiated as part of a computational workflow. The workflow or the data visualization process is optionally queued by registered instruments such as mass spectrometric or data analysis instruments. When data visualization is initiated or instructed, software such as an application programming interface (API) is usually tasked with performing the requisite computational steps. In various cases, the API comprises the visualization module that carries out data visualization. The data is typically acquired from a data source such as a mass spectrometry machine.

In some instances, the data visualization steps undergo quality assessment to assess successful data visualization. The quality assessment can include process control steps to ensure one or more of the various computational steps have been successfully carried out. The quality assessment can also include quality control steps for evaluating quality of the data generated by the various steps of proteomic processing. Sample data failing the quality assessment variously results in flagging the sample data, for example so as to indicate in its output that there is an issue in the analysis, or may result in pausing or cancelling the computational workflow so as to address the workflow or sample data issue, for example by re-attempting data visualization (or any of the steps comprising data visualization), or by discarding the sample data from the computational workflow.

### Utilities

Provided herein are systems, devices, and methods implementing computational pipelines for processing of data such as data generated by profile and DPS proteomics. Computational pipelines often provide utilities for enhancing data exploration, visualization, and/or monitoring. Computational pipelines often comprise one or more utilities provided by a utilities module. The utilities module provides one or more utilities for evaluating data (e.g., exploration, visualization, monitoring, etc.) such as mass spectrometric data. Sample data that is evaluated using a utility can be obtained from a preceding module. Utilities are sometimes used to evaluate sample data obtained from multiple data files corresponding to different samples that are acquired together or sequentially. The utilities module is often part of a product package.

Utilities can be used and/or initiated as part of a computational workflow. The workflow or the utilities is optionally queued by registered instruments such as mass spectrometric or data analysis instruments. When utilities are initiated or accessed, software such as an application programming interface (API) is usually tasked with performing the requisite computational steps to provide the utilities. In various cases, the API comprises the utilities module that carries out data evaluation using at least one utility. The data is typically acquired from a data source such as a mass spectrometry machine.

The utilities module comprises at least one helper utility. Helper utilities can perform at least one task such as calculating charged mass, calculating molecular weight, calculating peptide mass, calculating tandem pass, searching for sequence homology, determining column use, plotting spectra, determining pipeline status, checking machine status, tuning reports, controlling workflow, or annotating issues that arise.

Sometimes, the utilities module performs at least one computational step determining the neutral mass and the mass of the charged state(s) for a given molecular formula. For example, the utilities module can provide a utility that uses the mass to determine the neutral plus charge states such as from charge states 1 through 5. Sometimes, the utilities module performs at least one computational step calculating peptide mass.

The utilities module can provide a utility that calculates peptide mass such as by entering the peptide or protein sequence and determining the neutral mass and mass of charge states such as charge states 1 through 6.

The utilities module can provide a utility that calculates the tandem mass. In some instances, this step includes entering the peptide or protein sequence, showing the “y” and “b” components along with options for charge states with modifications in a tabular format

In certain cases, the utilities module searches peptides against at least one database (e.g., Human FASTA database) to identify matching proteins.

The utilities module sometimes assesses the remaining LCMS lifetime against a pre-defined threshold. For example, the LCMS column may have a pre-defined threshold after which the column may be no longer considered reliable and discarded as a quality control step.

In various aspects, the utilities module plots spectra from a file such as a CSV or MGF file.

The utilities module optionally calculates and/or provides a pipeline status, which can include a list of computational steps (e.g., valves), the machine registered to run those processes or computational steps, and the machine status (e.g., on or off, or whether a sample is being processed).

The utilities module often provides a machine status such as a list of machines participating and registered in the computational pipeline, and optionally includes membership and processing status.

The utilities module often provides reports indicating tune reports for the mass spectrometer instruments.

The utilities module can perform at least one computational step for controlling the workflow such as pausing and resetting process nodes (e.g., a digital processing device, a network-connected device, a processor, etc.).

Finally, the utilities module sometimes provides annotation of issues that are resolved but entail a situation in which the processing is unable to be completed. For example, an issue in which a critical failure of a necessary computational pipeline component could mean the processing cannot be completed. However, the issue may nonetheless be annotated to help diagnose and/or resolve the problem for subsequent processing runs.

In some instances, the utilities steps undergo quality assessment that can include process control steps to ensure one or more of the various computational steps have been successfully carried out. The quality assessment can also include computational steps providing various utilities for evaluating or manipulating sample data. Sample data failing the quality assessment variously results in flagging the sample data, for example so as to indicate in its output that there is an issue in the analysis, or may result in pausing or cancelling the computational workflow so as to address the workflow or sample data issue, for example by re-attempting utilities evaluation (or any of the steps comprising utilities evaluations), or by discarding the sample data from the computational workflow.

### Monitoring

Provided herein are systems, devices, and methods implementing computational pipelines for processing of data such as data generated by profile and DPS proteomics. Computational pipelines often comprise a monitoring process carried out by a monitoring module. The monitoring module carries out one or more computational steps for providing monitoring for users such as self-registration and opt-out email notifications for specific events. The monitoring process is often performed by at least one software module in a product package.

Sometimes, a monitoring module continuously monitors the system logbook (e.g., log book for the analytical computing system used for carrying out the various steps of the computational pipeline). The monitoring module may autonomously monitor for events coming off of instruments (e.g., by monitoring a SysLogbook) for errors and warnings that can be handled promptly or dealt with immediately such as without requiring an operator to manually monitor the instrument.

Sometimes, the monitoring module provides a quality control step such as checking if an error condition occurs (e.g., when maximum ultra violet time is shorter than expected) when a data file such as an IDFC data field is transferred to a database such as a central repository. The monitoring for error conditions can allow a lab technician to investigate further before proceeding with experimental protocols.

The monitoring module often reports resolution of primary data transfer verifications during disk space cleanup activities prior to computer removal. This process can be performed periodically to purge more data off the instrument.

The monitoring module can detect an error condition that stops the workflow. Next, activity to resolve the issue can be remediated in the laboratory or computationally to process the samples (e.g., processing the data to account for the error). Sometimes, the monitoring module measures data quality. For example, when process control samples result, metrics based on process control samples are often compared for proper instrument operations. Determination of a failure criteria may pause or postpone laboratory work until the issue is resolved or cause interpretation of the data to be excluded from later study due to poor quality (e.g., gating the data set to remove poor quality data).

In some instances, the monitoring module provides notification of pipeline processes being turned off or on (manually or automatically).

The monitoring module can provide notification of the failure of a process that may or may not be material, which is optionally investigated to ensure the sample data is processed.

The monitoring module can also send at least one of orbitrap report upon transfer of a directory instrument file.

The monitoring module, or alternatively, a cleanup module, often performs a cleanup step such as removing and/or compressing the data file (e.g., APIMS1 file) to save space on a shared drive.

Computational Pipeline for Targeted and iMRM Proteomics

Disclosed herein are computational pipelines for processing of data such as data generated by targeted and iMRM proteomics. The computational pipeline comprises a plurality of data processing modules that transform, convert, or otherwise manipulate data. The data is often mass spectrometric data such as protein mass spectrometric data generated from a sample. The data processing modules carry out computational steps that process the data from the preceding module. Data processing modules perform various data manipulation functions such as data acquisition, workflow determination, data extraction, feature extraction, proteomic processing, and quality analysis. The computational pipeline can utilize two or more of the data processing modules to generate usable data. In some instances, the computational pipeline uses at least 1, 2, 3, 4, 5, 6, 7, 8, 9, 10, 11, 12, 13, 14, 15, 16, 17, 18, 19, 20, 30, 40, 50, 60, 70, 80, 90, or 100 or more data processing modules, and/or no more than 1, 2, 3, 4, 5, 6, 7, 8, 9, 10, 11, 12, 13, 14, 15, 16, 17, 18, 19, 20, 30, 40, 50, 60, 70, 80, 90, or 100 or more data processing modules.

### Data Acquisition

Disclosed herein are computational pipelines for processing of data such as data generated by targeted and iMRM proteomics. Computational pipelines often comprise a data acquisition process carried out by a data acquisition module. The data acquisition module carries out one or more computational steps for acquiring data such as mass spectrometric data. The data acquisition module can start a queued workflow by polling registered instruments connected to mass spectrometer(s) and acquiring data generated by the mass spectrometer(s). The acquired data can be passed on to subsequent data processing modules for further manipulation and/or analysis. Multiple data sets corresponding to different samples are sometimes acquired together or sequentially. The data acquisition process is often performed by at least one software module in a product package.

Data acquisition can be initiated as part of a computational workflow. The workflow or the data acquisition is optionally queued by registered instruments such as mass spectrometric or data analysis instruments. When data acquisition is initiated or instructed, software such as an application programming interface (API) is usually tasked with performing the requisite computational steps. In various cases, the API comprises the data acquisition module that carries out data acquisition. The data is typically acquired from a data source such as a mass spectrometry machine.

The data acquisition module optionally includes a data transfer process following data acquisition. The data transfer process often entails copying and/or storage of the acquired data into a storage or memory (e.g., a database). The storage is sometimes shared primary data storage. In some instances, the data acquisition undergoes a quality assessment step to confirm that the instrument data has been copied to storage such as a shared repository (e.g., a database). The quality assessment can include a process control step to ensure the data acquisition and/or data transfer was carried out. The quality assessment can also include a quality control step for evaluating quality of the acquired data. Sample data failing the quality assessment variously results in flagging the sample data, for example so as to indicate in its output that there is an issue in the analysis, or may result in pausing or cancelling the computational workflow so as to address the workflow or sample data issue, for example by re-attempting data acquisition (or any of the steps comprising data acquisition), or by discarding the sample data from the computational workflow.

The data obtained for the computational workflow can be obtained from mass spectrometric processes incorporating various methodologies such as SIS, targeted proteomics, a protein quantification assay such as an antibody based or antibody-independent protein quantification assay, protein purification, sample fractionation, and other proteomics methodologies.

### Determining Workflow

Disclosed herein are computational pipelines for processing of data such as data generated by targeted and iMRM proteomics. Provided herein are systems, devices, and methods implementing computational pipelines (also referred to as computational workflow) for processing of data such as data generated by profile and DPS proteomics. Computational pipelines often comprise a workflow determination process carried out by a workflow module. The workflow module carries out one or more steps for determining a computational workflow for processing and/or analysis of data such as mass spectrometric data. The workflow module can carry out any of the steps described herein as part of a product package (e.g., a package for an end-to-end mass spectrometric workflow that includes study planning/experimental design, mass spectrometric sample processing and concurrent quality assessments, and computational workflow for data analysis). The workflow module often performs a parsing step such as on a worklist, also referred to as a protocol. The worklist serves to provide instructions for any or each step in a process and can also record experiment-specific data for samples. In some cases, worklists contain scripts used by devices such as computational devices and mass spectrometric devices. Worklists can include various workflow parameters or information relevant to workflow parameters such as random sample ordering and appropriate volumes used. Control samples are often processed in the same order for every worklist. This order can include control samples being used at the beginning, middle, and end of specific steps within the experiment. As such, control samples can help normalize samples and worklists during data analysis. This can include sample label information and reagents information, including concentrations and lot numbers used with a particular set of samples. Worklists used with a particular process can be stored with, archived, or associated with the corresponding experiment for later reference. In some instances, the worklist includes various parameters from a preceding experimental design workflow and/or a sample processing workflow. The parameters can include any of biomarkers or biomarker candidates, the method used to generate the biomarkers or biomarker candidates (e.g., manually curated, automated, or a combination thereof), precursors and/or ion transitions selected for mass spectrometric analysis, desired or threshold statistical metrics (e.g., p-value, CV) for the study result/output, number of samples, number of replicates, depletion of abundant proteins, the identity of depleted proteins, protein enrichment (e.g., by purification such as immunoprecipitation), liquid chromatography parameters, mass spectrometric instrument parameters, and other parameters relating to the overall mass spectrometric workflow. Alternatively, the preceding parameters can be obtained separately from the worklist and used to generate a corresponding computational workflow suitable for carrying out data analysis based on the parameters.

Control samples are often processed in the same order for every worklist. This order can include control samples being used at the beginning, middle, and end of specific steps within the experiment. As such, control samples can help normalize samples and worklists during data analysis. This can include sample label information and reagents information, including concentrations and lot numbers used with a particular set of samples. Worklists used with a particular process can be stored with, archived, or associated with the corresponding experiment for later reference.

The workflow module can read the worklist by parsing the worklist to extract workflow parameters and/or information relating to workflow parameters. Following parameter extraction, the workflow module usually sets the parameters for the workflow. The workflow module optionally determines appropriate parameters based on information extracted from the worklist. For example, a workflow parameter may be adjusted to account for worklist information indicating the sample is a dried blood spot or that the sample comprises reference biomarkers that require certain computational steps for accurate detection. Workflow parameters can include the mass spectrometric method, pump model number, sample type, sample name, data acquisition rate minimum and/or maximum, concentration, volume, plate position, plate barcode, and/or other parameters related to sample processing and/or analysis. The workflow module often performs an additional step such as a controller step wherein downstream analyses or computations are determined based on the method and parameters for the workflow. In some instances, the workflow module generates a workflow based on the extracted parameters and/or other information provided in the data file or by a user. The workflow is customized or pre-generated for the type of analysis to be performed. For example, Targeted and iMRM proteomics can require a different workflow than Profile and DPS proteomics.

Workflow determination can be initiated as part of a computational workflow. The computational workflow or the workflow determination is optionally queued by registered instruments such as mass spectrometric or data analysis instruments. When workflow determination is initiated or instructed, software such as an application programming interface (API) is usually tasked with performing the requisite computational steps. In various cases, the API comprises the workflow module that carries out workflow determination. The worklist is typically acquired from a data source such as a mass spectrometry machine or computing device.

The workflow module often performs a controller step for determining the pipeline computations and steps to run based on the method (e.g., LCMS method) used to generate the data file and parameters gathered from parsing the worklist. In some cases, the data file and parameters are defined in the instrument method and study such as a LCMS method. The pipeline computations and steps constitute a computation flow that is optionally set in a computational group. Computational groups allow modularization of pipeline computational flow such that each computational flow can be reconfigured, for example, by combining various computation flow modules. The modularization allows the reconfiguration of computational flow to be performed more easily compared to non-modular computational flow configurations. For example, the computational groups can be reconfigured depending on study requirements and/or the nature of the sample being processed such as whether the sample is a blank or QC sample.

The workflow module optionally includes a quality assessment process following workflow determination. In some instances, the workflow determination comprises a quality assessment step to confirm that the computation flow has been properly configured. The quality assessment can include a process control step to ensure the workflow determination step is carried out. The quality assessment can also include a quality control step for evaluating quality of the workflow determination. For example, information from the worklist may indicate issues such as incompatibility between information from the worklist and available workflow parameters or options. Workflow parameters failing the quality assessment variously results in flagging the sample data, for example so as to indicate in its output that there is an issue in the analysis, or may result in pausing or cancelling the computational workflow so as to address the workflow issue, for example by re-attempting workflow determination (or any of the steps comprising workflow determination), or by discarding the sample data from the computational workflow.

The workflow determination module may configure a computational workflow to perform a quality assessment on at least one of the subsequent data processing or computational steps carried out during the execution of the computational workflow. In some cases, the quality assessment evaluates the data output for a particular data processing step such as by using a quality control metric (e.g., elution time, signal-to-noise ratio (SNR), signal strength/intensity, pairwise fragment ratios, and various other QC metrics). The quality assessment can include an evaluation of the data processing step itself and/or the performance of a data processing module such as identifying an expected output or metric indicative of successful data processing/manipulation. In some cases, a mislabeled or corrupted file can result in the data not being correctly saved or rendered inaccessible.

The computational workflow can be informed by upstream quality assessments carried out during sample processing such as during mass spectrometric evaluation of a sample. For example, a quality assessment for elution time can be performed for one or more samples during mass spectrometric analysis. The elution time for measured sample proteins or peptides may vary between samples such as sample replicates or experimental and control samples. Accordingly, a quality assessment that measures or otherwise accounts for elution time can enable the computational workflow to normalize or adjust one or more data sets.

### Data Preparation

Disclosed herein are computational pipelines for processing of data such as data generated by targeted and iMRM proteomics. Computational pipelines often comprise a data preparation process carried out by a data preparation module. The data preparation module carries out one or more computational steps for preparing data such as mass spectrometric data for further analysis. Following data preparation, the sample data can be passed on to subsequent data processing modules for further manipulation and/or analysis. Sample data that is prepared by the data preparation module can be obtained from a preceding module. Data preparation is sometimes performed on sample data obtained from multiple data files corresponding to different samples that are acquired together or sequentially. The data preparation process is often performed by at least one software module in a product package.

The data preparation module can perform one or more computational steps to carry out data preparation. Sometimes, the data preparation module performs a step converting data into a standardized format such as mzML, optionally using ProteoWizard for the conversion.

Data preparation can be initiated as part of a computational workflow. The workflow or the data preparation is optionally queued by registered instruments such as mass spectrometric or data analysis instruments. When data preparation is initiated or instructed, software such as an application programming interface (API) is usually tasked with performing the requisite computational steps. In various cases, the API comprises the data preparation module that carries out data preparation. The data is typically obtained from a data source such as a mass spectrometry machine.

In some instances, the data preparation process undergoes a quality assessment step to assess successful data preparation and/or quality of the prepared data. The quality assessment can include a process control step to ensure the data preparation was carried out. The quality assessment can also include a quality control step for evaluating quality of the prepared data. Sample data failing the quality assessment variously results in flagging the sample data, for example so as to indicate in its output that there is an issue in the analysis, or may result in pausing or cancelling the computational workflow so as to address the workflow or sample data issue, for example by re-attempting data preparation (or any of the steps comprising data extraction), or by discarding the sample data from the computational workflow.

### Data Extraction

Disclosed herein are computational pipelines for processing of data such as data generated by targeted and iMRM proteomics. Computational pipelines often comprise a data extraction process carried out by a data extraction module. The data extraction module carries out one or more computational steps for extracting data such as mass spectrometric data. Data extraction can include reading raw data and extracting the raw data into a different format (e.g., a more easily consumable format). An example of data extraction is parsing mzML into CSV for peak data. The extracted data can be passed on to subsequent data processing modules for further manipulation and/or analysis. Sample data that is extracted by the data extraction module can be used for downstream processing. Sample data is sometimes extracted from multiple data files corresponding to different samples that are acquired together or sequentially. The data extraction process is often performed by at least one software module in a product package.

The data extraction module can perform one or more computational steps to carry out data extraction. In some cases, the data extraction module generates a location for the extracted information such as a directory for storage. The data acquisition module sometimes performs at least one computational step for extracting and converting spectral data into a different format such as from mzML files into CSV files for later processing.

Data extraction can be initiated as part of a computational workflow. The workflow or the data extraction is optionally queued by registered instruments such as mass spectrometric or data analysis instruments. When data extraction is initiated or instructed, software such as an application programming interface (API) is usually tasked with performing the requisite computational steps. In various cases, the API comprises the data extraction module that carries out data extraction. The data is typically acquired from a data source such as a mass spectrometry machine.

In some instances, the data extraction process undergoes a quality assessment step to assess successful data extraction and/or quality of the extracted data. The quality assessment can include a process control step to ensure the data extraction was carried out. The quality assessment can also include a quality control step for evaluating quality of the acquired data. Sample data failing the quality assessment variously results in flagging the sample data, for example so as to indicate in its output that there is an issue in the analysis, or may result in pausing or cancelling the computational workflow so as to address the workflow or sample data issue, for example by re-attempting data extraction (or any of the steps comprising data extraction), or by discarding the sample data from the computational workflow.

### Feature Extraction

Disclosed herein are computational pipelines for processing of data such as data generated by targeted and iMRM proteomics. Computational pipelines often comprise a feature extraction process carried out by a feature extraction module. The feature extraction module carries out one or more computational steps for extracting features from data such as mass spectrometric data such as identifying peaks and determining the areas of the identified peaks. For example, the feature extraction module can determine the area under the curve (AUC) for proteomic data of interest such as for heavy and light peptides based on the study and experiment. Following feature extraction, the sample data comprising extracted features can be passed on to subsequent data processing modules for further manipulation and/or analysis. Sample data that undergoes feature extraction by the feature extraction module can be obtained from a preceding module. Feature extraction is sometimes performed on sample data obtained from multiple data files corresponding to different samples that are acquired together or sequentially. The feature extraction process is often performed by at least one software module in a product package.

The feature extraction module can perform one or more computational steps to carry out feature extraction. Sometimes, the feature extraction module performs a step creating a defined directory for the extracted information. In certain cases, the feature extraction module identifies peaks for m/z trace files that signal proteomic data of interest.

Feature extraction can be initiated as part of a computational workflow. The workflow or the feature extraction is optionally queued by registered instruments such as mass spectrometric or data analysis instruments. When feature extraction is initiated or instructed, software such as an application programming interface (API) is usually tasked with performing the requisite computational steps. In various cases, the API comprises the feature extraction module that carries out feature extraction. The data is typically acquired from a data source such as a mass spectrometry machine.

In some instances, the feature extraction process undergoes a quality assessment step to assess successful feature extraction and/or quality of the extracted features. The quality assessment can include a process control step to ensure the feature extraction was carried out. The quality assessment can also include a quality control step for evaluating quality of the extracted features. Sample data failing the quality assessment variously results in flagging the sample data, for example so as to indicate in its output that there is an issue in the analysis, or may result in pausing or cancelling the computational workflow so as to address the workflow or sample data issue, for example by re-attempting feature extraction (or any of the steps comprising feature extraction), or by discarding the sample data from the computational workflow.

### Proteomic Processing

Disclosed herein are computational pipelines for processing of data such as data generated by targeted and iMRM proteomics. Computational pipelines often comprise a proteomic processing process carried out by a proteomic processing module. The proteomic processing module carries out one or more computational steps for proteomic processing of data such as mass spectrometric data. For example, proteomic processing can include inserting cluster peaks and linking heavy and light peaks to ensure the transition peaks are aligned. Following proteomic processing, the sample data can be passed on to subsequent data processing modules for further manipulation and/or analysis. Sample data that undergoes proteomic processing by the proteomic processing module can be obtained from a preceding module such as the feature extraction module. Proteomic processing is sometimes performed on sample data obtained from multiple data files corresponding to different samples that are acquired together or sequentially. Proteomic processing is often performed by at least one software module in a product package.

The proteomic processing module can perform one or more computational steps to carry out proteomic processing. Sometimes, the proteomic processing module performs at least one computational step determining the peak area for m/z peak “traces.” The proteomic processing module annotate or flag the identified peak and associate it to proteomic data items (e.g., for a sample).

Proteomic processing can be initiated as part of a computational workflow. The workflow or the proteomic processing is optionally queued by registered instruments such as mass spectrometric or data analysis instruments. When proteomic processing is initiated or instructed, software such as an application programming interface (API) is usually tasked with performing the requisite computational steps. In various cases, the API comprises the proteomic processing module that carries out proteomic processing. The data is typically acquired from a data source such as a mass spectrometry machine.

In some instances, the proteomic processing steps undergo quality assessment steps to assess successful proteomic processing and/or quality of the processed data. The quality assessment can include process control steps to ensure one or more of the various computational steps have been successfully carried out. The quality assessment can also include quality control steps for evaluating quality of the data generated by the various steps of proteomic processing. Sample data failing the quality assessment variously results in flagging the sample data, for example so as to indicate in its output that there is an issue in the analysis, or may result in pausing or cancelling the computational workflow so as to address the workflow or sample data issue, for example by re-attempting proteomic processing (or any of the steps comprising proteomic processing), or by discarding the sample data from the computational workflow.

### Quality Analysis

Disclosed herein are computational pipelines for processing of data such as data generated by targeted and iMRM proteomics. Computational pipelines often comprise quality analysis carried out by a quality control module. The quality control module carries out one or more computational steps for analyzing the quality of data such as mass spectrometric data. The quality analysis can access data related to quality assessments such as light and heavy peptides' signal-to-noise ratios (SNRs), transition counts, RT delta, and peak area. Following quality analysis, the sample data can be passed on to subsequent data processing modules for further manipulation and/or analysis. Sample data that undergoes quality analysis by the quality control module can be obtained from a preceding module such as the protein/proteomic processing module. Quality analysis is sometimes performed on sample data obtained from multiple data files corresponding to different samples that are acquired together or sequentially. The quality analysis is often performed by at least one software module in a product package.

The quality control module can perform one or more computational steps to carry out analyzing data quality. Sometimes, the proteomic processing module performs at least one computational step gathering the m/z peak trace data for examination according to certain quality control metrics. For example, scan quality (e.g., MS1, MS2, or both) can be evaluated by various factors such as probability, number of peaks, ratios, lag, noise, and size. In some instances, the quality control module generates metrics on the features of the m/z peak trace data that has been gathered and identified for regular and/or quality control samples.

Quality analysis can be initiated as part of a computational workflow. The workflow or the quality analysis is optionally queued by registered instruments such as mass spectrometric or data analysis instruments. When quality analysis is initiated or instructed, software such as an application programming interface (API) is usually tasked with performing the requisite computational steps. In various cases, the API comprises the quality control module that carries out quality analysis. The data is typically acquired from a data source such as a mass spectrometry machine.

In some instances, the quality analysis steps constitute quality assessment steps for assessing quality of the processed data. The quality assessment can include process control steps to ensure one or more of the various quality analysis steps have been successfully carried out. The quality assessment can also include quality control steps for evaluating quality of the data as described herein. Sample data failing the quality assessment variously results in flagging the sample data, for example so as to indicate in its output that there is an issue in the analysis, or may result in pausing or cancelling the computational workflow so as to address the workflow or sample data issue, for example by re-attempting quality analysis (or any of the steps comprising quality analysis), or by discarding the sample data from the computational workflow.

### Utilities

Disclosed herein are computational pipelines for processing of data such as data generated by targeted and iMRM proteomics. Such computational pipelines often include utilities for enhancing data exploration, visualization, and/or monitoring. Computational pipelines often comprise one or more utilities provided by a utilities module. The utilities module provides one or more utilities for evaluating data such as mass spectrometric data. Sample data that is evaluated using a utility can be obtained from a preceding module. Utilities are sometimes used to evaluate sample data obtained from multiple data files corresponding to different samples that are acquired together or sequentially. Sometimes, the utilities module visualizes m/z peak traces such as for heavy and light peptides (e.g., for samples with isotope labeled peptides/proteins). The utilities module is often part of a product package.

Utilities can be used and/or initiated as part of a computational workflow. The workflow or the utilities is optionally queued by registered instruments such as mass spectrometric or data analysis instruments. When utilities are initiated or accessed, software such as an application programming interface (API) is usually tasked with performing the requisite computational steps to provide the utilities. In various cases, the API comprises the utilities module that carries out data evaluation using at least one utility. The data is typically acquired from a data source such as a mass spectrometry machine.

In some instances, the utilities steps undergo quality assessment that can include process control steps to ensure one or more of the various computational steps have been successfully carried out. The quality assessment can also include computational steps providing various utilities for evaluating or manipulating sample data. Sample data failing the quality assessment variously results in flagging the sample data, for example so as to indicate in its output that there is an issue in the analysis, or may result in pausing or cancelling the computational workflow so as to address the workflow or sample data issue, for example by re-attempting utilities evaluation (or any of the steps comprising utilities evaluations), or by discarding the sample data from the computational workflow.

### Identification of Health Condition Indicators

Described herein are methods and apparatuses related to identification of a health condition indicator in response to receiving a biological input parameter. The input parameter variously comprises at least one of a protein or RNA biomarker or portion thereof, a gene, a pathway, a dataset generated from an individual run, and a health condition status. The health condition indicator provides as output at least one of a protein or RNA biomarker or portion thereof, a gene, a pathway, a dataset generated from an individual run, and a health condition status. That is, upon entry of comprises at least one of a protein or RNA biomarker or portion thereof, a gene, a pathway, a dataset generated from an individual run, and a health condition status by a user or input source, methods and devices consistent with the disclosure herein provide as output a different at least one of a protein or RNA biomarker or portion thereof, a gene, a pathway, a dataset generated from an individual run, and a health condition status, such that an at least one output protein or RNA biomarker or portion thereof, gene, pathway, dataset generated from an individual run, or health condition status is provided so as to identify interrelated members of the above lists. That is, for an inputted disorder, methods and systems as disclosed herein variously provide a related pathway or pathways, a related protein or proteins, a related gene or genes, a related marker or markers, related publicly available art and expression analysis data, related mass spectrometric or other existent datasets, related disorders and other related information, as well as second-order information related thereto. Similarly, for an inputted experimental dataset such as an experimental run, methods and systems herein provide related a related pathway or pathways, a related disorder or disorders, a related gene or genes, a related marker or markers, related public art and expression information, and related nonpublic data information involving the same or overlapping markers, proteins or genes. Any member of a list above may serve as an input, and any number of iterations of output may be generated. A disease input may as part of its output, for example, identify a pathway and common proteins, genes and markers for the pathway, as well as other diseases related to the pathway, proteins, genes or markers of the disorder. The input parameters and other data utilized for identification of a health condition or health condition indicator can be generated using mass spectrometric workflows and/or computational workflows as described throughout the present disclosure. In some cases, the mass spectrometric workflow and/or computational workflow comprises performing analytical steps for identification of the health condition indicator.

The interrelatedness indicator identification process variously comprises accessing a dataset comprising a collection of information which designates one or more associations between the input parameter and the health condition indicator or other output parameter. Some datasets comprise information which specifies presence of relationships between or among various biological indicators. Some datasets comprise information indicating predetermined associations between the input parameter and the output health condition indicator. Some datasets comprise information designating predetermined relationships between different biomarkers or portions thereof, health conditions, biological pathway and/or genes. Also included in some dataset inputs or outputs is availability of markers such that, for a given disorder, pathway or marker, one may determine what markers are readily available, and similarly for a given set of markers, one may determine what proteins, genes, pathways or disorders are readily assayed.

The dataset is in some cases a fixed or unchanging dataset comprising publicly available information such as is available form published papers and expression information at or up to a given period of time. Alternately, some datasets comprise private or nonpublic generated data or information, such as information related to private or unpublished experiments such as mass spectrometric results, or also may include information as to which proteins or genes implicated in an experiment or pathway have publicly available or privately obtained markers, such as suitable for mass spectrometric analysis.

The dataset is queried in response to receiving the input parameter such that one or more of a biomarker or portion thereof, a health condition status, and a biological pathway implicated by the input parameter can be generated and provided to a user. Queries are often ‘multi-directional,’ such that any particular feature, such as a disorder or disease, a pathway, a gene or protein implicated or otherwise associated with a disease or pathway, a marker informative of such a gene or protein, a distributor or lab source or location of such a marker, public art on the topic, public or undisclosed expression analysis or other expression data, or other dataset constituents may serve as either a query or an output. That is, one may query any position or category of information and receive as output information relevant to related categories of information.

A biomarker described herein can comprise a protein. In some cases, the biomarker is a non-protein biomarker. In some cases, the health condition indicator identification process can comprise generating as an output indicative of one or more proteins, polypeptides, health conditions and biological pathways which have a designated association with the input parameter, or one or more experimental result datasets that involve the protein or other marker. The one or more proteins, polypeptides, health conditions and biological pathways can be impacted by the input parameter. For example, one or more proteins, peptides and/or polypeptides can be identified based on the collection information of the dataset designating a positive or negative correlation between the one or more proteins, peptides, and/or polypeptides and the input parameter, such as an input biomarker or portion thereof. One or more health condition statuses, such as colorectal diseases (e.g., colorectal cancer) can be identified as being implicated by an input biomarker, or portion thereof, based on the collection information indicating existence of a relationship between the health condition status and the input biomarker or portion thereof. In some cases, biological pathways which result in generation, consumption and/or modification of the input biomarker or portion thereof are identified. In some cases, one or more other biomarkers or portions thereof are identified which have designated associations with the input biomarker or portion thereof. For example, the identification process can generate as output biomarkers or portions thereof which are implicated by the same health condition, biological pathway and/or genes as the input biomarker. Furthermore, the output in some cases indicates where or whether particular biomarkers are available, either as assets of a particular lab or as products offered for sale.

A biomarker parameter as designated herein can comprise a gene and the output generated in response can comprise one or more biomarkers or portions thereof, biological pathways, and/or health conditions implicated by the gene. For example, the gene can affect the level of biomarkers or portions thereof, the functioning of the biological pathways, and/or contribute to the presence of the health conditions. In some cases, the input parameter comprise a health condition and the output generated in response can comprises one or more biomarkers or portions thereof and/or biological pathways implicated by the health condition. For example, the output biomarkers or portions thereof can have a positive or negative correlation with the presence of the health condition, and/or the output biological pathways can contribute to the presence of the health condition.

An unpublished or a publicly available dataset may comprise data generated using particular biomarkers such as polypeptide biomarkers. In some cases the biomarkers comprise markers that are separately or independently searchable through the methods herein or displayed on the systems herein. Some datasets are generated using a biomarker collection, exclusively or in combination with other markers. Some datasets are directed to a particular disorder, a particular pathway, a particular set of genes, or a particular set of proteins. Datasets are identified by the markers used in their generation, or by the source material, or a putative classification of at least some individuals from which samples are obtained, or are otherwise identifiable. Often, databases are identified or are associated with particular markers such that one may find the database by assaying for a node or element that is associated with the dataset. These datasets can be incorporated into the mass spectrometric or computational workflows described herein such as, for example, in study planning or design for identifying biomarkers of interest.

Although the disclosure herein is primarily described with reference to colorectal cancer, it will be understood that the processes and/or apparatuses described herein can be applied to other biomarkers, portions thereof, disorders, pathways, marker providers, experimental result datasets, and/or health conditions.

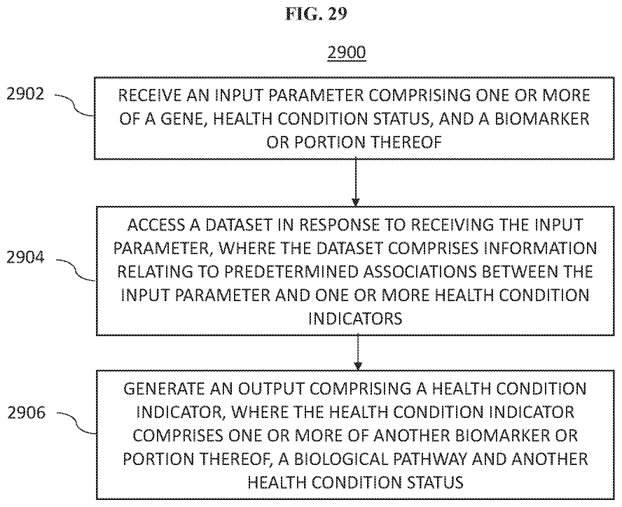

FIG. 29 is a process flow diagram of an example of a health condition indicator identification process 2900. The health condition indicator identification process 2900 can generate an output comprising one or more of a biomarker or portion thereof, a biological pathway, and a health condition status, which has a predetermined association with an input biological parameter. The input biological parameter can comprise one or more of another biomarker or portion thereof, a gene, and/or another health condition status.

Referring to FIG. 29, in block 2902, an input parameter can be received, where the input parameter comprises one or more of a gene, a health condition status, and a biomarker or portion thereof. In block 2904, a dataset can be accessed in response to receiving the input, where the dataset comprises information relating to predetermined associations between the input parameter and one or more health condition indicators. The health condition indicators can comprise one or more of another biomarker or portion thereof, a biological pathway and another health condition status. In block 2906, an output can be generated comprising a health condition indicator. The health condition indicator can have a predetermined association with the input parameter. For example, the output can comprise one or more of another biomarker or portion thereof, a biological pathway and another health condition status. The one or more of another biomarker or portion thereof, a biological pathway and another health condition status can be identified based on the predetermined associations as designated in the dataset.

A user can provide the input to a health condition indicator identification model, such that one or more of a biomarker or portion thereof, a biological pathway and a health condition status can be generated by the model in response to the input, where the biomarker or portion thereof, biological pathway and/or health condition status have a predetermined association with the input. In some cases, the model can be configured to access one or more datasets comprising information of the predetermined associations. In some cases, the one or more datasets comprise publicly available information (e.g., databases maintained by National Center for Biotechnology Information). The health condition indicator identification model can be configured to access the datasets and generate the output which has desired relationships with the input biological parameter.

In some cases, the input parameter comprises one or more genes. In response to receiving the one or more genes, one or more of a biological pathway, a biomarker or portion thereof, and a health condition implicated by the one or more genes, can be identified. The process can return more than one biological pathways, biomarkers or portions thereof, and/or health conditions. For example, the process can identify a protein, a peptide, and/or a polypeptide implicated by the genes, such as a protein, a peptide and/or a polypeptide generated, consumed and/or modified in a biological pathway affected by the genes. The process can be configured to identify a disease implicated by the genes, including for example a colorectal health status, such as colorectal cancer. In some cases, the input consists of the one or more genes.

In some cases, the input parameter comprises one or more biomarkers or portions thereof. For example, the input parameter can comprise one or more of a protein, peptide and polypeptide. In response to receiving the one or more biomarkers or portions thereof, one or more of a biological pathway, another biomarker or portion thereof, and a health condition implicated by the one or more biomarkers or portions thereof, can be identified. The process can return more than one biological pathways, biomarkers or portions thereof, and/or health conditions. For example, the process can identify a protein, peptide and/or a polypeptide implicated by the biomarkers or portions thereof, such as a protein peptide and/or a polypeptide generated, consumed and/or modified in a shared biological pathway. The process can be configured to identify a disease implicated by the biomarkers or portions thereof, including for example a colorectal health status, such as colorectal cancer. In some cases, the input consists of the one or more genes. In some cases, the input parameter consists of the one or more biomarkers or portions thereof.

In some cases, the input parameter comprises one or more health conditions. In response to receiving the one or more health conditions, one or more of a biological pathway, a biomarker or portion thereof, and another health condition implicated by the one or more health conditions, can be identified. The process can return more than one biological pathways, biomarkers or portions thereof, and/or health conditions. For example, the process can identify a protein, a peptide and/or a polypeptide implicated by the health conditions, such as a protein, a peptide and/or a polypeptide generated, consumed and/or modified in a biological pathway affected by the health conditions. The process can be configured to identify another health condition, such as a disease having a correlation with the input health condition. In some cases, the input consists of the one or more health conditions.

In some cases, one or more health condition identification models may further perform analysis of the health condition indicators and provide recommendations based on the health condition indicators.

An output of a health condition indicator identification model as described herein can be provided in one or more formats, including in text form, such as in an alphanumerical format, as a graph, a table, a chart and/or a diagram. In some cases, the output format can be predetermined. In some cases, the output format can be selected by the user. For example, the user can be solicited to select the format from a list of available formats.

In some cases, the user does not actively specify a type and/or a format of the output. A user may not need to select whether the output comprises a biological pathway, a health condition status and/or a biomarker or portion thereof, and/or whether the output is displayed as alphanumerical format, as a graph, chart, table and/or diagram. For example, the type and/or format of the output can be predetermined such that the predetermined output type and/or display format are provided automatically in response to receiving the user input. Alternatively, the user can specify a desired output type and/or format. For example, the user can indicate via a user interface a desired type and format, of the output.

In some cases, a user can provide an input parameter indicating a presence of a colorectal disease to a health condition indicator identification model, such that one or more of a biomarker or portion thereof, a biological pathway and a health condition status having a predetermined association with the colorectal disease can be generated by the model in response to the input. The model can be configured to access one or more datasets comprising information of the predetermined associations between the input parameter indicative of the presence of the colorectal disease and the output.

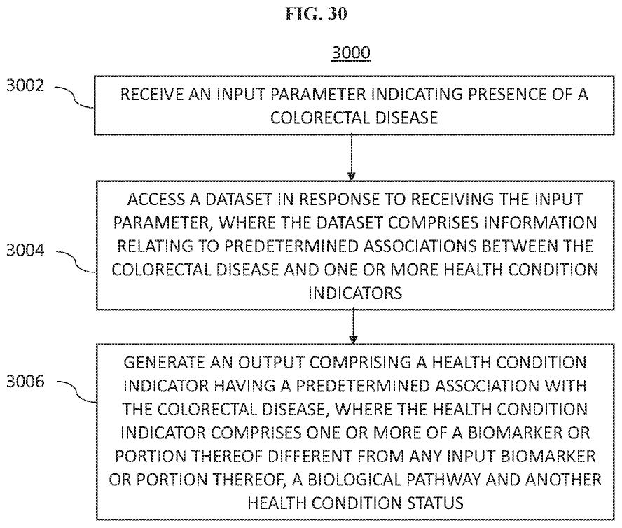

FIG. 30 is process flow diagram of an example of a process 3000 for identifying one or more of a biological pathway, a biomarker or portion thereof, and another health condition status in response to receiving an input parameter indicating a presence of colorectal disease. Colorectal disease can comprise a number of abnormalities of the colon, including colorectal cancer. In block 3002, an input parameter indicating a presence of colorectal disease can be received. The input parameter indicative of the presence of colorectal disease can comprise a biomarker or portion thereof that is implicated by colorectal disease. For example, the level of the biomarker or portion thereof can be known to be positively or negatively correlated with the presence of colorectal disease. In some cases, the input parameter can comprise another health condition implicated by colorectal disease, such as another disease correlated with the presence of the colorectal disease. In some cases, the input parameter can comprise a gene known to be associated with colorectal disease.

In block 3004, a dataset can be accessed in response to receiving the input parameter, where the dataset comprises information relating to predetermined associations between the colorectal disease and one or more health condition indicators. The one or more health condition indicators can comprise one or more of a biological pathway, a biomarker or portion thereof, and another health condition status other than the presence of colorectal disease.

In block 3006, an output comprising a health condition indicator having a predetermined association with the presence of the colorectal disease can be generated. The one or more health condition indicators can comprise a biomarker or portion thereof different from any input biomarker or portion thereof, a biological pathway and another health condition status can be identified based on the predetermined associations as designated in the dataset. For example, the output can comprise a biological pathway implicated by the colorectal disease, such as a biological pathway which is known to be linked to the colorectal disease. The biological pathway can comprise processes known to be correlated with the presence of the colorectal disease. The output can comprise a biomarker or portion thereof with a known correlation to the colorectal disease. In some cases, the output can comprise a health condition status known to be associated with the colorectal disease, such as another disease which has a predetermined association with the colorectal disease.

Any of the biomarkers described herein can be protein biomarkers. Furthermore, the group of biomarkers in this example can in some cases additionally comprise polypeptides with the characteristics found in Table 1.

Exemplary protein biomarkers and, when available, their human amino acid sequences, are listed in Table 1, below. Protein biomarkers comprise full length molecules of the polypeptide sequences of Table 1, as well as uniquely identifiable fragments of the polypeptide sequences of Table 1. Markers can be but do not need to be full length to be informative. In many cases, so long as a fragment is uniquely identifiable as being derived from or representing a polypeptide of Table 1, it is informative for purposes herein.

TABLE 1
Biomarkers and corresponding Descriptors

| No./Protein | |
|---|---|
| Name/ | |
| Protein | |
| Symbol and | Protein Sequence (N- to C-terminal |
| Synonyms/ | single-letter amino acid sequence) or |
| Uniprot ID | other Descriptor of Biomarker |
| No. 1/Alpha- | MALSWVLTVLSLLPLLEAQIPLCANLVPVPITNATLDQITGKWFYIASA |
| 1-acid | FRNEEYNKSVQEIQATFFYFTPNKTEDTIFLREYQTRQDQCIYNTTYLN |
| glycoprotein 1/ | VQRENGTISRYVGGQEHFAHLLILRDTKTYMLAFDVNDEKNWGLSVY |
| A1AG1/ | ADKPETTKEQLGEFYEALDCLRIPKSDVVYTDWKKDKCEPLEKQHEK |
| AlAG/ | ERKQEEGES |
| ORM1/ | |
| P02763 | |
| No. 2/Alpha- | MPSSVSWGILLLAGLCCLVPVSLAEDPQGDAAQKTDTSHHDQDHPTF |
| 1 Antitrypsin/ | NKITPNLAEFAFSLYRQLAHQSNSTNIFFSPVSIATAFAMLSLGTKADTH |
| A1AT, PI, | DEILEGLNFNLTEIPEAQIHEGFQELLRTLNQPDSQLQLTTGNGLFLSEG |
| SERPINA 1/ | LKLVDKFLEDVKKLYHSEAFTVNFGDTEEAKKQINDYVEKGTQGKIV |
| P01009 | DLVKELDRDTVFALVNYIFFKGKWERPFEVKDTEEEDFHVDQVTTVK |
| | VPMMKRLGMFNIQHCKKLSSWVLLMKYLGNATAIFFLPDEGKLQHLE |
| | NELTHDIITKFLENEDRRSASLHLPKLSITGTYDLKSVLGQLGITKVFSN |
| | GADLSGVTEEAPLKLSKAVHKAVLTIDEKGTEAAGAMFLEAIPMSIPPE |
| | VKFNKPFVFLMIEQNTKSPLFMGKVVNPTQK |
| No. 3/Alpha- | MERMLPLLALGLLAAGFCPAVLCHPNSPLDEENLTQENQDRGTHVDL |
| 1- | GLASANVDFAFSLYKQLVLKAPDKNVIFSPLSISTALAFLSLGAHNTTL |
| Antichymotrypsin/ | TEILKGLKFNLTETSEAEIHQSFQHLLRTLNQSSDELQLSMGNAMFVKE |
| AACT, | QLSLLDRFTEDAKRLYGSEAFATDFQDSAAAKKLINDYVKNGTRGKIT |
| SERPINA 3/ | DLIKDLDSQTMMVLVNYIFFKAKWEMPFDPQDTHQSRFYLSKKKWV |
| P01011 | MVPMMSLHHLTIPYFRDEELSCTVVELKYTGNASALFILPDQDKMEEV |
| | EAMLLPETLKRWRDSLEFREIGELYLPKFSISRDYNLNDILLQLGIEEAF |
| | TSKADLSGITGARNLAVSQVVHKAVLDVFEEGTEASAATAVKITLLSA |
| | LVETRTIVRFNRPFLMIIVPTDTQNIFFMSKVTNPKQA |
| No. 4/ | MQPSSLLPLALCLLAAPASALVRIPLHKFTSIRRTMSEVGGSVEDLIAK |
| Cathepsin D/ | GPVSKYSQAVPAVTEGPIPEVLKNYMDAQYYGEIGIGTPPQCFTVVFD |
| CATD, | TGSSNLWVPSIHCKLLDIACWIHHKYNSDKSSTYVKNGTSFDIHYGSGS |
| CTSD, CPSD/ | LSGYLSQDTVSVPCQSASSASALGGVKVERQVFGEATKQPGITFIAAKF |
| P07339 | DGILGMAYPRISVNNVLPVFDNLMQQKLVDQNIFSFYLSRDPDAQPGG |
| | ELMLGGTDSKYYKGSLSYLNVTRKAYWQVHLDQVEVASGLTLCKEG |
| | CEAIVDTGTSLMVGPVDEVRELQKAIGAVPLIQGEYMIPCEKVSTLPAI |
| | TLKLGGKGYKLSPEDYTLKVSQAGKTLCLSGFMGMDIPPPSGPLWILG |
| | DVFIGRYYTVFDRDNNRVGFAEAARL |
| No. 5/ | MESPSAPPHRWCIPWQRLLLTASLLTFWNPPTTAKLTIESTPFNVAEGK |
| Carcinoembryonic | EVLLLVHNLPQHLFGYSWYKGERVDGNRQIIGYVIGTQQATPGPAYSG |
| antigen- | REIIYPNASLLIQNIIQNDTGFYTLHVIKSDLVNEEATGQFRVYPELPKPS |
| related cell | ISSNNSKPVEDKDAVAFTCEPETQDATYLWWVNNQSLPVSPRLQLSNG |
| adhesion | NRTLTLFNVTRNDTASYKCETQNPVSARRSDSVILNVLYGPDAPTISPL |
| molecule 3/ | NTSYRSGENLNLSCHAASNPPAQYSWFVNGTFQQSTQELFIPNITVNNS |
| CEA | GSYTCQAHNSDTGLNRTTVTTITVYAEPPKPFITSNNSNPVEDEDAVAL |
| CAM5 (CEA)/ | TCEPEIQNTTYLWWVNNQSLPVSPRLQLSNDNRTLTLLSVTRNDVGPY |
| P06731 | ECGIQNKLSVDHSDPVILNVLYGPDDPTISPSYTYYRPGVNLSLSCHAA |
| | SNPPAQYSWLIDGNIQQHTQELFISNITEKNSGLYTCQANNSASGHSRT |
| | TVKTITVSAELPKPSISSNNSKPVEDKDAVAFTCEPEAQNTTYLWWVN |
| | GQSLPVSPRLQLSNGNRTLTLFNVTRNDARAYVCGIQNSVSANRSDPV |
| | TLDVLYGPDTPIISPPDSSYLSGANLNLSCHSASNPSPQYSWRINGIPQQ |
| | HTQVLFIAKITPNNNGTYACFVSNLATGRNNSIVKSITVSASGTSPGLSA |
| | GATVGIIVIIGVLVGVALI |
| No. 6/ | MMKTLLLFVGLLLTWESGQVLGDQTVSDNELQEMSNQGSKYVNKEI |
| Clusterin/ | QNAVNGVKQIKTLIEKTNEERKTLLSNLEEAKKKKEDALNETRESETK |
| CLUS, CLU, | LKELPGVCNETMMALWEECKPCLKQTCMKFYARVCRSGSGLVGRQL |
| APOJ, CLI, | EEFLNQSSPFYFWMNGDRIDSLLENDRQQTHMLDVMQDHFSRASSIID |
| KUB1/ | ELFQDRFFTREPQDTYHYLPFSLPHRRPHFFFPKSRIVRSLMPFSPYEPL |
| P10909 | NFHAMFQPFLEMIHEAQQAMDIHFHSPAFQHPPTEFIREGDDDRTVCR |
| | EIRHNSTGCLRMKDQCDKCREILSVDCSTNNPSQAKLRRELDESLQVA |
| | ERLTRKYNELLKSYQWKMLNTSSLLEQLNEQFNWVSRLANLTQGEDQ |
| | YYLRVTTVASHTSDSDVPSGVTEVVVKLFDSDPITVTVPVEVSRKNPK |
| | FMETVAEKALQEYRKKHREE |
| No. 7/ | MSACRSFAVAICILEISILTAQYTTSYDPELTESSGSASHIDCRMSPWSE |
| Complement | WSQCDPCLRQMFRSRSIEVFGQFNGKRCTDAVGDRRQCVPTEPCEDA |
| C9/C9, C09/ | EDDCGNDFQCSTGRCIKMRLRCNGDNDCGDFSDEDDCESEPRPPCRDR |
| P02748 | VVEESELARTAGYGINILGMDPLSTPFDNEFYNGLCNRDRDGNTLTYY |
| | RRPWNVASLIYETKGEKNFRTEHYEEQIEAFKSIIQEKTSNFNAAISLKF |
| | TPTETNKAEQCCEETASSISLHGKGSFRFSYSKNETYQLFLSYSSKKEK |
| | MFLHVKGEIHLGRFVMRNRDVVLTTTFVDDIKALPTTYEKGEYFAFLE |
| | TYGTHYSSSGSLGGLYELIYVLDKASMKRKGVELKDIKRCLGYHLDVS |
| | LAF SEISVGAEFNKDDCVKRGEGRAVNITSENLIDDVVSLIRGGTRKYA |
| | FELKEKLLRGTVIDVTDFVNWASSINDAPVLISQKLSPIYNLVP VKMKN |
| | AHLKKQNLERAIEDYINEFSVRKCHTCQNGGTVILMDGKCLCACPFKF |
| | EGIACEISKQKISEGLPALEFPNEK |
| No. 8/ | MKTPWKVLLGLLGAAALVTIITVPVVLLNKGTDDATADSRKTYTLTD |
| Dipeptidyl | YLKNTYRLKLYSLRWISDHEYLYKQENNILVFNAEYGNSVFLENSTFD |
| peptidase 4/ | EFGHSINDYSISPDGQFILLEYNYVKQWRHSYTASYDIYDLNKRQLITE |
| DPP4, | ERIPNNTQWVTWSPVGHKLAYVWNNDIYVKIEPNLPSYRITWTGKEDI |
| DPPIV, | IYNGITDWVYEEEVFSAYSALWWSPNGTFLAYAQFNDTEVPLIEYSFY |
| ADCP2, | SDESLQYPKTVRVPYPKAGAVNPTVKFFVVNTDSLSSVTNATSIQITAP |
| CD26/ | ASMLIGDHYLCDVTWATQERISLQWLRRIQNYSVMDICDYDESSGRW |
| P27487 | NCLVARQHIEMSTTGWVGRFRPSEPHFTLDGNSFYKIISNEEGYRHICY |
| | FQIDKKDCTFITKGTWEVIGIEALTSDYLYYISNEYKGMPGGRNLYKIQ |
| | LSDYTKVTCLSCELNPERCQYYSVSFSKEAKYYQLRCSGPGLPLYTLH |
| | SSVNDKGLRVLEDNSALDKMLQNVQMPSKKLDFIILNETKFWYQMILP |
| | PHFDKSKKYPLLLDVYAGPCSQKADTVFRLNWATYLASTENIIVASFD |
| | GRGSGYQGDKIMHAINRRLGTFEVEDQIEAARQFSKMGFVDNKRIAIW |
| | GWSYGGYVTSMVLGSGSGVFKCGIAVAPVSRWEYYDSVYTERYMGL |
| | PTPEDNLDHYRNSTVMSRAENFKQVEYLLIHGTADDNVHFQQSAQISK |
| | ALVDVGVDFQAMWYTDEDHGIASSTAHQHIYTHMSHFIKQCFSLP |
| No. 9/ | MAPHRPAPALLCALSLALCALSLPVRAATASRGASQAGAPQGRVPEA |
| Gelsolin/ | RPNSMVVEHPEFLKAGKEPGLQIWRVEKFDLVPVPTNLYGDFFTGDA |
| GELS, GSN/ | YVILKTVQLRNGNLQYDLHYWLGNECSQDESGAAAIFTVQLDDYLNG |
| P06396 | RAVQHREVQGFESATFLGYFKSGLKYKKGGVASGFKHVVPNEVVVQ |
| | RLFQVKGRRVVRATEVPVSWESFNNGDCFILDLGNNIHQWCGSNSNR |
| | YERLKATQVSKGIRDNERSGRARVHVSEEGTEPEAMLQVLGPKPALPA |
| | GTEDTAKEDAANRKLAKLYKVSNGAGTMSVSLVADENPFAQGALKS |
| | EDCFILDHGKDGKIFVWKGKQANTEERKAALKTASDFITKMDYPKQT |
| | QVSVLPEGGETPLFKQFFKNWRDPDQTDGLGLSYLSSHIANVERVPFD |
| | AATLHTSTAMAAQHGMDDDGTGQKQIWRIEGSNKVPVDPATYGQFY |
| | GGDSYIILYNYRHGGRQGQIIYNWQGAQSTQDEVAASAILTAQLDEEL |
| | GGTPVQSRVVQGKEPAHLMSLFGGKPMIIYKGGTSREGGQTAPASTRL |
| | FQVRANSAGATRAVEVLPKAGALNSNDAFVLKTPSAAYLWVGTGASE |
| | AEKTGAQELLRVLRAQPVQVAEGSEPDGFWEALGGKAAYRTSPRLKD |
| | KKMDAHPPRLFACSNKIGRFVIEEVPGELMQEDLATDDVMLLDTWDQ |
| | VFVWVGKDSQEEEKTEALTSAKRYIETDPANRDRRTPITVVKQGFEPP |
| | SFVGWFLGWDDDYWSVDPLDRAMAELAA |
| No. 10/ | MPMFIVNTNVPRASVPDGFLSELTQQLAQATGKPPQYIAVHVVPDQL |
| Macrophage | MAFGGSSEPCALCSLHSIGKIGGAQNRSYSKLLCGLLAERLRISPDRVYI |
| migration | NYYDMNAANVGWNNSTFA |
| inhibitory | |
| factor/MIF, | |
| GLIF, MMIF/ | |
| P14174 | |
| No. 11/ | MSKPHSEAGTAFIQTQQLHAAMADTFLEHMCRLDIDSPPITARNTGIIC |
| Pyruvate | TIGPASRSVETLKEMIKSGMNVARLNFSHGTHEYHAETIKNVRTATESF |
| kinase/PKM, | ASDPILYRPVAVALDTKGPEIRTGLIKGSGTAEVELKKGATLKITLDNA |
| OIP3, PK2, | YMEKCDENILWLDYKNICKVVEVGSKIYVDDGLISLQVKQKGADFLV |
| PK3, PKM2/ | TEVENGGSLGSKKGVNLPGAAVDLPAVSEKDIQDLKFGVEQDVDMVF |
| P14618 | ASFIRKASDVHEVRKVLGEKGKNIKIISKIENHEGVRRFDEILEASDGIM |
| | VARGDLGIEIPAEKVFLAQKMMIGRCNRAGKPVICATQMLESMIKKPR |
| | PTRAEGSDVANAVLDGADCIMLSGETAKGDYPLEAVRMQHLIAREAE |
| | AAIYHLQLFEELRRLAPITSDPTEATAVGAVEASFKCCSGAIIVLTKSGR |
| | SAHQVARYRPRAPIIAVTRNPQTARQAHLYRGIFPVLCKDPVQEAWAE |
| | DVDLRVNFAMNVGKARGFFKKGDVVIVLTGWRPGSGFTNTMRVVPV |
| | P |
| No. 12/ | >SAA1 |
| “SAA” Serum | MKLLTGLVFCSLVLGVSSRSFFSFLGEAFDGARDMWRAYSDMREANY |
| amyloid A-1 | IGSDKYFHARGNYDAAKRGPGGVWAAEAISDARENIQRFFGHGAEDS |
| protein/ | LADQAANEWGRSGKDPNHFRPAGLPEKY |
| Serum | >SAA2 |
| Amyloid A-2 | MKLLTGLVFCSLVLSVSSRSFFSFLGEAFDGARDMWRAYSDMREANYI |
| protein/ | GSDKYFHARGNYDAAKRGPGGAWAAEVISNARENIQRLTGRGAEDSL |
| SAA1. SAA2, | ADQAANKWGRSGRDPNHFRPAGLPEKY |
| SAA1/2/ | Note that unlike the other markers, marker ‘SAA’ |
| SAA2/4 | represents either or both of two closely related |
| P0DJI8/ | SAA proteins listed above. The proteins share 93% |
| P0DJI9. | identity over their common 122 residue length. |
| | An ‘SAA’ measurement variously refers to SAA1, |
| | SAA2, or a combined measurement of SAA1 and SAA2. |
| No. 13 / | MAPFEPLASGILLLLWLIAPSRACTCVPPHPQTAFCNSDLVIRAKFVGTP |
| Metalloproteinase | EVNQTTLYQRYEIKMTKMYKGFQALGDAADIRFVYTPAMESVCGYFH |
| inhibitor | RSHNRSEEFLIAGKLQDGLLHITTCSFVAPWNSLSLAQRRGFTKTYTVG |
| 1/ TIMP1, | CEECTVFPCLSIPCKLQSGTHCLWTDQLLQGSEKGFQSRHLACLPREPG |
| CLGI/ | LCTWQSLRSQIA |
| P01033 | |
| No. 14/ | MMDQARSAFSNLFGGEPLSYTRFSLARQVDGDNSHVEMKLAVDEEEN |
| Transferrin | ADNNTKANVTKPKRCSGSICYGTIAVIVFFLIGFMIGYLGYCKGVEPKT |
| Receptor | ECERLAGTESPVREEPGEDFPAARRLYWDDLKRKLSEKLDSTDFTGTI |
| Protein 1/ | KLLNENSYVPREAGSQKDENLALYVENQFREFKLSKVWRDQHFVKIQ |
| TFRC/ | VKDSAQNSVIIVDKNGRLVYLVENPGGYVAYSKAATVTGKLVHANFG |
| P02786 | TKKDFEDLYTPVNGSIVIVRAGKITFAEKVANAESLNAIGVLIYMDQTK |
| | FPIVNAELSFFGHAHLGTGDPYTPGFPSFNHTQFPPSRSSGLPNIPVQTIS |
| | RAAAEKLFGNMEGDCPSDWKTDSTCRMVTSESKNVKLTVSNVLKEIK |
| | ILNIFGVIKGFVEPDHYVVVGAQRDAWGPGAAKSGVGTALLLKLAQM |
| | FSDMVLKDGFQPSRSIIFASWSAGDFGSVGATEWLEGYLSSLHLKAFT |
| | YINLDKAVLGTSNFKVSASPLLYTLIEKTMQNVKHPVTGQFLYQDSNW |
| | ASKVEKLTLDNAAFPFLAYSGIPAVSFCFCEDTDYPYLGTTMDTYKELI |
| | ERIPELNKVARAAAEVAGQFVIKLTHDVELNLDYERYNSQLLSFVRDL |
| | NQYRADIKEMGLSLQWLYSARGDFFRATSRLTTDF |
| | GNAEKTDRFVMKKLNDRVMRVEYHFLSPYVSPKESPFRHVFWGSGSH |
| | TLPALLENLKLRKQNNGAFNETLFRNQLALATWTIQGAANALSGDVW |
| | DIDNEF |
| No. 15/ | MPGQELRTVNGSQMLLVLLVLSWLPHGGALSLAEASRASFPGPSELHS |
| Growth/ | EDSRFRELRKRYEDLLTRLRANQSWEDSNTDLVPAPAVRILTPEVRLG |
| differentiation | SGGHLHLRISRAALPEGLPEASRLHRALFRLSPTASRSWDVTRPLRRQL |
| factor 15/ | SLARPQAPALHLRLSPPPSQSDQLLAESSSARPQLELHLRPQAARGRRR |
| GDF 15, | ARARNGDHCPLGPGRCCRLHTVRASLEDLGWADWVLSPREVQVTMC |
| MIC1, PDF, | IGACPSQFRAANMHAQIKTSLHRLKPDTVPAPCCVPASYNPMVLIQKT |
| PLAB, | DTGVSLQTYDDLLAKDCHCI |
| PTGFB/ | |
| Q99988 | |
| No. 16 | Patient Age |
| No. 17 | Patient Gender |

Biomarkers contemplated herein also include polypeptides having an amino acid sequence identical to a listed marker of Table 1 over a span of 8 residues, 9, residues, 10 residues, 20 residues, 50 residues, or alternately 5%, 10%, 20%, 30%, 40%, 50%, 60%, 70% 80% 90%, 95% or greater than 95% of the sequence of the biomarker. Variant or alternative forms of the biomarker include for example polypeptides encoded by any splice-variants of transcripts encoding the disclosed biomarkers. In certain cases the modified forms, fragments, or their corresponding RNA or DNA, may exhibit better discriminatory power in diagnosis than the full-length protein.

Biomarkers contemplated herein also include truncated forms or polypeptide fragments of any of the proteins described herein. Truncated forms or polypeptide fragments of a protein can include N-terminally deleted or truncated forms and C-terminally deleted or truncated forms. Truncated forms or fragments of a protein can include fragments arising by any mechanism, such as, without limitation, by alternative translation, exo- and/or endo-proteolysis and/or degradation, for example, by physical, chemical and/or enzymatic proteolysis. Without limitation, a biomarker may comprise a truncated or fragment of a protein, polypeptide or peptide may represent about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% of the amino acid sequence of the protein.

Without limitation, a truncated or fragment of a protein may include a sequence of about 5-20 consecutive amino acids, or about 10-50 consecutive amino acids, or about 20-100 consecutive amino acids, or about 30-150 consecutive amino acids, or about 50-500 consecutive amino acid residues of the corresponding full length protein.

In some instances, a fragment is N-terminally and/or C-terminally truncated by between 1 and about 20 amino acids, such as, for example, by between 1 and about 15 amino acids, or by between 1 and about 10 amino acids, or by between 1 and about 5 amino acids, compared to the corresponding mature, full-length protein or its soluble or plasma circulating form.

Any protein biomarker of the present disclosure such as a peptide, polypeptide or protein and fragments thereof may also encompass modified forms of said marker, peptide, polypeptide or protein and fragments such as bearing post-expression modifications including but not limited to, modifications such as phosphorylation, glycosylation, lipidation, methylation, selenocystine modification, cysteinylation, sulphonation, glutathionylation, acetylation, oxidation of methionine to methionine sulphoxide or methionine sulphone, and the like.

In some instances, a fragmented protein is N-terminally and/or C-terminally truncated. Such fragmented protein can comprise one or more, or all transitional ions of the N-terminally (a, b, c-ion) and/or C-terminally (x, y, z-ion) truncated protein or peptide. Exemplary human markers such as nucleic acids, proteins or polypeptides as taught herein are as annotated under NCBI Genbank (accessible at the website ncbi.nlm.nih.gov) or Swissprot/Uniprot (accessible at the website uniprot.org) accession numbers. In some instances said sequences are of precursors (for example, pre-proteins) of the markers (e.g., nucleic acids, proteins or polypeptides, lipids, metabolites, and other biomolecules) as taught herein and may include parts which are processed away from mature molecules. In some instances although only one or more isoforms is disclosed, all isoforms of the sequences are intended.

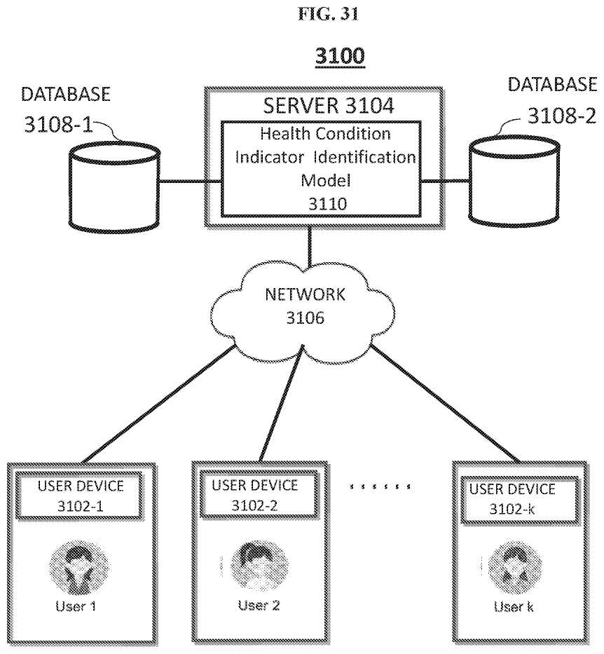

FIG. 31 shows an example of a network layout 3100 comprising one or more user devices 3102, a server 3104, a network 3106, and databases 3108. Each of the components 3102, 3104 and 3108 can be operatively connected to one another via the network 3106. A health condition indicator identification model 3110 can be maintained on the server 3104. FIG. 31 shows two databases 3108-1 and 3108-2. It will be understood more or fewer databases can be included in the network layout 3100. The network 3106 may comprise any type of communication links that allows transmission of data from one electronic component to another. A health condition indicator identification system can comprise one or more components of the network layout 3100. In some cases, a health condition indicator identification system can comprise the server 3104 on which a health condition indicator identification model 3110 is stored. In some cases, a health condition indicator identification system can comprise the server 3104 and the databases 3108. In some cases, a health condition indicator identification system comprises the user devices 3102, the server 3104, and the databases 3108.

In some embodiments, the health condition indicator identification system may comprise software that, when executed by processor(s), perform processes for generating the health condition indicators for a user. In certain configurations, the health condition indicator identification model 3110 may be software stored in memory accessible by the server 3104 (e.g., in a memory local to the server or remote memory accessible over a communication link, such as the network). Thus, in certain aspects, the health condition indicator identification model 3110 may be implemented as one or more computers, as software stored on a memory device accessible by the server 3104, or a combination thereof.

In some embodiments, the health condition indicator identification model or a portion thereof can be provided to a user device 3102 for generating a requested health condition indicator. For example, a software and/or application for implementing the health condition indicator identification model can be provided to a user device 3102. In one aspect, the software and/or applications can be downloaded to a local user device and executed on the local device for generating the requested health condition indicator. For example, the downloaded software and/or application can be configured to enable communication between the user device 3102 and the databases 3108 to generate one or more health condition indicators. In some embodiments, the software and/or applications may be maintained on a server remote from the user device, such as on a server at a geographical location different from that of the user device (e.g., in a different office, office building, city, and/or state). In some embodiments, the software and/or application for implementing the health condition indicator identification model can be implemented at the server 3104 such that the health condition indicator is generated at the server 3104 and the generated indicator is then provided to the user device 3102.

A user device 3102 may be, for example, one or more computing devices configured to perform one or more operations consistent with the disclosed embodiments. For example, a user device 3102 may be a computing device configured to execute software and/or applications for the health condition indicator identification model 3110. In some cases, the user device 3102 can be configured to communicate with the server 3104 and/or the databases 3108. A user device 3102 can include, among other things, desktop computers, laptops or notebook computers, mobile devices (e.g., smart phones, cell phones, personal digital assistants (PDAs), and tablets), or wearable devices (e.g., smartwatches). A user device 3102 can also include any other media content player, for example, a set-top box, a television set, a video game system, or any electronic device capable of providing or rendering data. A user device 3102 may include known computing components, such as one or more processors, and one or more memory devices storing software instructions executed by the processor(s) and data. In some cases, the user device may be portable. The user device may be handheld.

In some embodiments, the network layout 3100 may include a plurality of user devices 3102. Each user device may be associated with a user. Users may include any individual or groups of individuals using software and/or applications of the health condition indicator identification system. For example, the users may access a user device 3102 or a web account using an application programmable interface (API) provided by the health condition indicator identification system. In some embodiments, more than one user may be associated with a user device 3102. Alternatively, more than one user device 3102 may be associated with a user. The users may be located geographically at a same location, for example users working in a same office or a same geographical location. In some instances, some or all of the users and user devices 3102 may be at remote geographical locations (e.g., different office, office building, cities, states, etc.), although this is not a limitation of the invention.

The network layout may include a plurality of nodes. Each user device in the network layout may correspond to a node. If a “user device 3102” is followed by a number or a letter, it means that the “user device 3102” may correspond to a node sharing the same number or letter. For example, as shown in FIG. 31, user device 3102-1 may correspond to node 1 which is associated with user 1, user device 3102-2 may correspond to node 2 which is associated with user 2, and user device 3102-k may correspond to node k which is associated with user k, where k may be any integer greater than 1.

A node may be a logically independent entity in the network layout. Therefore, the plurality of nodes in the network layout can represent different entities. For example, each node may be associated with a user, a group of users, or groups of users. For example, in one embodiment, a node may correspond to an individual entity (e.g., an individual). In some particular embodiments, a node may correspond to multiple entities (e.g., a group of individuals).

A user may be registered or associated with an entity that provides services associated with one or more operations performed by the disclosed embodiments. For example, the user may be a registered user of an entity (e.g., a company, an organization, an individual, etc.) that provides one or more of the user devices 3102, the servers 3104, the databases 3108, and/or the health condition indicator identification model 3110 consistent with certain disclosed embodiments. The disclosed embodiments are not limited to any specific relationships or affiliations between the users and an entity, person(s), or entities providing the user devices, server 3104, databases 3108, and health condition indicator identification model 3110.

A user device may be configured to receive input from one or more users. A user may provide an input to a user device using a user interface, for example, a keyboard, a mouse, a touch-screen panel, voice recognition and/or dictation software, or any combination of the above. The input may include a user performing various virtual actions during a health condition indicator identification session. The input may include, for example, a user selecting a desired health condition indicator and/or a format of the health condition indicator to view from a plurality of options that are presented to the user during a health condition indicator identification session. In another example, the input may include a user providing user credentials such as password or biometrics to verify the identity of the user, for example in order to use the software and/or application and/or communicate with the server 3104 using the user device.

In the embodiment of FIG. 31, two-way data transfer capability may be provided between the server 3104 and each user device 3102. The user devices 3102 can also communicate with one another via the server 3104 (e.g., using a client-server architecture). In some embodiments, the user devices 3102 can communicate directly with one another via a peer-to-peer communication channel. The peer-to-peer communication channel can help to reduce workload on the server 3104 by utilizing resources (e.g., bandwidth, storage space, and/or processing power) of the user devices 3102.

The server 3104 may comprise one or more server computers configured to perform one or more operations consistent with disclosed embodiments. In one aspect, the server 3104 may be implemented as a single computer, through which a user device 3102 is able to communicate with other components of the network layout 3100. In some embodiments, a user device 3102 may communicate with the server 3104 through the network 3106. In some embodiments, the server 3104 may communicate on behalf of a user device 3102 with the database 3108 through the network 3106. The health condition indicator identification model 3110 may be maintained on the server 3104 such that user devices 3102 may access the health condition indicator identification model 3110 by communicating with the server 3104 via the network 3106. In some cases, the health condition indicator identification model 3110 may be software and/or hardware components included with the server 3104.

In some embodiments, a user device 3102 may be directly connected to the server 3104 through a separate link (not shown in FIG. 31). In certain embodiments, the server 3104 may be configured to operate as a front-end device configured to provide access to the health condition indicator identification model 3110 consistent with certain disclosed embodiments. The server 3104 may, in some embodiments, utilize the health condition indicator identification model 3110 to process input data from a user device 3102 in order to retrieve information from the database 3108 to generate the requested health condition indicator.

The server 3104 may include a web server, an enterprise server, or any other type of computer server, and can be computer programmed to accept requests (e.g., HTTP, or other protocols that can initiate data transmission) from a computing device (e.g., a user device) and to serve the computing device with requested data. In addition, a server can be a broadcasting facility, such as free-to-air, cable, satellite, and other broadcasting facility, for distributing data. The server 3104 may also be a server in a data network (e.g., a cloud computing network).

The server 3104 may include known computing components, such as one or more processors, one or more memory devices storing software instructions executed by the processor(s), and data. A server can have one or more processors and at least one memory for storing program instructions. The processor(s) can be a single or multiple microprocessors, field programmable gate arrays (FPGAs), or digital signal processors (DSPs) capable of executing particular sets of instructions. Computer-readable instructions can be stored on a tangible non-transitory computer-readable medium, such as a flexible disk, a hard disk, a CD-ROM (compact disk-read only memory), and MO (magneto-optical), a DVD-ROM (digital versatile disk-read only memory), a DVD RAM (digital versatile disk-random access memory), or a semiconductor memory. Alternatively, the methods disclosed herein can be implemented in hardware components or combinations of hardware and software such as, for example, ASICs, special purpose computers, or general purpose computers. While FIG. 31 illustrates the server as a single server, in some embodiments, multiple devices may implement the functionality associated with the server.

The network 3106 may be configured to provide communication between various components of the network layout 3100 depicted in FIG. 31. The network 3106 may be implemented, in some embodiments, as one or more networks that connect devices and/or components in the network layout 3100 for allowing communication between them. For example, as one of ordinary skill in the art will recognize, the network 306 may be implemented as the Internet, a wireless network, a wired network, a local area network (LAN), a Wide Area Network (WANs), Bluetooth, Near Field Communication (NFC), or any other type of network that provides communications between one or more components of the network layout. In some embodiments, the network 3106 may be implemented using cell and/or pager networks, satellite, licensed radio, or a combination of licensed and unlicensed radio. The network 3106 may be wireless, wired, or a combination thereof.

A health condition indicator identification system may be implemented as one or more computers storing instructions that, when executed by one or more processor(s), generate a plurality of health condition indicator. The health condition indicator identification system may generate one or more health condition indicators by accessing data from a database comprising information of predetermined associations between the health condition indicators and a user input parameter. A user can select to view the health condition indicators in a format that is defined by the user. Alternatively, the health condition indicators can be displayed to the user in a predetermined format. For example, the health condition indicator identification system may further display the health condition indicators to the user in a format predetermined by the health condition indicator identification system or by the user. The health condition indicator identification system or may not require user identification information in order to verify or authenticate the user to obtain the health condition indicators the user or perform the health condition indicator identification functions.

In some embodiments, the server 3104 is the computer in which the health condition indicator identification system is implemented. For example, all of the health condition indicator identification functions can be implemented on the server 3104 such that the health condition indicators are generated by the server 3104 and transmitted to the user device 3102. However, in some embodiments, at least some of the health condition indicator identification system may be implemented on separate computers. For example, a user device 3102 may send a user input to the server 3104, and the server 3104 may connect to other health condition indicator identification systems over the network 3106. In some cases, at least a part of the health condition indicator identification functions is implemented locally, such as using a user device 3102. For example, a part of a health condition indicator identification model can be implemented on a user device 3102 and a part of the health condition indicator identification model can be implemented on the server 3104 and/or another health condition indicator identification system in communication with the server 3104.

The user devices 3102 and the server 3104 may be connected or interconnected to one or more databases 3108-1, 3108-2. The databases 3108-1, 3108-2 may be one or more memory devices configured to store data (e.g., predetermined associations between genetic data, biomarkers, biological pathways, and/or health condition statuses, etc.). The databases 3108-1, 3108-2 may, in some embodiments, be implemented as a computer system with a storage device. In one aspect, the databases 3108-1, 3108-2 may be used by components of the network layout to perform one or more operations consistent with the disclosed embodiments. In certain embodiments, one or more the databases 3108-1, 3108-2 may be co-located with the server 3104, or may be co-located with one another on the network 3106. One of ordinary skill will recognize that the disclosed embodiments are not limited to the configuration and/or arrangement of the databases 3108-1, 3108-2.

Any of the user devices, the server, the database(s), and/or the frailty prediction system(s) may, in some embodiments, be implemented as a computer system. Additionally, while the network is shown in FIG. 31 as a “central” point for communications between components of the network layout 3100, the disclosed embodiments are not limited thereto. For example, one or more components of the network layout 3100 may be interconnected in a variety of ways, and may in some embodiments be directly connected to, co-located with, or remote from one another, as one of ordinary skill will appreciate. Additionally, while some disclosed embodiments may be implemented on the server 3104, the disclosed embodiments are not so limited. For instance, in some embodiments, other devices (such as one or more user devices 3102) may be configured to perform one or more of the processes and functionalities consistent with the disclosed embodiments, including embodiments described with respect to the server 3104 and the health condition indicator identification model.

Although particular computing devices are illustrated and networks described, it is to be appreciated and understood that other computing devices and networks can be utilized without departing from the spirit and scope of the embodiments described herein. In addition, one or more components of the network layout may be interconnected in a variety of ways, and may in some embodiments be directly connected to, co-located with, or remote from one another, as one of ordinary skill will appreciate.

A user can interact with the health condition indicator identification model via a user interface. The user interface can be a part of one or more user interfaces described herein. A user interface can comprise a graphical user interface through which the user can provide input and/or view an output of the health condition indicator identification model.

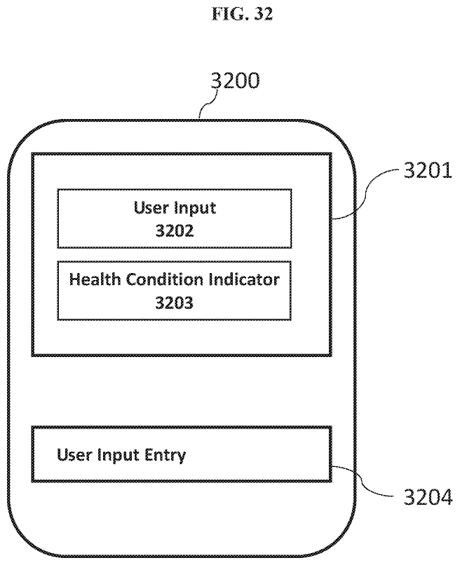

FIG. 32 shows a schematic diagram of an example of a user interface 3200 by which a user may provide input for the health condition indicator identification model and/or view output generated by the health condition indicator identification model. A user interface 3200 may be provided as part of a user device, for example, one or more computing devices configured to perform one or more operations consistent with the disclosed embodiments. The user device can have one or more features as described herein. For example, the user device may be a computer configured to execute software and/or applications for generating the requested health condition indicator. The software and/or applications may be configured to implement at least a portion of the health condition indicator identification model as described herein.

The user interface 3200 may comprise a display screen 3201 to display various identified biomarkers or portions thereof, biological pathways, and/or health condition statuses to the user. In some cases, the display screen 3201 may display input from the user to facilitate use of the device to input information for generating the desired health condition indicators. The display screen 3201 may comprise a graphical user interface. The graphical user interface may comprise a browser, software, and/or application that may aid in the user in using the user device for generating the desired health condition indicators. The user interface 3200 can be configured to facilitate the user's use of the user device to run the application and/or software for generating the desired health condition indicators. The user interface 3200 may be configured to receive user input as described elsewhere herein.

The display screen 3201 can comprise various features to enable visually illustrating information. The information shown on the display may be changeable. The display may include a screen, such as a liquid crystal display (LCD) screen, light-emitting diode (LED) screen, organic light-emitting diode (OLED) screen, plasma screen, electronic ink (e-ink) screen, touchscreen, or any other type of screen or display. The display may or may not accept user input.

The user interface 3200 may allow the user to set up a format of display. For instance, the user may be allowed to select a user preferred format to view the result (e.g., in the form of bar graphs, pie chart, histograms, line charts, alphanumerical format).

The user interface 3200 can comprise one or more components for entry of user input 3204. The user input entry 3204 can comprise a variety of user interactive devices, such as a keyboard, button, mouse, touchscreen, touchpad, joystick, trackball, camera, microphone, motion sensor, heat sensor, inertial sensor, and/or any other type of user interactive device. For instance, a user may input user information 3202 such as command to initiate the health condition indicator 3203 identification process and/or an input parameter through the user interactive device. The user input entry 3204 is shown in FIG. 32 as being a part of the user interface 3200. In some cases, the user input entry 3204 may be separate from the user interface 3200. For example, the user interface 3200 may be a part of a user device and the user input entry 3204 may not be a part of the user device, or vice versa.

As described herein, the user interface 3200 may be incorporated as a part of a user device. The user device may comprise one or more memory storage units which may comprise non-transitory computer readable medium comprising code, logic, or instructions for performing one or more steps. The user device may comprise one or more processors capable of executing one or more steps, for instance in accordance with the non-transitory computer readable media. The one or more memory storage units may store one or more software applications or commands relating to the software applications. The one or more processors may, individually or collectively, execute steps of the software application.

A communication unit may be provided on the device. The communication unit may allow the user device to communicate with an external device. The external device may be a device of a transaction entity, server, or may be a cloud-based infrastructure. The external device can comprise a server as described herein. The communications may include communications over a network or a direct communication. The communication unit may permit wireless or wired communications. Examples of wireless communications may include, but are not limited to WiFi, 3G, 4G, LTE, radiofrequency, Bluetooth, infrared, or any other type of communications.

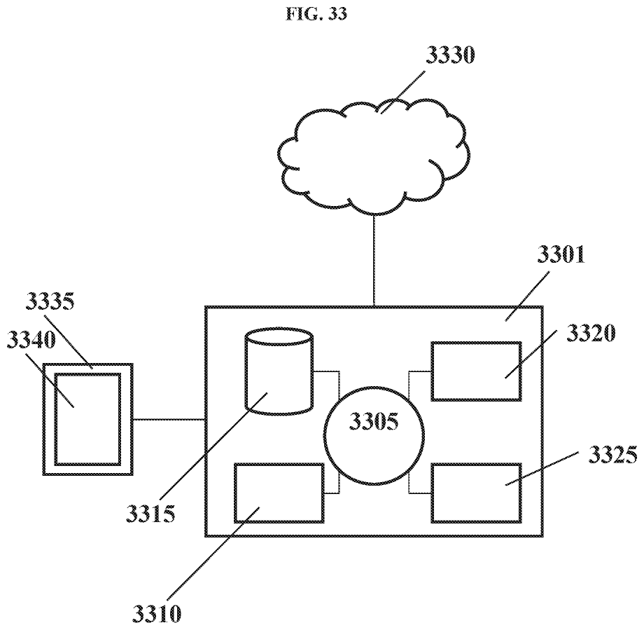

The present disclosure provides computer control systems that are programmed to implement methods of the disclosure. FIG. 33 shows a computer system 3301 that is programmed or otherwise configured to perform health condition indicator identification. In some cases, the computer system 3301 can be a part of a user device as described herein. The computer system 3301 can regulate various aspects of the identification analysis of the present disclosure. The computer system 3301 can be an electronic device of a user or a computer system that is remotely located with respect to the electronic device. The electronic device can be a mobile electronic device or a desktop computer.

The computer system 3301 includes a central processing unit (CPU, also “processor” and “computer processor” herein) 3305, which can be a single core or multi core processor, or a plurality of processors for parallel processing. The computer system 3301 also includes memory or memory location 3310 (e.g., random-access memory, read-only memory, flash memory), electronic storage unit 3315 (e.g., hard disk), communication interface 3320 (e.g., network adapter) for communicating with one or more other systems, and peripheral devices 3325, such as cache, other memory, data storage and/or electronic display adapters. The memory 3310, storage unit 3315, interface 3320 and peripheral devices 3325 are in communication with the CPU 3305 through a communication bus (solid lines), such as a motherboard. The storage unit 3315 can be a data storage unit (or data repository) for storing data. The computer system 3301 can be operatively coupled to a computer network (“network”) 3330 with the aid of the communication interface 3320. The network 3330 can be the Internet, an internet and/or extranet, or an intranet and/or extranet that is in communication with the Internet. The network 3330 in some cases is a telecommunication and/or data network. The network 3330 can include one or more computer servers, which can enable distributed computing, such as cloud computing. The network 3330, in some cases with the aid of the computer system 3301, can implement a peer-to-peer network, which may enable devices coupled to the computer system 3301 to behave as a client or a server.

The CPU 3305 can execute a sequence of machine-readable instructions, which can be embodied in a program or software. The instructions may be stored in a memory location, such as the memory 3310. The instructions can be directed to the CPU 3305, which can subsequently program or otherwise configure. The CPU 3305 to implement methods of the present disclosure. Examples of operations performed by the CPU 3305 can include fetch, decode, execute, and writeback.

The CPU 3305 can be part of a circuit, such as an integrated circuit. One or more other components of the system 3301 can be included in the circuit. In some cases, the circuit is an application specific integrated circuit (ASIC).

The storage unit 3315 can store files, such as drivers, libraries and saved programs. The storage unit 3315 can store user data, e.g., user preferences and user programs. The computer system 3301 in some cases can include one or more additional data storage units that are external to the computer system 3301, such as located on a remote server that is in communication with the computer system 3301 through an intranet or the Internet.

The computer system 3301 can communicate with one or more remote computer systems through the network 3330. For instance, the computer system 3301 can communicate with a remote computer system of a user (e.g., a physician). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 3301 via the network 3330.

Methods as described herein can be implemented by way of machine (e.g., computer processor) executable code stored on an electronic storage location of the computer system 3301, such as, for example, on the memory 3310 or electronic storage unit 3315. The machine executable or machine readable code can be provided in the form of software. During use, the code can be executed by the processor 3305. In some cases, the code can be retrieved from the storage unit 3315 and stored on the memory 3310 for ready access by the processor 3305. In some situations, the electronic storage unit 3315 can be precluded, and machine-executable instructions are stored on memory 3310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 3301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

The computer system 3301 can include or be in communication with an electronic display 3335 that comprises a user interface (UI) 3340 for providing, for example, information about the desired health condition indicators. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 3305. The algorithm can, for example, determine whether a cancer is present and/or progressing.

Systems and methods herein present data in a form readily accessible by a user, such as on a visual display. Such a display allows for complex data outputs to be presented so as to facilitate quick assessment of results. For instance, an input such as a disorder is depicted as a primary or foundational node of an output on a display screen, to which related proteins, peptide or other markers or genes, are configured so as to indicate their involvement in or relevance to the disorder. For the markers, there is in some cases visual or scroll-over indication of whether the marker is commercially available, and from which seller, or whether the marker is already available in one's lab, such as by prior purchase or synthesis.

The related proteins, peptide or other markers or genes are in turn often depicted as being connected to a pathway or pathways in which they are implicated and to disorders or diseases related to the pathways or to the related proteins, peptide or other markers or genes. Similarly, related proteins, peptide or other markers or genes, or related pathways, or related disorders, or indeed the input disorder is tagged via connectivity so as to indicate whether publicly available research results, other publications, or expression data related to any particular node of the display is available. Optionally, nodes related to nonpublic data such as recently generated mass spectrometric data or expression data are also indicated by connectivity to a node. Such a depiction facilitates the use of previously generated experimental results or survey results so as to assess the relevance of such results to, for example, a proposed course of study related to a particular disease or disorder, or marker, or any other category of input.

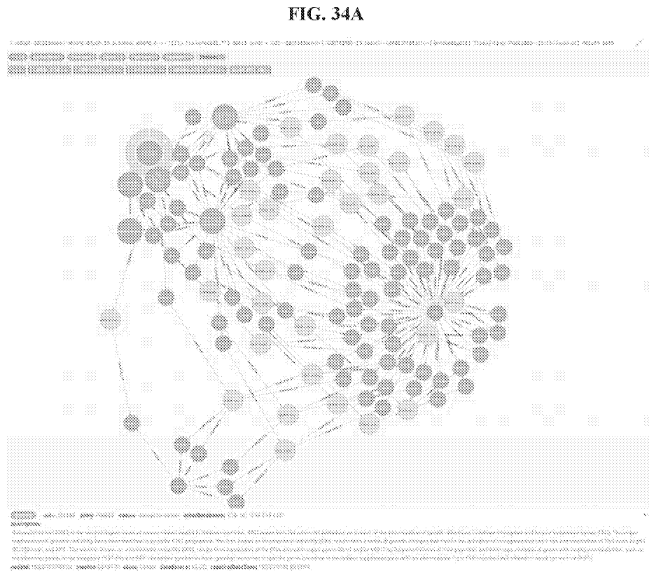

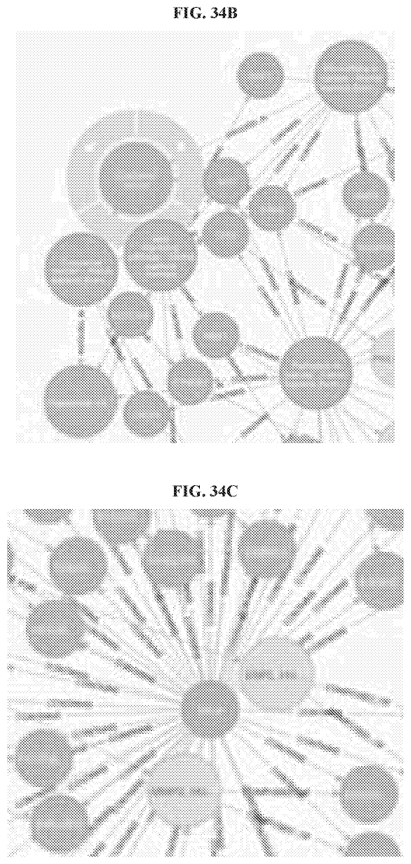

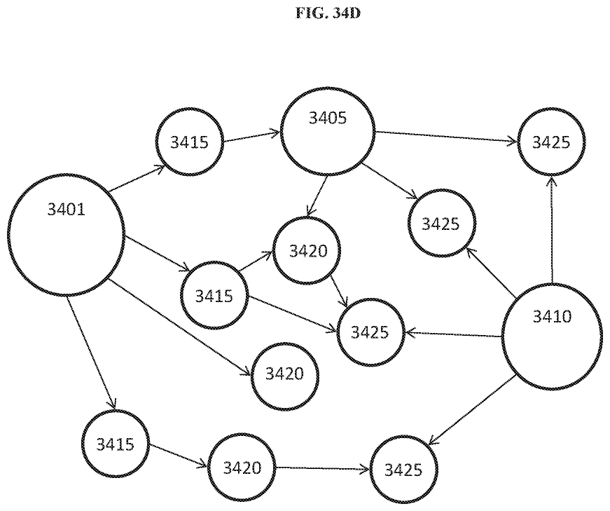

An example of data so displayed on a system screen is given in FIG. 34. The input disorder, colorectal cancer, is depicted at upper right as a pink node encircled by grey. The node is connected directly to three pathways and their related genes. A fourth pathway is implicated through its relationship to common proteins shared by at least some of the other three pathways. A separate disorder is identified through its relationship to three of the four pathways. An array of genes are identified by their involvement in the pathways, and proteins related to these genes are depicted. For the majority of these proteins, at least one and often two marker polypeptides are available. It is observed that the majority of the marker polypeptides map to a common polypeptide collection, in grey at center right. A second set of marker polypeptides map to a second polypeptide collection, at lower left.

Through analysis of the display, one sees that the systems and methods herein allow the rapid navigation of pathway, protein, gene and polypeptide marker data, so that one may readily go from a disease of interest to a marker polypeptide set most likely to be useful in assaying for the disease. One also learns which pathways may be involved in the disease, and which other diseases may share common or overlapping mechanisms. One is then directed to results of assays relating to these pathways or the second disorder, so as to assess data gathered related to these nodes as to their relevance for the input node, in this case colorectal cancer.

Through the display, one also sees alternative uses of the systems and methods herein. One could, for example, start with single collection of polypeptide markers, such as that of the collection at center right. Working form this collection node, one identifies markers in the collection, proteins to which the markers are relevant for assays, and then related genes, pathways and disorders for which the polypeptide collection may be relevant. Thus, the display allows one to identify both which markers may be beneficial in assays for a particular health condition, and which health conditions are most likely to be susceptible to data acquisition using a given collection of markers such as polypeptide markers.

A number of display software packages are consistent with the systems, methods and displays depicted herein. Common to many systems, methods and displays herein is an ability to identify or depict relatedness among biological data types so as to direct a user to particularly relevant marker sources from which to structure future experiments, so as to direct a user to particular pathways of particular interest in a particular disorder or likely to be informed by a particular marker set or antibody set, or particular proteins, genes or pathways likely to be relevant to analysis of a particular disorder.

Displays allow complex data to be presented rapidly, such that in some cases at least 5, 10, 15, 20, 25, 30, 35, 40, 45, 50, 55, 60, 65, 70, 75, 80, 85, 90, 95, 100, or more than 100 nodes are depicted. In some cases nodes are depicted in no more than 30, 25, 20, 15, 10, 5, 4, 3, 2, 1, or less than 1 minute following identification or input of an input node. In some cases nodes are depicted in no more than 30, 25, 20, 15, 10, 5, 4, 3, 2, 1, or less than 1 second following identification or input of an input node.

Methods, systems and displays as disclosed herein often convey a number of benefits as to the operation and use of biological information databases. Data is consolidated and filtered, so that relevant information is presented in a readily analyzable format so that a user may rapidly and with little effort identify relevant related information. At present, some biological data is available for computational searches, but does not incorporate disparate sources or data types, and it is not formatted so as to facilitate rapid assessment and analysis by a user. That is, one may computationally access information databases, such as the National Center for Biotechnology Information at the National Institutes of Health (online at the website ncbi.nlm.nih.gov) to learn about genes relevant to a disorder and pathways implicated in that disorder, and one may access provider catalogues so as to determine what polypeptide markers are commercially available. Such information is available for a computational search, although anecdotally searching particular data sources using particular topics is unlikely to be exhaustive. That is, one is likely to search a database until a piece of information is found, and then consider that a question is answered or an issue solved. Furthermore, searches must be done individually for various fields, and information sources are not often consolidated, such that one must independently search, for example, NCBI for academic information on a topic, independently search company manuals or websites to obtain information on available markers, and independently search one's own resources to determine what marker or other reagents may be already available in a lab, and what relevant experiments have already been run using these reagents. Such an approach is time consuming and rarely exhaustive, such that considerable time is spent to obtain what will very often be less than complete information available on a topic.

Graphic displays of biological database node information such as disclosed herein, alone or in combination with assembled multi-faceted databases comprising one or more than one of disorder information, pathway information, gene, protein and molecular marker information, molecular marker collection or provider information, and information regarding public or unpublished datasets involving a marker, protein, transcript or gene, or informative as to a pathway or condition, dramatically improve the performance of computational biological searches. Various graphic displays present biological data from multiple sources, including academic literature, assembled experimental results, and product catalogues. Interrelationships among relevant aspects of these biological data sources are depicted so as to allow one to readily identify these interrelationships and opportunities presented by these interrelationships. Accordingly one is enabled to consult interrelated literature or datasets involving markers of interest to a particular disorder, even in cases when the markers are used in an analysis that is nominally directed toward a distinct disorder or pathway.

### Certain Definitions

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the singular forms “a”, “an” and “the” include plural references unless the context clearly dictates otherwise. For example, the term “a sample” includes a plurality of samples, including mixtures thereof. Any reference to “or” herein is intended to encompass “and/or” unless otherwise stated.

As used herein, a “condition” is any condition, disease, status or other term for which an assay is to be developed or implemented to assess a patient.

As used herein, the terms “determining”, “measuring”, “evaluating”, “assessing,” “assaying,” and “analyzing” are often used interchangeably herein to refer to forms of measurement, and include determining if an element is present or not (for example, detection). These terms can include quantitative, qualitative or quantitative and qualitative determinations. Assessing is alternatively relative or absolute. “Detecting the presence of” includes determining the amount of something present, as well as determining whether it is present or absent.

As used herein, the terms “panel”, “biomarker panel”, “protein panel”, “classifier model”, and “model” are used interchangeably herein to refer to a set of biomarkers, wherein the set of biomarkers comprises at least two biomarkers. Exemplary biomarkers are proteins or polypeptide fragments of proteins that are uniquely or confidently mapped to particular proteins. However, additional biomarkers are also contemplated, for example age or gender of the individual providing a sample. The biomarker panel is often predictive and/or informative of a subject's health status, disease, or condition.

As used herein, the “level” of a biomarker panel refers to the absolute and relative levels of the panel's constituent markers and the relative pattern of the panel's constituent biomarkers.

As used herein, the term “mass spectrometer” can refer to a gas phase ion spectrometer that measures a parameter that can be translated into mass-to-charge (m/z) ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these. “Mass spectrometry” can refer to the use of a mass spectrometer to detect gas phase ions.

As used herein, the term “tandem mass spectrometer” can refer to any mass spectrometer that is capable of performing two successive stages of m/z-based discrimination or measurement of ions, including ions in an ion mixture. The phrase includes mass spectrometers having two mass analyzers that are capable of performing two successive stages of m/z-based discrimination or measurement of ions tandem-in-space. The phrase further includes mass spectrometers having a single mass analyzer that can be capable of performing two successive stages of m/z-based discrimination or measurement of ions tandem-in-time. The phrase thus explicitly includes Qq-TOF mass spectrometers, ion trap mass spectrometers, ion trap-TOF mass spectrometers, TOF-TOF mass spectrometers, Fourier transform ion cyclotron resonance mass spectrometers, electrostatic sector-magnetic sector mass spectrometers, and combinations thereof.

As used herein, the term “biomarker” and “marker” are used interchangeably herein, and can refer to a polypeptide, gene, nucleic acid (for example, DNA and/or RNA) which is differentially present in a sample taken from a subject having a disease for which a diagnosis is desired (for example, CRC), or to other data obtained from the subject with or without sample acquisition, such as patient age information or patient gender information, as compared to a comparable sample or comparable data taken from control subject that does not have the disease (for example, a person with a negative diagnosis or undetectable disease or condition state, a normal or healthy subject, or, for example, from the same individual at a different time point). Common biomarkers herein include proteins, or protein fragments that are uniquely or confidently mapped to a particular protein (or, in cases such as SAA, above, a pair or group of closely related proteins), transition ion of an amino acid sequence, or one or more modifications of a protein such as phosphorylation, glycosylation or other post-translational or co-translational modification. In addition, a protein biomarker can be a binding partner of a protein, protein fragment, or transition ion of an amino acid sequence.

As used herein, the terms “polypeptide,” “peptide” and “protein” are often used interchangeably herein in reference to a polymer of amino acid residues. A protein, generally, refers to a full-length polypeptide as translated from a coding open reading frame, or as processed to its mature form, while a polypeptide or peptide informally refers to a degradation fragment or a processing fragment of a protein that nonetheless uniquely or identifiably maps to a particular protein. A polypeptide can be a single linear polymer chain of amino acids bonded together by peptide bonds between the carboxyl and amino groups of adjacent amino acid residues. Polypeptides can be modified, for example, by the addition of carbohydrate, phosphorylation, etc. Proteins can comprise one or more polypeptides.

As used herein, the term “immunoassay” is an assay that uses an antibody to specifically bind an antigen (for example, a marker). The immunoassay can be characterized by the use of specific binding properties of a particular antibody to isolate, target, and/or quantify the antigen.

As used herein, the term “antibody” can refer to a polypeptide ligand substantially encoded by an immunoglobulin gene or immunoglobulin genes, or fragments thereof, which specifically binds and recognizes an epitope. Antibodies exist, for example, as intact immunoglobulins or as a number of well-characterized fragments produced by digestion with various peptidases. This includes, for example, Fab and F(ab′)₂ fragments. As used herein, the term “antibody” also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody can refer to that portion of an immunoglobulin heavy chain that comprises one or more heavy chain constant region domains, but does not include the heavy chain variable region.

As used herein, the term “tumor” can refer to a solid or fluid-filled lesion or structure that may be formed by cancerous or non-cancerous cells, such as cells exhibiting aberrant cell growth or division. The terms “mass” and “nodule” are often used synonymously with “tumor”. Tumors include malignant tumors or benign tumors. An example of a malignant tumor can be a carcinoma which is known to comprise transformed cells.

As used herein, the term “binding partners” can refer to pairs of molecules, typically pairs of biomolecules that exhibit specific binding. Protein-protein interactions can occur between two or more proteins, when bound together they often to carry out their biological function. Interactions between proteins are important for the majority of biological functions. For example, signals from the exterior of a cell are mediated via ligand receptor proteins to the inside of that cell by protein-protein interactions of the signaling molecules. For example, molecular binding partners include, without limitation, receptor and ligand, antibody and antigen, biotin and avidin, and others.

As used herein, the term “control reference” can refer to a known or determined amount of a biomarker associated with a known condition that can be used to compare to an amount of the biomarker associated with an unknown condition. A control reference can also refer to a steady-state molecule which can be used to calibrate or normalize values of a non-steady state molecule. A control reference value can be a calculated value from a combination of factors or a combination of a range of factors, such as a combination of biomarker concentrations or a combination of ranges of concentrations.

As used herein, the terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. The disease can be cancer. In some cases, the subject is not necessarily diagnosed or suspected of being at high risk for the disease.

As used herein, the term “in vivo” is used to describe an event that takes place in a subject's body.

As used herein, the term “ex vivo” is used to describe an event that takes place outside of a subject's body. An “ex vivo” assay is not performed on a subject. Rather, it is performed upon a sample separate from a subject. An example of an ‘ex vivo’ assay performed on a sample is an ‘in vitro’ assay.

As used herein, the term “in vitro” is used to describe an event that takes places contained in a container for holding laboratory reagent such that it is separated from the living biological source organism from which the material is obtained. In vitro assays can encompass cell-based assays in which cells alive or dead are employed. In vitro assays can also encompass a cell-free assay in which no intact cells are employed.

As used herein, the term “specificity”, or “true negative rate”, can refer to a test's ability to exclude a condition correctly. For example, in a diagnostic test, the specificity of a test is the proportion of patients known not to have the disease, who will test negative for it. In some cases, this is calculated by determining the proportion of true negatives (i.e. patients who test negative who do not have the disease) to the total number of healthy individuals in the population (i.e., the sum of patients who test negative and do not have the disease and patients who test positive and do not have the disease).

As used herein, the term “sensitivity”, or “true positive rate”, can refer to a test's ability to identify a condition correctly. For example, in a diagnostic test, the sensitivity of a test is the proportion of patients known to have the disease, who will test positive for it. In some cases, this is calculated by determining the proportion of true positives (i.e. patients who test positive who have the disease) to the total number of individuals in the population with the condition (i.e., the sum of patients who test positive and have the condition and patients who test negative and have the condition).

The quantitative relationship between sensitivity and specificity can change as different diagnostic cut-offs are chosen. This variation can be represented using ROC curves. The x-axis of a ROC curve shows the false-positive rate of an assay, which can be calculated as (1−specificity). The y-axis of a ROC curve reports the sensitivity for an assay. This allows one to easily determine a sensitivity of an assay for a given specificity, and vice versa.

As used herein, the term ‘about’ a number refers to that number plus or minus 10% of that number. The term ‘about’ a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, the phrase “at least one of a, b, c, and d” refers to a, b, c, or d, and any and all combinations comprising two or more than two of a, b, c, and d.

As used herein, the term “node” refers to an individual element depicted on a search output, and may also refer to a particular input used to drive or direct a search. A node may be of any category searched, such as a disorder, a pathway, a gene, a transcript, a protein, a polypeptide marker, a collection of polypeptide markers, an oligonucleotide, or a dataset generated using polypeptide markers, oligonucleotides or other data.

### FIGURE DESCRIPTIONS

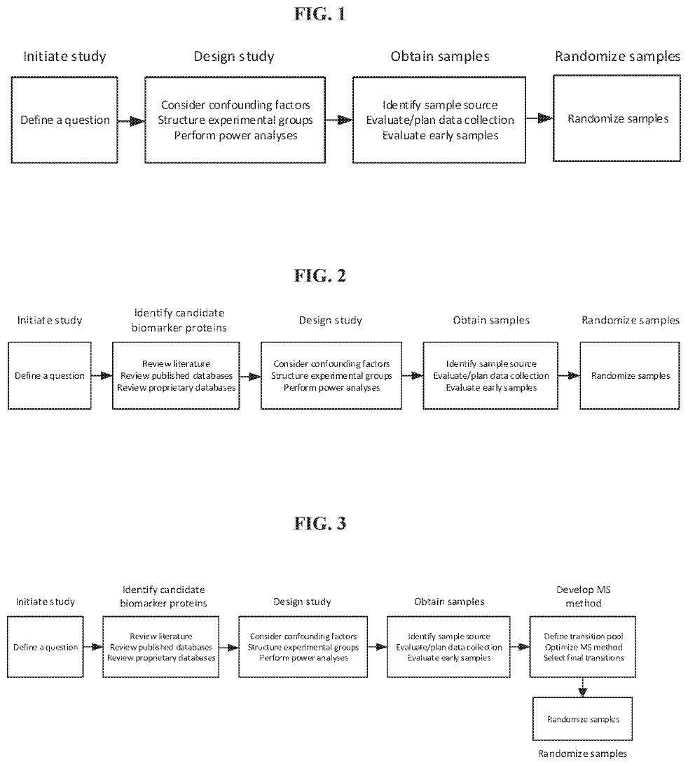

FIG. 1 shows an embodiment of a planning workflow for profile proteomics studies comprising the steps of initializing a study, designing a study, obtaining samples, and randomizing samples. Initiating the study can comprise defining a question (e.g., a biological question such as whether a protein or biomarker is involved in a particular cancer). Designing the study can comprise considering confounding factors, structuring experimental groups, and performing power analyses. Obtaining samples can comprise identifying the sample source, evaluating/planning data collection, and evaluating early samples. Randomizing samples can comprise automated randomization that hides the identity or information of the samples from the user (e.g., the researcher, lab technician, or clinician).

FIG. 2 shows another embodiment of a planning workflow for DPS proteomics studies comprising the steps of initializing a study, identifying candidate biomarker proteins, designing a study, obtaining samples, and randomizing samples. Initiating the study can comprise defining a question (e.g., a biological question such as whether a protein or biomarker is involved in a particular cancer). Identifying the candidate biomarker proteins can comprise reviewing the literature, reviewing one or more published databases, and reviewing one or more proprietary databases. Designing the study can comprise considering confounding factors, structuring experimental groups, and performing power analyses. Obtaining samples can comprise identifying the sample source, evaluating/planning data collection, and evaluating early samples. Randomizing samples can comprise automated randomization that hides the identity or information of the samples from the user (e.g., the researcher, lab technician, or clinician).

FIG. 3 shows an embodiment of a planning workflow for targeted proteomics and iMRM studies comprising the steps of initializing a study, identifying candidate biomarker proteins, designing a study, obtaining samples, developing a mass spectrometry procedure, and randomizing samples. Initiating the study can comprise defining a question (e.g., a biological question such as whether a protein or biomarker is involved in a particular cancer). Identifying the candidate biomarker proteins can comprise reviewing the literature, reviewing one or more published databases, and reviewing one or more proprietary databases. Designing the study can comprise considering confounding factors, structuring experimental groups, and performing power analyses. Obtaining samples can comprise identifying the sample source, evaluating/planning data collection, and evaluating early samples. Developing the mass spectrometry procedure can comprise defining a transition pool, optimizing the MS method, and selecting final transit ions. Randomizing samples can comprise automated randomization that hides the identity or information of the samples from the user (e.g., the researcher, lab technician, or clinician).

FIG. 4 shows an embodiment of a study analysis workflow for profile proteomics studies comprising initial data evaluation, feature processing, data exploration, and at least one of classifier-based analysis and personal proteome browsing. The initial data evaluation can comprise visually assessing starfields and counting features. The feature processing can comprise clustering, fill-in blanks, normalize, filter peaks, propose IDs (e.g., peptide/protein IDs), and finalize data matrices. The data exploration can comprise exploring a signal related to study goals and/or exploring other aspects of the data, and transforming the data. Classifier-based analysis can include building and validating classifiers based on the collected sample data. The workflow can also include visualizing the proteome for personal proteome browsing.

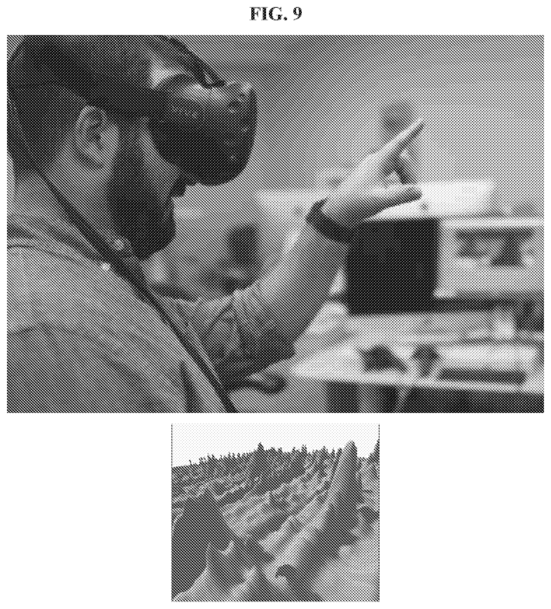

FIG. 5 shows an embodiment of a study analysis workflow for DPS proteomics studies comprising initial data evaluation, feature processing, data exploration, and at least one of classifier-based analysis and personal proteome browsing. The initial data evaluation can comprise visually assessing starfields and counting features. The feature processing can comprise clustering, fill-in blanks, normalize, filter peaks, find targeted peaks, calculate concentrations, and finalize data matrices. The data exploration can comprise exploring a signal related to study goals and/or exploring other aspects of the data, and transforming the data. Classifier-based analysis can include building and validating classifiers based on the collected sample data. The workflow can also include visualizing the proteome for personal proteome browsing.

FIG. 6 shows an embodiment of a study analysis workflow for targeted proteomics and iMRM studies comprising initial data evaluation, feature processing, data exploration, and at least one of classifier-based analysis and personal proteome browsing. The initial data evaluation can comprise visually assessing starfields and counting features. The feature processing can comprise filter peaks, filter transitions, calculate concentrations, and finalize data matrices. The data exploration can comprise exploring a signal related to study goals and/or exploring other aspects of the data, and transforming the data. Classifier-based analysis can include building and validating classifiers based on the collected sample data. The workflow can also include visualizing the proteome for personal proteome browsing.

FIG. 7 shows an embodiment of a low-resolution pipeline-generated starfield image. Data from the starfield is assessed for quality control, and actions taken based on identifiable aberrations. In one aspect, low-resolution pipeline-generated starfield images are visually assessed to identify runs with obvious large-scale aberrations. If any aberrant runs are found, root cause analysis is performed. Aberrant runs are then reprocessed through the pipeline, repeated, removed from further analysis, or flagged for later evaluation depending on the outcome of the root cause analysis.

FIG. 8 shows an embodiment of a high-resolution starfield image. Data in some aspects is also visualized with a medium-resolution starfield images that are scrolled through quickly, with their order determined by a selected annotation field. Sequential images are viewed independently and well-aligned, so that visual persistence enables comparison of feature groups across images. This allows exploration of feature cluster patterns associated with annotations. In some cases, high-resolution starfield images are visually assessed to check that peaks have expected isotope structure, and appear with the expected density across the image.

FIG. 9 shows an embodiment of a high-resolution 3-D starfield images being visually assessed using a 3-D viewing platform. Starfields can be used to count features for quality evaluation of the data.

FIG. 10 shows an embodiment of a visualization to assess and filter standard curves from multiple injections based on measures of spike-in standards (SIS). The visualization is implemented on an SIS Spike-In Experimental Explorer. The visualization includes columns in order from left to right showing protein ID number, peptide sequence, #Obs. Conc. Lvls., R-Squared, Adj. R-Squared, Slope, Slope p-value, Intercept, Intercept p-value, and Cal. Curve.

FIG. 11 shows an embodiment of an interactive high-resolution starfield image on a touchable or touchscreen computer system. A user can manually manipulate the starfield image using the touchable or touchscreen.

FIG. 12 shows embodiment of a starfield thumbnail images across samples grouped and filtered by sample annotation using an Om—The API Data Exploration Center computer program. The program includes columns of information for various samples and the subject from which the sample was derived, including from left to right, external ID, sample barcode, study division (e.g., discovery), age, weight, height, gender, disease status (e.g., Y/N), ethnicity, annotations (e.g., control, disease/type of disease), current medications (e.g., over-the-counter, prescription, supplements, etc.), source (e.g., Promedex), and data for one or more protein fractions (e.g., starfield images for Prot Frac 3/6/8/9/10). The program allows for a user to select entries/rows for further analysis and/or data export.

FIG. 13 shows an embodiment of a visual exploration of longitudinal data with a feature explorer computer program. The program can include various user configurable parameters such as data level (all, reference clusters, or ID′d), day window 1 (e.g., can set any range between 1 and 31 days), day window 2 (e.g., can set any range between 1 and 31 that comes after day window 1), difference threshold (log 2; e.g., can set any threshold between 0 and 5 on a log 2 scale), m/z range (e.g., between 398 and 1,600 m/z), LC time range (e.g., between 0-600 s). The program can also include a diagram showing visual results of the analysis according to the selected parameters (see right side of FIG. 13). The diagram shows m/z on the x-axis and LC time (sec) on the y-axis. The spots on the diagram are color-coded to indicate the change in m/z signal clusters (ranging from negative 5-fold change represented by purple, negative 2.5-fold change by green, no change by yellow, positive 2.5-fold change by orange, and positive 5-fold change by red). Thus, this diagram provides an intuitive and informative presentation of information relevant to showing changes between samples (in this case, obtained at different time points).

FIG. 14 shows an embodiment of a visual exploration of comparative data with a proteomic barcode browser computer program. In some instances, the browser identifies protein abundance (normalized) from multiple individuals in a graphical format which enables ready visual detection of individual differences. The program lists various proteins along the x-axis from left to right: A1AG1_human, A1AG2_human, A1AT_human, A1BG_human, A2MG_human, A4_human, AACT_human, ADAM9_human, ADDG_human, AFAM_human, ALBU_human, ALS_human, ANGT_human, and ANT3_human. The y-axis shows from top down: XYZ, ME, B, and PIG.

FIG. 15 shows an embodiment of a visual exploration of longitudinal data with a personal proteomics data computer browser program. Often proteomics data is observed by reviewing identified peptide/protein abundance (normalized) for a single individual over a study period. A graphical format enables ready visual detection of time-related changes, and a line plot of a given peptide's abundance over the entire study period often is generated for a more detailed examination. In this case, the program shows an illustrative chart with abundance data for LAC_human determined over time from multiple samples. The x-axis is time (0-30 days), and the y-axis is Log 2 abundance (normalized). The fluctuations in abundance as shown provides an example of how a biomarker may be monitored over time based on abundance.

FIG. 16 shows an embodiment of a visual exploration of longitudinal data with a personal proteomics data sphere computer program. This visualization method allows analysis of one individual's MS features, using polar coordinates, with m/z as the angle and LC as the radius. In some instances, multi-day data is displayed by stepping through one day at a time. Other visualizations consistent with the specification are also utilized to visual MS and mass spectrometric data over time and across individuals or populations.

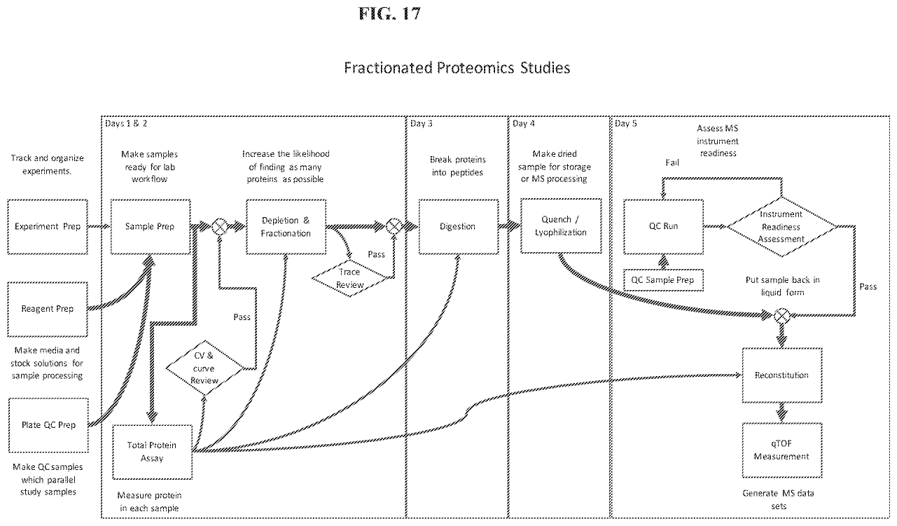

FIG. 17 illustrates an exemplary workflow for fractionated proteomics studies in accordance with an embodiment. The experiments are tracked and organized including experiment preparation, reagent preparation (e.g., making media and stock solutions for sample processing), and plate QC preparation (e.g., preparing QC samples that parallel study samples). Samples are prepared for the workflow including measuring protein concentration. A gating step may be performed following sample prep. Depletion and fractionation is then carried out to increase the likelihood of finding as many proteins of interest as possible. A gating step such as a trace review may be performed following depletion and fractionation. The protein sample is then digested, and later quenched and lyophilized for storage or MS processing. The MS instrument is assessed for readiness (e.g., another gating step). In case of failure of the assessment, the MS instrument may be re-evaluated or re-tested using another QC run with new QC samples. Once the MS instrument is ready (e.g., passes assessment), the lyophilized sample is solubilized/reconstituted and subjected to MS analysis (e.g., qTOF measurement) to generate MS data sets.

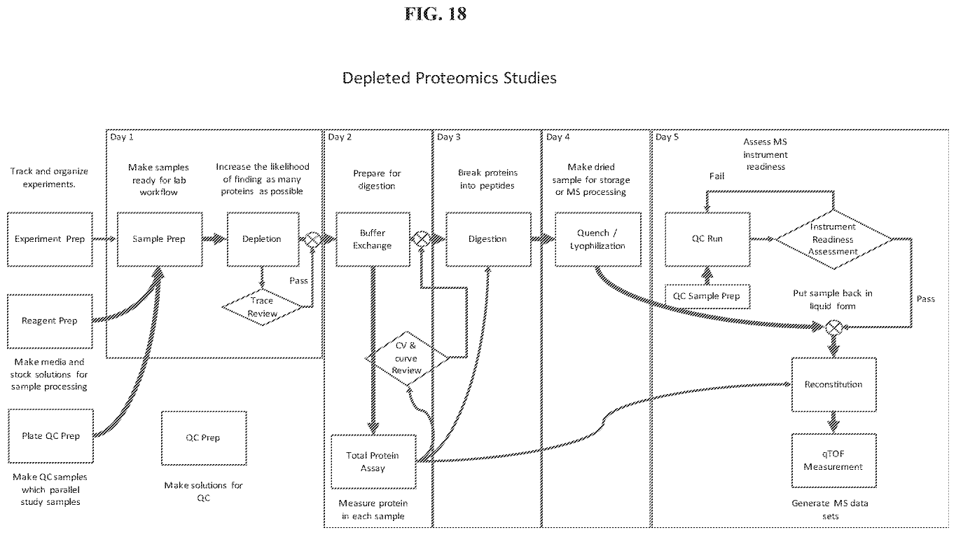

FIG. 18 illustrates an exemplary workflow for depleted proteomics studies in accordance with an embodiment. The experiments are tracked and organized including experiment preparation, reagent preparation (e.g., making media and stock solutions for sample processing), and plate QC preparation (e.g., preparing QC samples that parallel study samples). Samples are prepared for the workflow including measuring protein concentration. A gating step may be performed following sample prep. Depletion is then carried out to increase the likelihood of finding as many proteins of interest as possible. A gating step such as a trace review may be performed following depletion. The sample then undergoes buffer exchange prior to digestion. A gating step may be performed following buffer exchange to assess protein concentration. The protein sample is then digested, and later quenched and lyophilized for storage or MS processing. The MS instrument is assessed for readiness (e.g., another gating step). In case of failure of the assessment, the MS instrument may be re-evaluated or re-tested using another QC run with new QC samples. Once the MS instrument is ready (e.g., passes assessment), the lyophilized sample is solubilized/reconstituted and subjected to MS analysis (e.g., qTOF measurement) to generate MS data sets.

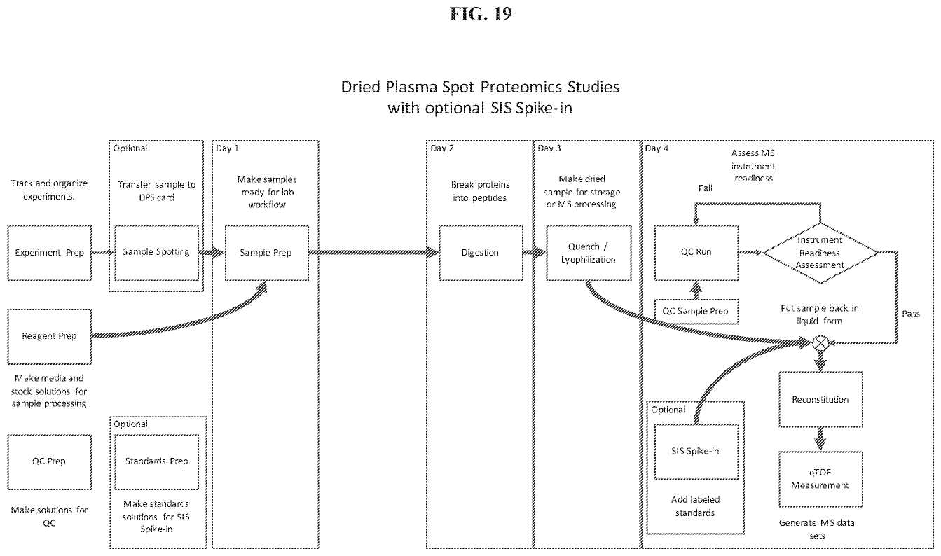

FIG. 19 illustrates an exemplary workflow for dried plasma spot (DPS) proteomics studies with optional SIS spike-in in accordance with an embodiment. The experiments are tracked and organized including experiment preparation, reagent preparation (e.g., making media and stock solutions for sample processing), and plate QC preparation (e.g., preparing QC samples that parallel study samples). Standard solutions are optionally prepared for the SIS spike-in. The sample may be collected on as a dried plasma spot spotted on a DPS card. Samples are prepared for the workflow. A gating step may be performed following sample prep. The protein sample is then digested, and later quenched and lyophilized for storage or MS processing. The MS instrument is assessed for readiness (e.g., another gating step). In case of failure of the assessment, the MS instrument may be re-evaluated or re-tested using another QC run with new QC samples. Once the MS instrument is ready (e.g., passes assessment), the lyophilized sample is solubilized/reconstituted and subjected to MS analysis (e.g., qTOF measurement) to generate MS data sets. When solubilizing the protein sample, SIS may be spiked in, including labeled standards, to enhance MS data analysis.

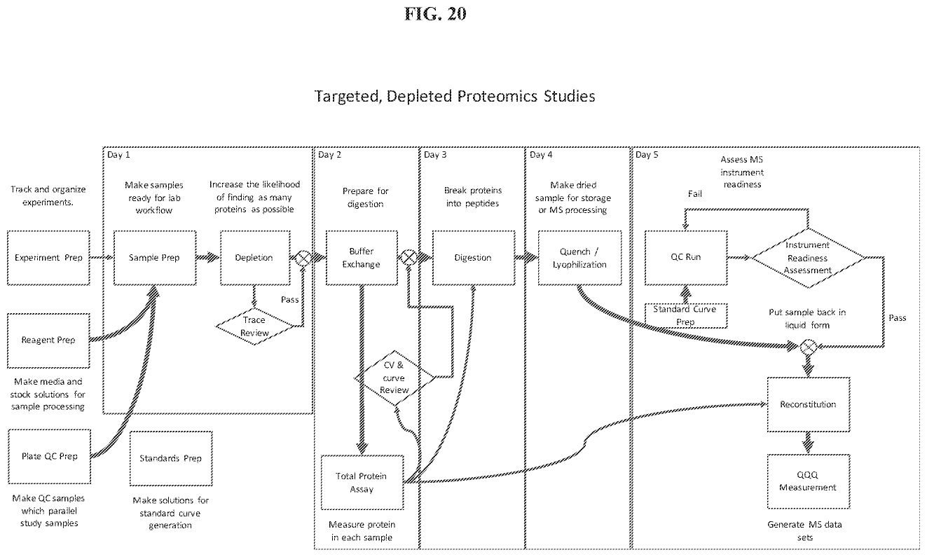

FIG. 20 illustrates an exemplary workflow for targeted, depleted proteomics studies in accordance with an embodiment. The experiments are tracked and organized including experiment preparation, reagent preparation (e.g., making media and stock solutions for sample processing), and plate QC preparation (e.g., preparing QC samples that parallel study samples). Samples are prepared for the workflow. Depletion is then carried out to increase the likelihood of finding as many proteins of interest as possible. A gating step such as a trace review may be performed following depletion. The sample then undergoes buffer exchange prior to digestion. A gating step may be performed following buffer exchange to assess protein concentration. The protein sample is then digested, and later quenched and lyophilized for storage or MS processing. The MS instrument is assessed for readiness (e.g., another gating step). In case of failure of the assessment, the MS instrument may be re-evaluated or re-tested using another QC run with new QC samples. Once the MS instrument is ready (e.g., passes assessment), the lyophilized sample is solubilized/reconstituted and subjected to MS analysis (e.g., QQQ measurement) to generate MS data sets.

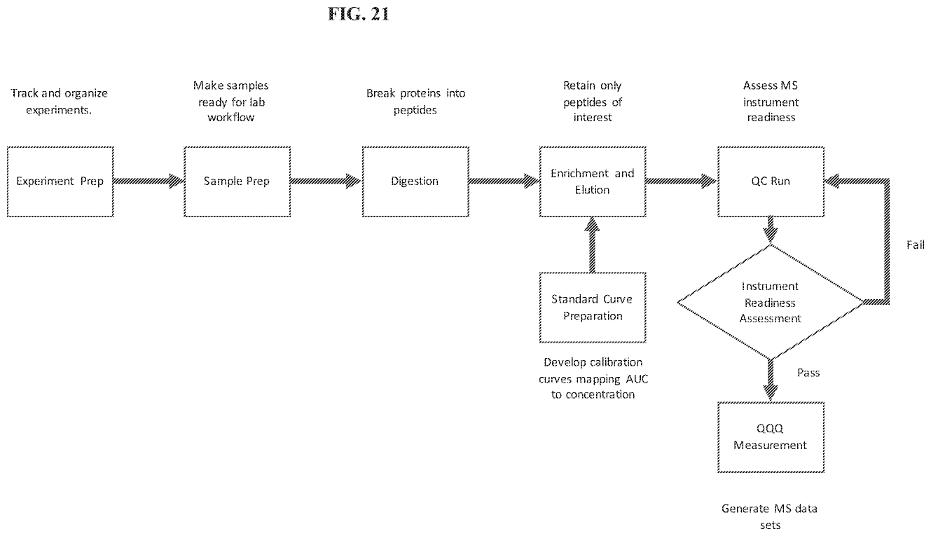

FIG. 21 illustrates an exemplary workflow in accordance with an embodiment. The workflow includes experimental preparation (e.g., track and organize experiments), sample preparation (e.g., make samples ready for the lab workflow), digestion of the sample (e.g., trypsinization), enrichment and elution (e.g., for retaining only peptides of interest) with an optional step for determining protein concentration, perform a QC run to assess MS instrument readiness, and measure the sample using the instrument (e.g., QQQ) to generate MS data sets.

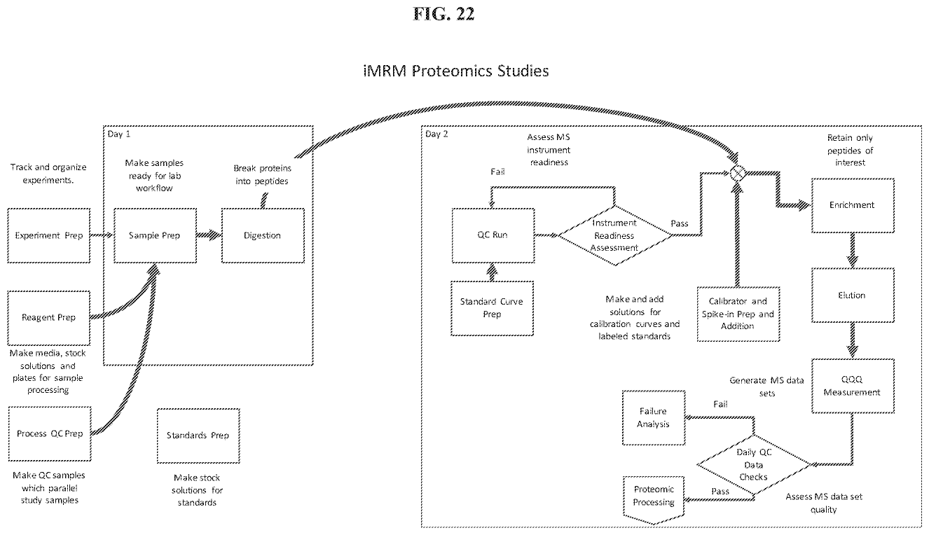

FIG. 22 illustrates an exemplary workflow for iMRM proteomics studies in accordance with an embodiment. The experiments are tracked and organized including experiment preparation, reagent preparation (e.g., making media and stock solutions for sample processing), and plate QC preparation (e.g., preparing QC samples that parallel study samples). Samples are prepared for the workflow. The protein sample is then digested. Meanwhile, the MS instrument is assessed for readiness (e.g., another gating step). In case of failure of the assessment, the MS instrument may be re-evaluated or re-tested using another QC run with new QC samples. Once the MS instrument is ready (e.g., passes assessment), calibrator and spike-in prep and addition is carried out (e.g., spiking reference biomarkers/controls into the sample). The sample then undergoes enrichment, elution, and finally measurement by the MS instrument (e.g., QQQ) to generate the MS data set. The MS data is assessed for quality (e.g., daily QC data checks as samples are processed pursuant to the workflow). Failed QC assessment leads to analysis failure (optionally terminating/suspending the workflow if analysis failure is indicated for an ongoing processing of the sample). Conversely, passing the QC assessment leads to continued proteomic processing.

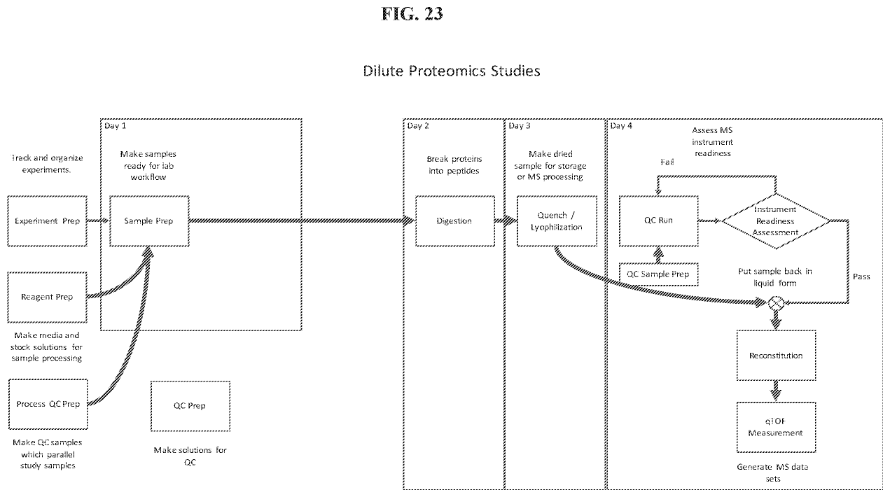

FIG. 23 illustrates an exemplary workflow for dilute proteomic studies in accordance with an embodiment. The experiments are tracked and organized including experiment preparation, reagent preparation (e.g., making media and stock solutions for sample processing), and plate QC preparation (e.g., preparing QC samples that parallel study samples). Samples are prepared for the workflow. The protein sample is then digested, and then quenched and lyophilized for storage or MS processing. Meanwhile, the MS instrument is assessed for readiness (e.g., another gating step). In case of failure of the assessment, the MS instrument may be re-evaluated or re-tested using another QC run with new QC samples. Once the MS instrument is ready (e.g., passes assessment), the sample is put back in liquid form and reconstituted, followed by measurement by the MS instrument (e.g., qTOF) to generate the MS data set.

FIG. 24 illustrates an exemplary series of standard curves. The x axis shows a series of 12 standard curves. Each series includes five points of standard dilutions containing 337 stable isotope sample peptides in a constant plasma background. The Y axis shows peak area under the curve on a log₁₀ scale. These data show the reproducibility of a standard curve using the provided methods.

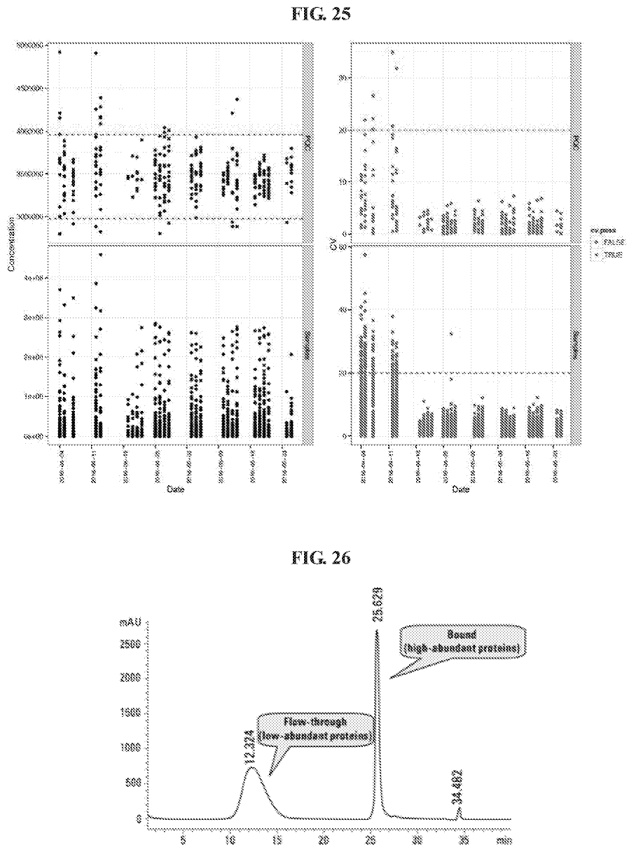

FIG. 25 illustrates an exemplary series of quality control metrics. The X axis in each plot shows a date the experiment was run. The Y axis for the plots on the left shows the concentration. The Y axis in the top left plot is a linear scale ranging between 3,000,000 and 5,000,000 and each dot represents a process quality control data point. The Y axis in the bottom left plot is a natural logarithmic scale ranging between 0e+00 to 4e+08 and each dot represents a sample. The Y axis for the plots on the right shows a coefficient of variation (CV). The Y axis in the top right plot ranges from 0 to 30 and each dot represents a process quality control data point. Dots appearing above the line did not pass the quality control test. The Y axis in the bottom right plot ranges from 0 to 60 and each dot represents a sample data point. Dots appearing above the line did not pass the quality control test.

FIG. 26 illustrates an exemplary trace from a depletion and fractionation experiment. The x axis shows time in minutes between 0 and 40. The y axis shows UV intensity between 0 and 3000 mAU. The first peak contains flow-through of low abundance proteins at 12.324 minutes. The second peak shows elution of high abundance proteins initially bound by a depletion system at 25.629 minutes.

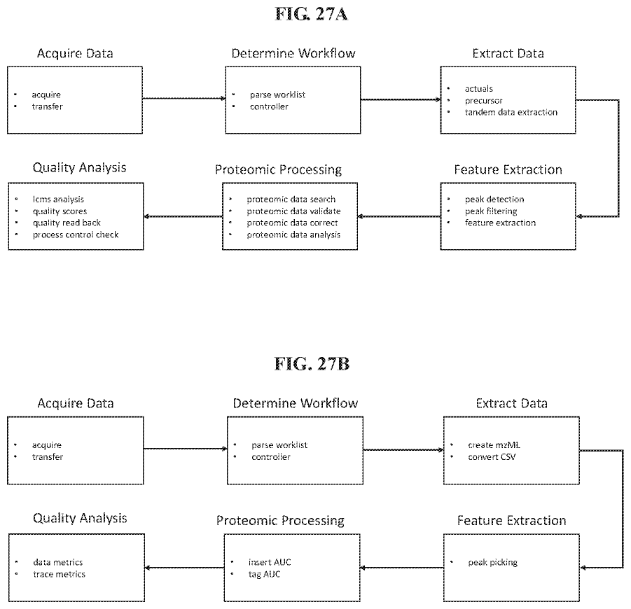

FIG. 27A illustrates an exemplary computational workflow for data analysis in accordance with an embodiment. A data acquisition module acquires data and generates a single LCMS data file for each sample well for a registered study. The data acquisition process includes initiating a workflow queued by registered instruments and verifying that each LCMS data file was copied to shared primary data storage.

FIG. 27B illustrates an exemplary computational workflow for data analysis in accordance with an embodiment. Data is acquired by a data acquisition module, which initiates a workflow queued by polling registered instruments connected to mass spectrometers gathering study data. The acquired instrument data is copied/transferred to a shared repository (in this case, a shared database), which is then verified.

FIG. 28 shows an embodiment of a software application for carrying out the computational workflow described herein. The software application comprises at least one software module for performing the computational pipeline or workflow such as, for example, a series of data processing modules such as one or more of a data acquisition module 2802, a workflow determination module 2804, a data extraction module 2806, a feature extraction module 2808, a proteomic processing module 2810, a quality analysis module 2812, a visualization module 2814, a utility module 2816, or any other data processing module. The modules can be part of a software application or package 2801, which is optionally implemented on a digital processing device or the cloud.

FIG. 29 is a process flow diagram of an example of a health condition indicator identification process.

FIG. 30 is a process flow diagram of another example of a health condition indicator identification process.

FIG. 31 is a schematic diagram of an example of a network layout comprising a health condition indicator identification system.

FIG. 32 is a schematic diagram of an example of a user interface for implementing a health condition indicator identification process.

FIG. 33 is a schematic diagram of an example of a computer system that is programmed or otherwise configured to perform at least a portion of the health condition indicator identification process as described herein.

FIG. 34A is a depiction of a display indicating interrelatedness among disorders (pink), genes (green), pathways (blue), proteins (blue), peptide markers (purple) and peptide collections stored in common or available from a common source (grey).

FIG. 34B shows a zoomed in view of a major node on the left side of the display from FIG. 34A. The view centers on the major node representing colorectal cancer, which connects to surrounding nodes such as pathways (blue).

FIG. 34C shows a zoomed in view of a major node on the right side of the display from FIG. 34A. The view centers on the major node representing a mass spectrometry peptide data collection (gray), which connects to surrounding nodes, in this case, peptide markers (purple).

FIG. 34D shows a simplified representative diagram corresponding to a display such as seen in FIG. 34A that can be generated according to the systems and methods disclosed herein. The major nodes include disorders 3401 which may connect to pathways 3405 implicated in the development and/or pathogenesis of the disorder. The pathways 3405 may connect with various genes 3415 known to operate or function in the pathways. The genes 3415 can connect with the corresponding proteins 3420 (e.g., proteins identified from mass spectrometry data). The proteins 3420 may be identified based on identified peptides 3425 that are derived from the protein 3420, for example, identified peptides 3425 for a data set 3410 from a particular sample. The arrangement of relationships in this figure are intended as an illustrative embodiment of the visualization tool described throughout the present disclosure, and should not be construed as limiting on the possible arrangements of different types of nodes.

### Digital Processing Device

In some embodiments, the platforms, systems, media, methods and applications for carrying out the computational workflow described herein include a digital processing device, a processor, or use of the same. In some cases, the digital processing device is a server. The digital processing device is capable of performing analysis of image-based data such as mass spectra data. Oftentimes, the server comprises at least one database storing mass spectra data and/or peptide sequence information such as, for example, a MySQL database. Sometimes, the server comprises a peptide sequence database such as, for example, a MongoDB. In addition, a digital processing device is a computer in some cases. In certain cases, the digital processing device includes one or more hardware central processing units (CPU) that carry out the device's functions. The digital processing device has a single CPU or processor in many cases. Alternatively, in some cases, the digital processing device has multiple CPUs or processors, which are optionally used for analyzing mass spectra data via parallel processing. Sometimes, the digital processing device further comprises an operating system configured to perform executable instructions. The digital processing device is optionally connected a computer network. In many cases, the digital processing device is connected to the Internet such that it accesses the World Wide Web. The digital processing device is optionally connected to a cloud computing infrastructure. Sometimes, the digital processing device is optionally connected to an intranet. The digital processing device is optionally connected to a data storage device, in many cases. In some cases, a digital processing device is a remote digital processing device used by a user to remotely access a computer system to provide instructions for carrying out mass spectra data analysis.

In accordance with the description herein, suitable digital processing devices include, by way of non-limiting examples, server computers, desktop computers, laptop computers, notebook computers, sub-notebook computers, netbook computers, netpad computers, set-top computers, handheld computers, mobile smartphones, tablet computers, and personal digital assistants. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions including the execution of a plurality of micro-processes for performing analysis of image-based data such as mass spectra data. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, the device includes a storage and/or memory device. The storage and/or memory device is one or more physical apparatuses used to store data or programs on a temporary or permanent basis. In some cases, the device is volatile memory and requires power to maintain stored information. Oftentimes, the device is non-volatile memory and retains stored information when the digital processing device is not powered. For example, sometimes, the non-volatile memory comprises flash memory. The non-volatile memory comprises dynamic random-access memory (DRAM), in various cases. Sometimes, the non-volatile memory comprises ferroelectric random access memory (FRAM). In other cases, the non-volatile memory comprises phase-change random access memory (PRAM). In some cases, the non-volatile memory comprises magnetoresistive random-access memory (MRAM). Oftentimes, the device is a storage device including, by way of non-limiting examples, CD-ROMs, DVDs, flash memory devices, magnetic disk drives, magnetic tapes drives, optical disk drives, and cloud computing based storage. In various cases, the storage and/or memory device is a combination of devices such as those disclosed herein.

In some embodiments, the digital processing device includes a display to send visual information to a subject. Occasionally, the display is a cathode ray tube (CRT). In many cases, the display is a liquid crystal display (LCD). Sometimes, the display is a thin film transistor liquid crystal display (TFT-LCD). In certain cases, the display is an organic light emitting diode (OLED) display. The OLED display is usually a passive-matrix OLED (PMOLED) or active-matrix OLED (AMOLED) display. Sometimes, the display is a plasma display. On occasion, the display is E-paper or E ink. On rare instances, the display is a video projector. In some cases, the display is a combination of devices such as those disclosed herein.

Oftentimes, the digital processing device includes an input device to receive information from a subject. The input device is frequently a keyboard. The input device is sometimes a pointing device including, by way of non-limiting examples, a mouse, trackball, track pad, joystick, or stylus. The input device is often a touch screen or a multi-touch screen. In certain cases, the input device is a microphone to capture voice or other sound input. On occasion, the input device is a video camera or other sensor to capture motion or visual input. The input device is optionally a combination of devices such as those disclosed herein.

### Non-Transitory Computer Readable Storage Medium

Oftentimes, the platforms, media, methods and applications described herein include one or more non-transitory computer readable storage media encoded with a program including instructions executable by the operating system of an optionally networked digital processing device to carrying out a computational pipeline for data analysis. In some cases, a computer readable storage medium is a tangible component of a digital processing device. Occasionally, a computer readable storage medium is optionally removable from a digital processing device. Oftentimes, a computer readable storage medium includes, by way of non-limiting examples, CD-ROMs, DVDs, flash memory devices, solid state memory, magnetic disk drives, magnetic tape drives, optical disk drives, cloud computing systems and services, and the like. The program and instructions are usually permanently, substantially permanently, semi-permanently, or non-transitorily encoded on the media.

### Computer Program

Sometimes, the platforms, media, methods and applications described herein include at least one computer program, or use of the same for executing a plurality of micro-processes for carrying out data analysis of image-based data such as mass spectrometric data. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. Typically, a computer program comprises one sequence of instructions. Oftentimes, a computer program comprises a plurality of sequences of instructions. A computer program is frequently provided from one location. In certain instances, a computer program is provided from a plurality of locations. Sometimes, a computer program includes one or more software modules. A computer program optionally includes, in part or in whole, one or more web applications, one or more mobile applications, one or more standalone applications, one or more web browser plug-ins, extensions, add-ins, or add-ons, or combinations thereof.

### Web Application

In some cases, a computer program includes a web application. In light of the disclosure provided herein, those of skill in the art will recognize that a web application, in various embodiments, utilizes one or more software frameworks and one or more database systems. Sometimes, a web application is created upon a software framework such as Microsoft® .NET or Ruby on Rails (RoR). Oftentimes, a web application utilizes one or more database systems including, by way of non-limiting examples, relational, non-relational, object oriented, associative, and XML database systems. Suitable relational database systems include, by way of non-limiting examples, Microsoft® SQL Server, mySQL™, and Oracle®. Those of skill in the art will also recognize that a web application is written in one or more versions of one or more languages. A web application is capable of being written in one or more markup languages, presentation definition languages, client-side scripting languages, server-side coding languages, database query languages, or combinations thereof. A web application is often written to some extent in a markup language such as Hypertext Markup Language (HTML), Extensible Hypertext Markup Language (XHTML), or eXtensible Markup Language (XML). Sometimes, a web application is written to some extent in a presentation definition language such as Cascading Style Sheets (CSS). Sometimes, a web application is written to some extent in a client-side scripting language such as Asynchronous Javascript and XML (AJAX), Flash® Actionscript, Javascript, or Silverlight®. In various cases, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. On occasion, a web application is written to some extent in a database query language such as Structured Query Language (SQL). Sometimes, a web application integrates enterprise server products such as IBM® Lotus Domino®. On occasion, a web application includes a media player element. The media player element often utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some cases, a computer program includes a mobile application provided to a mobile digital processing device. Sometimes, the mobile application enables the mobile digital processing device to carry out analysis of mass spectra data, for example, as part of a distributed network. In other cases, the mobile application allows the mobile digital processing device to remotely control or send instructions to a computer system for carrying out mass spectra analysis. For example, the mobile application optionally allows a command to be sent to the computer system to initiate, suspend, or terminate at least one micro-process. The mobile application is sometimes provided to a mobile digital processing device at the time it is manufactured. Oftentimes, the mobile application is provided to a mobile digital processing device via a computer network such as the Internet.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C #, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, and Samsung® Apps.

### Standalone Application

In many cases, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Software Modules

In some cases, the platforms, media, methods and applications described herein include software, server, and/or database modules, or use of the same. In view of the disclosure provided herein, software modules are created by techniques known to those of skill in the art using machines, software, and languages known to the art. Sometimes, a software module controls and/or monitors one or more micro-processes. The software modules disclosed herein are implemented in a multitude of ways. In various instances, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. Typically, the one or more software modules comprise, by way of non-limiting examples, a web application, a mobile application, and a standalone application. Oftentimes, software modules are in one computer program or application. Alternatively, in some instances, software modules are in more than one computer program or application. In many cases, software modules are hosted on one machine. Alternatively, sometimes, software modules are hosted on more than one machine. In certain cases, software modules are hosted on cloud computing platforms. Sometimes, software modules are hosted on one or more machines in one location. Alternatively, some software modules are hosted on one or more machines in more than one location.

### Databases

In some embodiments, the platforms, systems, media, and methods disclosed herein include one or more databases, or use of the same, such as, for example, a MySQL database storing mass spectra data and/or a MongoDB peptide sequence database. In view of the disclosure provided herein, those of skill in the art will recognize that many databases are suitable for storage and retrieval of barcode, route, parcel, subject, or network information. In various instances, suitable databases include, by way of non-limiting examples, relational databases, non-relational databases, object oriented databases, object databases, entity-relationship model databases, associative databases, and XML databases. Sometimes, a database is internet-based. In some cases, a database is web-based. On occasion, a database is cloud computing-based. In certain instances, a database is based on one or more local computer storage devices.

### Web Browser Plug-in

Sometimes, the computer program includes a web browser plug-in. In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. Typically, the toolbar comprises one or more web browser extensions, add-ins, or add-ons. In certain instances, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some cases, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Numbered Embodiments

The following embodiments recite nonlimiting permutations of combinations of features disclosed herein. Other permutations of combinations of features are also contemplated. In particular, each of these numbered embodiments is contemplated as depending from or relating to every previous or subsequent numbered embodiment, independent of their order as listed. 1. A system for automated mass spectrometric analysis comprising a plurality of protein processing modules positioned in series; and a plurality of mass spectrometric sample analysis modules; wherein each of said protein processing modules is separated by a mass spectrometric sample analysis module; and wherein each mass spectrometric sample analysis module operates without ongoing supervision. 2. A system for automated mass spectrometric analysis comprising: a plurality of workflow planning modules positioned in series; a plurality of protein processing modules positioned in series; and a plurality of mass spectrometric sample analysis modules; wherein each of said protein processing modules is separated by a mass spectrometric sample analysis module; and at least one of said modules is separated by a gating module; wherein the output data of at least one module has passed a gating module evaluation prior to becoming input data for a subsequent module. 3. The system of embodiment 2, wherein at least one step is performed without ongoing supervision. 4. The system of embodiment 2, wherein at least two steps are performed without ongoing supervision. 5. The system of embodiment 2, wherein all steps are performed without ongoing supervision. 6. The system of any one of embodiments 2-5, wherein at least 90% of the steps are performed without ongoing supervision. 7. The system of any one of embodiments 2-5, wherein at least 75% of the steps are performed without ongoing supervision. 8. The system of any one of embodiments 2-5, wherein at least 50% of the steps are performed without ongoing supervision. 9. A computer-implemented method for automated mass spectrometric workflow planning comprising: a) receiving operating instructions, wherein the operating instructions comprise a study question; b) generating a plurality of candidate biomarker proteins by searching at least one database; and c) designing a mass spectrometric study workflow using the candidate biomarker proteins; wherein the method does not require supervision. 10. The method of embodiment 9, further comprising evaluating early samples prior to execution of the study workflow. 11. The method of any one of embodiments 9-10, further comprising a step selected from the group consisting of analyzing the presence or absence of compounding factors, structuring experimental groups, performing a power analysis, or combinations thereof 12. The method of any one of embodiments 9-11, further comprising randomizing samples. 13. The method of any one of embodiments 9-12, further comprising modifying a downstream experimental step in a workflow plan based on the sample source, to reduce interference by at least one signal. 14. The method of any one of embodiments 9-13, further comprising searching an inventory for corresponding candidate biomarker protein standards. 15. A method for automated mass spectrometric analysis comprising a) defining a transition pool; b) optimizing a mass spectrometric method, wherein optimizing comprises a maximizing signal to noise, reducing method time, minimizing solvent usage, minimizing coefficient of variation, or any combination thereof; c) selecting final transitions; and d) analyzing a mass spectrometric experiment using the final transitions and the optimized mass spectrometric method; wherein at least one of the steps are further separated by a gating step, wherein the gating step evaluates the outcome of a step before performing the next step. 16. The method of embodiment 15, wherein defining a transition pool further comprises performing an in-silico tryptic digestion, selecting proteotypic peptides, predicting peptide ionization/fragmentation in a mass spectrometer, or peptide filtering. 17. The method of any one of embodiments 15-16, wherein the transition pool is identified from a previously optimized mass spectrometric method. 18. A computer-implemented method for automated mass spectrometric analysis comprising: a) receiving operating instructions, wherein the operating instructions comprise variables informative of at least 50 biomarker protein peak quality assignments; b) automatically translating the variables into a machine-learning algorithm; and c) automatically assigning peak quality assignments of subsequent samples using the machine-learning algorithm. 19. The computer-implemented method of embodiment 18, wherein at least 100 biomarker protein peak quality assignments are assigned by a human reviewer. 20. The computer-implemented method of embodiment 18, wherein at least 200 biomarker protein peak quality assignments are assigned by a human reviewer. 21. A method for automated mass spectrometric analysis comprising: a) acquiring at least one mass spectrometric data set from at least two different sample runs; b) generating a visual representation of the data comprising identified features from the at least two sample runs; c) defining an area of the visual representation comprising at least a portion of the identified features; and d) discontinuing analysis because a threshold of at least one QC metric is not met based on a comparison between features of the sample runs; wherein the method is performed on a computer system without user supervision. 22. The method of embodiment 21, wherein the at least two sample runs are from an identical sample source. 23. The method of any one of embodiments 21-22, wherein the number of sample runs for comparison is two. 24. The method of any one of embodiments 21-23, further comprising discontinuing analysis because more than 30,000 features are identified. 25. The method of any one of embodiments 21-24, further comprising discontinuing analysis because more than 10,000 features are identified. 26. The method of any one of embodiments 21-24, further comprising discontinuing analysis because more than 5,000 features are identified. 27. The method of any one of embodiments 21-24, further comprising discontinuing analysis because more than 1,000 features are identified. 28. The method of any one of embodiments 21-27, wherein the area comprises no more than 30,000 features. 29. The method of any one of embodiments 21-27, wherein the area comprises no more than 10,000 features. 30. The method of any one of embodiments 21-27, wherein the area comprises no more than 5,000 features. 31. The method of any one of embodiments 21-27, wherein the area comprises no more than 1,000 features. 32. The method of any one of embodiments 21-27, wherein the threshold is no more than 30,000 total features per sample run. 33. The method of any one of embodiments 21-27, wherein the threshold is no more than 10,000 total features per sample run. 34. The method of any one of embodiments 21-27, wherein the threshold is no more than 5,000 total features per sample run. 35. The method of any one of embodiments 21-27, wherein the threshold is no more than 1,000 total features per sample run. 36. The method of any one of embodiments 21-27, wherein the threshold is no more than 500 total features per sample run. 37. The method of any one of embodiments 21-27, wherein the threshold is no more than 100 total features per sample run. 38. The method of any one of embodiments 21-27, wherein the threshold is no more than 100 total features per sample run. 39. The method of any one of embodiments 21-27, comprising discarding data sets comprising at least 1% non-corresponding features between the sample runs. 40. The method of any one of embodiments 21-39, comprising discarding data sets comprising at least 5% non-corresponding features between the sample runs. 41. The method of any one of embodiments 21-39, comprising discarding data sets comprising at least 10% non-corresponding features between the sample runs. 42. The method of any one of embodiments 21-39, wherein at least one of the steps is performed without ongoing supervision. 43. The method of any one of embodiments 21-39, wherein all of the steps are performed without ongoing supervision. 44. A system for feature processing comprising: a) a plurality of visualization modules positioned in series; and b) a plurality of feature processing modules positioned in series; wherein at least one of the feature processing modules is separated by a gating module; wherein the output data of at least some feature processing modules has passed a gating module evaluation prior to becoming input data for a subsequent feature processing module; wherein the output data of at least some visualization modules has passed a gating evaluation prior to becoming input data for a subsequent visualization module, and wherein at least some gating evaluation occurs without user supervision. 45. The system of embodiment 44, wherein the feature processing module is a clustering module. 46. The system of embodiment 44, wherein the feature processing module is a fill-in-the-blanks module. 47. The system of any one of embodiments 44-46, wherein the feature processing module is a normalization module. 48. The system of any one of embodiments 44-46, wherein the feature processing module is a filtering module. 49. The system of any one of embodiments 44-48, wherein the modules operate without supervision. 50. The system of any one of embodiments 44-49, further comprising a module for finding targeted peaks. 51. The system of any one of embodiments 44-50, further comprising a module for generating data matrices. 52. The system of any one of embodiments 44-51 further comprising a module for building classifiers. 53. A system for proteome visualization comprising: a) a proteomics data set obtained from any of the preceding embodiments; and b) a human interface device capable of visualizing the proteomics data set. 54. The system of embodiment 53, wherein the human interface device comprises a touchable interface. 55. The system of any one of embodiments 53-54, wherein the human interface device comprises a virtual reality interface. 56. The system of any one of embodiments 53-55, wherein the human interface device comprises a personal proteomics data sphere. 57. The system of any one of embodiments 53-56, wherein the human interface device comprises a proteomics genome data browser. 58. The system of any one of embodiments 53-57, wherein the human interface device comprises a proteomics barcode browser. 59. The system of any one of embodiments 53-58, wherein the human interface device comprises a feature explorer. 60. A system for marker candidate identification comprising: a) an input module configured to receive a condition term; b) a search module configured to identify text reciting the condition term and to identify marker candidate text in proximity to the condition term; and c) an experimental design module configured to identify a reagent suitable for detection of the marker candidate. 61. The system of embodiment 60, wherein the reagent comprises a mass-shifted polypeptide. 62. The system of any one of embodiments 60-61 wherein the condition term is a disease. 63. The system of any one of embodiments 60-62, wherein the marker candidate text is a protein identifier. 64. The system of any one of embodiments 60-63, wherein the output data of at least some input, search, or experimental design modules has passed a gating evaluation prior to becoming input data for a subsequent search or experimental design module, and wherein at least some gating evaluation occurs without user supervision. 65. The system of embodiment 1, wherein the system further comprises protein processing modules not separated by a mass spectrometric sample analysis module. 66. The system of any one of embodiments 1 and 65, wherein the system further comprises protein processing modules not positioned in series. 67. The system of any one of embodiments 1 and 65-66, wherein the system further comprises at least one mass spectrometric sample analysis module subject to ongoing supervision. 68. The system of any one of embodiments 1 and 65-67, wherein the sample analysis modules are configured to evaluate performance of an immediately prior protein processing module. 69. The system of any one of embodiments 1 and 65-68, wherein the sample analysis modules are configured to evaluate an effect of an immediately prior protein processing module on a sample selected for mass spectrometric analysis. 70. The system of any one of embodiments 1 and 65-69, wherein the sample analysis modules are configured to stop sample analysis when evaluation indicates that a quality control metric is not met. 71. The system of any one of embodiments 1 and 65-70, wherein the sample analysis modules are configured to tag a sample analysis output when evaluation indicates that a quality control metric is not met for at least one sample analysis module. 72. The system of any one of embodiments 1 and 65-71, wherein the plurality of protein processing modules positioned in series comprises at least four modules. 73. The system of any one of embodiments 1 and 65-72, wherein the plurality of protein processing modules positioned in series comprises at least eight modules. 74. The system of any one of embodiments 1 and 65-73, wherein a sample analysis module evaluates a protein processing module that digests proteins into polypeptide fragments. 75. The system of embodiment 74, wherein the protein processing module that digests proteins contacts proteins to a protease. 76. The system of embodiment 75, wherein the protease comprises trypsin. 77. The system of any one of embodiments 1 and 65-76, wherein a sample analysis module evaluates a protein processing module that volatilizes polypeptides. 78. The system of any one of embodiments 1 and 65-77, wherein a sample analysis module evaluates volatilized polypeptide input mass. 79. The system of any one of embodiments 1 and 65-78, wherein a sample analysis module assesses output of a mass spectrometry mass spectrometry detector module, wherein the output comprises signals detected by a mass spectrometry detector. 80. A system for automated mass spectrometric analysis comprising a plurality of workflow planning modules positioned in series; a plurality of protein processing modules positioned in series; and a plurality of mass spectrometric sample analysis modules; wherein each of said protein processing modules is separated by a mass spectrometric sample analysis module; and wherein each mass spectrometric sample analysis module operates without ongoing supervision. 81. The system of embodiment 80, wherein the plurality of workflow planning modules comprises consideration of confounding factors. 82. The system of any one of embodiments 80-81, wherein the plurality of workflow planning modules comprises structuring experimental groups. 83. The system of any one of embodiments 80-82, wherein the plurality of workflow planning modules comprises performing power analyses. 84. The system of any one of embodiments 80-83, wherein the plurality of workflow planning modules comprises a plan for sample collection. 85. The system of any one of embodiments 80-84, wherein the plurality of workflow planning modules comprises early sample analyses. 86. The system of any one of embodiments 80-85, wherein the plurality of workflow planning modules comprises randomizing samples. 87. The system of any one of embodiments 80-86, wherein the plurality of workflow planning modules comprises identifying candidate biomarker proteins. 88. The system of embodiment 87, identifying candidate biomarker proteins comprises searching literature databases. 89. The system of any one of embodiments 80-88, wherein the plurality of workflow planning modules comprises defining a transition pool. 90. The system of any one of embodiments 80-89, wherein the plurality of workflow planning modules comprises optimizing a mass spectrometric method. 91. The system of any one of embodiments 80-90, wherein the plurality of workflow planning modules comprises selecting final transitions. 92. The system of any one of embodiments 80-91, wherein the plurality of workflow planning modules positioned in series comprises at least two modules. 93. The system of any one of embodiments 80-92, wherein the plurality of workflow planning modules positioned in series comprises at least four modules. 94. The system of any one of embodiments 80-93, wherein the plurality of workflow planning modules positioned in series comprises at least eight modules. 95. A method of mass spectrometric sample analysis comprising subjecting a mass spectrometric sample to a series of manipulations pursuant to workflow planning; wherein at least some of said manipulations pursuant workflow planning are gated by automated evaluation of an outcome of a prior step. 96. The method of embodiment 95, wherein at least some of said manipulations pursuant to workflow planning are gated by automated evaluation of an outcome of a prior step such that analysis is stopped when an automated evaluation does not meet a threshold. 97. The method of any one of embodiments 95-96, wherein at least some of said manipulations pursuant workflow planning are gated by automated evaluation of an outcome of a prior step such that analysis output is flagged when an automated evaluation does not meet a threshold 98. The method of any one of embodiments 95-97, wherein at least some of said manipulations pursuant to workflow planning are gated by automated evaluation of an outcome of a prior step such that the mass spectrometric sample is discarded when an automated evaluation does not meet a threshold. 99. The method of any one of embodiments 95-98, wherein automated evaluation of an outcome of at least one prior step does not comprise user assessment. 100. A method of mass spectrometric sample analysis comprising subjecting a mass spectrometric sample to a series of manipulations pursuant to mass spectrometric analysis; wherein at least some of said manipulations pursuant to mass spectrometric analysis are gated by automated evaluation of an outcome of a prior step. 101. The method of embodiment 100, wherein at least some of said manipulations pursuant to mass spectrometric analysis are gated by automated evaluation of an outcome of a prior step such that analysis is stopped when an automated evaluation does not meet a threshold. 102. The method of embodiment 100, wherein at least some of said manipulations pursuant to mass spectrometric analysis are gated by automated evaluation of an outcome of a prior step such that analysis output is flagged when an automated evaluation does not meet a threshold 103. The method of embodiment 100, wherein at least some of said manipulations pursuant to mass spectrometric analysis are gated by automated evaluation of an outcome of a prior step such that the mass spectrometric sample is discarded when an automated evaluation does not meet a threshold. 104. The method of any one of embodiments 100-103, wherein automated evaluation of an outcome of at least one prior step does not comprise user assessment. 105. A system for automated mass spectrometric analysis comprising a plurality of protein processing modules positioned in series; and a plurality of mass spectrometric sample analysis modules; wherein at least some of said protein processing modules are separated by a mass spectrometric sample analysis module; and wherein at least some mass spectrometric sample analysis modules operate without ongoing supervision. 106. The system of embodiment 105, wherein the system further comprises protein processing modules not separated by a mass spectrometric sample analysis module. 107. The system of any one of embodiments 105-106, wherein the system further comprises protein processing modules not positioned in series. 108. The system of any one of embodiments 105-107, wherein the system further comprises at least one mass spectrometric sample analysis module subject to ongoing supervision. 109. The system of any one of embodiments 105-107, wherein the system does not require user supervision. 110. The system of any one of embodiments 105-109, wherein the sample analysis modules are configured to evaluate performance of an immediately prior protein processing module. 111. The system of any one of embodiments 105-110, wherein the sample analysis modules are configured to evaluate an effect of an immediately prior protein processing module on a sample selected for mass spectrometric analysis. 112. The system of any one of embodiments 105-112, wherein the sample analysis modules are configured to stop sample analysis when evaluation indicates that a quality control metric is not met. 113. The system of any one of embodiments 105-112, wherein the sample analysis modules are configured to tag a sample analysis output when evaluation indicates that a quality control metric is not met for at least one sample analysis module. 114. The system of any one of embodiments 105-113, wherein the plurality of protein processing modules positioned in series comprises at least four modules. 115. The system of any one of embodiments 105-113, wherein the plurality of protein processing modules positioned in series comprises at least eight modules. 116. The system of any one of embodiments 105-115, wherein a sample analysis module evaluates a protein processing module that digests proteins into polypeptide fragments. 117. The system of embodiment 116, wherein the protein processing module that digests proteins contacts proteins to a protease. 118. The system of embodiment 117, wherein the protease comprises trypsin. 119. The system of any one of embodiments 105-118, wherein a sample analysis module evaluates a protein processing module that volatilizes polypeptides. 120. The system of any one of embodiments 105-119, wherein a sample analysis module evaluates volatilized polypeptide input mass. 121. The system of any one of embodiments 105-120, wherein a sample analysis module assesses output of a mass spectrometry mass spectrometry detector module, wherein the output comprises signals detected by a mass spectrometry detector. 122. A method of mass spectrometric sample analysis comprising subjecting a mass spectrometric sample to a series of manipulations pursuant to mass spectrometric analysis; wherein at least some of said manipulations pursuant to mass spectrometric analysis are gated by automated evaluation of an outcome of a prior step. 123. The method of embodiment 122, wherein at least some of said manipulations pursuant to mass spectrometric analysis are gated by automated evaluation of an outcome of a prior step such that analysis is stopped when an automated evaluation does not meet a threshold. 124. The method of any one of embodiments 122-123, wherein at least some of said manipulations pursuant to mass spectrometric analysis are gated by automated evaluation of an outcome of a prior step such that analysis output is flagged when an automated evaluation does not meet a threshold 125. The method of any one of embodiments 122-124, wherein at least some of said manipulations pursuant to mass spectrometric analysis are gated by automated evaluation of an outcome of a prior step such that the mass spectrometric sample is discarded when an automated evaluation does not meet a threshold. 126. The method of any one of embodiments 122-125, wherein automated evaluation of an outcome of at least one prior step does not comprise user assessment. 127. A system comprising a) a marker candidate generation module configured to receive a condition input, to search a literature database to identify references reciting the condition, to identify marker candidates recited in the references, and to assemble the marker candidates into a marker candidate panel; and 2) a data analysis module, configured to assess a correlation between the condition and the marker candidate panel in at least one gated mass spectrometric dataset. 128. The system of embodiment 127, comprising a sample analysis module comprising a plurality of protein processing modules positioned in series; and a plurality of mass spectrometric sample analysis modules; wherein at least some of said protein processing modules are separated by a mass spectrometric sample analysis module; and wherein at least some mass spectrometric sample analysis modules operate without ongoing supervision, so as to produce a gated data set. 129. The system of embodiment 127 or embodiment 128, wherein said system operates without user supervision. 130. The system of embodiment 127 or embodiment 128, wherein said system operates with user supervision at no more than 5 steps. 131. The system of embodiment 127 or embodiment 128, wherein said system operates with user supervision at no more than 4 steps. 132. The system of embodiment 127 or embodiment 128, wherein said system operates with user supervision at no more than 3 steps. 133. The system of embodiment 127 or embodiment 128, wherein said system operates with user supervision at no more than 2 steps. 134. The system of any one of embodiments 127-133, comprising a workflow generation module that selects at least one reagent to facilitate marker candidate assessment. 135. The system of embodiment 134, wherein the at least one reagent comprises at least one mass-shifted polypeptide. 136. The system of embodiment 135, wherein the at least one mass-shifted polypeptide facilitates mass spectrometric identification of a marker candidate polypeptide. 137. The system of embodiment 135, wherein the at least one mass-shifted polypeptide facilitates mass spectrometric quantification of a marker candidate polypeptide. 138. The system of any one of embodiments 127-137, wherein the references comprise peer-reviewed academic references. 139. The system of any one of embodiments 127-137, wherein the references comprise medical references. 140. The system of any one of embodiments 127-137, wherein the references comprise patent application publications. 141. The system of any one of embodiments 127-137, wherein the references comprise patents. 142. A system for automated mass spectrometric analysis comprising a plurality of protein processing modules positioned in series; and a plurality of mass spectrometric sample analysis modules; wherein each of said protein processing modules is separated by a mass spectrometric sample analysis module; and wherein each mass spectrometric sample analysis module operates without ongoing supervision. 143. The system of embodiment 142, wherein the system further comprises protein processing modules not separated by a mass spectrometric sample analysis module. 144. The system of embodiment 142, wherein one of the sample analysis modules comprises an instrument configured to measure the concentration of protein in a sample. 145. The system of embodiment 144, wherein the sample analysis module comprises an instrument configured to measure the optical density of a protein sample. 146. The system of embodiment 145, wherein the sample analysis module comprises a spectrophotometer. 147. The system of any one of embodiments 145-146, wherein the system is configured to analyze the coefficient of variation of optical density values obtained from replicates derived from a protein sample. 148. The system of any one of embodiments 145-147, wherein the system is configured to analyze an optical density curve generated by measuring the optical density of known dilutions generated from a protein sample. 149. The system of any one of embodiments 144-148, wherein the system is configured to calculate a protein concentration from the measured optical density of a sample. 150. The system of any one of embodiments 144-149, wherein the system is configured to flag a sample that fails to meet a set of protein concentration criteria. 151. The system of embodiment 150, wherein a criterion is percent recovery. 152. The system of embodiment 150, wherein a criterion is estimated protein content. 153. The system of embodiment 150, wherein a criterion is the coefficient of variation calculated from protein concentrations determined for a plurality of replicates aliquoted from a sample. 154. The system of any one of embodiments 142-153, wherein one of the protein processing modules utilizes gas chromatography, liquid chromatography, capillary electrophoresis, or ion mobility to fractionate a sample, and wherein the system is configured to analyze data generated by the detector and flag samples that do not meet a set of chromatography QC metrics comprising at least one of peak shifting, peak area, peak shape, peak height, wavelength absorption, or wavelength of fluorescence detected in the biological sample. 155. The system of embodiment 154, wherein the liquid chromatograph comprises a detector that detects the amount of sample emerging from the liquid chromatograph. 156. The system of embodiment 155, wherein the detector comprises an electromagnetic absorbance detector. 157. The system of embodiment 156, wherein the electromagnetic absorbance detector comprises an ultraviolet absorbance detector. 158. The system of embodiment 156, wherein the electromagnetic absorbance detector comprises an ultraviolet/visible absorbance detector. 159. The system of embodiment 156, wherein the electromagnetic absorbance detector comprises an infrared absorbance detector. 160. The system of embodiment 155, wherein the detector comprises a charged aerosol detector. 161. The system of embodiment 155, wherein the system is configured to analyze data generated by the detector and flag samples that do not meet a set of chromatography criteria. 162. The system of embodiment 161, wherein one criterion is an amount of lipids detected in the sample. 163. The system of embodiment 161, wherein one criterion is an amount of hemoglobin detected in the sample. 164. The system of embodiment 161, wherein one criterion is a peak shift detected in the sample. 165. The system of any one of embodiments 142-164, wherein one of the sample analysis modules comprises an instrument configured to measure an amount of lipids in a sample. 166. The system of any one of embodiments 142-165, wherein one of the sample analysis modules comprises an instrument configured to measure an amount of hemoglobin in a sample. 167. The system of any one of embodiments 142-166, wherein one of the protein processing modules is configured to deplete a protein sample by removing pre-selected proteins from the sample. 168. The system of any one of embodiments 142-167, wherein one of the protein processing modules comprises an instrument configured to compute and add an amount of a protease to the sample. 169. The system of embodiment 168, wherein the amount of a protease added to the sample is dynamically calculated by the amount of protein estimated to be present in the sample. 170. The system of any one of embodiments 142-169, wherein the system can assess the readiness of one or more of the modules present in the system. 171. The system of embodiment 170, wherein one of the modules the system can assess the readiness of comprises a mass spectrometer. 172. The system of embodiment 171, wherein the system assesses the readiness of the mass spectrometer by determining if data generated by the mass spectrometer from a sample are consistent with data previously generated from the same sample. 173. The system of embodiment 171, wherein the system assesses the readiness of the mass spectrometer by determining if data generated by the mass spectrometer from a sample indicates detection of a minimum number of features that possess a specific charge state, a minimum number of features, selected analyte signal that meets at least one threshold, presence of known contaminants, mass spectrometer peak shape, chromatographic peak shape, or any combination thereof 174. The system of embodiment 173, wherein the charge state is selected from the group consisting of 2, 3, and 4. 175. The system of any one of embodiments 142-174, wherein the system comprises a processor that can generate worklists for use by modules present in the system. 176. The system of any one of embodiments 142-175, wherein one of the mass spectrometric sample analysis modules comprises a qTOF mass spectrometer. 177. The system of any one of embodiments 142-176, wherein one of the mass spectrometric sample analysis modules comprises a liquid chromatograph. 178. The system of any one of embodiments 142-177, wherein the sample analysis modules are configured to stop sample analysis when evaluation indicates that a quality control metric is not met. 179. The system of any one of embodiments 142-178, wherein the plurality of protein processing modules comprises a quality control check prior to a mass spectrometric sample analysis module. 180. The system of any one of embodiments 142-179, wherein the plurality of protein processing modules comprises a quality control check prior to running a sample. 181. The system of any one of embodiments 142-180, wherein the plurality of protein processing modules comprises a quality control check prior to a depletion/fractionation module. 182. The system of any one of embodiments 142-181, wherein the plurality of protein processing modules comprises a quality control check after a digestion module. 183. The system of any one of embodiments 142-182, wherein at least some of said manipulations pursuant to mass spectrometric analysis processing are gated by automated evaluation of an outcome of a prior step such that analysis is stopped when an automated evaluation does not meet a threshold. 184. The method of any one of embodiments 142-182, wherein at least some of said manipulations pursuant to mass spectrometric analysis are gated by automated evaluation of an outcome of a prior step such that the mass spectrometric sample is discarded when an automated evaluation does not meet a threshold. 185. The method of any one of embodiments 142-182, wherein at least some of said manipulations pursuant to mass spectrometric analysis are gated by automated evaluation of an outcome of a prior step such that the modules in the analysis are repeated under new conditions, changed, or removed as a result of the evaluation. 186. A method of mass spectrometric sample analysis comprising subjecting a mass spectrometric sample to a series of manipulations pursuant to mass spectrometric analysis, wherein at least some of said manipulations pursuant to mass spectrometric analysis are gated by automated evaluation of an outcome of a prior step. 187. The method of embodiment 186, wherein the method is performed by any one of the systems of embodiments 142-185. 188. The system of any one of embodiments 186-187, wherein one of the sample analysis modules comprises an instrument configured to measure the concentration of protein in a sample. 189. The method of embodiment 188, wherein the sample analysis module comprises an instrument configured to measure the optical density of a protein sample. 190. The method of any one of embodiments 188-189, wherein the sample analysis module comprises a spectrophotometer. 191. The method of any one of embodiments 188-190, wherein the system is configured to analyze the coefficient of variation of optical density values obtained from replicates derived from a protein sample. 192. The method of any one of embodiments 188-191, wherein the system is configured to analyze an optical density curve generated by measuring the optical density of known dilutions generated from a protein sample. 193. The method of any one of embodiments 186-192, wherein the system is configured to calculate a protein concentration from the measured optical density of a sample. 194. The method of any one of embodiments 186-193, wherein the system is configured to flag a sample that fails to meet a set of protein concentration criteria. 195. The method of embodiment 194, wherein a criterion is percent recovery. 196. The method of embodiment 194, wherein a criterion is estimated protein content. 197. The method of embodiment 194, wherein a criterion is the coefficient of variation calculated from protein concentrations determined for a plurality of replicates aliquoted from a sample. 198. The method of any one of embodiments 186-197, wherein one of the protein processing modules utilizes gas chromatography, liquid chromatography, capillary electrophoresis, or ion mobility to fractionate a sample, and wherein the system is configured to analyze data generated by the detector and flag samples that do not meet a set of chromatography QC metrics comprising at least one of peak shifting, peak area, peak shape, peak height, wavelength absorption, or wavelength of fluorescence detected in the biological sample. 199. The method of embodiment 198, wherein the liquid chromatograph comprises a detector that detects the amount of sample emerging from the liquid chromatograph. 200. The method of embodiment 199, wherein the detector comprises an electromagnetic absorbance detector. 201. The method of embodiment 200, wherein the electromagnetic absorbance detector comprises an ultraviolet absorbance detector. 202. The method of embodiment 200, wherein the electromagnetic absorbance detector comprises an ultraviolet/visible absorbance detector. 203. The method of embodiment 200, wherein the electromagnetic absorbance detector comprises an infrared absorbance detector. 204. The method of any one of embodiments 199-203, wherein the detector comprises a charged aerosol detector. 205. The method of any one of embodiments 198-204, wherein the system is configured to analyze data generated by the detector and flag samples that do not meet a set of chromatography criteria. 206. The method of embodiment 205, wherein one criterion is an amount of lipids detected in the sample. 207. The method of embodiment 205, wherein one criterion is an amount of hemoglobin detected in the sample. 208. The method of embodiment 205, wherein one criterion is a peak shift detected in the sample. 209. The method of any one of embodiments 186-208, wherein one of the sample analysis modules comprises an instrument configured to measure an amount of lipids in a sample. 210. The method of any one of embodiments 186-209, wherein one of the sample analysis modules comprises an instrument configured to measure an amount of hemoglobin in a sample. 211. The method of any one of embodiments 186-210, wherein one of the protein processing modules is configured to deplete a protein sample by removing pre-selected proteins from the sample. 212. The method of any one of embodiments 186-211, wherein one of the protein processing modules comprises an instrument configured to compute and add an amount of a protease to the sample. 213. The method of embodiment 212, wherein the amount of a protease added to the sample is dynamically calculated by the amount of protein estimated to be present in the sample. 214. The method of any one of embodiments 186-213, wherein the system can assess the readiness of one or more of the modules present in the system. 215. The method of embodiment 214, wherein one of the modules the system can assess the readiness of comprises a mass spectrometer. 216. The method of embodiment 215, wherein the system assesses the readiness of the mass spectrometer by determining if data generated by the mass spectrometer from a sample are consistent with data previously generated from the same sample. 217. The method of embodiment 215, wherein the system assesses the readiness of the mass spectrometer by determining if data generated by the mass spectrometer from a sample indicates detection of a minimum number of features that possess a specific charge state, a minimum number of features, selected analyte signal that meets at least one threshold, presence of known contaminants, mass spectrometer peak shape, chromatographic peak shape, or any combination thereof. 218. The method of embodiment 217, wherein the charge state is selected from the group consisting of 2, 3, and 4. 219. The method of any one of embodiments 186-218, wherein the system comprises a processor that can generate worklists for use by modules present in the system. 220. The method of any one of embodiments 186-219, wherein one of the mass spectrometric sample analysis modules comprises a qTOF mass spectrometer. 221. The method of any one of embodiments 186-220, wherein one of the mass spectrometric sample analysis modules comprises a liquid chromatograph. 222. The method of any one of embodiments 186-221, wherein the sample analysis modules are configured to stop sample analysis when evaluation indicates that a quality control metric is not met. 223. The method of any one of embodiments 186-222, wherein the plurality of protein processing modules comprises a quality control check prior to a mass spectrometric sample analysis module. 224. The method of any one of embodiments 186-223, wherein the plurality of protein processing modules comprises a quality control check prior to running a sample. 225. The method of any one of embodiments 186-224, wherein the plurality of protein processing modules comprises a quality control check prior to a depletion/fractionation module. 226. The method of any one of embodiments 186-225, wherein the plurality of protein processing modules comprises a quality control check after a digestion module. 227. The method of any one of embodiments 186-226, wherein at least some of said manipulations pursuant to mass spectrometric analysis processing are gated by automated evaluation of an outcome of a prior step such that analysis is stopped when an automated evaluation does not meet a threshold. 228. The method of any one of embodiments 186-226, wherein at least some of said manipulations pursuant to mass spectrometric analysis are gated by automated evaluation of an outcome of a prior step such that the mass spectrometric sample is discarded when an automated evaluation does not meet a threshold. 229. The method of any one of embodiments 186-226, wherein at least some of said manipulations pursuant to mass spectrometric analysis are gated by automated evaluation of an outcome of a prior step such that the modules in the analysis are repeated under new conditions, changed, or removed as a result of the evaluation. 230. A system for automated mass spectrometric analysis of a data set, comprising: a) a plurality of mass spectrometric data processing modules; and b) a workflow determination module generating a computational workflow comprising the plurality of data processing modules positioned in series to analyze the data set, wherein the computational workflow is configured based on at least one of a worklist and at least one quality assessment performed during mass spectrometric sample processing. 231. The system of embodiment 230, wherein the workflow determination module generates the computational workflow based on a mass spectrometric method used to process a sample and sample processing parameters. 232. The system of any of embodiments 230-231, wherein generating the computational workflow comprises extracting a methodology and parameters from the worklist and assembling data processing modules suitable for processing the data set based on the methodology and parameters. 233. The system of any of embodiments 230-232, wherein generating the computational workflow comprises adding at least one quality assessment step to be performed during the computational workflow. 234. The system of any of embodiments 230-233, wherein the system further comprises at least one mass spectrometric data processing module subject to ongoing supervision. 235. The system of any of embodiments 230-234, wherein at least one mass spectrometric data processing module is configured to evaluate performance of an immediately prior mass spectrometric data processing module. 236. The system of any of embodiments 230-235, wherein at least one mass spectrometric data processing module is configured to evaluate an effect of an immediately prior mass spectrometric data processing module on sample data. 237. The system of any of embodiments 230-236, wherein at least one mass spectrometric data processing module is configured to evaluate sample data using a quality control metric after the sample data has been processed by at least one mass spectrometric data processing module. 238. The system of any of embodiments 230-237, wherein the mass spectrometric data processing modules are configured to stop sample data analysis when evaluation indicates that a quality control metric is not met. 239. The system of any of embodiments 230-238, wherein the mass spectrometric data processing modules are configured to tag a sample data analysis output when evaluation indicates that the output has failed a quality control metric. 240. The system of any of embodiments 230-239, wherein the mass spectrometric data processing modules comprise a data acquisition module. 241. The system of embodiment 240, wherein the data acquisition module obtains the data set and copies it into a primary storage for downstream analysis. 242. The system of any one of embodiments 240-241, wherein the data acquisition module stores the data set in one or more data files. 243. The system of any one of embodiments 240-242, wherein the data acquisition module generates a single data file for each sample. 244. The system of any one of embodiments 240-243, wherein a quality assessment of data acquisition comprises confirming the processed data set has been successfully acquired and copied into a data storage. 245. The system of any one of embodiments 230-244, wherein the computational workflow is a pre-set workflow based on the type of mass spectrometric data analysis selected. 246. The system of any one of embodiments 230-245, wherein the computational workflow is a pre-set workflow based on parameters extracted from a work list for the mass spectrometric data. 247. The system of any one of embodiments 230-246, wherein the computational workflow is a customized workflow based on parameters extracted from a work list for the mass spectrometric data. 248. The system of any one of embodiments 230-247, wherein the computational workflow is configured to process mass spectrometric data generated by Profile and DPS proteomics. 249. The system of any one of embodiments 230-248, wherein the computational workflow is configured to process data generated by Targeted and iMRM proteomics. 250. The system of any one of embodiments 230-249, wherein the mass spectrometric data processing modules comprise a data extraction module. 251. The system of embodiment 250, wherein the data extraction module extracts information from at least one data file for the data set for subsequent analysis during the computational workflow. 252. The system of any one of embodiments 250-251, wherein the data extraction module extracts at least one of total ion chromatogram, retention time, acquired time range, fragment voltage, ionization mode, ion polarity, mass units, scan type, spectrum type, threshold, sampling period, total data points, and total scan counts. 253. The system of any one of embodiments 250-252, wherein the data extraction module extracts the MS2 information from the data set and converts the MS2 information into a suitable format. 254. The system of embodiment 253, wherein the MS2 information is converted into Mascot generic format using an application library. 255. The system of any one of embodiments 250-254, wherein a quality assessment performed on data extraction determines if the data set has been successfully extracted and converted. 256. The system of any one of embodiments 230-255, wherein the mass spectrometric data processing modules comprise a feature extraction module. 257. The system of embodiment 256, wherein the feature extraction module extracts molecular features for peak detection. 258. The system of any one of embodiments 256-257, wherein the feature extraction module stores extracted features in parallel sections to a java serialized file for downstream analysis. 259. The system of any one of embodiments 256-258, wherein the feature extraction module extracts initial molecular features and refines the features using LC and isotopic profiles. 260. The system of any one of embodiments 256-259, wherein the feature extraction module filters and deisotopes MS1 peaks extracted from the data set. 261. The system of any one of embodiments 256-260, wherein the feature extraction module applies filtering and clustering techniques to evaluate raw extracted molecular peaks. 262. The system of any one of embodiments 256-261, wherein a quality assessment of feature extraction comprises evaluating the extracted data set using at least one quality control metric. 263. The system of any one of embodiments 230-262, wherein the mass spectrometric data processing modules comprise a proteomic processing module. 264. The system of embodiment 263, wherein the proteomic processing module creates at least one list for targeted data acquisition. 265. The system of any one of embodiments 263-264, wherein the proteomic processing module performs a correction on the data set by incorporating at least one of mass difference and charge. 266. The system of any one of embodiments 263-265, wherein the proteomic processing module compares precursor mass and charge from an MGF file with refined values developed by a feature extraction module and correcting the MGF file when the precursor mass and charge differ with the refined values. 267. The system of any one of embodiments 263-266, wherein the proteomic processing module performs a forward proteomic data search for peptides or proteins against a protein database. 268. The system of any one of embodiments 263-267, wherein the proteomic processing module performs forward proteomic database search and a reverse proteomic database search, wherein the reverse proteomic database search allows generation of a false discovery rate. 269. The system of any one of embodiments 263-268, wherein the proteomic processing module generates proposed peptides based on a proteomic database search and filters the proposed peptide based on an RT model generated from the data set. 270. The system of any one of embodiments 263-269, wherein a quality assessment of proteomic processing comprises evaluating an output of the proteomic processing against at least one quality control metric. 271. The system of any one of embodiments 230-271, wherein the mass spectrometric data processing modules comprise a quality control module. 272. The system of embodiment 271, wherein the quality control module performs at least one quality assessment of some of the data processing modules or steps in the computational workflow. 273. The system of any one of embodiments 271-272, wherein the quality control module performs a gating step removing at least a portion of the data set from subsequent analysis based on at least one quality assessment of at least one data processing module or step in the computational workflow. 274. The system of any one of embodiments 271-273, wherein the quality control module terminates the computational workflow for the data set based on a quality assessment of at least one data processing module or step in the computational workflow. 275. The system of any one of embodiments 271-274, wherein the quality control module flags at least a portion of the data set based on a quality assessment of at least one data processing module or step in the computational workflow. 276. The system of any one of embodiments 271-275, wherein the quality control module performs at least one quality assessment of the computational workflow by evaluating at least one output of a data processing module against at least one quality control metric. 277. The system of any one of embodiments 230-276, wherein the plurality of protein processing modules positioned in series comprises at least two modules. 278. The system of any one of embodiments 230-277, wherein the plurality of protein processing modules positioned in series comprises at least four modules. 279. The system of any one of embodiments 230-278, wherein the plurality of protein processing modules positioned in series comprises at least six modules. 280. The system of any one of embodiments 230-279, wherein the plurality of protein processing modules positioned in series comprises at least eight modules. 281. The system of any one of embodiments 230-280, wherein the mass spectrometric data processing modules comprise a visualization module. 282. The system of embodiment 281, wherein the visualization module generates a visualization of the data set at any step during the computational workflow. 283. The system of any one of embodiments 281-282, wherein the visualization module generates a star field visualization of the data set. 284. The system of any one of embodiments 281-283, wherein the visualization module generates a star field visualization of the data set showing signal intensity plotted against m/z with isotopic features appearing as points or dots. 285. The system of any one of embodiments 281-284, wherein the visualization module generates a star field visualization of the data set showing a 4-dimensional m/z over liquid chromatography time showing isotopic feature views of peaks as points of light. 286. The system of any one of embodiments 230-285, wherein the mass spectrometric data processing modules comprise a utility module. 287. The system of embodiment 286, wherein the utility module provides at least one utility function for monitoring or supervising the computational workflow. 288. The system of any one of embodiments 286-287, wherein the utility module provides at least one utility function for monitoring or supervising an end-to-end mass spectrometric workflow comprising the computational workflow, an experimental design workflow, and a mass spectrometric data processing workflow. 289. The system of any one of embodiments 286-288, wherein the utility module provides at least one utility function for visualizing the data set, calculating charged mass, calculating molecular weight, calculating peptide mass, calculating tandem pass, searching for sequence homology, determining column usage, plotting spectra, determining pipeline status, checking machine status, tuning reports, controlling workflow, or annotating issues that arise during the computational workflow. 290. A system for automated mass spectrometric analysis of a data set, comprising: a) a plurality of mass spectrometric data processing modules; and b) a workflow determination module extracting a mass spectrometric method and parameters from a worklist associated with the data set and using the mass spectrometric method and parameters to generate a computational workflow comprising the plurality of data processing modules positioned in series to analyze the data set. 291. A system for automated mass spectrometric analysis of a data set, comprising: a) a plurality of mass spectrometric data processing modules; and b) a workflow determination module generating a computational workflow comprising the plurality of data processing modules positioned in series to analyze the data set, wherein at least one of the plurality of data processing modules in the workflow is selected based on quality assessment information obtained during mass spectrometric sample processing. 292. A system for automated mass spectrometric analysis of a data set obtained from a sample, comprising: a) a plurality of mass spectrometric data processing modules; and b) a workflow determination module generating a computational workflow comprising the plurality of data processing modules positioned in series to perform data analysis of the data set, wherein the data analysis is informed by at least one automated quality assessment carried out during sample processing. 293. The system of embodiment 292, wherein the data analysis comprises deciding between discarding and retaining a portion of the data set for downstream analysis based on a tag applied to the portion of the data set by the at least one automated quality assessment. 294. The system of embodiment 293, wherein the tag indicates the portion of the data set is uninformative. 295. The system of embodiment 293, wherein the tag indicates the portion of the data set is low quality according to at least one quality control metric. 296. The system of embodiment 293, wherein the tag indicates the portion of the data set is uninformative of a category of proteins. 297. The system of embodiment 296, wherein the category of proteins is low abundance proteins, medium abundance proteins, or high abundance proteins. 298. The system of embodiment 296, wherein the category of proteins comprises structural proteins, signaling proteins, phospho-proteins, post-translationally modified proteins, membrane proteins, intracellular proteins, secreted proteins, extracellular matrix proteins, housekeeping proteins, immunoglobulins, or any combination thereof 299. The system of embodiment 292, wherein the data analysis comprises detecting a tag applied to the data set by the at least one automated quality assessment indicating the data set is uninformative and discarding the entire data set from downstream analysis. 300. A system for automated mass spectrometric analysis of a data set obtained from a sample, comprising: a) a plurality of mass spectrometric data processing modules; and b) a workflow determination module generating a computational workflow comprising the plurality of data processing modules positioned in series to perform data analysis of the data set, wherein the data analysis is informed by at least one quality control metric generated by at least one quality assessment carried out during sample processing. 301. A system for automated mass spectrometric analysis of a data set, comprising: a) a plurality of mass spectrometric data processing modules for carrying out a computational workflow analyzing the data set; and b) a quality control module performing a quality assessment for a data analysis output of at least one of the plurality of data processing modules, wherein the output failing the gated quality assessment results in at least one of the computational workflow being paused, the output being flagged as deficient, and the output being discarded. 302. A system for automated mass spectrometric analysis of a data set comprising a plurality of mass spectrometric data processing modules; a workflow determination module parsing a worklist associated with the data set to extract parameters for a workflow for downstream data analysis of the data set by the plurality of data processing modules; and a quality control module assessing at least one quality control metric for some of the plurality of data processing modules and tagging the output when the output fails the at least one quality control metric, wherein the tagging informs downstream data analysis. 303. A system for automated mass spectrometric analysis comprising a plurality of mass spectrometric data processing modules for processing mass spectrometric data; wherein each mass spectrometric data processing module operates without ongoing supervision. 304. The system of embodiment 303, wherein the system further comprises at least one mass spectrometric data processing module subject to ongoing supervision. 305. The system of any one of embodiments 303-304, wherein at least one mass spectrometric data processing module is configured to evaluate performance of an immediately prior mass spectrometric data processing module. 306. The system of any one of embodiments 303-305, wherein at least one mass spectrometric data processing module is configured to evaluate an effect of an immediately prior mass spectrometric data processing module on sample data. 307. The system of any one of embodiments 303-306, wherein at least one mass spectrometric data processing module is configured to evaluate sample data using a quality control metric after the sample data has been processed by at least one mass spectrometric data processing module. 308. The system of any one of embodiments 303-307, wherein the mass spectrometric data processing modules are configured to stop sample data analysis when evaluation indicates that a quality control metric is not met. 309. The system of any one of embodiments 303-308, wherein the mass spectrometric data processing modules are configured to tag a sample data analysis output when evaluation indicates that a quality control metric is not met for at least one sample analysis module. 310. The system of any one of embodiments 303-309, wherein the mass spectrometric data processing modules comprise a data acquisition module. 311. The system of any one of embodiments 303-310, wherein the mass spectrometric data processing modules comprise a workflow determination module generating a workflow for downstream data processing by subsequent data processing modules. 312. The system of embodiment 311, wherein the workflow is a pre-set workflow based on the type of mass spectrometric data analysis selected. 313. The system of any one of embodiments 311-312, wherein the workflow is a pre-set workflow based on parameters extracted from a work list for the mass spectrometric data. 314. The system of any one of embodiments 311-313, wherein the workflow is a customized workflow based on parameters extracted from a work list for the mass spectrometric data. 315. The system of any one of embodiments 311-314, wherein the workflow is configured to process mass spectrometric data generated by Profile and DPS proteomics. 316. The system of any one of embodiments 311-315, wherein the workflow is configured to process data generated by Targeted and iMRM proteomics. 317. The system of any one of embodiments 303-316, wherein the mass spectrometric data processing modules comprise a data extraction module. 318. The system of any one of embodiments 303-317, wherein the mass spectrometric data processing modules comprise a feature extraction module. 319. The system of any one of embodiments 303-318, wherein the mass spectrometric data processing modules comprise a proteomic processing module. 320. The system of any one of embodiments 303-319, wherein the mass spectrometric data processing modules comprise a quality control module. 321. The system of any one of embodiments 303-320, wherein the plurality of protein processing modules positioned in series comprises at least four modules. 322. The system of any one of embodiments 303-321, wherein the plurality of protein processing modules positioned in series comprises at least eight modules. 323. A computer-implemented method for carrying out the steps according to any of the preceding systems. 324. A method for automated mass spectrometric analysis of a data set, comprising: a) providing a plurality of mass spectrometric data processing modules; and b) providing a workflow determination module generating a computational workflow comprising the plurality of data processing modules positioned in series to analyze the data set, wherein the computational workflow is configured based on at least one of a worklist and at least one quality assessment performed during mass spectrometric sample processing. 325. A method for automated mass spectrometric analysis of a data set, comprising: a) providing a plurality of mass spectrometric data processing modules; and b) providing a workflow determination module extracting a mass spectrometric method and parameters from a worklist associated with the data set and using the mass spectrometric method and parameters to generate a computational workflow comprising the plurality of data processing modules positioned in series to analyze the data set. 326. A method for automated mass spectrometric analysis of a data set, comprising: a) providing a plurality of mass spectrometric data processing modules; and b) providing a workflow determination module generating a computational workflow comprising the plurality of data processing modules positioned in series to analyze the data set, wherein at least one of the plurality of data processing modules in the workflow is selected based on quality assessment information obtained during mass spectrometric sample processing. 327. A method for automated mass spectrometric analysis of a data set obtained from a sample, comprising: a) providing a plurality of mass spectrometric data processing modules; and b) providing a workflow determination module generating a computational workflow comprising the plurality of data processing modules positioned in series to perform data analysis of the data set, wherein the data analysis is informed by at least one automated quality assessment carried out during sample processing. 328. A method for automated mass spectrometric analysis of a data set obtained from a sample, comprising: a) providing a plurality of mass spectrometric data processing modules; and b) providing a workflow determination module generating a computational workflow comprising the plurality of data processing modules positioned in series to perform data analysis of the data set, wherein the data analysis is informed by at least one quality control metric generated by at least one quality assessment carried out during sample processing. 329. A method for automated mass spectrometric analysis of a data set, comprising: a) providing a plurality of mass spectrometric data processing modules for carrying out a computational workflow analyzing the data set; and b) providing a quality control module performing a quality assessment for a data analysis output of at least one of the plurality of data processing modules, wherein the output failing the gated quality assessment results in at least one of the computational workflow being paused, the output being flagged as deficient, and the output being discarded. 330. A method for automated mass spectrometric analysis of a data set comprising: providing a plurality of mass spectrometric data processing modules; providing a workflow determination module parsing a worklist associated with the data set to extract parameters for a workflow for downstream data analysis of the data set by the plurality of data processing modules; and providing a quality control module assessing at least one quality control metric for some of the plurality of data processing modules and tagging the output when the output fails the at least one quality control metric, wherein the tagging informs downstream data analysis. 331. A method for automated mass spectrometric analysis, comprising providing a plurality of mass spectrometric data processing modules for processing mass spectrometric data; wherein each mass spectrometric data processing module operates without ongoing supervision. 332. A health condition indicator identification process, comprising: receiving an input parameter; accessing a dataset in response to receiving the input, the dataset comprising information relating to at least one predetermined association between the input parameter and at least one health condition indicator; and generating an output comprising a health condition indicator having a predetermined association with the input parameter. 333. The process of embodiment 332, wherein the input parameter comprises a biomarker or portion thereof. 334. The process of embodiment 333, wherein the biomarker comprises a protein. 335. The process of embodiment 333, wherein the biomarker comprises a peptide. 336. The process of embodiment 333, wherein the biomarker comprises a polypeptide. 337. The process of embodiment 332, wherein the input parameter comprises a gene. 338. The process of embodiment 332, wherein the input parameter comprises a health condition status. 339. The process of embodiment 338, wherein the heath condition status indicates presence of a colorectal disease. 340. The process of embodiment 339, wherein the colorectal disease is colorectal cancer. 341. The process of any one of embodiments 332-340, wherein the health condition indicator comprises a biological pathway. 342. The process of any one of embodiments 332-340, wherein the health condition indicator comprises a health condition status. 343. The process of any one of embodiments 332-340, wherein the health condition indicator comprises a biomarker or portion thereof 344. The process of any one of embodiments 332-343, wherein generating the output comprises performing automated mass spectrometric analysis utilizing a computational workflow comprising a plurality of mass spectrometric data processing modules positioned in series to perform data analysis of the dataset. 345. The process of any one of embodiments 332-343, wherein the dataset is obtained using automated mass spectrometric analysis utilizing a series of protein processing modules positioned in series and at least one mass spectrometric sample analysis module positioned between two protein processing modules. 346. A tangible storage medium comprising instructions configured to: receive an input parameter; access a dataset in response to receiving the input, the dataset comprising information relating to at least one predetermined association between the input parameter and at least one health condition indicator; and generate an output comprising a health condition indicator having a predetermined association with the input parameter. 347. A computer system comprising the tangible medium of embodiment 346. 348. A health condition indicator identification process, comprising: receiving an input parameter; transmitting the input parameter to a server; receiving an output generated in response to the input parameter, the output comprising a health condition indicator comprising a predetermined association with the input parameter; and displaying the output to a user. 349. The process of embodiment 348, wherein the input parameter comprises a health condition status. 350. The process of embodiment 349, wherein the health condition status indicates presence of a colorectal disease. 351. A display monitor configured to present biological data, said display monitor presenting at least two disorder nodes, at least one gene node, at least one protein node, at least one pathway node, and markings indicating relationships among at least some of said nodes. 352. The display monitor of embodiment 351, said display monitor presenting at least ten protein nodes. 353. The display monitor of any one of embodiments 351-352, said display monitor presenting at least ten polypeptide marker nodes. 354. The display monitor of embodiment 353, wherein said at least ten polypeptide marker nodes map to a common polypeptide marker collection node. 355. The display monitor of any one of embodiments 351-354, wherein one of said at least two disorder nodes is an input disorder node. 356. The display of any one of embodiments 351-355, wherein all nodes comprise public information. 357. The display of any one of embodiments 351-356, wherein at least one node comprises unpublished experimental results. 358. The display of any one of embodiments 351-357, wherein the display monitor presents at least 50 nodes. 359. The display monitor of any one of embodiments 351-358, wherein nodes and node relationships are presented in no more than 1 minute following node input. 360. The method of any one of embodiments 21-39, wherein the threshold of at least one QC metric is not met when no more than 10 non-corresponding features between the sample runs is identified. 361. The method of any one of embodiments 21-39, wherein the identified features comprise charge state, chromatographic time, overall peak shape, analyte signal strength, presence of known contaminants, or any combination thereof

Much of the present disclosure is focused on protein or polypeptide fragments thereof. Nevertheless, the approaches described herein can be used on other biomolecules such as lipids, metabolites, and other biomolecules as described throughout the specification. For example, analytical instruments described herein such as mass spectrometers can be used for the analysis of various biomolecules in addition to proteins or polypeptide fragments.

Further understanding of the disclosure herein is gained in light of the Examples provided below and throughout the present disclosure. Examples are illustrative but are not necessarily limiting on all embodiments herein.

### EXAMPLES

### Example 1. Ungated Workflows Generate Data that Incorporates Systemic Biases

A researcher is interested in identifying circulating biomarkers relevant to colorectal cancer (CRC). Samples from 100 individuals later determined to have CRC and 100 individuals later determined to be free of CRC are subjected to analysis. 80 of the CRC positive samples are obtained from a 30 year old sample collection, whole the CRC negative samples are freshly taken. The storage of the 30 year old sample results in substantial internal cleavage of proteins of the samples, such that total protein amount is unaffected but many proteins are cleaved into fragments.

The samples are subjected to ungated analysis. Polypeptides underrepresented in the CRC positive samples are identified and selected for use in a panel for CRC. The panel does not accurately detect CRC.

This example illustrates the risks in workflows that are not subjected to automated gating.

### Example 2. Automated Gating of Mass Spectrometric Workflows Generate Data that is Readily Comparable

A researcher is interested in identifying circulating biomarkers relevant to colorectal cancer (CRC). Samples from 100 individuals later determined to have CRC and 100 individuals later determined to be free of CRC are subjected to analysis. 80 of the CRC positive samples are obtained from a 30 year old sample collection, whole the CRC negative samples are freshly taken. The storage of the 30 year old sample results in substantial internal cleavage of proteins of the samples, such that total protein amount is unaffected but many proteins are cleaved into fragments.

The samples are subjected to automated gated analysis. Automated analysis of a proteolysis step such as tryptic digestion identifies that the digest has resulted in disproportionately small polypeptide fragments in the 80 CRC samples taken from a 30 year old sample collection. These samples are flagged, and their outputs are excluded from further analysis.

Polypeptides that vary between the 100 healthy samples and the 20 recently collected CRC positive samples are identified and selected for use in a panel for CRC. The panel accurately detects CRC.

This example illustrates the benefits in workflows that are subjected to automated gating.

### Example 3. Automated Gating of Mass Spectrometric Workflows Identifies a Workflow Step to Revise

A researcher is interested in identifying circulating biomarkers relevant to colorectal cancer (CRC). Samples from 100 individuals later determined to have CRC and 100 individuals later determined to be free of CRC are subjected to analysis. 80 of the CRC positive samples are obtained from a 30 year old sample collection, whole the CRC negative samples are freshly taken. The storage of the 30 year old sample results in substantial internal cleavage of proteins of the samples due to specific cleavage at Arginine residues, such that total protein amount is unaffected but many proteins are cleaved into fragments.

The samples are subjected to automated gated analysis. Automated analysis of a trypsin proteolysis step identifies that the trypsin digest has resulted in disproportionately small polypeptide fragments in the 80 CRC samples taken from a 30 year old sample collection. These samples are flagged, and their outputs are excluded from further analysis. The trypsin digest step is identified as a step leading to flagging.

The trypsin digest step is replaced by a proteolytic digest step comprising treatment using a protease that specifically cleaves at arginine residues.

The workflow is repeated, and it is observed that the 30 year old samples are no longer flagged at the protease digestion step. Differences between CRC positive and CRC negative samples are used to develop a CRC assay. The assay is determined to be more accurate than the assay of example 2.

This example illustrates the benefits in performing automated gating to identify manipulation steps warranting further attention.

### Example 4. Automated Gating of Mass Spectrometric Workflows Facilitates Rapid Generation of Comparable Data

A researcher is interested in identifying circulating biomarkers relevant to colorectal cancer (CRC). Samples from 100 individuals later determined to have CRC and 100 individuals later determined to be free of CRC are subjected to analysis. 80 of the CRC positive samples are obtained from a 30 year old sample collection, whole the CRC negative samples are freshly taken. The storage of the 30 year old sample results in substantial internal cleavage of proteins of the samples due to specific cleavage at Arginine residues, such that total protein amount is unaffected but many proteins are cleaved into fragments.

The samples are subjected to automated gated analysis. Automated analysis of a trypsin proteolysis step identifies that the trypsin digest has resulted in disproportionately small polypeptide fragments in the 80 CRC samples taken from a 30 year old sample collection. These samples are flagged, and their outputs are excluded from further analysis. The trypsin digest step is identified as a step leading to flagging.

The trypsin digest step is replaced by a proteolytic digest step comprising treatment using a protease that specifically cleaves at arginine residues.

The workflow is repeated, and it is observed that the 30 year old samples are no longer flagged at the protease digestion step.

Researcher analysis is required only at the step of selection of a trypsin alternative, and in steps in analysis that occur subsequent to mass spectrometric data generation that occur selection. A CRC researcher without specific training in mass spectrometric machinery or workflows performs all steps of the analysis leading to generation of the CRC panel.

This example illustrates that automated gating of particular manipulation steps allows mass spectrometric analysis to be performed, assessed and workflows to be improved without relying on a particular set of skills related to sample generation, processing and analysis related to mass spectrometry, such that the technique is available to specialists in a particular disorder rather than to specialists in mass spectrometry workflows.

### Example 5. Gated Data is Readily Compared or Combined in Support of or in Place of New Sample Analyses

A condition, early non-small cell clung cancer, is identified and an automated search is executed to identify candidate markers indicative of the condition. Candidate makers are assembled into a list. An automated search indicates that gated data is available from a previous analysis of a patient population informative of a different condition, emphysema. It is observed that a substantial number of participants in the previous analysis are found to develop early non-small cell lung cancer.

The data is analyzed to assess relevance of the candidate markers. Markers are identified that correlate with presence of the condition. However, the sample size of positive individuals is insufficient to generate the desired level of statistical confidence.

Samples are collected from a limited number of individuals positive and negative for the condition. The number is insufficient to generate a result of the desired statistical significance. Sample data is generated through a gated workflow, so as to generate gated data for subsequent analysis. The data is confirmed to satisfy gating in its generation but to be insufficient to generate a verified panel having the desired level of significance.

The datasets are combined. As both are gated, there is sufficient similarity in data quality to allow their merger into a single set for a downstream analysis.

Analysis is performed on the merged gated datasets, and a statistically significant signal is obtained for a subset of the candidate markers. A panel is derived from the subset of candidate markers, and is used in a noninvasive test for the disorder.

### Example 6. Manual CRC Study Planning

A researcher wishes to identify potential proteins for evaluating a CRC (colorectal cancer) proteomics signature. The researcher performs extensive literature searching of about 100 references, which takes weeks, and identifies a list of candidate biomarkers for the study. The researcher creates a study plan including protocols, sample size, and planned data analysis, and executes the study plan. The study plan does not take into account the quality of references used to identify the biomarkers, and after the study is concluded, it is found that the study design chosen as a result of this oversight results in insufficient statistical power to accurately identify proteins that correlate to CRC. This examples illustrates the challenges of identifying potential protein biomarkers and designing proteomics studies that are likely to succeed in finding clinically relevant correlations.

### Example 7. CRC Study Planning with a Text Search

A question was defined to evaluate potential proteins for evaluating a CRC (colorectal cancer). Literature and internal databases were searched automatically for potential proteomics targets using keywords, distance between keywords, and associated pathways known to be involved in a disease. The quality of the references was evaluated, and reference meeting predetermined quality thresholds were further analyzed. Studies and data sets mentioned in the references were further evaluated quality including sample sizes and statistical metrics. References that passed these gating steps comprise 187 potential proteins involved in CRC. Targets not meeting predetermined quality standards were removed or flagged before the data was used for further study design and empirical evaluation. An in-silico tryptic digestion predicted 77,772 predicted peptides, and the in-silico digestion results were evaluated for quality standards. Peptides not meeting quality standards were removed from the analysis, or flagged for later evaluation. Peptides with the potential for chemical modification were removed from the set, leaving 24,413 peptides. The threshold for chemical modification potential was used as a quality control measure to evaluate the results of the filter; peptides meeting a threshold for likely chemical modification were removed from the analysis. Further filtering steps were carried out in an analogous manner: removing homologous peptides (leaving 13,995 peptides), verifying LCMS compatibility (leaving 9,447 peptides), choosing the top 5 predicted peptides per protein from the model, and finally subjecting these to empirical evaluation. Each of the previous steps is gated for quality control, ensuring that each peptide filtering step is controlled based on a previously determined threshold. Peptides not meeting this standard were either removed from the set, or flagged for later review. All of the operations in the study plan were performed without human supervision.

### Example 8. Automated CRC Study Planning

A question was defined to evaluate potential proteins for evaluating a CRC (colorectal cancer) proteomics signature, and literature and internal databases are searched for potential proteomics targets from 312 known protein isoforms. 187 potential proteins involved in CRC are identified from the search, and the quality of these potential targets was evaluated. Targets not meeting predetermined quality standards were removed or flagged before the data was used for further study design and empirical evaluation. An in-silico tryptic digestion predicted 77,772 predicted peptides, and the in-silico digestion results were evaluated for quality standards. Peptides not meeting quality standards were removed from the analysis, or flagged for later evaluation. Peptides with the potential for chemical modification were removed from the set, leaving 24,413 peptides. The threshold for chemical modification potential was used as a quality control measure to evaluate the results of the filter; peptides meeting a threshold for likely chemical modification were removed from the analysis. Further filtering steps were carried out in an analogous manner: removing homologous peptides (leaving 13,995 peptides), verifying LCMS compatibility (leaving 9,447 peptides), choosing the top 5 predicted peptides per protein from the model, and finally subjecting these to empirical evaluation. Each of the previous steps is gated for quality control, ensuring that each peptide filtering step is controlled based on a previously determined threshold. Peptides not meeting this standard were either removed from the set, or flagged for later review.

### Example 9. CRC Study Planning with a Human Review Step

A researcher designs a study plan using the general methods of Example 7, with the modification that the researcher reviews the references that were accepted and rejected by the gating step. The researcher adjusts the thresholds for the gating step to be more stringent, and reduces the number of references passing the gating step. The remaining steps in workflow planning are then executed without further human intervention or review.

### Example 10. Study Planning without Search Gating

A researcher designs a study plan using the general methods of Example 7, with the modification that no steps are gated to control the quality of the results. The research finds that several peptides identified in a search for candidate biomarkers of lung cancer in humans are proteins that are found only in bacteria. The researcher then spends hours manually evaluating all references corresponding to 2,000 potential protein biomarkers identified by the search, and finds the protein sequences and names were improperly entered into a public database. This example illustrates that disparities or errors in databases can hinder workflow planning.

### Example 11. Study Planning with Search Gating

A researcher designs a study plan using the general methods of Example 10, with the modification that the study workflow planning method comprises one or more gating modules. A gating module determines that some of the identified candidate biomarkers are bacterial proteins which are inconsistent with other candidate biomarkers found, and these suspect candidate biomarker proteins are flagged for later review. Unflagged candidate biomarkers are identified, and reagents suitable for detection of the marker candidate are identified and optionally located in an inventory. The workflow plan is successfully executed without using the flagged candidate biomarker proteins, and the references containing the incorrect sequences are flagged for future searches.

### Example 12. Study Planning with Signal Gating

A researcher designs a study plan using the general methods of Example 7, with the modification that the study workflow planning method comprises one or more gating modules. A gating module determines that some of the identified candidate biomarkers are bacterial proteins which are inconsistent with other biomarkers found, and these suspect candidate biomarker proteins are flagged for later review. The workflow plan is successfully executed without using the flagged candidate biomarker proteins.

### Example 13. CRC Study Planning without Sample Evaluation

A researcher designs a study plan using the general methods of Example 7, with the modification that after identifying potential protein candidates, experimental designs are generated based on evaluation of confounding factors, and power analyses are performed. Sample sources are identified, and data collection is evaluated. Early samples are evaluated, a transition pool is defined, the MS method is optimized, and final transitions are selected. However, the sample source is whole blood, and signals from hemoglobin are interfering with evaluation of the desired biomarkers. The study fails to identify biomarkers in early samples due to this interference, and the study plan is abandoned.

### Example 14. CRC Study Planning with Sample Evaluation

A researcher designs a study plan using the general methods of Example 13, with the modification that after identifying potential protein candidates, experimental designs are generated based on evaluation of confounding factors, and power analyses are performed. Sample sources are identified, and data collection is evaluated. A gating module identifies likely interference of hemoglobin from the sample source, and the experimental design is adjusted to compensate for the interference of hemoglobin signals. Early samples are evaluated, a transition pool is defined, the MS method is optimized, and final transitions are selected. Finally, samples are randomized in preparation for running the full-scale proteomics experiment. The full-scale proteomics experiment succeeds in identifying biomarkers by eliminating at least some of the interference from hemoglobin at all subsequent mass spectrometry and analysis steps.

### Example 15. CRC Study with Prior Study Data Integration

A researcher wishes to identify potential proteins for evaluating a CRC (colorectal cancer) proteomics signature, and designs a study plan using the general methods of Example 14. During the search for candidate biomarker proteins, a previous study concerning a different disease with at least one of the same candidate biomarker proteins is found. This previous study was conducted with gating steps and the high quality data obtained from the study is integrated into the current workflow plan. As a result of integration, the workflow plan reduces the number of samples needed to obtain a statistically significant result for the current study, and selects previously well-performing proteins that were reliable markers in the previous study. This example illustrates how evaluation and integration of previous, high quality, gated data sets can significantly reduce the time and resources needed for a subsequent study.

### Example 16: Fractionated Proteomics

The following example describes an exemplary workflow and devices for use in a fractionated proteomics study. Experiments are tracked and organized by a LIMS. The LIMS has automated uploads and downloads. The LIMS sets up previously computed sample ordering and randomization and tracks experimental worksheets and worklists. Sample ordering is determined as part of the overall study design. The LIMS computes parameters applied in ChemStation software. LC trace data is processed and normalized, then written to a CSV file. Optical density measurements are made to measure the protein concentration in each sample. Controls of known protein concentration are measured to determine the parameters applied in computation of sample concentrations. Samples that do not fall within desired parameters are flagged. The LIMS computes parameters of LC traces into protein mass estimates. Controls of known protein mass are fractionated and then measured to determine the parameters applied in computation of fraction mass distribution.

Bulk reagents and stock solutions are prepared prior to sample process start and stored appropriately for use over the experiment days. Plate QC samples are derived from known sample pools and are processed in parallel with study samples so that they undergo exactly the same laboratory actions.

Sample mixtures are determined, including aliquot count and volume.

Samples are initially processed by ordering them according to data preloaded into LIMS. This includes the process quality control samples. The samples are thawed and examined. A user assesses the sample for features that would compromise its ability to be analyzed, including hyperlipidemia and the presence of large amounts of hemoglobin. Samples that fail this analysis are flagged.

Buffer is added to the samples for protein depletion. The samples are run through a multiple affinity removal column. Particles and lipids are filtered. Samples are assessed for particles and lipids and samples in which particles and lipids are not adequately filtered are flagged.

The amount of protein in each sample is determined so that correct amounts of reagents and buffers can be added. This is accomplished using a total protein assay to estimate the total amount of protein in each sample. Each plate has 3 replicates of 8 standard dilutions. A subset of standard measurements of 4 dilution values is chosen. These include 400, 300, 200 and 100 μg/μl concentrations. The samples are optically scanned. These measurements are used to generate the slope and intercept of a linear model of the concentration/OD measurement relationship. If the absolute value of the error (difference from model prediction) of any group of 3 replicates is >10%, the experiment is flagged. The operator then uses standards associated with the previously unused dilution values to find an acceptable standard. The entire set of measurements is flagged when an acceptable standard is not found.

Each experimental sample has 5 replicates. A sample is flagged if there are not at least 4 values read for each sample. A sample is also flagged if the computed mass value has a coefficient of variation greater than 10%. Samples are flagged individually on a plate and other samples on the plate can continue.

In this example, a sample is flagged because the computed mass value calculated from 5 replicates has a coefficient of variation that is greater than 10%. One of the replicates is deemed to be problematic because a tip used to prepare the replicate became clogged, and thus the replicate wasn't processed properly. This replicate is excluded from subsequent analysis and the coefficient of variation is recalculated and determined to be acceptable. The sample is not flagged.

Another sample is flagged because the total protein assay was only able to calculate protein concentrations for three out of the five replicates. Flagged samples are rerun through the total protein assay or scheduled for reprocessing.

Worklists for automated fractionation, digestion and reconstitution are customized for every sample. The LIMS estimates sample protein concentration based on uploaded optical density measurements. LIMS also assesses OD measurement quality and flags out-of-spec results. Next, LIMS computes the amount of each sample to inject into IDFC to achieve constant protein amounts for digestion. The accuracy of this step can help ensure reproducibility of the depletion.

The samples are then depleted and fractionated in triplicate. Depletion removes the most abundant proteins from the sample so that lower concentration proteins are detectable. In this example, albumin, IgG, antitrypsin, IgA, transferrin, haptoglobin, fibrinogen, alpha 2-macroglobulin, alpha1-acid glycoprotein, IgM, apolipoprotein A1, apolipoprotein A2, complement C3, and transthyretin are depleted from the samples. Fractionation further divides each sample to 1) increase the total number of proteins detected and 2) separate isoforms for individual proteins based on protein hydrophobicity. Both actions are accomplished using a customized Immuno-Depletion Fractionation (IDFC) LC system.

Samples are assessed for fractionation and depletion by analyzing the chromatography traces and comparing chromatography traces between replicates. The process includes generating a worklist file, putting the samples into a 96 well plate, double checking to ensure sample locations are correct, and fractionating the wells by liquid chromatography. Based on values in the uploaded CSV file, the earlier estimate of total sample protein mass is distributed among the samples' fractions.

The traces are evaluated for uniformity. A peak that shifted and eluted at an unexpected time in one of the three replicates is evaluated and a pump leak is detected. The trace is automatically corrected. Fractions from each replicate determined to contain excessive amounts of the abundant proteins listed above are discarded. Fractions from each replicate determined to contain analytes of interest are retained. An exemplary trace is shown in FIG. 26. The x axis shows time and the y axis shows absorbance of UV. Proteins with low abundance flow off the column at an earlier time point and those fractions are collected for subsequent analysis. More abundant proteins removed by the depletion system elute at a later time point and those fractions are discarded.

Samples that are not properly fractionated or depleted are flagged and subjected to an additional round of fractionation and depletion, as appropriate. The replicates of one sample are assessed and flagged because the peaks are not uniform between each replicate. The reason for the non-uniformity cannot be determined and the sample traces cannot be corrected. The sample is reprocessed through the depletion and fractionation step and new traces generated. These traces are determined to be sufficiently uniform and meet quality control standards. The appropriate fractions proceed through the workflow.

Next, LIMS computes appropriate volumes of trypsin and recon buffer for each sample fraction based on protein mass estimates. These data are used to generate a worklist, which is uploaded to a Tecan workstation. Trypsin is added to each well based on the calculated amounts determined by the LIMS. The resulting samples are analyzed for digest quality, including average fragment size, fragment size range, fragment size distribution, and incomplete digestion. Digestion of a second aliquot using the same or different restriction enzyme is repeated for samples flagged for failing any of these tests. Volumes are controlled to match instrument configurations.

The samples are then dried for storage or processing for mass spectroscopy. This includes quenching the samples and drying them, washing them with SPE buffer to maximize recover, and lyophilizing them. The samples can be frozen at this point if the mass spectrometer is not available for use.

The readiness of the mass spectrometer is assessed prior to use. Each run of digested samples is preceded by a quality control run to determine if the LCMS is functioning within defined tolerances. An aliquot from a previously-characterized sample is run through the liquid chromatograph and a trace is generated. The trace is compared to previous traces generated using other aliquots of the previously-characterized sample. The quality of the column, the pressure of the column, and the quality of the trace are assessed. The trace is determined to vary from the previously collected trace. It is determined that nearly 500 samples have previously been run through the column. The column is replaced and a new trace is generated using the previously-characterized sample. The trace and pressure measurements are deemed acceptable.

The previously-characterized sample is fed from the new chromatography column into the mass spectrometer. Features are counted and compared to data generated in previous mass spec runs using the same sample. It is determined that the mass spec detected a minimum acceptable number of features with each of charge states 2, 3, and 4. Retention times are also calculated using total ion current. The experiment is a multi-part study, so these data are compared to previous runs from this experiment and from other experiments. A major shift in the retention time is detected compared to previous data. The experiment is postponed. A leak in the liquid chromatograph is detected and fixed. The previously-characterized sample is run through the LCMS again and the column and mass spectrometer are determined to be working properly. The mass spec is deemed ready and the experiment continues to process patient samples.

LIMS uses a template to generate a LCMS worklist with randomized sample ordering and appropriate injection volumes for each sample to standardize the mass loaded onto the LC column. The quality control run samples are processed in the same order for every worklist (e.g. first, middle and last) to provide sample/worklist normalization during data analysis. The worklist file is archived automatically. The generated LCMS worklist is imported into the LCMS control software. The worklist name, along with sample injection order, is loaded into the LCMS control software and confirmed by a user. After confirmation of the loaded worklist file, the worklist is started through the instrument control software. The resulting data are then assessed for quality using pre-defined metrics. Data that does not meet or exceed quality standards are flagged.

Lyophilized samples are reconstituted in an appropriate buffer for injection on the LCMS. LIMS dynamically computes the individual sample buffer volume for reconstitution to yield standardized peptide loading on the LCMS across all sample wells. This is used to generate a worklist for reconstituting the samples using Tecan. The worklist is archived automatically. Reconstitution buffer is volumetrically dispensed with a Tecan liquid handling robot. Samples receiving an incorrect volume of buffer or that are otherwise mishandled are flagged. The plate is then centrifuged. Samples containing bubbles are flagged and the centrifugation is repeated. LIMS uses a template to create an MS worklist with appropriate settings for each well. Blanks are inserted as appropriate. Sample positions are randomized within specified parameters to prevent plate position effects. At the LCMS workstation the worklist is imported to automatically define the processing parameters for each well. Samples are injected into the liquid chromatograph and subsequently analyzed by qTOF mass spectrometry.

An exemplary workflow for fractionated proteomics studies is shown in FIG. 17.

### Example 17: Depleted Proteomics

The following example describes an exemplary workflow and devices for use in a depleted proteomics study. Experiments are tracked and organized by a LIMS. The LIMS has automated uploads and downloads. The LIMS sets up previously computed sample ordering and randomization and tracks experimental worksheets and worklists. Sample ordering is determined as part of the overall study design. The LIMS computes parameters applied in ChemStation software. LC trace data is processed and normalized, then written to a CSV file. Optical density measurements are made to measure the protein concentration in each sample. Controls of known protein concentration are measured to determine the parameters applied in computation of sample concentrations. Samples that do not fall within desired parameters are flagged. The LIMS computes parameters of LC traces into protein mass estimates. Controls of known protein mass are fractionated and then measured to determine the parameters applied in computation of fraction mass distribution.

Bulk reagents and stock solutions are prepared prior to sample process start and stored appropriately for use over the experiment days. Plate QC samples are derived from known sample pools and are processed in parallel with study samples so that they undergo exactly the same laboratory actions.

Sample mixtures are determined, including aliquot count and volume.

Samples are initially processed by ordering them according to data preloaded into LIMS. This includes the process quality control samples. The samples are thawed and examined. A user assesses the sample for features that would compromise its ability to be analyzed, including hyperlipidemia and the presence of large amounts of hemoglobin. Samples that fail this analysis are flagged.

Buffer is added to the samples for protein depletion. The samples are run through a multiple affinity removal column. Particles and lipids are filtered. Samples are assessed for particles and lipids and samples in which particles and lipids are not adequately filtered are flagged.

The amount of protein in each sample is determined so that correct amounts of reagents and buffers can be added. This is accomplished using a total protein assay to estimate the total amount of protein in each sample. The samples are optically scanned. Worklists for automated fractionation, digestion and reconstitution are customized for every sample. The LIMS estimates sample protein concentration based on uploaded optical density measurements. LIMS also assesses OD measurement quality and flags out-of-spec results. Next, LIMS computes the amount of each sample to inject into IDFC to achieve constant protein amounts for digestion. The accuracy of this step can help ensure reproducibility of the depletion.

The samples are then depleted. Depletion removes the most abundant proteins from the sample so that lower concentration proteins are detectable. This is accomplished using a customized Immuno-Depletion Fractionation (IDFC) LC system. Samples are assessed for depletion by detecting the concentration of proteins that should be removed or reduced by the IDFC-LC system or by analyzing the chromatography trace. Samples that are not properly depleted are flagged and subjected to an additional round of depletion, as appropriate. The process includes generated a worklist file, putting the samples into a 96 well plate, double checking to ensure sample locations are correct, and depleting the samples. Based on values in the uploaded CSV file, the earlier estimate of total sample protein mass is distributed among the samples' fractions.

Next, LIMS computes appropriate volumes of trypsin and recon buffer for each sample fraction based on protein mass estimates. These data are used to generate a worklist, which is uploaded to a Tecan workstation. Trypsin is added to each well based on the calculated amounts determined by the LIMS. The resulting samples are analyzed for digest quality, including average fragment size, fragment size range, fragment size distribution, and incomplete digestion. Digestion of a second aliquot using the same or different restriction enzyme is repeated for samples flagged for failing any of these tests. Volumes are controlled to match instrument configurations.

The samples are then dried for storage or processing for mass spectroscopy. This includes quenching the samples and drying them, washing them with SPE buffer to maximize recover, and lyophilizing them. The samples can be frozen at this point if the mass spectrometer is not available for use.

The readiness of the mass spectrometer is assessed prior to use. Each run of digested samples is preceded by a quality control run to determine if the LCMS is functioning within defined tolerances. If the instrument is outside of defined performance tolerances, the sample run is postponed until the instrument performance is within defined performance tolerances. LIMS uses a template to generate a LCMS worklist with randomized sample ordering and appropriate injection volumes for each sample to standardize the mass loaded onto the LC column. The quality control run samples are processed in the same order for every worklist (e.g. first, middle and last) to provide sample/worklist normalization during data analysis. The worklist file is archived automatically. The generated LCMS worklist is imported into the LCMS control software. The worklist name, along with sample injection order, is loaded into the LCMS control software and confirmed by a user. After confirmation of the loaded worklist file, the worklist is started through the instrument control software. The resulting data are then assessed for quality using pre-defined metrics. Data that does not meet or exceed quality standards are flagged.

Lyophilized samples are reconstituted in an appropriate buffer for injection on the LCMS. LIMS dynamically computes the individual sample buffer volume for reconstitution to yield standardized peptide loading on the LCMS across all sample wells. This is used to generate a worklist for reconstituting the samples using Tecan. The worklist is archived automatically. Reconstitution buffer is volumetrically dispensed with a Tecan liquid handling robot. Samples receiving an incorrect volume of buffer or that are otherwise mishandled are flagged. The plate is then centrifuged. Samples containing bubbles are flagged and the centrifugation is repeated. LIMS uses template to create an MS worklist with appropriate settings for each well. Blanks are inserted as appropriate. Sample positions are randomized within specified parameters to prevent plate position effects. At the LCMS workstation the worklist is imported to automatically define the processing parameters for each well. Samples are injected into the liquid chromatograph and subsequently analyzed by qTOF mass spectrometry.

An exemplary workflow for depleted proteomics studies is shown in FIG. 18.

### Example 18: Dried Plasma Spot Proteomics

The following example describes an exemplary workflow and devices for use in a dried plasma spot proteomic study. Experiments are tracked and organized by a LIMS. The LIMS has automated uploads and downloads. The LIMS sets up previously computed sample ordering and randomization and tracks experimental worksheets and worklists. Sample ordering is determined as part of the overall study design.

Bulk reagents and stock solutions are prepared prior to sample process start and stored appropriately for use over the experiment days.

Sample plasma is loaded onto a DPS card. Stock solutions of heavy peptides of interest at known concentrations are prepared for SIS spike-in. The samples are cut from filter paper and loaded into wells on plate. The samples are digested, lyophilized, and frozen as described above.

The readiness of the instruments is assessed as described above. The generated LCMS worklist is imported into the LCMS control software. The worklist name, along with sample injection order, is loaded into the LCMS control software and confirmed by a user. After confirmation of the loaded worklist file, the worklist is started through the instrument control software. The instruments are determined to be ready based on quality control metrics.

Lyophilized samples are reconstituted in 6PRB buffer for injection on the LCMS. For experiments with SIS peptide spike-in, the appropriate buffer containing precomputed heavy peptide amounts is added instead. Reconstitution buffer is volumetrically dispensed with a Tecan liquid handling robot. Reconstituted samples are centrifuged to remove bubble and settle samples at the bottom of each well.

LIMS uses template to create an MS worklist with appropriate settings for each well. Blanks are inserted as appropriate. Sample positions are randomized within specified parameters to prevent plate position effects. At the LCMS workstation the worklist is imported to automatically define the processing parameters for each well. Samples are injected into the liquid chromatograph and subsequently analyzed by qTOF mass spectrometry.

An exemplary workflow for dried plasma spot proteomics studies is shown in FIG. 19.

### Example 19: Targeted Proteomics

The following example describes an exemplary workflow and devices for use in a targeted proteomic study. Experiments are tracked and organized by a LIMS. The LIMS has automated uploads and downloads. The LIMS sets up previously computed sample ordering and randomization and tracks experimental worksheets and worklists. Sample ordering is determined as part of the overall study design.

Bulk reagents and stock solutions are prepared prior to sample process start and stored appropriately for use over the experiment days. Plate QC samples are derived from known sample pools and are processed in parallel with study samples so that they undergo exactly the same laboratory actions.

Sample mixtures are determined, including aliquot count and volume.

Samples are initially processed by ordering them according to data preloaded into LIMS. This includes the process quality control samples. The samples are thawed and examined. A user assesses the sample for features that would compromise its ability to be analyzed, including hyperlipidemia and the presence of large amounts of hemoglobin. Samples that fail this analysis are flagged.

Buffer is added to the samples for protein depletion. The samples are run through a multiple affinity removal column. Particles and lipids are filtered.

The amount of protein in each sample is determined so that correct amounts of reagents and buffers can be added. This is accomplished using a total protein assay to estimate the total amount of protein in each sample. The samples are optically scanned. Worklists for automated fractionation, digestion and reconstitution are customized for every sample. The LIMS estimates sample protein concentration based on uploaded optical density measurements. LIMS also assesses OD measurement quality and flags out-of-spec results. Next, LIMS computes the amount of each sample to inject into IDFC to achieve constant protein amounts for digestion. The accuracy of this step can help ensure reproducibility of the depletion.

The samples are then depleted. Depletion removes the most abundant proteins from the sample so that lower concentration proteins are detectable. This action is accomplished using a customized Immuno-Depletion Fractionation (IDFC) LC system. First, sample locations are double checked to ensure correct sample location. A worklist is generated by the LIMS, uploaded to the IDFC workstation, and archived automatically. The LC captures raw trace data, which is processed into a CSV file using a macro. The CSV file is uploaded to LIMS and archived automatically.

The LIMS then calculates trypsin and reconstitution buffer volumes for each sample based on protein mass estimates. Next, the samples are prepared for digestion using a buffer exchange. Samples completing the depletion task are transferred to buffers appropriate to the follow-on TPA and digestion tasks. Before trypsin addition, the total amount of protein in each sample is measure so that correct amounts of reagents and buffers can be added. This is accomplished via an optical scan. LIMS estimates each sample protein concentration based on uploaded optical density measurements. LIMS also assesses OD measurement quality and flags out-of-spec results. A worklist is generated for automated fractionation, digestion, and reconstitution of each individual sample. The worklist includes trypsin volumes to match expected protein amounts for each sample.

The worklist is sent to a Tecan workstation and also archived automatically in LIMS. The Tecan workstation adds trypsin to each well on a per-well basis. Volumes are controlled to match instrument configurations. Samples are lyophilized and stored as described above.

Instrument readiness is assessed as described above. If the mass spectrometer passes quality control tests, samples are reconstituted using either 6PRB buffer or buffer containing stable isotope standards. This process is described above. The samples are centrifuged to remove bubbles and settle the samples at the bottom of each well. The samples are then analyzed by LCMS as described above.

An exemplary workflow for targeted proteomics studies is shown in FIG. 20.

### Example 20: Immunoaffinity Enrichment of Peptides Coupled to Targeted, Multiple Reaction Monitoring-Mass Spectrometry (Immuno-MRM)

The following example describes an exemplary workflow and devices for use in a dried plasma spot proteomic study. Samples are prepared as described in Example 16. However, after diluted samples are added to the appropriate wells on the pate and stable isotope standards are added to the samples, the samples are enriched for peptides of interest using antibodies. Antibodies that specifically bind to peptides of interest are bound to magnetic beads. The samples and controls are mixed with beads, which allows the antibodies to bind to targeted peptides. The beads are washed and unbound peptides are washed away. The beads are then eluted and the antibodies release the peptides of interest. This results in a sample enriched for the peptides of interest. The samples are then analyzed by LCMS as described in Example 16.

An exemplary workflow for immuno-MRM experiments is shown in FIG. 22.

### Example 21: Dilute Proteomics

The following example describes an exemplary workflow and devices for use in a dilute proteomics study. Experiments are tracked and organized by a LIMS. The LIMS has automated uploads and downloads. The LIMS sets up previously computed sample ordering and randomization and tracks experimental worksheets and worklists. Sample ordering is determined as part of the overall study design. The LIMS computes parameters applied in ChemStation software. LC trace data is processed and normalized, then written to a CSV file. Optical density measurements are made to measure the protein concentration in each sample. Controls of known protein concentration are measured to determine the parameters applied in computation of sample concentrations. The LIMS computes parameters of LC traces into protein mass estimates. Controls of known protein mass are fractionated and then measured to determine the parameters applied in computation of fraction mass distribution.

Bulk reagents and stock solutions are prepared prior to sample process start and stored appropriately for use over the experiment days. Plate QC samples are derived from known sample pools and are processed in parallel with study samples so that they undergo exactly the same laboratory actions.

Sample mixtures are determined, including aliquot count and volume.

Samples are initially processed by ordering them according to data preloaded into LIMS. This includes the process quality control samples. The samples are thawed and examined. A user assesses the sample for features that would compromise its ability to be analyzed, including hyperlipidemia and the presence of large amounts of hemoglobin. Samples that fail this analysis are flagged.

Buffer is added to the samples for protein depletion. The samples are run through a multiple affinity removal column. Particles and lipids are filtered.

The samples are then depleted. Depletion removes the most abundant proteins from the sample so that lower concentration proteins are detectable. This is accomplished using a customized Immuno-Depletion Fractionation (IDFC) LC system. The process includes generated a worklist file, putting the samples into a 96 well plate, double checking to ensure sample locations are correct, and depleting the samples. Based on values in the uploaded CSV file, the earlier estimate of total sample protein mass is distributed among the samples' fractions.

The amount of protein in each sample is determined so that correct amounts of reagents and buffers can be added. This is accomplished using a total protein assay to estimate the total amount of protein in each sample. The samples are optically scanned. Worklists for automated fractionation, digestion and reconstitution are customized for every sample. The LIMS estimates sample protein concentration based on uploaded optical density measurements. LIMS also assesses OD measurement quality and flags out-of-spec results.

Next, LIMS computes appropriate volumes of trypsin and recon buffer for each sample fraction based on protein mass estimates. These data are used to generate a worklist, which is uploaded to a Tecan workstation. Trypsin is added to each well based on the calculated amounts determined by the LIMS. Volumes are controlled to match instrument configurations.

The samples are then dried for storage or processing for mass spectroscopy. This includes quenching the samples and drying them, washing them with SPE buffer to maximize recover, and lyophilizing them. The samples can be frozen at this point if the mass spectrometer is not available for use.

The readiness of the mass spectrometer is assessed prior to use. Each run of digested samples is preceded by a quality control run to determine if the LCMS is functioning within defined tolerances. If the instrument is outside of defined performance tolerances, the sample run is postponed until the instrument performance is within defined performance tolerances. LIMS uses a template to generate a LCMS worklist with randomized sample ordering and appropriate injection volumes for each sample to standardize the mass loaded onto the LC column. The quality control run samples are processed in the same order for every worklist (e.g. first, middle and last) to provide sample/worklist normalization during data analysis. The worklist file is archived automatically. The generated LCMS worklist is imported into the LCMS control software. The worklist name, along with sample injection order, is loaded into the LCMS control software and confirmed by a user. After confirmation of the loaded worklist file, the worklist is started through the instrument control software. The resulting data are then assessed for quality using pre-defined metrics.

Lyophilized samples are reconstituted in an appropriate buffer for injection on the LCMS. LIMS dynamically computes the individual sample buffer volume for reconstitution to yield standardized peptide loading on the LCMS across all sample wells. This is used to generate a worklist for reconstituting the samples using Tecan. The worklist is archived automatically. Reconstitution buffer is volumetrically dispensed with a Tecan liquid handling robot. The samples are added to a plate along with standards or controls with varying concentrations of known peptides. The plate is then centrifuged. LIMS uses template to create an MS worklist with appropriate settings for each well. Blanks are inserted as appropriate. Sample positions are randomized within specified parameters to prevent plate position effects. At the LCMS workstation the worklist is imported to automatically define the processing parameters for each well. Samples are injected into the liquid chromatograph and subsequently analyzed by a triple-quadrupole (QqQ) mass spectrometer mass spectrometry.

Data from each run or from each day are evaluated for quality. Among the quality tests performed is an evaluation of standard curves and processes. The standard curves for spiked standards pass quality control assessments if peak areas and retention times fall within pre-defined area and retention time ranges. The process quality control evaluation includes determining if Coefficients of Variation or other measures of consistency are below a pre-defined threshold, retention times are within a pre-defined range, and peak areas are within expected ranges. If the quality checks fail, the samples are flagged, root cause analysis is performed, and the affected samples are re-run.

### Example 22: Computational Pipeline for Profile and DPS Proteomics

9.1—Data Acquisition

A computational workflow is initiated for mass spectra data obtained from Profile and DPS proteomics and processed as shown in FIG. 27A. A data acquisition module acquires data and generates a single LCMS data file for each sample well for a registered study. The data acquisition process includes initiating a workflow queued by registered instruments and verifying that each LCMS data file was copied to shared primary data storage.

9.2—Workflow Determination

Next, a workflow determination module reads the associated worklist for this study and sets parameters for the workflow. In this case, the parameters include the method, pump model number, sample type, sample name, data acquisition rate minimum and maximum, concentration, volume, plate position, plate barcode, and others. The workflow determination module uses the LCMS method used to generate the data file and the parameters gathered from parsing the worklist to determine the pipeline computations and steps to run. In this case, the particular computation flow is set in a computational group that allows modularization of pipeline computational flow, which allows each computational flow to be easily reconfigured depending on study requirements and the nature of the sample being processed.

9.3—Data Extraction

The data extraction module then extracts the data from each LCMS data file for downstream processing. This includes extracting the total ion chromatogram using calculations determined by the chromatography group. The data extraction process includes using an API to extract the LCMS instrument chromatograms into an “actuals” file for downstream use and then extracting and converting the spectral data to APIMS1 format for acquired time range, device name and type, fragment voltage, ionization mode, ion polarity, mass units, scan type, spectrum type, threshold, sampling period, total data point and total scan counts.

The data extraction module then extracts MS2 data (since this data set includes tandem mass spectral data) and converts the data through an application library to Mascot generic format (MGF). Finally, the data extraction module determines the chromatography group collected from the preceding extraction and conversion step, and obtains the TIC using an algorithm, which is then saved to a database.

9.4—Data Preparation

Next, a data preparation module converts the APIMS1 file into a java serialized format ready for downstream processing. The data preparation module then puts the scans and the read backs during those scans into the database.

9.5—Feature Extraction

A feature extraction module then caries out extraction of initial molecular features using an algorithm for peak detection, which is stored in parallel sections to a java serialized file for downstream processing.

The feature extraction module subsequently refines the initial molecular features using LC and isotopic profiles, and then computes the properties of those features. This process includes combining each molecular feature extraction section from the preceding steps for analysis, then applying a combination of filtering and clustering techniques to raw peaks, writing the evaluated peaks to the database, and computing the MS1 properties associated with a given set of molecular features storing them in the database. The feature extraction module also interpolates the MS1 data points, sets the quality data for each, and saves the data to the database. Finally, the feature extraction module cleans up the MS1 peak detection files and computes the MS1 peak cleanup, and removes temporary files from the computing machines.

9.6—Proteomic Processing

Next, a proteomic processing module proposes peptide sequences and possible protein matches for the MS2 data. This step comprises creating lists for targeted data acquisition for neutral mass clustering and molecular feature extractions and correcting the MGF file by incorporating mass differences and charge (e.g., matching precursor masses and charges from the MGF file to the refined values developed in the preceding refinement of molecular features). Next, the proteomic processing module searches for peptides in the UniProt Human/Mouse/Rat/Bovine (HMRB) FASTA database using the OMSSA engine. The search is conducted by matching against the database itself and a reversed version; and results from the latter search are used to develop false discovery rate (FDR) statistics.

For OMSSA searching, the proteomic processing module sets the search mode to OMSSA, sets the forward database (HMRB) for search in OMSSA, performs the forward OMSSA search, sets up the reversed database (HMRB reversed) for the search in OMSSA, and performs the reverse search in OMSSA.

For X! Tandem engine searching, the proteomic processing module sets the search mode to X! Tandem, sets the forward database (HMRB) for search in X! Tandem, performs the forward X! Tandem search, sets up the reversed database (HMRB reversed) for the search in X! Tandem, and performs the reverse search in X! Tandem.

Next, the proteomic processing module performs validation on the search results. When using the OMSSA forward and reverse search results, the proteomic processing module computes expectation values for a range of FDRs for peptides identified within a sample, models RTs for proposed peptides, and filters out those which are at significant variance with the model. This process comprises setting the search mode to OMSSA, setting up the forward database (HMRB) for validation, calculating the FDR and associated expectation values, developing an RT model from the sample's data, and then performing RT filtering to reject proposed peptides that differ from the model.

For validation of search results generated by X! Tandem forward and reverse search results, the proteomic processing module computes expectation values for a range of FDRs for peptides identified within a sample, models RTs for proposed peptides, and filters out those which are at significant variance with the model. This process comprises setting the search mode to X! Tandem, setting up the forward database (HMRB) for validation, calculating the FDR and associated expectation values, developing an RT model from the sample's data, and then performing RT filtering to reject proposed peptides that differ from the model.

Next, the proteomic processing module analyzes validation results and saves the results to the database. This process includes setting up the forward database (HMRB) for review, evaluating the OMSSA and X! Tandem search, validating the search, and reporting filtering statistics.

The proteomic processing module then maps the peptide results from X! Tandem and/or OMSSA searches to UniProt HMRB FASTA proteins using BlastP. The hit scores and ranks are then saved. The mapping process for OMSSA comprises setting up the forward database (HMRB) for searching, searching for protein matches to the OMSSA-based peptides using BlastP, assigning BlastP scores and ranks to the OMSSA-based peptides, and summarizing and saving information about the protein matches found for the OMSSA-based peptides.

The mapping process for X! Tandem comprises setting up the forward database (HMRB) for searching, searching for protein matches to the X! Tandem-based peptides using BlastP, assigning BlastP scores and ranks to the X! Tandem-based peptides, and summarizing and saving information about the protein matches found for the X! Tandem-based peptides.

Finally, the proteomic processing module determines the targeted proteomic results for statistical review.

9.7—Quality Analysis

A quality control module performs quality control analysis through TIC comparison, protein map, molecular feature tolerance validations, peptide clustering, and other methods for carrying out quality control analysis of LCMS. The quality control module then assesses each scan's quality and computes quality metrics, including the number of peaks, peak relative sizes, abundance ratios, signal to noise ratio (SNR), and sequence tag length derived from the MGF and spectral features files. Finally, the standard quality metrics are determined.

9.8—Visualization

A visualization module creates a visual representation such as a starfield thumbnail that is a visualization of signal intensity plotted for LC RT vs. m/z, in which low resolution isotopic features appear as points of light (e.g. the points resembles stars).

9.9—Utilities

A utilities module provides various helper utilities for data exploration, visualization, and monitoring. In this case, the utilities carry out tasks including using the mass to determine the neutral mass and the mass of charge states 1 through 5. Mass calculation comprises entering the molecular formula through the periodic table of elements, and determining the neutral mass and the mass of charge states 1 through 5. In addition, the peptide mass is calculated by entering the peptide or protein sequence, optionally adding modifications, and determining the neutral mass plus those of charge states 1 through 6. For calculating tandem mass, the peptide or protein sequence is entered showing the “y” and “b” components along with options for charge states with modifications in a tabular format. Finally, the peptides are searched against a database (e.g., Human FASTA database) to return proteins that match.

Moreover, the utilities module provides a utility showing the remaining LCMS lifetime against a pre-defined threshold (e.g., a pre-set “lifetime” for the LCMS column), a utility that plots spectra using CSV or MGF files, and a utility showing pipeline status, which includes the list of computational steps, the machine registered to run those steps/processes, and the machine status (e.g., on or off, whether a sample is being processed, etc.). The utilities also provides tune reports for the mass spectrometer, the ability to pause and reset process nodes, and annotation of issues that are resolved in which processing cannot be completed. In this case, no issues are detected that prevents completion of data processing, and the computational workflow is able to run to completion.

9.10—Monitoring

Next, a monitoring module provides monitoring of the system and/or instruments. The monitoring module continuously and automatically monitors the SysLogbook for events coming directly off instruments and looks for errors and warnings that can be handled quickly. When an IDFC data file is transferred to a central repository, and an error condition occurs (e.g., maximum ultra violet time is shorter than expected), a lab technician then investigates prior to proceeding with experimental protocols. The monitoring module allows for registration (e.g., self-registration) and email notification for specific events (including opt out of email notification) that are detected during monitoring.

During disk space cleanup activities, the monitoring module reports resolving primary data transfer verifications prior to computer removal. This is performed periodically to purge more data off the instruments.

The monitoring module allows detection of errors and providing notification regarding said errors to allow prompt remediation of the issue. When a process hits an error condition stopping the workflow, the error is identified and notification is provided. A lab technician then resolves the issue in the laboratory (e.g., modifying/changing lab protocol), or the issue is fixed computationally (e.g., removing bad data from subsequent analysis). For example, when process control samples result, metrics based on process control samples are historically compared for proper instrument operations. Determination of failure criteria then pauses or postpones the laboratory procedure until resolution, or cause interpretation of the data to be excluded from study later due to poor quality.

Notification for pipeline processes being manually turned on or off is also provided.

When the failure of a process is not material (e.g., does not requiring stopping the pipeline), the monitoring module still provides notification to allow investigation of the issue to ensure the sample data is processed properly.

Finally, an orbitrap report is sent upon transfer of a directory instrument file.

9.11—Cleanup

A cleanup module (or alternatively, the monitoring module) optionally compresses (or deletes) the APIMS1 file in place to save space on the shared drive or database.

### Example 23: Computational Pipeline for Targeted and iMRM Proteomics

10.1—Data Acquisition

A computational workflow is initiated for mass spectra data obtained from Targeted and iMRM proteomics and processed as shown in FIG. 27B. Data is acquired by a data acquisition module, which initiates a workflow queued by polling registered instruments connected to mass spectrometers gathering study data. The acquired instrument data is copied/transferred to a shared repository (in this case, a shared database), which is then verified.

10.2—Workflow Determination

Next, a workflow determination module reads the worklist for this sample set and sets parameters for the workflow in which the computations for the workflow are determined based on the method and parameters obtained from the worklist.

10.3—Data Preparation

A data preparation module then enters the data into a proteomic mzML standardized format using ProteoWizard.

10.4—Data Extraction

Next, a data extraction module reads the raw data and extracts it into a different format and parses the mzML into CSV for peaks. This entails preparing a directory for storage of the extracted information, reading the mzML file, and extracting the trace data into a CSV file for later processing.

10.5—Feature Extraction

A feature extraction module then identifies peaks and determines their areas by preparing a defined directory for the extracted information and finding peaks for m/z trace files, which signal proteomic data of interest.

10.6—Proteomic Processing

A proteomic processing module then inserts cluster peaks and links heavy and light peaks to ensure the transition peaks are aligned. This is accomplished by determining the peak area for m/z peak traces, and then annotating (e.g., “tagging”) the identified peaks and associating them to proteomic data items.

10.7—Quality Analysis

Next, a quality control module accesses data related to quality assessments such as light and heavy peptides' SNR, transition counts, RT delta, and peak area. This process includes formatting, storing, and gathering the m/z peak trace data. Then the quality control module generates metrics on the features of the m/z peak trace data for both regular and quality control samples.

10.8—Utilities

Finally, a utilities module provides visualization of m/z peak traces for both heavy and light peptides.

While preferred embodiments of the present invention have been shown and described herein, it will be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A system for automated mass spectrometric analysis comprising
a) a plurality of protein processing modules positioned in series; and
b) a plurality of mass spectrometric sample analysis modules;
wherein at least two of said protein processing modules are separated by a mass spectrometric sample analysis module; and
wherein each mass spectrometric sample analysis module operates without ongoing supervision.

2. The system of claim 1, wherein the system further comprises protein processing modules not separated by a mass spectrometric sample analysis module, wherein the modules are configured to carry out an experimental workflow.

3. The system of claim 2, wherein the system further comprises protein processing modules not positioned in series.

4. The system of claim 2, wherein the system further comprises at least one mass spectrometric sample analysis module subject to ongoing supervision.

5. The system of claim 1, wherein the mass spectrometric sample analysis modules are configured to evaluate performance of an immediately prior protein processing module.

6. The system of claim 1, wherein the sample analysis modules are configured to evaluate an effect of an immediately prior protein processing module on a sample selected for mass spectrometric analysis.

7. The system of claim 6, wherein the sample analysis modules are configured to stop sample analysis when an evaluation indicates that a quality control metric is not met.

8. The system of claim 1, wherein the sample analysis modules are configured to tag a sample analysis output when the evaluation indicates that a quality control metric is not met for at least one sample analysis module.

9. The system of claim 8, wherein the tag indicative of the quality control metric not being met is incorporated into at least one of downstream sample processing by a subsequent protein processing module or downstream sample evaluation by a subsequent data analysis module.

10. The system of claim 9, wherein the tag corresponds to at least one rule determining downstream sample processing or data evaluation, wherein the at least one rule comprises continuing the workflow, terminating the workflow, suspending the workflow, or restarting the workflow.

11. The system of claim 10, wherein the at least one rule comprises terminating, suspending, or restarting the workflow when the quality control metric indicates an insufficient quantity, insufficient concentration, insufficient signal strength, background, or contamination that disrupts detection of at least one target peptide.

12. The system of any one of claims 1-11, wherein the plurality of protein processing modules positioned in series comprises at least four modules.

13. The system of any one of claims 1-11, wherein the plurality of protein processing modules positioned in series comprises at least eight modules.

14. The system of any one of claims 1-11, wherein a sample analysis module evaluates a protein processing module that digests proteins into polypeptide fragments.

15. The system of claim 14, wherein the protein processing module that digests proteins contacts proteins to a protease.

16. The system of any one of claims 1-11, wherein a sample analysis module evaluates a protein processing module that volatilizes polypeptides.

17. The system of any one of claims 1-11, wherein a sample analysis module evaluates volatilized polypeptide input mass.

18. The system of any one of claims 1-11, wherein a sample analysis module assesses output of a mass spectrometry detector module, wherein the output comprises signals detected by a mass spectrometry detector.

19. The system of any one of claims 1-11, wherein a sample analysis module comprises an instrument configured to measure the optical density of a protein sample, and wherein the system is configured to calculate a protein concentration from the measured optical density of a sample.

20. The system of any one of claims 1-11, wherein one of the protein processing modules utilizes gas chromatography, liquid chromatography, capillary electrophoresis, or ion mobility to fractionate a sample, and wherein the system is configured to analyze data generated by the detector and flag samples that do not meet a set of chromatography QC metrics comprising at least one of peak shifting, peak area, peak shape, peak height, wavelength absorption, or wavelength of fluorescence detected in the biological sample.

21. The system of any one of claims 1-11, wherein one of the protein processing modules is configured to deplete a protein sample by removing pre-selected proteins from the sample.

22. The system of any one of claims 1-11, wherein one of the protein processing modules comprises an instrument configured to compute and add an amount of a protease to the sample, and wherein the amount of protease added to the sample is dynamically calculated by the amount of protein estimated to be present in the sample.

23. The system of any one of claims 1-11, wherein the system assesses the readiness of the mass spectrometer by determining if data generated by the mass spectrometer from a sample indicates detection of a minimum number of features that possess a specific charge state, a minimum number of features, selected analyte signal that meets at least one threshold, presence of known contaminants, mass spectrometer peak shape, chromatographic peak shape, or any combination thereof.

24. A system for feature processing comprising:
a) a plurality of visualization modules positioned in series; and
b) a plurality of feature processing modules positioned in series;
wherein at least one of the feature processing modules is separated by a gating module;
wherein the output data of at least some feature processing modules has passed a gating module evaluation prior to becoming input data for a subsequent feature processing module;
wherein the output data of at least some visualization modules has passed a gating evaluation prior to becoming input data for a subsequent visualization module, and
wherein at least some gating evaluation occurs without user supervision.

25. The system of claim 24, wherein the plurality of feature processing modules comprises a clustering module.

26. The system of any one of claims 24-25, wherein the plurality of feature processing modules comprises a normalization module.

27. The system of any one of claims 24-25, wherein the plurality of feature processing modules comprises a filtering module.

28. A method for automated mass spectrometric analysis comprising:
a) acquiring at least one mass spectrometric data set from at least two different sample runs;
b) generating a visual representation of the data comprising identified features from the at least two sample runs;
c) defining an area of the visual representation comprising at least a portion of the identified features; and
d) discontinuing analysis because a threshold of at least one QC metric is not met based on a comparison between features of the sample runs
wherein the method is performed on a computer system without user supervision.

29. The method of claim 28, wherein the threshold of at least one QC metric is not met when no more than 10 non-corresponding features between the sample runs is identified.

30. The method of claim 28, wherein the identified features comprise charge state, chromatographic time, overall peak shape, analyte signal strength, presence of known contaminants, or any combination thereof.
